(19) United States
(12) Patent Application Publication
(10) Pub. No.: US 2020/0054651 A1
(43) Pub. Date: Feb. 20, 2020

(54) COMPOSITIONS AND METHODS FOR MODULATING PPP2R1A

(71) Applicant: The Regents of the University of California, Oakland, CA (US)

(72) Inventors: Daniel K. Nomura, Berkeley, CA (US); Elizabeth A. Moore, Berkeley, CA (US); Carl C. Ward, Berkeley, CA (US); Leslie A. Bateman, Boston, MA (US); Tucker R. Huffman, El Dorado Hills, CA (US); David K. Miyamoto, Cambridge, MA (US); Jessica Spradlin, Berkeley, CA (US)

(21) Appl. No.: 16/482,952

(22) PCT Filed: Feb. 2, 2018
(86) PCT No.: PCT/US2018/016651

§ 371 Date: Aug. 1, 2019

Related U.S. Application Data
(60) Provisional application No. 62/454,700, filed on Feb. 3, 2017.
(60) Provisional application No. 62/530,021, filed on Jul. 7, 2017.

(51) Int. Cl. A61K 31/585; A61K 31/357; A61K 31/404; A61K 31/167; A61K 31/245; A61K 31/341; A61K 31/165; A61K 31/36; A61K 31/495; A61K 31/40; A61K 31/5375; A61K 31/445; A61K 31/16; A61P 35/00; C12N 9/16

(52) U.S. Cl. CPC A61K 31/585; C12Y 301/03016; A61K 31/404; A61K 31/167; A61K 31/245; A61K 31/341; A61K 31/165; A61K 31/36; A61K 31/495; A61K 31/40; A61K 31/5375; A61K 31/445; A61K 31/16; A61P 35/00; C12N 9/16; A61K 31/357

(57) ABSTRACT

Disclosed herein, inter alia, are compositions and methods useful for modulating PPP2R1 A and for the treatment of cancer.

### CROSS-REFERENCES TO RELATED APPLICATIONS

This application claims the benefit of U.S. Provisional Application No. 62/454,700, filed Feb. 3, 2017, and U.S. Provisional Application No. 62/530,021, filed Jul. 7, 2017, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with Government support under grant nos. CA172667 awarded by the National Institutes of Health, and W81XWH-15-1-0050 awarded by the U.S. Army Medical Research and Materiel Command. The Government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 052103-504001WO Sequence Listing_ST25.txt, created Jan. 31, 2018 16,455 bytes, machine format IBM-PC, MS Windows operating system, is hereby incorporated by reference.

### BACKGROUND

Female breast cancer is the fourth leading cause of cancer death in the United States. It is estimated that about 1 in 8 U.S. women (about 12%) will develop invasive breast cancer over the course of her lifetime and the number of deaths was 21.5 per 100,000 women per year based on 2009-2013. In 2017, an estimated 255,180 new cases of invasive breast cancer are expected to be diagnosed in women in the U.S., along with 63,410 new cases of non-invasive (in situ) breast cancer. Current therapeutic strategies for breast cancer include resection and non-specific therapies such as radiation or chemotherapy. Unfortunately, these treatment strategies are insufficient for highly aggressive triple-negative breast cancer (TNBC) and thus better strategies are needed to discover both novel anti-cancer agents and targets for combatting triple-negative breast cancer. Towards this goal, identifying new anti-cancer targets, druggable nodes, and lead small-molecules are critical for combatting breast cancer. Disclosed herein, inter alia, are solutions to these and other problems in the art.

### BRIEF SUMMARY

Herein are provided, inter alia, compounds capable of modulating the level of activity of the subunit PPP2R1A of the tumor suppressor protein phosphatase 2A (PP2A) complex and methods of using the same.

In an aspect is provided a compound having the formula:

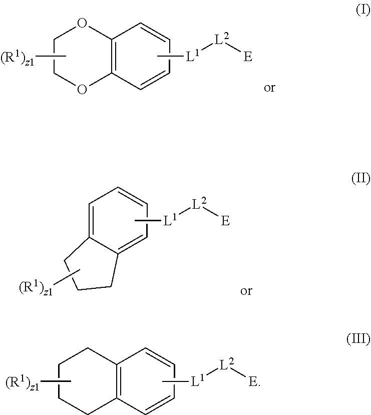

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl. The symbol z1 is an integer from 0 to 7. L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L² is a
bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. E is an electrophilic moiety. Each R^{1A}, R^{1B}, R^{1C}, R^{1D}, R^{4A}, R^{4B}, R^{5A}, and R^{5B} is independently
hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A} and R^{1B} substituents bonded to the same nitrogen atom may optionally be joined to form a substituted or unsubstituted heterocycloalkyl or substituted or unsubstituted heteroaryl. R^{4A} and R^{4B} substituents bonded to the same nitrogen atom may optionally be joined to form a substituted or unsubstituted heterocycloalkyl or substituted or unsubstituted heteroaryl. R^{5A} and R^{5B} substituents bonded to the same nitrogen atom may optionally be joined to form a substituted or unsubstituted heterocycloalkyl or substituted or unsubstituted heteroaryl. Each X, X¹, X⁴, and X⁵ is independently —F, —Cl, —Br, or —I. The symbols n1, n4, and n5 are independently an integer from 0 to 4. The symbols m1, m4, m5, v1, v4, and v5 are independently an integer from 1 to 2.

In an aspect is provided a pharmaceutical composition including a Serine/threonine-protein phosphatase 2A 65 kDa regulatory subunit A alpha isoform (PPP2R1A) modulator and a pharmaceutically acceptable excipient.

In an aspect is provided a pharmaceutical composition including a compound described herein, or pharmaceutically acceptable salt thereof, and a pharmaceutically acceptable excipient.

In an aspect is provided a method of modulating a serine/threonine-protein phosphatase 2A 65 kDa regulatory subunit A alpha isoform (PPP2R1A) protein, the method including contacting the Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator.

In an aspect is provided a method of modulating a serine/threonine-protein phosphatase 2A 65 kDa regulatory subunit A alpha isoform (PPP2R1A) protein, said method including contacting the Serine/threonine-protein phosphatase 2A 65 kDa regulatory subunit A alpha isoform (PPP2R1A) protein with an effective amount of a compound described herein.

In an aspect is provided a method of treating cancer, said method including administering to a subject in need thereof an effective amount of a serine/threonine-protein phosphatase 2A 65 kDa regulatory subunit A alpha isoform (PPP2R1A) modulator.

In an aspect is provided a method of treating cancer, said method including administering to a subject in need thereof an effective amount of a compound described herein.

In an aspect is provided a serine/threonine-protein phosphatase 2A 65 kDa regulatory subunit A alpha isoform (PPP2R1A) protein covalently bonded to a PPP2R1A modulator.

In an aspect is provided a serine/threonine-protein phosphatase 2A 65 kDa regulatory subunit A alpha isoform (PPP2R1A) protein covalently bonded to a compound described herein.

### BRIEF DESCRIPTION OF THE DRAWINGS

FIGS. 1A-1D: Withaferin A impairs breast cancer cell pathogenicity. (FIG. 1A) Structure of withaferin A. (FIG. 1B) Withaferin A (10 μM) impairs cell proliferation and serum-free cell survival after 48 h in MCF7, 231MFP, and HCC38 cells.

FIGS. 2A-2F. Using isoTOP-ABPP platforms to map proteome-wide targets of withaferin A in breast cancer cells. (FIG. 2A) Competitive isoTOP-ABPP method. We mapped the cysteine-reactivity of withaferin A by pre-incubating withaferin A (10 μM) for 30 min in 231MFP breast cancer cell proteomes, prior to labeling with the cysteine-reactive iodoacetamide-alkyne (IAyne) probe (100 μM, 30 min). Probe labeled proteins were then tagged with an isotopically light (for control) or heavy (for withaferin A-treated) biotin-azide tag bearing a TEV protease recognition site by CuAAC. Control and treated proteomes were then mixed in a 1:1 ratio, probe labeled proteins were avidin-enriched and tryptically digested, probe-labeled tryptic peptides were avidin-enriched again, and released by TEV protease and analyzed by quantitative proteomic methods and light to heavy peptide ratios were quantified. FIG. 2B: Competitive isoTOP-ABPP analysis of withaferin A cysteine-reactivity in 231MFP breast cancer cell proteomes in vitro. Light to heavy ratios of ˜1 indicate peptides that were labeled by IAyne, but not bound by withaferin A. We designate light to heavy ratios of >5 as targets that were bound by withaferin A. Also shown are the peptide sequences and sites of modification of probe-modified peptides identified for PPP2R1A and the light to heavy ratios of C377 and C390 on PPP2R1A. The sequences in the Figure are C377: DNTIEHLLPLFLAQLKDEC*PEVR, which correspond to SEQ ID NO:2; and C390: LNIISNLDC*VNEVIGIR, which corresponds to SEQ ID NO:3. FIG. 2C: Validation of PPP2R1A as a target of withaferin A. Withaferin A was pre-incubated with pure human PPP2R1A protein followed by IAyne. Probe-labeled proteins conjugated to rhodamine-azide by CuAAC and analyzed by SDS/PAGE and in-gel fluorescence. FIG. 2D: Crystal structure of PP2A complex showing C377 of PPP2R1A (shown in white), the catalytic subunit, and another regulatory subunit. PDB structure used is 2IAE. FIG. 2E: PP2A activity assay with PP2A complex proteins PPP2R1A wild-type (WT) or C377A mutant and PPP2R2A and PPP2CA subunits measuring phosphate release from a PP2A substrate phosphopeptide. This PP2A complex was treated in vitro with DMSO or withaferin A (10 μM) for 30 min prior to initiation of the assay. FIG. 2F: Withaferin A (10 μM, 4 h) treatment significantly reduces phospho-AKT levels in 231MFP breast cancer cells and this reduction is rescued by cotreatment with cantharidin (10 μM, 4 h). Data in FIG. 2B: is average ratios from n=3. Gel in FIG. 2C is a representative gel from n=3. Data in FIGS. 2E-2F are presented as mean±sem, n=3. Significance expressed as *p<0.05 compared to vehicle-treated controls and #p<0.05 compared to withaferin A-treated control. NS refers to not significant compared to the vehicle-treated C377A PPP2R1A group. Additional data (e.g. Withaferin A in situ) and isoTOP-ABPP analysis can be found in FIGS. 7A-7G.

FIGS. 3A-3C: Screening of covalent ligand libraries in breast cancer cells. (FIG. 3A): Coupled screening of a cysteine-reactive covalent ligand library in 231MFP breast cancer cells with competitive isoTOP-ABPP platforms to identify anti-cancer lead compounds, targets, and ligandable hotspots within these targets. (FIG. 3B): screened a cysteine-reactive fragment library consisting of acrylamides and chloroacetamides in 231MFP breast cancer cells (100 μM) to identify any leads that significantly impaired 231MFP breast cancer cell proliferation. Cell viability was assessed 48 h after treatment by Hoescht staining. The compounds tested, from left to right of FIG. 3B, are DKM 2-91, DKM 2-90, DKM 2-101, TRH 1-53, DKM 2-52, DKM 2-76, DKM 2-79, DKM 2-71, DKM 3-30, DKM 3-22, TRH 1-17, TRH 1-51, DKM 2-72, DKM 3-70, TRH 1-50, DKM 2-76, TRH 1-23, DKM 3-42, DKM 2-94, DKM 2-93, DKM 2-114, TRH 1-12, DKM 3-3, DKM 2-59, DKM 2-107, DKM 2-98, DKM 2-85, DKM 2-119, DKM 2-83, TRH 1-55, DKM 2-97, DKM 2-117, DKM 2-80, DKM 3-10, DKM 3-43, DKM 3-31, DKM 2-40, DKM 3-41, DKM 2-87, TRH 1-32, DKM 2-47, DKM 2-43, DKM 3-7, DKM 2-116, DKM 2-106, DKM 2-120, DKM 2-102, DKM 3-29, DKM 3-16, DKM 2-111, DKM 1-19, DKM 3-36, DKM 2-109, DKM 3-4, DKM 3-13, DKM 2-32, TRH 1-54, DKM 2-113, DKM 3-5, DKM 2-110, DKM 2-37, DKM 2-84, DKM 2-60, DKM 48, TRH 1-20, DKM 2-67, DKM 2-31, DKM 2-103, TRH 1-13, DKM 2-49, DKM 2-62, DKM 2-42, TRH 1-27, DKM 2-100, DKM 3-32, DKM 3-11, DKM 3-8, DKM 3-15, DKM 2-95, DKM 2-50, DKM 2-108, DKM 2-58, DKM 2-86, DKM 3-12, DKM 2-34, DKM 3-9, DKM 2-33, and DKM 2-39. (FIG. 3C): Validation of PPP2R1A as a target of withaferin A. Withaferin A was pre-incubated with pure human PPP2R1A protein followed by IAyne. Probe-labeled proteins conjugated to rhodamine-azide by CuAAC and analyzed by SDS/PAGE and in-gel fluorescence. Data in (FIG. 3B) are presented as mean±sem, n=3. Significance expressed as *p<0.05 compared to vehicle-treated controls.

FIGS. 4A-4E: Target identification of DKM 2-90 using competitive isoTOP-ABPP platforms. (FIG. 4A): Dose-responsive effects of DKM 2-90 and DKM 2-91 on cell proliferation in 231MFP breast cancer cells. 231MFP cells were treated with DMSO or DKM 2-90 or DKM 2-91 and proliferation was assessed 48 h after treatment by Hoescht staining. (FIG. 4B): Effect of DKM 2-90 and DKM 2-91 on cell proliferation in MCF 10A mammary epithelial cells assessed 48 h after treatment by Hoescht staining. (FIG. 4C): Isotop-ABPP analysis of DKM 2-90 in 231MFP cell proteomes. 231MFP proteomes were pre-treated with DMSO or DKM 2-90 (100 μM) for 30 min prior to labeling proteomes with IAyne (100 μM), followed by appendage of a biotin-azide tag bearing an isotopically light (control) or heavy (treated) handle and TEV protease recognition site. Control and treated proteomes were mixed in a 1:1 ratio and probe labeled proteins tryptic peptides were subsequently enriched and analyzed by quantitative proteomic approaches. A light to heavy ratio of 1 indicates that the probe-labeled cysteine-bearing peptide was not bound by the covalent ligand, whereas a ratio >10 indicates bound sites. (FIG. 4D): Competition of DKM 2-90 against IAyne labeling of pure human PPP2R1A protein. DKM 2-90 was pre-incubated with pure PPP2R1A protein for 30 min prior to labeling with IAyne (100 μM) for 30 min. Rhodamine-azide was appended on by copper-catalyzed azide-alkyne cycloaddition and proteins were separated by SDS/PAGE and analyzed by in-gel fluorescence. (FIG. 4E): Levels of total and phosphorylated AKT (p-AKT) and vinculin as a loading control in 231MFP breast cancer cells. 231MFP cells were treated with vehicle, DKM 2-90 (100 μM), or cantharidin (10 μM) and DKM 2-90 (100 μM) for 5 h. Proteins were blotted for p-AKT, total AKT, and vinculin loading control. All data shown represents n=3-5/group.

FIGS. 5A-5C: Withaferin A and DKM 2-90 mediated changes in cellular metabolism in breast cancer cells. (FIG. 5A): Metabolomic profiling of withaferin A and DKM 2-90 in 231MFP breast cancer cells. 231MFP breast cancer cells were treated with vehicle DMSO or withaferin A (10 μM) or DKM 2-90 (100 μM) for 5 h and metabolites were measured using SRM-based LC-MS/MS. (FIG. 5B): Representative metabolite levels showing common metabolic changes conferred by withaferin A and DKM 2-90 on glycolytic and phospholipid metabolism. (FIG. 5C): Model for proposed actions of withaferin A and DKM 2-90 in binding to C377 on PPP2R1A to activate PP2A activity, impair AKT signaling, impair PFK1 activity, and inhibit glycolytic and lipid metabolism and ATP levels. Withaferin A and DKM 2-90 activates PP2A to inhibit AKT signaling and glycolytic and lipid metabolism in breast cancer cells. Data in (FIG. 5B) is presented as mean±sem, n=5/group. Significance is shown as *p<0.05 compared to vehicle-treated controls.

FIG. 6: Residues of protein phosphatase 2A regulatory subunit A alpha isoform (PPP2R1A); protein phosphatase 2A catalytic subunit alpha isoform (PPP2CA); and protein phosphatase 2A regulatory subunit gamma isoform (PPP2R5C) in the protein phosphatase 2A (PP2A) complex.

FIGS. 7A-7G: Investigating the interactions of withaferin A and DKM 2-90. (FIG. 7A) Anti-proliferative dose-response of withaferin A in 231MFP cells. Cells were treated with DMSO or withaferin A for 48 h in serum-containing media and cell viability was assessed by Hoechst staining. (FIG. 7B) IsoTOP-ABPP analysis of withaferin A treatment in 231MFP cells. 231MFP cells were treated with DMSO or withaferin A (10 μM) for 4 h. Proteomes were subsequently labeled ex situ with IAyne for 1 h and subjected to the isoTOP-ABPP method. Light to heavy ratios of probe-modified peptides are shown. (FIG. 7C) Gel-based ABPP analysis of withaferin A competition against IAyne labeling of pure human KEAP1 and vimentin. Purified proteins were pre-treated with DMSO or withaferin A (10 μM) for 30 min at 37° C. before IAyne labeling (10 μM) for 30 min at room temperature. Probe labeled proteins were subsequently appended to rhodamine-azide by CuAAC and analyzed by SDS/PAGE and in-gel fluorescence. (FIG. 7D) PPP2R1A expression as assessed by qPCR. 231MFP cells were transfected with siControl or siPPP2R1A oligonucleotides and cells were harvested for qPCR analysis after 48 h. (FIG. 7E) 231MFP cell proliferation. 231MFP cells were transfected with siControl or siPPP2R1A oligonucleotides for 48 h and then cells were seeded and treated with either DMSO or withaferin A (10 μM) for an additional 48 h and cell viability was assessed by Hoechst staining. (FIG. 7F) IsoTOP-ABPP analysis of DKM 2-90 treatment in 231MFP cells. 231MFP cells were treated with DMSO or DKM 2-90 (100 μM) for 4 h. Proteomes were subsequently labeled ex situ with IAyne for 1 h and subjected to the isoTOP-ABPP method. Light to heavy ratios of probe-modified peptides are shown. (FIG. 7G) 231MFP cell proliferation. 231MFP cells were transfected with siControl or siPPP2R1A oligonucleotides for 48 h and then cells were seeded and treated with either DMSO or DKM 2-90 (100 μM) for an additional 48 h and cell viability was assessed by Hoechst staining. Data in (FIGS. 7A, 7D, 7E, and 7G) is presented as mean±sem, n=3-5/group. Significance in (FIGS. 7D, 7E, and 7G) is expressed as *p<0.05 compared to vehicle-treated siControl cells and #p<0.05 compared to withaferin A or DKM 2-90-treated siControl cells.

FIGS. 8A-8C. Characterization of DKM 2-90 analogs JNS 1-37 and JNS 1-40. (FIG. 8A) Structure of JNS 1-37 and gel-based ABPP analysis of its potency against PPP2R1A. Pure human PPP2R1A was pre-treated with DMSO or JNS 1-37 for 30 min at 37° C. prior to IAyne labeling for 30 min at room temperature. Probe-labeled proteins were appended to rhodamine-azide by CuAAC and analyzed by SDS/PAGE and in-gel fluorescence. (FIG. 8B) IsoTOP-ABPP analysis of JNS 1-40 treatment in 231MFP cells. 231MFP cells were treated with DMSO or JNS 1-40 (100 μM) for 4 h. Proteomes were subsequently labeled ex situ with IAyne for 1 h and subjected to the isoTOP-ABPP method. Light to heavy ratios of probe-modified peptides are shown. (FIG. 8C) 231MFP cell proliferation. 231MFP cells were transfected with siControl or siPPP2R1A oligonucleotides for 48 h and then cells were seeded and treated with either DMSO or JNS 1-40 (100 μM) for an additional 48 h and cell viability was assessed by Hoechst staining. Data in (FIG. 8C) is presented as mean±sem, n=5/group. Significance in (FIG. 8C) is expressed as *p<0.05 compared to vehicle-treated siControl cells and #p<0.05 compared to JNS 1-40-treated siControl cells.

FIGS. 9A-9G. Covalent ligand JNS 1-40 selectively targets C377 of PPP2R1A to activate PP2A activity and impair breast cancer pathogenicity. Structure of JNS 1-40 and gel-based ABPP analysis of its potency against PPP2R1A. Pure human PPP2R1A was pre-treated with DMSO or JNS 1-40 for 30 min at 37° C. prior to IAyne labeling for 30 min at room temperature. Probe-labeled proteins were appended to rhodamine-azide by CuAAC and analyzed by SDS/PAGE and in-gel fluorescence. (FIG. 9B) IsoTOP-ABPP analysis of JNS 1-40 treatment in 231MFP cells. 231MFP proteomes were treated in vitro with DMSO or JNS 1-40 (100 μM) for 30 min prior to IAyne labeling for 1 h and subjected to the isoTOP-ABPP method. Light to heavy ratios of probe-modified peptides are shown. (FIG. 9C) PP2A activity assay with PP2A complex proteins PPP2R1A wild-type (WT) or C377A mutant and PPP2R2A and PPP2CA subunits measuring phosphate release from a PP2A substrate phosphopeptide. This PP2A complex was treated in vitro with DMSO or JNS 1-40 (100 μM) for 30 min prior to initiation of the assay. (FIG. 9D) Levels of total and phosphorylated AKT (p-AKT) and vinculin as a loading control in 231MFP breast cancer cells. 231MFP cells were treated with vehicle or JNS 1-40 (100 μM) (100 μM) for 5 h. (FIGS. 9E, 9F) JS 1-40 (100 μM) impairs cell proliferation and serum-free cell survival after 48 h in 231MFP cells. (FIG. 9G) 231MFP tumor xenograft growth in immune-deficient SCID mice. 231MFP cells were subcutaneously injected into mice. Daily once per day treatment with vehicle or JNS 1-40 (50 mg/kg ip) was initiated 15 days after tumor implantation. Data in (FIGS. 9C-9G) are presented as mean±sem, n=3-7/group. Data in (FIG. 9B) is average ratios from n=3. Significance is shown as *p<0.05 compared to vehicle-treated controls. NS indicates not significant (p>0.05) compared to the vehicle-treated C377A PPP2R1A group.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, or S), and wherein the nitrogen and sulfur atoms may optionally be oxidized, and the nitrogen heteroatom may optionally be quaternized. The heteroatom(s) (e.g., O, N, P, S, B, As, or Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′-represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

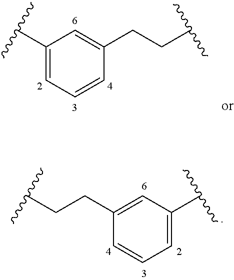 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
(i) oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from:
(a) oxo, halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OC HBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl, heteroalkyl, cycloalkyl, heterocycloalkyl, aryl, heteroaryl, substituted with at least one substituent selected from: oxo,
halogen, —CCl₃, —CBr₃, —CF₃, —CI₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “covalent cysteine modifier moiety” as used herein refers to a substituent that is capable of reacting with the sulfhydryl functional group of a cysteine amino acid (e.g. cysteine corresponding to C377 of the human PPP2R1A) to form a covalent bond. Thus, the covalent cysteine modifier moiety is typically electrophilic.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

A “PPP2R1A modulator” and “serine/threonine-protein phosphatase 2A 65 kDa regulatory subunit A alpha isoform modulator” is a substance (e.g., oligonucleotide, protein, composition, or compound) that changes the physical state of PPP2R1A relative to the physical state of PPP2R1A in the absence of the modulator (e.g., wherein the PPP2R1A modulator binds PPP2R1A, covalently modifies PPP2R1A, covalently modifies a cysteine of PPP2R1A). In embodiments, a PPP2R1A modulator binds PPP2R1A protein in a protein phosphatase 2A complex (PP2A). A protein phosphatase 2A complex (PP2A) is a heteromeric complex including a catalytic protein (e.g., PPP2CA) and a regulatory A or structural A protein (e.g., PPP2R1A), and optionally a regulatory B protein (e.g., PPP2R5C), having protein phosphatase activity. In embodiments, a PPP2R1A modulator increases PP2CA activity. In embodiments, a PPP2R1A modulator binds PPP2R1A and increases the level of PP2CA activity (e.g., phosphatase activity). In embodiments, a PPP2R1A modulator binds PPP2R1A and increases the level of PP2CA activity (e.g., phosphatase activity) of the PP2CA including the PPP2R1A contacting the PPP2R1A modulator. A “serine/threonine-protein phosphatase 2A 65 kDa regulatory subunit A alpha isoform modulator compound” or “PPP2R1A modulator compound” refers to a compound (e.g. compounds described herein) that modulates the physical state (e.g., covalently modifies the protein or a cysteine of the protein) of PPP2R1A when compared to a control, such as absence of the compound or a compound with known inactivity.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

An amino acid residue in a protein “corresponds” to a given residue when it occupies the same essential structural and/or spatial position within the protein as the given residue in a reference sequence. For example, a selected residue in a selected protein corresponds to Cys377 when the selected residue occupies the same essential structural and/or spatial position as Cys377 in SEQ ID NO:4. In some embodiments, where a selected protein is aligned for maximum homology with the human PPP2R1A protein, the position in the aligned selected protein aligning with Cys377 is said to correspond to Cys377. Instead of a primary sequence alignment, a three dimensional structural alignment can also be used, e.g., where the three dimensional structure of the selected protein is aligned for maximum correspondence with the human PPP2R1A protein (reference sequence) and the overall structures compared. In this case, the amino acid that occupies the same essential structural position as Cys377 in the structural model relative to the reference sequence is said to correspond to the Cys377 residue.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease.

As defined herein, the term “inhibitor”, “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “serine/threonine-protein phosphatase 2A 65 kDa regulatory subunit A alpha isoform” and “PPP2R1A” refer to a protein (including homologs, isoforms, and functional fragments thereof) with PPP2R1A activity. The term includes any recombinant or naturally-occurring form of PPP2R1A or variants thereof that maintain PPP2R1A activity (e.g. within at least 30%, 40%, 50%, 60%, 70%, 80%, 90%, 95%, or 100% activity compared to wildtype PPP2R1A). In embodiments, the PPP2R1A protein encoded by the PPP2R1A gene has the amino acid sequence set forth in or corresponding to Entrez 5518, UniProt P30153, or RefSeq (protein) NP_055040. In embodiments, the PPP2R1A gene has the nucleic acid sequence set forth in RefSeq (mRNA) NM_014225. In embodiments, the amino acid sequence or nucleic acid sequence is the sequence known at the time of filing of the present application. In embodiments, the sequence corresponds to NP_055040.2. In embodiments, the sequence corresponds to NM_014225.5. In embodiments, the PPP2R1A is a human PPP2R1A, such as a human cancer causing PPP2R1A. In embodiments, PPP2R1A has the following sequence:

(SEQ ID NO: 4)
MAAADGDDSLYPIAVLIDELRNEDVQLRLNSIKKLSTIALALGVERTRSE
LLPFLTDTIYDEDEVLLALAEQLGTFTTLVGGPEYVHCLLPPLESLATVE
ETVVRDKAVESLRAISHEHSPSDLEAHFVPLVKRLAGGDWFTSRTSACGL
FSVCYPRVSSAVKAELRQYFRNLCSDDTPMVRRAAASKLGEFAKVLELDN
VKSEIIPMFSNLASDEQDSVRLLAVEACVNIAQLLPQEDLEALVMPTLRQ
AAEDKSWRVRYMVADKFTELQKAVGPEITKTDLVPAFQNLMKDCEAEVRA
AASHKVKEFCENLSADCRENVIMSQILPCIKELVSDANQHVKSALASVIM
GLSPILGKDNTIEHLLPLFLAQLKDECPEVRLNIISNLDCVNEVIGIRQL
SQSLLPAIVELAEDAKWRVRLAIIEYMPLLAGQLGVEFFDEKLNSLCMAW
LVDHVYAIREAATSNLKKLVEKFGKEWAHATIIPKVLAMSGDPNYLHRMT
TLFCINVLSEVCGQDITTKHMLPTVLRMAGDPVANVRFNVAKSLQKIGPI
LDNSTLQSEVKPILEKLTQDQDVDVKYFAQEALTVLSLA

The terms “Serine/threonine-protein phosphatase 2A 56 kDa regulatory subunit gamma isoform” and “PPP2R5C” refer to a protein (including homologs, isoforms, and functional fragments thereof) with PPP2R5C activity. The term includes any recombinant or naturally-occurring form of PPP2R5C or variants thereof that maintain PPP2R5C activity (e.g. within at least 30%, 40%, 50%, 60%, 70%, 80%, 90%, 95%, or 100% activity compared to wildtype PPP2R5C). In embodiments, the PPP2R5C protein encoded by the PPP2R5C gene has the amino acid sequence set forth in or corresponding to Entrez 5527, UniProt Q13362, or RefSeq (protein) NP_002710. In embodiments, the PPP2R5C gene has the nucleic acid sequence set forth in RefSeq (mRNA) NM_002719. In embodiments, the amino acid sequence or nucleic acid sequence is the sequence known at the time of filing of the present application. In embodiments, the sequence corresponds to NP_002710.2. In embodiments, the sequence corresponds to NM_002719.3. In embodiments, the PPP2R5C is a human PPP2R5C, such as a human cancer causing PPP2R5C. In embodiments, PPP2R5C has the following sequence:

(SEQ ID NO: 5)
MLTCNKAGSRMVVDAANSNGPFQPVVLLHIRDVPPADQEKLFIQKLRQCC
VLFDFVSDPLSDLKWKEVKRAALSEMVEYITHNRNVITEPIYPEVVHMFA
VNMFRTLPPSSNPTGAEFDPEEDEPTLEAAWPHLQLVYEFFLRFLESPDF
QPNIAKKYIDQKFVLQLLELFDSEDPRERDFLKTTLHRIYGKFLGLRAYI
RKQINNIFYRFIYETEHHNGIAELLEILGSIINGFALPLKEEHKIFLLKV
LLPLHKVKSLSVYHPQLAYCVVQFLEKDSTLTEPVVMALLKYWPKTHSPK
EVMFLNELEEILDVIEPSEFVKIMEPLFRQLAKCVSSPHFQVAERALYYW
NNEYIMSLISDNAAKILPIMFPSLYRNSKTHWNKTIHGLIYNALKLFMEM
NQKLFDDCTQQFKAEKLKEKLKMKEREEAWVKIENLAKANPQYTVYSQAS
TMSIPVAMETDGPLFEDVQMLRKTVKDEAHQAQKDPKKDRPLARRKSELP
QDPHTKKALEAHCRADELASQDGR

The terms “Serine/threonine-protein phosphatase 2A catalytic subunit alpha isoform” and “PPP2CA” refer to a protein (including homologs, isoforms, and functional fragments thereof) with PPP2CA activity. The term includes any recombinant or naturally-occurring form of PPP2CA or variants thereof that maintain PPP2CA activity (e.g. within at least 30%, 40%, 50%, 60%, 70%, 80%, 90%, 95%, or 100% activity compared to wildtype PPP2CA). In embodiments, the PPP2CA protein encoded by the PPP2CA gene has the amino acid sequence set forth in or corresponding to Entrez 5515, UniProt P67775, or RefSeq (protein) NP_002706. In embodiments, the PPP2CA gene has the nucleic acid sequence set forth in RefSeq (mRNA) NM_002715. In embodiments, the amino acid sequence or nucleic acid sequence is the sequence known at the time of filing of the present application. In embodiments, the sequence corresponds to NP_002706.1. In embodiments, the sequence corresponds to NM_002715.2. In embodiments, the PPP2CA is a human PPP2CA, such as a human cancer causing PPP2CA. In embodiments, PPP2CA has the following sequence:

(SEQ ID NO: 6)
MDEKVFTKELDQWIEQLNECKQLSESQVKSLCEKAKEILTKESNVQEVRC
PVTVCGDVHGQFHDLMELFRIGGKSPDTNYLFMGDYVDRGYYSVETVTLL
VALKVRYRERITILRGNHESRQITQVYGFYDECLRKYGNANVWKYFTDLF
DYLPLTALVDGQIFCLHGGLSPSIDTLDHIRALDRLQEVPHEGPMCDLLW
SDPDDRGGWGISPRGAGYTFGQDISETFNHANGLTLVSRAHQLVMEGYNW
CHDRNVVTIFSAPNYCYRCGNQAAIMELDDTLKYSFLQFDPAPRRGEPHV
TRRTPDYFL

The terms “protein phosphatase 2” and “PP2” and “PP2A” “PP2A protein complex” refer to a protein (including homologs, isoforms, and functional fragments thereof) encoded by the PPP2CA gene. PP2A is a heterotrimeric protein phosphatase which is comprised of structural, catalytic, and regulatory subunits. The subunits which comprise PP2A include PP2A 65 kDa regulatory subunit A alpha isoform (PPP2R1A), PP2A 65 kDa regulatory subunit A beta isoform (PPP2R1B), PP2A 55 kDa regulatory subunit B beta isoform (PPP2R2B), PP2A 55 kDa regulatory subunit B gamma isoform (PPP2R2C), PP2A 55 kDa regulatory subunit B delta isoform (PPP2R2D), PP2A 72/130 kDa regulatory subunit B (PPP2R3A), PP2A 48 kDa regulatory subunit B (PPP2R3B), PP2A regulatory subunit B″ subunit gamma (PPP2R3C), PP2A regulatory subunit B′ (PPP2R4), PP2A 56 kDa regulatory subunit alpha isoform (PPP2R5A), PP2A 56 kDa regulatory subunit beta isoform (PPP2R5B), PP2A 56 kDa regulatory subunit gamma isoform (PPP2R5C), PP2A 56 kDa regulatory subunit delta isoform (PPP2R5D), PP2A 56 kDa regulatory subunit epsilon isoform (PPP2R5E), catalytic subunit alpha isoform (PPP2CA), and catalytic subunit beta isoform (PPP2CB). The term includes any recombinant or naturally-occurring form of PP2A or variants thereof that maintain PPP2CA activity (e.g. within at least 30%, 40%, 50%, 60%, 70%, 80%, 90%, 95%, or 100% activity compared to wildtype PP2A). In embodiments, the PP2A protein encoded by the PPP2CA gene has the amino acid sequence set forth in or corresponding to Entrez 5516 or UniProt P62714. In embodiments, PP2A has the following sequence:

(SEQ ID NO: 7)
MDDKAFTKELDQWVEQLNECKQLNENQVRTLCEKAKEILTKESNVQEVRC
PVTVCGDVHGQFHDLMELFRIGGKSPDTNYLFMGDYVDRGYYSVETVTLL
VALKVRYPERITILRGNHESRQITQVYGFYDECLRKYGNANVWKYFTDLF
DYLPLTALVDGQIFCLHGGLSPSIDTLDHIRALDRLQEVPHEGPMCDLLW
SDPDDRGGWGISPRGAGYTFGQDISETFNHANGLTLVSRAHQLVMEGYNW
CHDRNVVTIFSAPNYCYRCGNQAAIMELDDTLKYSFLQFDPAPRRGEPHV
TRRTPDYFL

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The terms “disease” or “condition” refer to a state of being or health status of a patient or subject capable of being treated with the compounds or methods provided herein. The disease may be a cancer. The disease may be stroke. The disease may be an inflammatory disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include brain cancer, glioma, glioblastoma, neuroblastoma, prostate cancer, colorectal cancer, pancreatic cancer, cervical cancer, gastric cancer, ovarian cancer, lung cancer, and cancer of the head. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. In embodiments, the treating or treatment is no prophylactic treatments.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “modulator” **discuss**refers to a substance (e.g., oligonucleotide, protein, composition, or compound) that changes the physical state of the target molecule (e.g., PPP2R1A or PP2A) relative to the physical state of the target molecule in the absence of the modulator (e.g., wherein the modulator binds the target molecule, covalently modifies the target molecule, covalently modifies a cysteine of the molecule). In some embodiments, a PPP2R1A associated disease modulator is a compound that reduces the severity of one or more symptoms of a disease associated with PPP2R1A (e.g. cancer). In embodiments, a PPP2R1A modulator is a compound that changes the physical state of PPP2R1A by covalently modifying a cysteine of PPP2R1A. In embodiments, a PPP2R1A modulator is a compound that changes the physical state of PPP2R1A by covalently modifying a cysteine of PPP2R1A, which due it it being a subunit of PP2A results in activation of PP2A (e.g., increasing PP2A activity). In embodiments, the modulator is an inhibitor of PPP2R1A. In embodiments, the modulator is an activator of PPP2R1A.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule or the physical state of the molecule. In embodiments, modulating is activating. In embodiments, modulating is inhibiting.

The term “associated” or “associated with” in the context of a substance or substance activity or function associated with a disease (e.g. a protein associated disease, a cancer associated with PPP2R1A activity, PPP2R1A associated cancer, PPP2R1A associated disease) means that the disease (e.g. cancer) is caused by (in whole or in part), or a symptom of the disease is caused by (in whole or inpart) the substance or substance activity or function. For example, a cancer associated with PPP2R1A activity or function may be a cancer that results (entirely or partially) from aberrant PPP2R1A function (e.g. enzyme activity, protein-protein interaction, signaling pathway) or a cancer wherein a particular symptom of the disease is caused (entirely or partially) by aberrant PPP2R1A activity or function. As used herein, what is described as being associated with a disease, if a causative agent, could be a target for treatment of the disease. For example, a cancer associated with PPP2R1A activity or function or a PPP2R1A associated cancer, may be treated with a PPP2R1A modulator, in the instance where PPP2R1A activity or function (e.g. signaling pathway activity) causes the cancer.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propogated to other signaling pathway components. For example, binding of a PPP2R1A protein with a compound as described herein may reduce the interactions between the PPP2R1A protein and downstream effectors (e.g., PPP2CA) or signaling pathway components, resulting in changes in cell growth, proliferation, or survival.

The term “electrophilic chemical moiety” is used in accordance with its plain ordinary chemical meaning and refers to a chemical group (e.g., monovalent chemical group) that is electrophilic.

The term “nucleophilic chemical moiety” is used in accordance with its plain ordinary chemical meaning and refers to a chemical group (e.g., monovalent chemical group) that is nucleophilic.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

An “antisense nucleic acid” as referred to herein is a nucleic acid (e.g., DNA or RNA molecule) that is complementary to at least a portion of a specific target nucleic acid (e.g., a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid (e.g., a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5). In embodiments, the antisense nucleic acid hybridizes to the target nucleic acid (e.g. a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) in vitro. In embodiments, the antisense nucleic acid hybridizes to the target nucleic acid (e.g. a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) in a cell. In embodiments, the antisense nucleic acid hybridizes to the target nucleic acid (e.g. a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) in an organism. In embodiments, the antisense nucleic acid hybridizes to the target nucleic acid (e.g. a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 51) under physiological conditions. Antisense nucleic acids may comprise naturally occurring nucleotides or modified nucleotides such as, e.g., phosphorothioate, methylphosphonate, and -anomeric sugar-phosphate, backbonemodified nucleotides.

In the cell, the antisense nucleic acids hybridize to the corresponding RNA (e.g., a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) forming a double-stranded molecule. The antisense nucleic acids interfere with the endogenous behavior of the RNA (e.g., a nucleic acid coding for one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5) and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab; while the terms “variable light chain,” “V_{L}” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allyl amino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyl eleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

The term “protein phosphatase 2A (PP2A) activity” as used herein refers to the biological activity of the protein. Protein phosphatase 2A (PP2A) activity may be quantified by measuring the amount of PP2A (e.g., PPP2CA) binding to another protein (e.g., Akt), PP2A (e.g., PPP2CA) de-phosphorylation of a protein (e.g., Akt), measuring the rate of cell division, cell survival, cell migration, actin cytoskeleton polymerization, actin cytoskeleton stabilization, or epithelial-mesenchymal transition rates.

The term “a PPP2R1A protein-PPP2R1A modulator complex” as used herein refers to a PPP2R1A protein bonded (e.g., covalently bonded) to a PPP2R1A modulator (e.g., a compound described herein).

### II. Compounds

In an aspect is provided a compound having the formula:

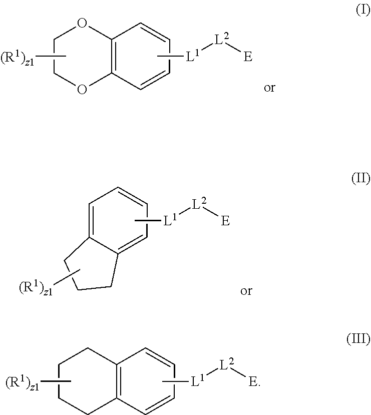

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl.

The symbol z1 is an integer from 0 to 7.

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

E is an electrophilic moiety.

Each R^{1A}, R^{1B}, R^{1C}, R^{1D}, R^{4A}, R^{4B}, R^{5A}, and R^{5B} is independently

hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{1A} and R^{1B} substituents bonded to the same nitrogen atom may optionally be joined to form a substituted or unsubstituted heterocycloalkyl or substituted or unsubstituted heteroaryl. R^{4A} and R^{4B} substituents bonded to the same nitrogen atom may optionally be joined to form a substituted or unsubstituted heterocycloalkyl or substituted or unsubstituted heteroaryl. R^{5A} and R^{5B} substituents bonded to the same nitrogen atom may optionally be joined to form a substituted or unsubstituted heterocycloalkyl or substituted or unsubstituted heteroaryl.

Each X, X¹, X⁴, and X⁵ is independently —F, —Cl, —Br, or —I.

The symbols n1, n4, and n5 are independently an integer from 0 to 4.

The symbols m1, m4, m5, v1, v4, and v5 are independently an integer from 1 to 2.

In embodiments, the compound has the formula:

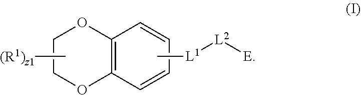

R¹, L¹, z1, L², and E are as described herein.

In embodiments, the compound has the formula:

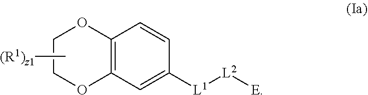

R¹, L¹, z1, L², and E are as described herein.

In embodiments, the compound has the formula:

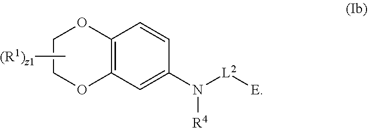

R¹, z1, R⁴, L², and E are as described herein.

In embodiments, the compound has the formula:

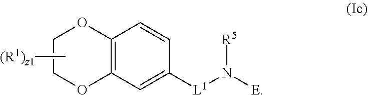

R¹, z1, R⁵, L¹, and E are as described herein.

In embodiments, the compound has the formula:

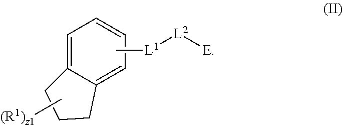

R¹, L¹, z1, L², and E are as described herein.

In embodiments, the compound has the formula:

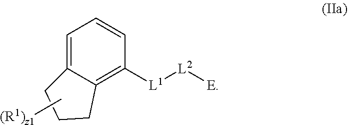

R¹, L¹, z1, L², and E are as described herein.

In embodiments, the compound has the formula:

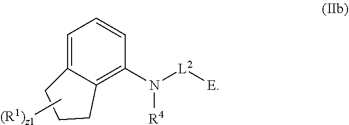

R¹, R⁴, z1, L², and E are as described herein.

In embodiments, the compound has the formula:

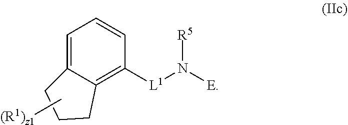

R¹, L¹, z1, R⁵, and E are as described herein.

In embodiments, the compound has the formula:

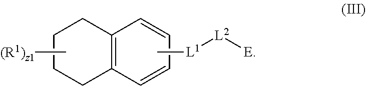

R¹, L¹, z1, L², and E are as described herein.

In embodiments, the compound has the formula:

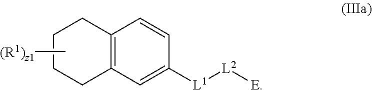

R¹, L¹, z1, L², and E are as described herein.

In embodiments, the compound has the formula:

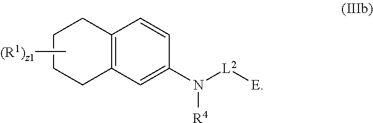

R¹, R⁴, z1, L², and E are as described herein.

In embodiments, the compound has the formula:

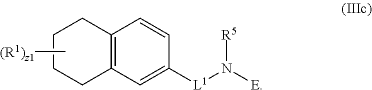

R¹, R⁵, z1, L¹, and E are as described herein.

It will be understood that R¹ is a floating substituent and may be attached to either of the fused rings in the formulae shown herein above. For example, the two formulae below are equivalent:

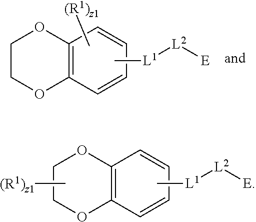

The two formulae shown below are equivalent:

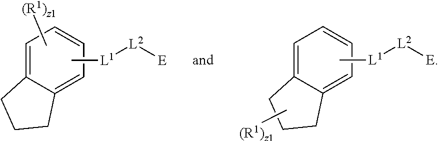

The two formulae shown below are equivalent:

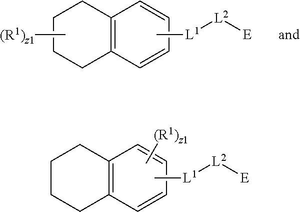

It will be further understood that a plurality of floating substitutents may be bonded to either of the fused rings shown above, or one or more substituents maybe bonded to one ring and one or more other substituents may be bonded to a different ring.

In embodiments, R¹ is independently halogen, —CX¹₃, —CHX¹₂, —CH₂X, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ aryl, or substituted or unsubstituted 5 to 12 membered heteroaryl.

In embodiments, R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, two adjacent R¹ substituents are joined to form a substituted or unsubstituted cycloalkyl, substituted or unsubstituted heterocycloalkyl, substituted or unsubstituted aryl, or substituted or unsubstituted heteroaryl. In embodiments, two adjacent R¹ substituents are joined to form an unsubstituted cycloalkyl. In embodiments, two adjacent R¹ substituents are joined to form an unsubstituted C₃-C₆ cycloalkyl.

In embodiments, R¹ is independently unsubstituted methyl. In embodiments, R¹ is independently unsubstituted ethyl. In embodiments, R¹ is independently unsubstituted propyl. In embodiments, R¹ is independently unsubstituted isopropyl. In embodiments, R¹ is independently unsubstituted n-propyl. In embodiments, R¹ is independently unsubstituted butyl. In embodiments, R¹ is independently unsubstituted n-butyl. In embodiments, R¹ is independently unsubstituted t-butyl. In embodiments, R¹ is independently unsubstituted pentyl. In embodiments, R¹ is independently unsubstituted n-pentyl. In embodiments, R¹ is independently unsubstituted hexyl. In embodiments, R¹ is independently unsubstituted n-hexyl. In embodiments, R¹ is independently unsubstituted heptyl. In embodiments, R¹ is independently unsubstituted n-heptyl. In embodiments, R¹ is independently unsubstituted octyl. In embodiments, R¹ is independently unsubstituted n-octyl. In embodiments, R¹ is independently unsubstituted benzyl. In embodiments, R¹ is independently unsubstituted C₁-C₈ alkyl. In embodiments, R¹ is independently halo-substituted methyl. In embodiments, R¹ is independently halo-substituted ethyl. In embodiments, R¹ is independently halo-substituted isopropyl. In embodiments, R¹ is independently halo-substituted n-propyl. In embodiments, R¹ is independently halo-substituted n-butyl. In embodiments, R¹ is independently halo-substituted t-butyl. In embodiments, R¹ is independently halo-substituted n-pentyl. In embodiments, R¹ is independently halo-substituted benzyl. In embodiments, R¹ is independently halo-substituted C₁-C₈ alkyl. In embodiments, R¹ is independently unsubstituted 2 to 6 membered heteroalkyl. In embodiments, R¹ is independently unsubstituted 2 to 7 membered heteroalkyl. In embodiments, R¹ is independently unsubstituted 2 to 8 membered heteroalkyl. In embodiments, R¹ is independently unsubstituted 2 to 9 membered heteroalkyl. In embodiments, R¹ is independently unsubstituted 2 to 10 membered heteroalkyl. In embodiments, R¹ is independently unsubstituted 3 to 10 membered heteroalkyl. In embodiments, R¹ is independently unsubstituted 4 to 10 membered heteroalkyl. In embodiments, R¹ is independently unsubstituted 5 to 10 membered heteroalkyl. In embodiments, R¹ is independently unsubstituted 6 to 10 membered heteroalkyl. In embodiments, R¹ is independently unsubstituted 7 to 10 membered heteroalkyl. In embodiments, R¹ is independently unsubstituted 8 to 10 membered heteroalkyl. In embodiments, R¹ is independently unsubstituted 6 to 10 membered heteroalkyl. In embodiments, R¹ is independently unsubstituted 7 to 9 membered heteroalkyl.

In embodiments, two adjacent R¹ substituents are joined to form an unsubstituted C₃-C₆ cycloalkyl. In embodiments, two adjacent R¹ substituents are joined to form an unsubstituted C₄-C₆ cycloalkyl. In embodiments, two adjacent R¹ substituents are joined to form an unsubstituted C₃-C₅ cycloalkyl. In embodiments, two adjacent R¹ substituents are joined to form an unsubstituted C₅-C₆ cycloalkyl. In embodiments, two adjacent R¹ substituents are joined to form an unsubstituted C₄ cycloalkyl.

In embodiments, R¹ is independently unsubstituted 5 membered heteroaryl. In embodiments, R¹ is independently unsubstituted 6 membered heteroaryl. In embodiments, R¹ is independently unsubstituted pyridyl. In embodiments, R¹ is independently unsubstituted 2-pyridyl. In embodiments, R¹ is independently unsubstituted 3-pyridyl. In embodiments, R¹ is independently unsubstituted 4-pyridyl. In embodiments, R¹ is independently unsubstituted pyridazinyl. In embodiments, R¹ is independently unsubstituted pyrimidinyl. In embodiments, R¹ is independently unsubstituted pyrazinyl. In embodiments, R¹ is independently unsubstituted triazinyl. In embodiments, R¹ is independently unsubstituted pyrrolyl. In embodiments, R¹ is independently unsubstituted 2-pyrrolyl. In embodiments, R¹ is independently unsubstituted 3-pyrrolyl. In embodiments, R¹ is independently unsubstituted furanyl. In embodiments, R¹ is independently unsubstituted 2-furanyl. In embodiments, R¹ is independently unsubstituted 3-furanyl. In embodiments, R¹ is independently unsubstituted thienyl. In embodiments, R¹ is independently unsubstituted 2-thienyl. In embodiments, R¹ is independently unsubstituted 3-thienyl. In embodiments, R¹ is independently unsubstituted pyrazolyl. In embodiments, R¹ is independently unsubstituted isoxazolyl. In embodiments, R¹ is independently unsubstituted isothiazolyl. In embodiments, R¹ is independently unsubstituted imidazolyl. In embodiments, R¹ is independently unsubstituted oxazolyl. In embodiments, R¹ is independently unsubstituted thiazolyl. In embodiments, R¹ is independently unsubstituted phenyl. In embodiments, R¹ is independently unsubstituted biphenyl. In embodiments, R¹ is independently unsubstituted 2-biphenyl. In embodiments, R¹ is independently unsubstituted 3-biphenyl. In embodiments, R¹ is independently unsubstituted 4-biphenyl.

In embodiments, R¹ is independently —CX¹₃. In embodiments, R¹ is independently —CHX¹₂. In embodiments, R¹ is independently —CH₂X¹. In embodiments, R¹ is independently —OCX¹₃. In embodiments, R¹ is independently —OCH₂X¹. In embodiments, R¹ is independently —OCHX¹₂. In embodiments, R¹ is independently —CN. In embodiments, R¹ is independently —SOₙ₁R^{1D}. In embodiments, R¹ is independently —SOᵥ₁NR^{1A}R^{1B}. In embodiments, R¹ is independently —NHC(O)NR^{1A}R^{1B}. In embodiments, R¹ is independently —N(O)ₘ₁. In embodiments, R¹ is independently —NR^{1A}R^{1B}. In embodiments, R¹ is independently —C(O)R^{1C}. In embodiments, R¹ is independently —C(O)—OR^{1C}. In embodiments, R¹ is independently —C(O)NR^{1A}R^{1B}. In embodiments, R¹ is independently —OR^{1D}. In embodiments, R¹ is independently —NR^{1A}SO₂R^{1D}. In embodiments, R¹ is independently —NR^{1A}C(O)R^{1C}. In embodiments, R¹ is independently —NR^{1A}C(O)OR^{1C}In embodiments, R¹ is independently —NR^{1A}OR^{1C}. In embodiments, R¹ is independently —OH. In embodiments, R¹ is independently —NH₂. In embodiments, R¹ is independently —COOH. In embodiments, R¹ is independently —CONH₂. In embodiments, R¹ is independently —NO₂. In embodiments, R¹ is independently —SH. In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —CF₃. In embodiments, R¹ is independently —CHF₂. In embodiments, R¹ is independently —CH₂F. In embodiments, R¹ is independently —OCF₃. In embodiments, R¹ is independently —OCH₂F. In embodiments, R¹ is independently —OCHF₂. In embodiments, R¹ is independently —OCH₃. In embodiments, R¹ is independently —OCH₂CH₃. In embodiments, R¹ is independently —OCH₂CH₂CH₃. In embodiments, R¹ is independently —OCH(CH₃)₂. In embodiments, R¹ is independently —OC(CH₃)₃. In embodiments, R¹ is independently —SCH₃. In embodiments, R¹ is independently —SCH₂CH₃. In embodiments, R¹ is independently —SCH₂CH₂CH₃. In embodiments, R¹ is independently —SCH(CH₃)₂. In embodiments, R¹ is independently —SC(CH₃)₃.

In embodiments, R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12, 5 to 10 membered, 5 to 9 membered, or 5 to 6 membered).

In embodiments, R¹ is independently substituted or unsubstituted alkyl (e.g., C₁-C₈, C₁-C₆, C₁-C₄, or C₁-C₂). In embodiments, R¹ is independently substituted alkyl (e.g., C₁-C₈, C₁-C₆, C₁-C₄, or C₁-C₂). In embodiments, R¹ is independently unsubstituted alkyl (e.g., C₁-C₈, C₁-C₆, C₁-C₄, or C₁-C₂). In embodiments, R¹ is independently unsubstituted methyl. In embodiments, R¹ is independently unsubstituted ethyl. In embodiments, R¹ is independently unsubstituted propyl. In embodiments, R¹ is independently unsubstituted isopropyl. In embodiments, R¹ is independently unsubstituted tert-butyl. In embodiments, R¹ is independently substituted or unsubstituted heteroalkyl (e.g., 2 to 8 membered, 2 to 6 membered, 4 to 6 membered, 2 to 3 membered, or 4 to 5 membered). In embodiments, R¹ is independently substituted heteroalkyl (e.g., 2 to 8 membered, 2 to 6 membered, 4 to 6 membered, 2 to 3 membered, or 4 to 5 membered). In embodiments, R¹ is independently unsubstituted heteroalkyl (e.g., 2 to 8 membered, 2 to 6 membered, 4 to 6 membered, 2 to 3 membered, or 4 to 5 membered). In embodiments, R¹ is independently substituted or unsubstituted cycloalkyl (e.g., C₃-C₈, C₃-C₆, C₄-C₆, or C₅-C₆). In embodiments, R¹ is independently substituted cycloalkyl (e.g., C₃-C₈, C₃-C₆, C₄-C₆, or C₅-C₆). In embodiments, R¹ is independently unsubstituted cycloalkyl (e.g., C₃-C₈, C₃-C₆, C₄-C₆, or C₅-C₆). In embodiments, R¹ is independently substituted or unsubstituted heterocycloalkyl (e.g., 3 to 8 membered, 3 to 6 membered, 4 to 6 membered, 4 to 5 membered, or 5 to 6 membered). In embodiments, R¹ is independently substituted heterocycloalkyl (e.g., 3 to 8 membered, 3 to 6 membered, 4 to 6 membered, 4 to 5 membered, or 5 to 6 membered). In embodiments, R¹ is independently unsubstituted heterocycloalkyl (e.g., 3 to 8 membered, 3 to 6 membered, 4 to 6 membered, 4 to 5 membered, or 5 to 6 membered). In embodiments, R¹ is independently substituted or unsubstituted aryl (e.g., C₆-C₁₂, C₆-C₁₀, or phenyl). In embodiments, R¹ is independently substituted aryl (e.g., C₆-C₁₂, C₆-C₁₀, or phenyl). In embodiments, R¹ is independently unsubstituted aryl (e.g., C₆-C₁₂, C₆-C₁₀, or phenyl). In embodiments, R¹ is independently substituted or unsubstituted heteroaryl (e.g., 5 to 12 membered, 5 to 10 membered, 5 to 9 membered, or 5 to 6 membered). In embodiments, R¹ is independently substituted heteroaryl (e.g., 5 to 12 membered, 5 to 10 membered, 5 to 9 membered, or 5 to 6 membered). In embodiments, R¹ is independently unsubstituted heteroaryl (e.g., 5 to 12 membered, 5 to 10 membered, 5 to 9 membered, or 5 to 6 membered).

In embodiments, two adjacent R¹ substituents may optionally be joined to form a substituted or unsubstituted cycloalkyl (e.g., C₃-C₈, C₃-C₆, C₄-C₆, or C₅-C₆). In embodiments, two adjacent R¹ substituents may optionally be joined to form a substituted cycloalkyl (e.g., C₃-C₈, C₃-C₆, C₄-C₆, or C₅-C₆). In embodiments, two adjacent R¹ substituents may optionally be joined to form an unsubstituted cycloalkyl (e.g., C₃-C₈, C₃-C₆, C₄-C₆, or C₅-C₆). In embodiments, two adjacent R¹ substituents may optionally be joined to form a substituted or unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a substituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a substituted or unsubstituted aryl (e.g., C₆-C₁₂, C₆-C₁₀, or phenyl). In embodiments, two adjacent R¹ substituents may optionally be joined to form a substituted aryl (e.g., C₆-C₁₂, C₆-C₁₀, or phenyl). In embodiments, two adjacent R¹ substituents may optionally be joined to form an unsubstituted aryl (e.g., C₆-C₁₂, C₆-C₁₀, or phenyl). In embodiments, two adjacent R¹ substituents may optionally be joined to form a substituted or unsubstituted heteroaryl (e.g., 5 to 12 membered, 5 to 10 membered, 5 to 9 membered, or 5 to 6 membered). In embodiments, two adjacent R¹ substituents may optionally be joined to form a substituted heteroaryl (e.g., 5 to 12 membered, 5 to 10 membered, 5 to 9 membered, or 5 to 6 membered). In embodiments, two adjacent R¹ substituents may optionally be joined to form an unsubstituted heteroaryl (e.g., 5 to 12 membered, 5 to 10 membered, 5 to 9 membered, or 5 to 6 membered).

In embodiments, R^{1A} is independently hydrogen. In embodiments, R^{1A} is independently —CX^{1A}₃. In embodiments, R^{1A} is independently —CHX^{1A}₂. In embodiments, R^{1A} is independently —CH₂X^{1A}. In embodiments, R^{1A} is independently —CN. In embodiments, R^{1A} is independently —COOH. In embodiments, R^{1A} is independently —CONH₂. In embodiments, X^{1A} is independently —F, —Cl, —Br, or —I.

In embodiments, R^{1A} is independently substituted or unsubstituted alkyl (e.g., C₁-C₈, C₁-C₆, C₁-C₄, or C₁-C₂). In embodiments, R^{1A} is independently substituted alkyl (e.g., C₁-C₈, C₁-C₆, C₁-C₄, or C₁-C₂). In embodiments, R^{1A} is independently unsubstituted alkyl (e.g., C₁-C₈, C₁-C₆, C₁-C₄, or C₁-C₂). In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted isopropyl. In embodiments, R^{1A} is independently unsubstituted tert-butyl. In embodiments, R^{1A} is independently substituted or unsubstituted heteroalkyl (e.g., 2 to 8 membered, 2 to 6 membered, 4 to 6 membered, 2 to 3 membered, or 4 to 5 membered). In embodiments, R^{1A} is independently substituted heteroalkyl (e.g., 2 to 8 membered, 2 to 6 membered, 4 to 6 membered, 2 to 3 membered, or 4 to 5 membered). In embodiments, R^{1A} is independently unsubstituted heteroalkyl (e.g., 2 to 8 membered, 2 to 6 membered, 4 to 6 membered, 2 to 3 membered, or 4 to 5 membered). In embodiments, R^{1A} is independently substituted or unsubstituted cycloalkyl (e.g., C₃-C₈, C₃-C₆, C₄-C₆, or C₅-C₆). In embodiments, R^{1A} is independently substituted cycloalkyl (e.g., C₃-C₈, C₃-C₆, C₄-C₆, or C₅-C₆). In embodiments, R^{1A} is independently unsubstituted cycloalkyl (e.g., C₃-C₈, C₃-C₆, C₄-C₆, or C₅-C₆). In embodiments, R^{1A} is independently substituted or unsubstituted heterocycloalkyl (e.g., 3 to 8 membered, 3 to 6 membered, 4 to 6 membered, 4 to 5 membered, or 5 to 6 membered). In embodiments, R^{1A} is independently substituted heterocycloalkyl (e.g., 3 to 8 membered, 3 to 6 membered, 4 to 6 membered, 4 to 5 membered, or 5 to 6 membered). In embodiments, R^{1A} is independently unsubstituted heterocycloalkyl (e.g., 3 to 8 membered, 3 to 6 membered, 4 to 6 membered, 4 to 5 membered, or 5 to 6 membered). In embodiments, R^{1A} is independently substituted or unsubstituted aryl (e.g., C₆-C₁₂, C₆-C₁₀, or phenyl). In embodiments, R^{1A} is independently substituted aryl (e.g., C₆-C₁₂, C₆-C₁₀, or phenyl). In embodiments, R^{1A} is independently unsubstituted aryl (e.g., C₆-C₁₂, C₆-C₁₀, or phenyl). In embodiments, R^{1A} is independently substituted or unsubstituted heteroaryl (e.g., 5 to 12 membered, 5 to 10 membered, 5 to 9 membered, or 5 to 6 membered). In embodiments, R^{1A} is independently substituted heteroaryl (e.g., 5 to 12 membered, 5 to 10 membered, 5 to 9 membered, or 5 to 6 membered). In embodiments, R^{1A} is independently unsubstituted heteroaryl (e.g., 5 to 12 membered, 5 to 10 membered, 5 to 9 membered, or 5 to 6 membered).

In embodiments, R^{1B} is independently hydrogen. In embodiments, R^{1B} is independently —CX^{1B}₃. In embodiments, R^{1B} is independently —CHX^{1B}₂. In embodiments, R1B is independently —CH₂X^{1B}. In embodiments, R^{1B} is independently —CN. In embodiments, R^{1B} is independently —COOH. In embodiments, R^{1B} is independently —CONH₂. In embodiments, X^{1B} is independently —F, —Cl, —Br, or —I.

In embodiments, R^{1B} is independently substituted or unsubstituted alkyl (e.g., C₁-C₈, C₁-C₆, C₁-C₄, or C₁-C₂). In embodiments, R^{1B} is independently substituted alkyl (e.g., C₁-C₈, C₁-C₆, C₁-C₄, or C₁-C₂). In embodiments, R^{1B} is independently unsubstituted alkyl (e.g., C₁-C₈, C₁-C₆, C₁-C₄, or C₁-C₂). In embodiments, R^{1B} is independently unsubstituted methyl. In embodiments, R^{1B} is independently unsubstituted ethyl. In embodiments, R^{1B} is independently unsubstituted propyl. In embodiments, R^{1B} is independently unsubstituted isopropyl. In embodiments, R^{1B} is independently unsubstituted tert-butyl. In embodiments, R^{1B} is independently substituted or unsubstituted heteroalkyl (e.g., 2 to 8 membered, 2 to 6 membered, 4 to 6 membered, 2 to 3 membered, or 4 to 5 membered). In embodiments, R^{1B} is independently substituted heteroalkyl (e.g., 2 to 8 membered, 2 to 6 membered, 4 to 6 membered, 2 to 3 membered, or 4 to 5 membered). In embodiments, R^{1B} is independently unsubstituted heteroalkyl (e.g., 2 to 8 membered, 2 to 6 membered, 4 to 6 membered, 2 to 3 membered, or 4 to 5 membered). In embodiments, R^{1B} is independently substituted or unsubstituted cycloalkyl (e.g., C₃-C₈, C₃-C₆, C₄-C₆, or C₅-C₆). In embodiments, R^{1B} is independently substituted cycloalkyl (e.g., C₃-C₈, C₃-C₆, C₄-C₆, or C₅-C₆). In embodiments, R^{1B} is independently unsubstituted cycloalkyl (e.g., C₃-C₈, C₃-C₆, C₄-C₆, or C₅-C₆). In embodiments, R^{1B} is independently substituted or unsubstituted heterocycloalkyl (e.g., 3 to 8 membered, 3 to 6 membered, 4 to 6 membered, 4 to 5 membered, or 5 to 6 membered). In embodiments, R^{1B} is independently substituted heterocycloalkyl (e.g., 3 to 8 membered, 3 to 6 membered, 4 to 6 membered, 4 to 5 membered, or 5 to 6 membered). In embodiments, R^{1B} is independently unsubstituted heterocycloalkyl (e.g., 3 to 8 membered, 3 to 6 membered, 4 to 6 membered, 4 to 5 membered, or 5 to 6 membered). In embodiments, R^{1B} is independently substituted or unsubstituted aryl (e.g., C₆-C₁₂, C₆-C₁₀, or phenyl). In embodiments, R^{1B} is independently substituted aryl (e.g., C₆-C₁₂, C₆-C₁₀, or phenyl). In embodiments, R^{1B} is independently unsubstituted aryl (e.g., C₆-C₁₂, C₆-C₁₀, or phenyl). In embodiments, R^{1B} is independently substituted or unsubstituted heteroaryl (e.g., 5 to 12 membered, 5 to 10 membered, 5 to 9 membered, or 5 to 6 membered). In embodiments, R^{1B} is independently substituted heteroaryl (e.g., 5 to 12 membered, 5 to 10 membered, 5 to 9 membered, or 5 to 6 membered). In embodiments, R^{1B} is independently unsubstituted heteroaryl (e.g., 5 to 12 membered, 5 to 10 membered, 5 to 9 membered, or 5 to 6 membered).

In embodiments, R^{1A} and R^{1B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{1A} and R^{1B} substituents bonded to the same nitrogen atom may be joined to form a substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may be joined to form a substituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may be joined to form an unsubstituted heteroaryl (e.g., 5 to 12 membered, 5 to 10 membered, 5 to 9 membered, or 5 to 6 membered).

In embodiments, R^{1C} is independently hydrogen. In embodiments, R^{1C} is independently —CX^{1C}₃. In embodiments, R^{1C} is independently —CHX^{1C}₂. In embodiments, R^{1C} is independently —CH₂X^{1C}. In embodiments, R^{1C} is independently —CN. In embodiments, R^{1C} is independently —COOH. In embodiments, R^{1C} is independently —CONH₂. In embodiments, X^{1C} is independently —F, —Cl, —Br, or —I.

In embodiments, R^{1C} is independently substituted or unsubstituted alkyl (e.g., C₁-C₈, C₁-C₆, C₁-C₄, or C₁-C₂). In embodiments, R^{1C} is independently substituted alkyl (e.g., C₁-C₈, C₁-C₆, C₁-C₄, or C₁-C₂). In embodiments, R^{1C} is independently unsubstituted alkyl (e.g., C₁-C₈, C₁-C₆, C₁-C₄, or C₁-C₂). In embodiments, R^{1C} is independently unsubstituted methyl. In embodiments, R^{1C} is independently unsubstituted ethyl. In embodiments, R^{1C} is independently unsubstituted propyl. In embodiments, R^{1C} is independently unsubstituted isopropyl. In embodiments, R^{1C} is independently unsubstituted tert-butyl. In embodiments, R^{1C} is independently substituted or unsubstituted heteroalkyl (e.g., 2 to 8 membered, 2 to 6 membered, 4 to 6 membered, 2 to 3 membered, or 4 to 5 membered). In embodiments, R^{1C} is independently substituted heteroalkyl (e.g., 2 to 8 membered, 2 to 6 membered, 4 to 6 membered, 2 to 3 membered, or 4 to 5 membered). In embodiments, R^{1C} is independently unsubstituted heteroalkyl (e.g., 2 to 8 membered, 2 to 6 membered, 4 to 6 membered, 2 to 3 membered, or 4 to 5 membered). In embodiments, R^{1C} is independently substituted or unsubstituted cycloalkyl (e.g., C₃-C₈, C₃-C₆, C₄-C₆, or C₅-C₆). In embodiments, R^{1C} is independently substituted cycloalkyl (e.g., C₃-C₈, C₃-C₆, C₄-C₆, or C₅-C₆). In embodiments, R^{1C} is independently unsubstituted cycloalkyl (e.g., C₃-C₈, C₃-C₆, C₄-C₆, or C₅-C₆). In embodiments, R^{1C} is independently substituted or unsubstituted heterocycloalkyl (e.g., 3 to 8 membered, 3 to 6 membered, 4 to 6 membered, 4 to 5 membered, or 5 to 6 membered). In embodiments, R^{1C} is independently substituted heterocycloalkyl (e.g., 3 to 8 membered, 3 to 6 membered, 4 to 6 membered, 4 to 5 membered, or 5 to 6 membered). In embodiments, R^{1C} is independently unsubstituted heterocycloalkyl (e.g., 3 to 8 membered, 3 to 6 membered, 4 to 6 membered, 4 to 5 membered, or 5 to 6 membered). In embodiments, R^{1C} is independently substituted or unsubstituted aryl (e.g., C₆-C₁₂, C₆-C₁₀, or phenyl). In embodiments, R^{1C} is independently substituted aryl (e.g., C₆-C₁₂, C₆-C₁₀, or phenyl). In embodiments, R^{1C} is independently unsubstituted aryl (e.g., C₆-C₁₂, C₆-C₁₀, or phenyl). In embodiments, R^{1C} is independently substituted or unsubstituted heteroaryl (e.g., 5 to 12 membered, 5 to 10 membered, 5 to 9 membered, or 5 to 6 membered). In embodiments, R^{1C} is independently substituted heteroaryl (e.g., 5 to 12 membered, 5 to 10 membered, 5 to 9 membered, or 5 to 6 membered). In embodiments, R^{1C} is independently unsubstituted heteroaryl (e.g., 5 to 12 membered, 5 to 10 membered, 5 to 9 membered, or 5 to 6 membered).

In embodiments, R^{1D} is independently hydrogen. In embodiments, R^{1D} is independently —CX^{1D}₃. In embodiments, R^{1D} is independently —CHX^{1D}₂. In embodiments, R^{1D} is independently —CH₂X^{1D}. In embodiments, R^{1D} is independently —CN. In embodiments, R^{1D} is independently —COOH. In embodiments, R^{1D} is independently —CONH₂. In embodiments, X^{1D} is independently —F, —Cl, —Br, or —I.

In embodiments, R^{1D} is independently substituted or unsubstituted alkyl (e.g., C₁-C₈, C₁-C₆, C₁-C₄, or C₁-C₂). In embodiments, R^{1D} is independently substituted alkyl (e.g., C₁-C₈, C₁-C₆, C₁-C₄, or C₁-C₂). In embodiments, R^{1D} is independently unsubstituted alkyl (e.g., C₁-C₈, C₁-C₆, C₁-C₄, or C₁-C₂). In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted isopropyl. In embodiments, R^{1D} is independently unsubstituted tert-butyl. In embodiments, R^{1D} is independently substituted or unsubstituted heteroalkyl (e.g., 2 to 8 membered, 2 to 6 membered, 4 to 6 membered, 2 to 3 membered, or 4 to 5 membered). In embodiments, R^{1D} is independently substituted heteroalkyl (e.g., 2 to 8 membered, 2 to 6 membered, 4 to 6 membered, 2 to 3 membered, or 4 to 5 membered). In embodiments, R^{1D} is independently unsubstituted heteroalkyl (e.g., 2 to 8 membered, 2 to 6 membered, 4 to 6 membered, 2 to 3 membered, or 4 to 5 membered). In embodiments, R^{1D} is independently substituted or unsubstituted cycloalkyl (e.g., C₃-C₈, C₃-C₆, C₄-C₆, or C₅-C₆). In embodiments, R^{1D} is independently substituted cycloalkyl (e.g., C₃-C₈, C₃-C₆, C₄-C₆, or C₅-C₆). In embodiments, R^{1D} is independently unsubstituted cycloalkyl (e.g., C₃-C₈, C₃-C₆, C₄-C₆, or C₅-C₆). In embodiments, R^{1D} is independently substituted or unsubstituted heterocycloalkyl (e.g., 3 to 8 membered, 3 to 6 membered, 4 to 6 membered, 4 to 5 membered, or 5 to 6 membered). In embodiments, R^{1D} is independently substituted heterocycloalkyl (e.g., 3 to 8 membered, 3 to 6 membered, 4 to 6 membered, 4 to 5 membered, or 5 to 6 membered). In embodiments, R^{1D} is independently unsubstituted heterocycloalkyl (e.g., 3 to 8 membered, 3 to 6 membered, 4 to 6 membered, 4 to 5 membered, or 5 to 6 membered). In embodiments, R^{1D} is independently substituted or unsubstituted aryl (e.g., C₆-C₁₂, C₆-C₁₀, or phenyl). In embodiments, R^{1D} is independently substituted aryl (e.g., C₆-C₁₂, C₆-C₁₀, or phenyl). In embodiments, R^{1D} is independently unsubstituted aryl (e.g., C₆-C₁₂, C₆-C₁₀, or phenyl). In embodiments, R^{1D} is independently substituted or unsubstituted heteroaryl (e.g., 5 to 12 membered, 5 to 10 membered, 5 to 9 membered, or 5 to 6 membered). In embodiments, R^{1D} is independently substituted heteroaryl (e.g., 5 to 12 membered, 5 to 10 membered, 5 to 9 membered, or 5 to 6 membered). In embodiments, R^{1D} is independently unsubstituted heteroaryl (e.g., 5 to 12 membered, 5 to 10 membered, 5 to 9 membered, or 5 to 6 membered).

In embodiments, R¹ is independently

halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²⁰-substituted or unsubstituted alkyl (e.g., C₁-C₈, C₁-C₆, C₁-C₄, Or C₁-C₂), R²⁰-substituted or unsubstituted heteroalkyl (e.g., 2 to 8 membered, 2 to 6 membered, 4 to 6 membered, 2 to 3 membered, or 4 to 5 membered), R²⁰-substituted or unsubstituted cycloalkyl (e.g., C₃-C₈, C₃-C₆, C₄-C₆, or C₅-C₆), R²⁰-substituted or unsubstituted heterocycloalkyl (e.g., 3 to 8 membered, 3 to 6 membered, 4 to 6 membered, 4 to 5 membered, or 5 to 6 membered), R²⁰-substituted or unsubstituted aryl (e.g., C₆-C₁₂, C₆-C₁₀, or phenyl), or R²⁰-substituted or unsubstituted heteroaryl (e.g., 5 to 12 membered, 5 to 10 membered, 5 to 9 membered, or 5 to 6 membered). In embodiments, R¹ is independently
halogen, —CX¹₃, —CHX¹₂, —CH₂X¹, —OCX¹₃, —OCH₂X¹, —OCHX¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X¹ is independently —F, —Cl, —Br, or —I. In embodiments, R¹ is independently unsubstituted methyl. In embodiments, R¹ is independently unsubstituted ethyl.

In embodiments, two adjacent R¹ substituents may optionally be joined to form a R²⁰-substituted or unsubstituted cycloalkyl (e.g., C₃-C₈, C₃-C₆, C₄-C₆, or C₅-C₆). In embodiments, two adjacent R¹ substituents may optionally be joined to form a R²⁰-substituted cycloalkyl (e.g., C₃-C₈, C₃-C₆, C₄-C₆, or C₅-C₆). In embodiments, two adjacent R¹ substituents may optionally be joined to form an unsubstituted cycloalkyl (e.g., C₃-C₈, C₃-C₆, C₄-C₆, or C₅-C₆). In embodiments, two adjacent R¹ substituents may optionally be joined to form a R²⁰-substituted or unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a R²⁰-substituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form an unsubstituted heterocycloalkyl (e.g., 3 to 8 membered, 3 to 6 membered, 4 to 6 membered, 4 to 5 membered, or 5 to 6 membered). In embodiments, two adjacent R¹ substituents may optionally be joined to form a R²⁰-substituted or unsubstituted aryl (e.g., C₆-C₁₂, C₆-C₁₀, or phenyl). In embodiments, two adjacent R¹ substituents may optionally be joined to form a R²⁰-substituted aryl (e.g., C₆-C₁₂, C₆-C₁₀, or phenyl). In embodiments, two adjacent R¹ substituents may optionally be joined to form an unsubstituted aryl (e.g., C₆-C₁₂, C₆-C₁₀, or phenyl). In embodiments, two adjacent R¹ substituents may optionally be joined to form a R²⁰-substituted or unsubstituted heteroaryl (e.g., 5 to 12 membered, 5 to 10 membered, 5 to 9 membered, or 5 to 6 membered). In embodiments, two adjacent R¹ substituents may optionally be joined to form a R²⁰-substituted heteroaryl (e.g., 5 to 12 membered, 5 to 10 membered, 5 to 9 membered, or 5 to 6 membered). In embodiments, two adjacent R¹ substituents may optionally be joined to form an unsubstituted heteroaryl (e.g., 5 to 12 membered, 5 to 10 membered, 5 to 9 membered, or 5 to 6 membered).

R²⁰ is independently oxo,

halogen, —CX²⁰₃, —CHX²⁰₂, —CH₂X²⁰, —OCX²⁰₃, —OCH₂X²⁰, —OCHX²⁰₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, R²¹-substituted or unsubstituted alkyl (e.g., C₁-C₈, C₁-C₆, C₁-C₄, or C₁-C₂), R²¹-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R²¹-substituted or unsubstituted heterocycloalkyl (e.g., 3 to 8 membered, 3 to 6 membered, 4 to 6 membered, 4 to 5 membered, or 5 to 6 membered), R²¹-substituted or unsubstituted aryl (e.g., C₆-C₁₂, C₆-C₁₀, or phenyl), or R²¹-substituted or unsubstituted heteroaryl (e.g., 5 to 12 membered, 5 to 10 membered, 5 to 9 membered, or 5 to 6 membered). In embodiments, R²⁰ is independently oxo,
halogen, —CX²⁰₃, —CHX²⁰₂, —CH₂X²⁰, —OCX²⁰₃, —OCH₂X²⁰, —OCHX²⁰₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²⁰ is independently —F, —Cl, —Br, or —I. In embodiments, R²⁰ is independently unsubstituted methyl. In embodiments, R²⁰ is independently unsubstituted ethyl.

R²¹ is independently oxo,

halogen, —CX²¹₃, —CHX²¹₂, —CH₂X²¹, —OCX²¹₃, —OCH₂X²¹, —OCHX²¹₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R²²-substituted or unsubstituted alkyl (e.g., C₁-C₈, C₁-C₆, C₁-C₄, or C₁-C₂), R²²-substituted or unsubstituted heteroalkyl (e.g., 2 to 8 membered, 2 to 6 membered, 4 to 6 membered, 2 to 3 membered, or 4 to 5 membered), R²²-substituted or unsubstituted cycloalkyl (e.g., C₃-C₈, C₃-C₆, C₄-C₆, or C₅-C₆), R²²-substituted or unsubstituted heterocycloalkyl (e.g., 3 to 8 membered, 3 to 6 membered, 4 to 6 membered, 4 to 5 membered, or 5 to 6 membered), R²²-substituted or unsubstituted aryl (e.g., C₆-C₁₂, C₆-C₁₀, or phenyl), or R²²-substituted or unsubstituted heteroaryl (e.g., 5 to 12 membered, 5 to 10 membered, 5 to 9 membered, or 5 to 6 membered). In embodiments, R²¹ is independently oxo,
halogen, —CX²¹₃, —CHX²¹₂, —CH₂X²¹, —OCX²¹₃, —OCH₂X²¹, —OCHX²¹₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²¹ is independently —F, —Cl, —Br, or —I. In embodiments, R²¹ is independently unsubstituted methyl. In embodiments, R²¹ is independently unsubstituted ethyl.

R²² is independently oxo,

halogen, —CX²²₃, —CHX²²₂, —CH₂X²², —OCX²²₃, —OCH₂X²², —OCHX²²₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X²² is independently —F, —Cl, —Br, or —I. In embodiments, R²² is independently unsubstituted methyl. In embodiments, R²² is independently unsubstituted ethyl.

In embodiments, R^{1A} is independently

hydrogen, —CX^{1A}₃, —CHX^{1A}₂, —CH₂X^{1A}, —CN, —COOH, —CONH₂, R^{20A}-substituted or unsubstituted alkyl (e.g., C₁-C₈, C₁-C₆, C₁-C₄, or C₁-C₂), R^{20A}-substituted or unsubstituted heteroalkyl (e.g., 2 to 8 membered, 2 to 6 membered, 4 to 6 membered, 2 to 3 membered, or 4 to 5 membered), R^{20A}-substituted or unsubstituted cycloalkyl (e.g., C₃-C₈, C₃-C₆, C₄-C₆, or C₅-C₆), R^{20A}-substituted or unsubstituted heterocycloalkyl (e.g., 3 to 8 membered, 3 to 6 membered, 4 to 6 membered, 4 to 5 membered, or 5 to 6 membered), R^{20A}-substituted or unsubstituted aryl (e.g., C₆-C₁₂, C₆-C₁₀, or phenyl), or R^{20A}-substituted or unsubstituted heteroaryl (e.g., 5 to 12 membered, 5 to 10 membered, 5 to 9 membered, or 5 to 6 membered). In embodiments, R^{1A} is independently
hydrogen, —CX^{1A}₃, —CHX^{1A}₂, —CH₂X^{1A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1A} is independently —F, —Cl, —Br, or —I. In embodiments, R^{1A} is independently hydrogen. In embodiments, R^{1A} is independently unsubstituted methyl. In embodiments, R^{1A} is independently unsubstituted ethyl.

In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered) or R^{20A}-substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form a R^{20A}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{20A} is independently oxo,

halogen, —CX^{20A}₃, —CHX^{20A}₂, —CH₂X^{20A}, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{21A}-substituted or unsubstituted alkyl (e.g., C₁-C₈, C₁-C₆, C₁-C₄, or C₁-C₂), R^{21A}-substituted or unsubstituted heteroalkyl (e.g., 2 to 8 membered, 2 to 6 membered, 4 to 6 membered, 2 to 3 membered, or 4 to 5 membered), R^{21A}-substituted or unsubstituted cycloalkyl (e.g., C₃-C₈, C₃-C₆, C₄-C₆, or C₅-C₆), R^{21A}-substituted or unsubstituted heterocycloalkyl (e.g., 3 to 8 membered, 3 to 6 membered, 4 to 6 membered, 4 to 5 membered, or 5 to 6 membered), R^{21A}-substituted or unsubstituted aryl (e.g., C₆-C₁₂, C₆-C₁₀, or phenyl), or R^{21A}-substituted or unsubstituted heteroaryl (e.g., 5 to 12 membered, 5 to 10 membered, 5 to 9 membered, or 5 to 6 membered). In embodiments, R^{20A} is independently oxo,
halogen, —CX^{20A}₃, —CHX^{20A}₂, —CH₂X²⁰A, —OCX^{20A}₃, —OCH₂X^{20A}, —OCHX^{20A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20A} is independently —F, —Cl, —Br, or —I. In embodiments, R^{20A} is independently unsubstituted methyl. In embodiments, R^{20A} is independently unsubstituted ethyl.

R^{21A} is independently oxo,

halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{22A}-substituted or unsubstituted alkyl (e.g., C₁-C₈, C₁-C₆, C₁-C₄, or C₁-C₂), R^{22A}-substituted or unsubstituted heteroalkyl (e.g., 2 to 8 membered, 2 to 6 membered, 4 to 6 membered, 2 to 3 membered, or 4 to 5 membered), R^{22A}-substituted or unsubstituted cycloalkyl (e.g., C₃-C₈, C₃-C₆, C₄-C₆, or C₅-C₆), R^{22A}-substituted or unsubstituted heterocycloalkyl (e.g., 3 to 8 membered, 3 to 6 membered, 4 to 6 membered, 4 to 5 membered, or 5 to 6 membered), R^{22A}-substituted or unsubstituted aryl (e.g., C₆-C₁₂, C₆-C₁₀, or phenyl), or R^{22A}-substituted or unsubstituted heteroaryl (e.g., 5 to 12 membered, 5 to 10 membered, 5 to 9 membered, or 5 to 6 membered). In embodiments, R^{21A} is independently oxo, halogen, —CX^{21A}₃, —CHX^{21A}₂, —CH₂X^{21A}, —OCX^{21A}₃, —OCH₂X^{21A}, —OCHX^{21A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21A} is independently —F, —Cl, —Br, or —I. In embodiments, R^{21A} is independently unsubstituted methyl. In embodiments, R^{21A} is independently unsubstituted ethyl.

R^{22A} is independently oxo,

halogen, —CX^{22A}₃, —CHX^{22A}₂, —CH₂X^{22A}, —OCX^{22A}₃, —OCH₂X^{22A}, —OCHX^{22A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22A} is independently —F, —Cl, —Br, or —I. In embodiments, R^{22A} is independently unsubstituted methyl. In embodiments, R^{22A} is independently unsubstituted ethyl.

In embodiments, R^{1B} is independently

hydrogen, —CX^{1B}₃, —CHX^{1B}₂, —CH₂X^{1B}, —CN, —COOH, —CONH₂, R^{20B}-substituted or unsubstituted alkyl (e.g., C₁-C₈, C₁-C₆, C₁-C₄, or C₁-C₂), R^{20B}-substituted or unsubstituted heteroalkyl (e.g., 2 to 8 membered, 2 to 6 membered, 4 to 6 membered, 2 to 3 membered, or 4 to 5 membered), R^{20B}-substituted or unsubstituted cycloalkyl (e.g., C₃-C₈, C₃-C₆, C₄-C₆, or C₅-C₆), R^{20B}-substituted or unsubstituted heterocycloalkyl (e.g., 3 to 8 membered, 3 to 6 membered, 4 to 6 membered, 4 to 5 membered, or 5 to 6 membered), R^{20B}-substituted or unsubstituted aryl (e.g., C₆-C₁₂, C₆-C₁₀, or phenyl), or R^{20B}-substituted or unsubstituted heteroaryl (e.g., 5 to 12 membered, 5 to 10 membered, 5 to 9 membered, or 5 to 6 membered). In embodiments, R^{1B} is independently
hydrogen, —CX^{1B}₃, —CHX^{1B}₂, —CH₂X^{1B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1B} is independently —F, —Cl, —Br, or —I. In embodiments, R^{1B} is independently hydrogen. In embodiments, R^{1B} is independently unsubstituted methyl. In embodiments, R^{1B} is independently unsubstituted ethyl.

In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered) or R^{20B}-substituted or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 12 membered, 5 to 10 membered, 5 to 9 membered, or 5 to 6 membered). In embodiments, R^{1A} and R^{1B} substituents bonded to the same nitrogen atom may optionally be joined to form a R^{20B}-substituted or unsubstituted heterocycloalkyl (e.g., 3 to 8 membered, 3 to 6 membered, 4 to 6 membered, 4 to 5 membered, or 5 to 6 membered). In embodiments, R^{1A} and R^{1B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{20B} is independently oxo,

halogen, —CX^{20B}₃, —CHX^{20B}₂, —CH₂X^{20B}, —OCX^{20B}₃, —OCH₂X^{20B}, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{21B}-substituted or unsubstituted alkyl (e.g., C₁-C₈, C₁-C₆, C₁-C₄, or C₁-C₂), R^{21B}-substituted or unsubstituted heteroalkyl (e.g., 2 to 8 membered, 2 to 6 membered, 4 to 6 membered, 2 to 3 membered, or 4 to 5 membered), R^{21B}-substituted or unsubstituted cycloalkyl (e.g., C₃-C₈, C₃-C₆, C₄-C₆, or C₅-C₆), R^{21B}-substituted or unsubstituted heterocycloalkyl (e.g., 3 to 8 membered, 3 to 6 membered, 4 to 6 membered, 4 to 5 membered, or 5 to 6 membered), R^{21B}-substituted or unsubstituted aryl (e.g., C₆-C₁₂, C₆-C₁₀, or phenyl), or R^{21B}-substituted or unsubstituted heteroaryl (e.g., 5 to 12 membered, 5 to 10 membered, 5 to 9 membered, or 5 to 6 membered). In embodiments, R^{20B} is independently oxo,
halogen, —CX^{20B}₃, —CHX^{20B}₂, —CH₂X^{20B}, —OCX^{20B}₃, —OCH₂X^{20B}, —OCHX^{20B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20B} is independently —F, —Cl, —Br, or —I. In embodiments, R^{20B} is independently unsubstituted methyl. In embodiments, R^{20B} is independently unsubstituted ethyl.

R^{21B} is independently oxo,

halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{22B}-substituted or unsubstituted alkyl (e.g., C₁-C₈, C₁-C₆, C₁-C₄, or C₁-C₂), R^{22B}-substituted or unsubstituted heteroalkyl (e.g., 2 to 8 membered, 2 to 6 membered, 4 to 6 membered, 2 to 3 membered, or 4 to 5 membered), R^{22B}-substituted or unsubstituted cycloalkyl (e.g., C₃-C₈, C₃—C₆, C₄-C₆, or C₅-C₆), R^{22B}-substituted or unsubstituted heterocycloalkyl (e.g., 3 to 8 membered, 3 to 6 membered, 4 to 6 membered, 4 to 5 membered, or 5 to 6 membered), R^{22B}-substituted or unsubstituted aryl (e.g., C₆-C₁₂, C₆-C₁₀, or phenyl), or R^{22B}-substituted or unsubstituted heteroaryl (e.g., 5 to 12 membered, 5 to 10 membered, 5 to 9 membered, or 5 to 6 membered). In embodiments, R^{21B} is independently oxo,
halogen, —CX^{21B}₃, —CHX^{21B}₂, —CH₂X^{21B}, —OCX^{21B}₃, —OCH₂X^{21B}, —OCHX^{21B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21B} is independently —F, —Cl, —Br, or —I. In embodiments, R^{21B} is independently unsubstituted methyl. In embodiments, R^{21B} is independently unsubstituted ethyl.

R^{22B} is independently oxo,

halogen, —CX^{22B}₃, —CHX^{22B}₂, —CH₂X^{22B}, —OCX^{22B}₃, —OCH₂X^{22B}, —OCHX^{22B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22B} is independently —F, —Cl, —Br, or —I. In embodiments, R^{22B} is independently unsubstituted methyl. In embodiments, R^{22B} is independently unsubstituted ethyl.

In embodiments, R^{1C} is independently

hydrogen, —CX^{1C}₃, —CHX^{1C}₂, —CH₂X^{1C}, —CN, —COOH, —CONH₂, R^{20C}-substituted or unsubstituted alkyl (e.g., C₁-C₈, C₁-C₆, C₁-C₄, or C₁-C₂), R^{20C}-substituted or unsubstituted heteroalkyl (e.g., 2 to 8 membered, 2 to 6 membered, 4 to 6 membered, 2 to 3 membered, or 4 to 5 membered), R^{20C}-substituted or unsubstituted cycloalkyl (e.g., C₃-C₈, C₃-C₆, C₄-C₆, or C₅—C₆), R^{20C}-substituted or unsubstituted heterocycloalkyl (e.g., 3 to 8 membered, 3 to 6 membered, 4 to 6 membered, 4 to 5 membered, or 5 to 6 membered), R^{20C}-substituted or unsubstituted aryl (e.g., C₆-C₁₂, C₆-C₁₀, or phenyl), or R^{20C}-substituted or unsubstituted heteroaryl (e.g., 5 to 12 membered, 5 to 10 membered, 5 to 9 membered, or 5 to 6 membered). In embodiments, R^{1C} is independently
hydrogen, —CX^{1C}₃, —CHX^{1C}₂, —CH₂X^{1C}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1C} is independently —F, —Cl, —Br, or —I. In embodiments, R^{1C} is independently hydrogen. In embodiments, R^{1C} is independently unsubstituted methyl. In embodiments, R^{1C} is independently unsubstituted ethyl.

R^{20C} is independently oxo,

halogen, —CX^{20C}₃, —CHX^{20C}₂, —CH₂X^{20C}, —OCX^{20C}₃, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{21C}-substituted or unsubstituted alkyl (e.g., C₁-C₈, C₁-C₆, C₁-C₄, or C₁-C₂), R^{21C}-substituted or unsubstituted heteroalkyl (e.g., 2 to 8 membered, 2 to 6 membered, 4 to 6 membered, 2 to 3 membered, or 4 to 5 membered), R²¹-substituted or unsubstituted cycloalkyl (e.g., C₃-C₈, C₃-C₆, C₄-C₆, or C₅-C₆), R^{21C}-substituted or unsubstituted heterocycloalkyl (e.g., 3 to 8 membered, 3 to 6 membered, 4 to 6 membered, 4 to 5 membered, or 5 to 6 membered), R^{21C}-substituted or unsubstituted aryl (e.g., C₆-C₁₂, C₆-C₁₀, or phenyl), or R^{21C}-substituted or unsubstituted heteroaryl (e.g., 5 to 12 membered, 5 to 10 membered, 5 to 9 membered, or 5 to 6 membered). In embodiments, R^{20C} is independently oxo,
halogen, —CX^{20C}₃, —CHX^{20C}₂, —CH₂X^{20C}, —OCX^{20C}₃, —OCH₂X^{20C}, —OCHX^{20C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20C} is independently —F, —Cl, —Br, or —I. In embodiments, R^{20C} is independently unsubstituted methyl. In embodiments, R^{20C} is independently unsubstituted ethyl.

R^{21C} is independently oxo,

halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{22C}-substituted or unsubstituted alkyl (e.g., C₁-C₈, C₁-C₆, C₁-C₄, or C₁-C₂), R^{22C}-substituted or unsubstituted heteroalkyl (e.g., 2 to 8 membered, 2 to 6 membered, 4 to 6 membered, 2 to 3 membered, or 4 to 5 membered), R^{22C}-substituted or unsubstituted cycloalkyl (e.g., C₃-C₈, C₃-C₆, C₄-C₆, or C₅-C₆), R^{22C}-substituted or unsubstituted heterocycloalkyl (e.g., 3 to 8 membered, 3 to 6 membered, 4 to 6 membered, 4 to 5 membered, or 5 to 6 membered), R^{22C}-substituted or unsubstituted aryl (e.g., C₆-C₁₂, C₆-C₁₀, or phenyl), or R^{22C}-substituted or unsubstituted heteroaryl (e.g., 5 to 12 membered, 5 to 10 membered, 5 to 9 membered, or 5 to 6 membered). In embodiments, R^{21C} is independently oxo,
halogen, —CX^{21C}₃, —CHX^{21C}₂, —CH₂X^{21C}, —OCX^{21C}₃, —OCH₂X^{21C}, —OCHX^{21C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21C} is independently —F, —Cl, —Br, or —I. In embodiments, R^{21C} is independently unsubstituted methyl. In embodiments, R^{21C} is independently unsubstituted ethyl.

R^{22C} is independently oxo,

halogen, —CX^{22C}₃, —CHX^{22C}₂, —CH₂X^{22C}, —OCX²²C₃, —OCH₂X^{22C}, —OCHX^{22C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22C} is independently —F, —Cl, —Br, or —I. In embodiments, R^{22C} is independently unsubstituted methyl. In embodiments, R^{22C} is independently unsubstituted ethyl.

In embodiments, R^{1D} is independently

hydrogen, —CX^{1D}₃, —CHX^{1D}₂, —CH₂X^{1D}, —CN, —COOH, —CONH₂, R^{20D}-substituted or unsubstituted alkyl (e.g., C₁-C₈, C₁-C₆, C₁-C₄, or C₁-C₂), R^{20D}-substituted or unsubstituted heteroalkyl (e.g., 2 to 8 membered, 2 to 6 membered, 4 to 6 membered, 2 to 3 membered, or 4 to 5 membered), R^{20D}-substituted or unsubstituted cycloalkyl (e.g., C₃-C₈, C₃-C₆, C₄-C₆, or C₅-C₆), R^{20D}-substituted or unsubstituted heterocycloalkyl (e.g., 3 to 8 membered, 3 to 6 membered, 4 to 6 membered, 4 to 5 membered, or 5 to 6 membered), R^{20D}-substituted or unsubstituted aryl (e.g., C₆-C₁₂, C₆-C₁₀, or phenyl), or R^{20D}-substituted or unsubstituted heteroaryl (e.g., 5 to 12 membered, 5 to 10 membered, 5 to 9 membered, or 5 to 6 membered). In embodiments, R^{1D} is independently
hydrogen, —CX^{1D}₃, —CHX^{1D}₂, —CH₂X^{1D}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{1D} is independently —F, —Cl, —Br, or —I. In embodiments, R^{1D} is independently hydrogen. In embodiments, R^{1D} is independently unsubstituted methyl. In embodiments, R^{1D} is independently unsubstituted ethyl.

R^{20D} is independently oxo,

halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{21D}-substituted or unsubstituted alkyl (e.g., C₁-C₈, C₁-C₆, C₁-C₄, or C₁-C₂), R^{21D}-substituted or unsubstituted heteroalkyl (e.g., 2 to 8 membered, 2 to 6 membered, 4 to 6 membered, 2 to 3 membered, or 4 to 5 membered), R^{21D}-substituted or unsubstituted cycloalkyl (e.g., C₃-C₈, C₃—C₆, C₄-C₆, or C₅-C₆), R^{21D}-substituted or unsubstituted heterocycloalkyl (e.g., 3 to 8 membered, 3 to 6 membered, 4 to 6 membered, 4 to 5 membered, or 5 to 6 membered), R^{21D}-substituted or unsubstituted aryl (e.g., C₆-C₁₂, C₆-C₁₀, or phenyl), or R^{21D}-substituted or unsubstituted heteroaryl (e.g., 5 to 12 membered, 5 to 10 membered, 5 to 9 membered, or 5 to 6 membered). In embodiments, R^{20D} is independently oxo,
halogen, —CX^{20D}₃, —CHX^{20D}₂, —CH₂X^{20D}, —OCX^{20D}₃, —OCH₂X^{20D}, —OCHX^{20D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{20D} is independently —F, —Cl, —Br, or —I. In embodiments, R^{20D} is independently unsubstituted methyl. In embodiments, R^{20D} is independently unsubstituted ethyl.

R^{21D} is independently oxo,

halogen, —CX^{21D}₃, —CHX^{21D}₂, —CH₂X^{21D}, —OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{22D}-substituted or unsubstituted alkyl (e.g., C₁-C₈, C₁-C₆, C₁-C₄, or C₁-C₂), R^{22D}-substituted or unsubstituted heteroalkyl (e.g., 2 to 8 membered, 2 to 6 membered, 4 to 6 membered, 2 to 3 membered, or 4 to 5 membered), R^{22D}-substituted or unsubstituted cycloalkyl (e.g., C₃-C₈, C₃-C₆, C₄-C₆, or C₅-C₆), R^{22D}-substituted or unsubstituted heterocycloalkyl (e.g., 3 to 8 membered, 3 to 6 membered, 4 to 6 membered, 4 to 5 membered, or 5 to 6 membered), R^{22D}-substituted or unsubstituted aryl (e.g., C₆-C₁₂, C₆-C₁₀, or phenyl), or R^{22D}-substituted or unsubstituted heteroaryl (e.g., 5 to 12 membered, 5 to 10 membered, 5 to 9 membered, or 5 to 6 membered). In embodiments, R^{21D} is independently oxo,
halogen, —CX^{21D}₃, —CHX^{21D}₂, —CH₂X^{21D}, —OCX^{21D}₃, —OCH₂X^{21D}, —OCHX^{21D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{21D} is independently —F, —Cl, —Br, or —I. In embodiments, R^{21D} is independently unsubstituted methyl. In embodiments, R^{21D} is independently unsubstituted ethyl.

R^{22D} is independently oxo,

halogen, —CX^{22D}₃, —CHX^{22D}₂, —CH₂X^{22D}, —OCX^{22D}₃, —OCH₂X^{22D}, —OCHX^{22D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{22D} is independently —F, —Cl, —Br, or —I. In embodiments, R^{22D} is independently unsubstituted methyl. In embodiments, R^{22D} is independently unsubstituted ethyl.

In embodiments, z1 is 0. In embodiments, z1 is 0 and the compound has the formula of

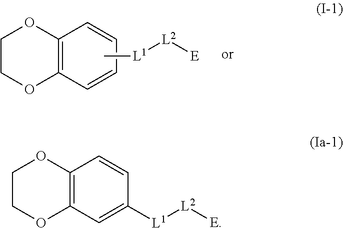

In embodiments, z1 is 0 and the compound has the formula of

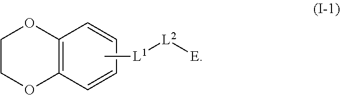

In embodiments, z1 is 0 and the compound has the formula of

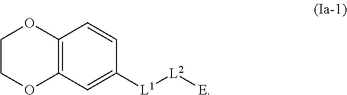

In embodiments, z1 is 0 and the compound has the formula of

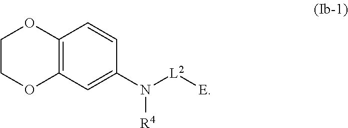

In embodiments, z1 is 0 and the compound has the formula of

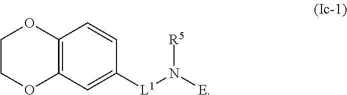

In embodiments, z is 0 and the compound has the formula of

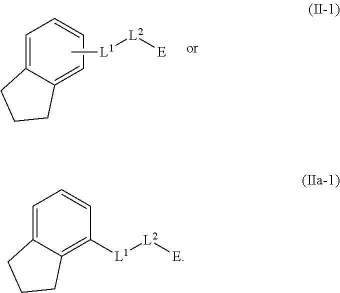

In embodiments, z1 is 0 and the compound has the formula of

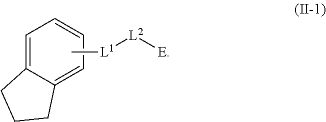

In embodiments, z1 is 0 and the compound has the formula of

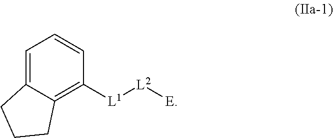

In embodiments, z1 is 0 and the compound has the formula of

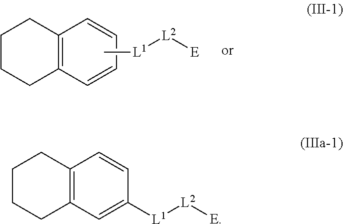

In embodiments, z1 is 0 and the compound has the formula of

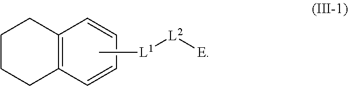

In embodiments, z1 is 0 and the compound has the formula of

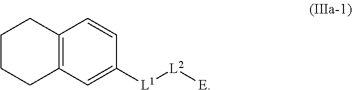

In embodiments, z1 is 0 and the compound has the formula of

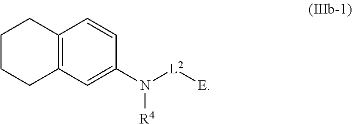

In embodiments, z1 is 0 and the compound has the formula of

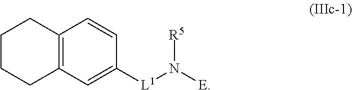

In embodiments, z1 is 1. In embodiments, z1 is 2. In embodiments, z1 is 3. In embodiments, z1 is 4. In embodiments, z1 is 5.

In embodiments, the compound has the formula:

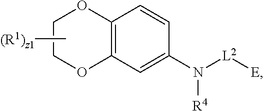

wherein R⁴ substituted or unsubstituted C₁-C₈ alkyl. In embodiments, R⁴ is independently unsubstituted C₁-C₈ alkyl. In embodiments, R⁴ is phenyl-substituted methyl. In embodiments, R⁴ is unsubstituted benzyl. In embodiments, the compound has the formula:

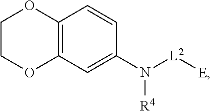

wherein R⁴ is substituted or unsubstituted C₁-C₈ alkyl. In embodiments, the compound has the formula:

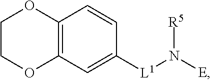

wherein R⁵ is substituted or unsubstituted C₁-C₈ alkyl. In embodiments, the compound has the formula:

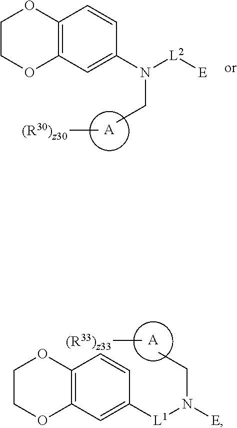

wherein Ring A is independently substituted or unsubstituted cycloalkyl, substituted or unsubstituted heterocycloalkyl, substituted or unsubstituted aryl, or substituted or unsubstituted heteroaryl; and z30 and z33 are independently an integer from 0 to 10. In embodiments, z30 is 0. In embodiments, z30 is 1. In embodiments, z30 is 2. In embodiments, z30 is 3. In embodiments, z30 is 4. In embodiments, z30 is 5. In embodiments, z30 is 6. In embodiments, z30 is 7. In embodiments, z30 is 8. In embodiments, z30 is 9. In embodiments, z30 is 10. In embodiments, z33 is 0. In embodiments, z33 is 1. In embodiments, z33 is 2. In embodiments, z33 is 3. In embodiments, z33 is 4. In embodiments, z33 is 5. In embodiments, z33 is 6. In embodiments, z33 is 7. In embodiments, z33 is 8. In embodiments, z33 is 9. In embodiments, z33 is 10.

In embodiments, the compound has the formula:

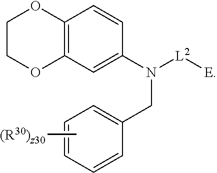

In embodiments, the compound has the formula:

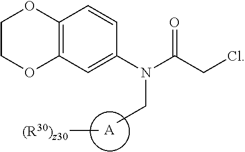

In embodiments, the compound has the formula:

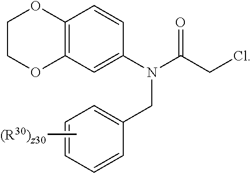

In embodiments, the compound has the formula:

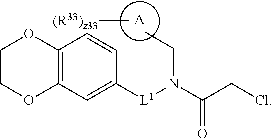

In embodiments, the compound has the formula:

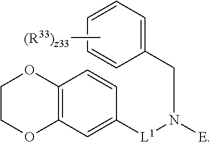

In embodiments, the compound has the formula:

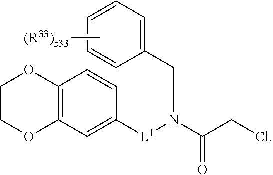

In embodiments, Ring A is substituted or unsubstituted cycloalkyl. In embodiments, Ring A is substituted or unsubstituted heterocycloalkyl. In embodiments, Ring A is substituted or unsubstituted aryl. In embodiments, Ring A is substituted or unsubstituted heteroaryl. In embodiments, Ring A is substituted or unsubstituted (C₃-C₁₀) cycloalkyl, substituted or unsubstituted 3 to 10 membered heterocycloalkyl, substituted or unsubstituted (C₆-C₁₀) aryl, or substituted or unsubstituted 5 to 10 membered heteroaryl. In embodiments, Ring A is substituted or unsubstituted (C₃-C₁₀) cycloalkyl. In embodiments, Ring A is substituted or unsubstituted 3 to 10 membered heterocycloalkyl. In embodiments, Ring A is substituted or unsubstituted (C₆-C₁₀) aryl. In embodiments, Ring A is substituted or unsubstituted 5 to 10 membered heteroaryl. In embodiments, Ring A is substituted or unsubstituted (C₃-C₆) cycloalkyl. In embodiments, Ring A is substituted or unsubstituted 3 to 6 membered heterocycloalkyl. In embodiments, Ring A is substituted or unsubstituted phenyl. In embodiments, Ring A is substituted or unsubstituted naphthyl. In embodiments, Ring A is substituted or unsubstituted 5 to 9 membered heteroaryl. In embodiments, Ring A is substituted or unsubstituted 5 to 6 membered heteroaryl. In embodiments, Ring A is an unsubstituted 5 to 6 membered heteroaryl. In embodiments, Ring A is substituted or unsubstituted 5 membered heteroaryl. In embodiments, Ring A is a substituted 5 membered heteroaryl. In embodiments, Ring A is an unsubstituted 5 membered heteroaryl.

In embodiments, Ring A is R³⁰-substituted or unsubstituted (C₃-C₁₀) cycloalkyl, R³⁰-substituted or unsubstituted 5 to 10 membered heterocycloalkyl, R³⁰-substituted or unsubstituted (C₆-C₁₀) aryl, or R³⁰-substituted or unsubstituted 5 to 10 membered heteroaryl. In embodiments, Ring A is R³⁰-substituted or unsubstituted (C₃-C₁₀) cycloalkyl or R³⁰-substituted or unsubstituted 5 to 10 membered heterocycloalkyl. In embodiments, Ring A is R³⁰-substituted or unsubstituted (C₃-C₁₀) cycloalkyl. In embodiments, Ring A is R³⁰-substituted or unsubstituted 3 to 10 membered heterocycloalkyl. In embodiments, Ring A is R³⁰-substituted or unsubstituted (C₆-C₁₀) aryl. In embodiments, Ring A is R³⁰-substituted or unsubstituted 5 to 10 membered heteroaryl. In embodiments, Ring A is R³⁰-substituted or unsubstituted (C₃-C₆) cycloalkyl. In embodiments, Ring A is R³⁰-substituted or unsubstituted 3 to 6 membered heterocycloalkyl. In embodiments, Ring A is R³⁰-substituted or unsubstituted phenyl. In embodiments, Ring A is R³⁰-substituted or unsubstituted naphthyl. In embodiments, Ring A is R³⁰-substituted or unsubstituted 5 to 9 membered heteroaryl. In embodiments, Ring A is R³⁰-substituted or unsubstituted 5 to 6 membered heteroaryl. In embodiments, Ring A is R³⁰-substituted or unsubstituted thienyl. In embodiments, Ring A is R³⁰-substituted or unsubstituted phenyl. In embodiments, Ring A is R³⁰-substituted or unsubstituted benzothienyl. In embodiments, Ring A is R³⁰-substituted or unsubstituted naphthyl. In embodiments, Ring A is R³⁰-substituted or unsubstituted benzofuranyl. In embodiments, Ring A is R³⁰-substituted or unsubstituted furanyl. In embodiments, Ring A is R³⁰-substituted or unsubstituted pyrrolyl.

In embodiments, Ring A is substituted cycloalkyl. In embodiments, Ring A is substituted heterocycloalkyl. In embodiments, Ring A is substituted aryl. In embodiments, Ring A is substituted heteroaryl. In embodiments, Ring A is substituted (C₃-C₁₀) cycloalkyl, substituted 3 to 10 membered heterocycloalkyl, substituted (C₆-C₁₀) aryl, or substituted 5 to 10 membered heteroaryl. In embodiments, Ring A is substituted (C₃-C₁₀) cycloalkyl. In embodiments, Ring A is substituted 3 to 10 membered heterocycloalkyl. In embodiments, Ring A is substituted (C₆-C₁₀) aryl. In embodiments, Ring A is substituted 5 to 10 membered heteroaryl. In embodiments, Ring A is substituted (C₃-C₆) cycloalkyl. In embodiments, Ring A is substituted 3 to 6 membered heterocycloalkyl. In embodiments, Ring A is substituted phenyl. In embodiments, Ring A is substituted naphthyl. In embodiments, Ring A is substituted 5 to 9 membered heteroaryl. In embodiments, Ring A is substituted 5 to 6 membered heteroaryl. In embodiments, Ring A is R³⁰-substituted (C₃-C₁₀) cycloalkyl, R³⁰-substituted 5 to 10 membered heterocycloalkyl, R³⁰-substituted (C₆-C₁₀) aryl, or R³⁰-substituted 5 to 10 membered heteroaryl. In embodiments, Ring A is R³⁰-substituted (C₃-C₁₀) cycloalkyl or R³⁰-substituted 5 to 10 membered heterocycloalkyl. In embodiments, Ring A is R³⁰-substituted (C₃-C₁₀) cycloalkyl. In embodiments, Ring A is R³⁰-substituted 3 to 10 membered heterocycloalkyl. In embodiments, Ring A is R³⁰-substituted (C₆-C₁₀) aryl. In embodiments, Ring A is R³⁰-substituted 5 to 10 membered heteroaryl. In embodiments, Ring A is R³⁰-substituted (C₃-C₆) cycloalkyl. In embodiments, Ring A is R³⁰-substituted 3 to 6 membered heterocycloalkyl. In embodiments, Ring A is R³⁰-substituted phenyl. In embodiments, Ring A is R³⁰-substituted naphthyl. In embodiments, Ring A is R³⁰-substituted 5 to 9 membered heteroaryl. In embodiments, Ring A is R³⁰-substituted 5 to 6 membered heteroaryl. In embodiments, Ring A is R³⁰-substituted thienyl. In embodiments, Ring A is R³⁰-substituted phenyl. In embodiments, Ring A is R³⁰-substituted benzothienyl. In embodiments, Ring A is R³⁰-substituted naphthyl. In embodiments, Ring A is R³⁰-substituted benzofuranyl. In embodiments, Ring A is R³⁰-substituted furanyl. In embodiments, Ring A is R³⁰-substituted pyrrolyl. In embodiments, Ring A is R³⁰-substituted 2,3-dihydro-1H-indenyl.

In embodiments, Ring A is unsubstituted cycloalkyl. In embodiments, Ring A is unsubstituted heterocycloalkyl. In embodiments, Ring A is unsubstituted aryl. In embodiments, Ring A is unsubstituted heteroaryl. In embodiments, Ring A is unsubstituted (C₃-C₁₀) cycloalkyl, unsubstituted 3 to 10 membered heterocycloalkyl, unsubstituted (C₆-C₁₀) aryl, or unsubstituted 5 to 10 membered heteroaryl. In embodiments, Ring A is unsubstituted (C₃-C₁₀) cycloalkyl. In embodiments, Ring A is unsubstituted 3 to 10 membered heterocycloalkyl. In embodiments, Ring A is unsubstituted (C₆-C₁₀) aryl. In embodiments, Ring A is unsubstituted 5 to 10 membered heteroaryl. In embodiments, Ring A is unsubstituted (C₃-C₆) cycloalkyl. In embodiments, Ring A is unsubstituted 3 to 6 membered heterocycloalkyl. In embodiments, Ring A is unsubstituted phenyl. In embodiments, Ring A is unsubstituted naphthyl. In embodiments, Ring A is unsubstituted 5 to 9 membered heteroaryl. In embodiments, Ring A is unsubstituted 5 to 6 membered heteroaryl. In embodiments, Ring A is unsubstituted (C₃-C₁₀) cycloalkyl, unsubstituted 5 to 10 membered heterocycloalkyl, unsubstituted (C₆-C₁₀) aryl, or unsubstituted 5 to 10 membered heteroaryl. In embodiments, Ring A is unsubstituted (C₃-C₁₀) cycloalkyl or unsubstituted 5 to 10 membered heterocycloalkyl. In embodiments, Ring A is unsubstituted (C₃-C₁₀) cycloalkyl. In embodiments, Ring A is unsubstituted 3 to 10 membered heterocycloalkyl. In embodiments, Ring A is unsubstituted (C₆-C₁₀) aryl. In embodiments, Ring A is unsubstituted 5 to 10 membered heteroaryl. In embodiments, Ring A is unsubstituted (C₃-C₆) cycloalkyl. In embodiments, Ring A is unsubstituted 3 to 6 membered heterocycloalkyl. In embodiments, Ring A is unsubstituted phenyl. In embodiments, Ring A is unsubstituted naphthyl. In embodiments, Ring A is unsubstituted 5 to 9 membered heteroaryl. In embodiments, Ring A is unsubstituted 5 to 6 membered heteroaryl. In embodiments, Ring A is unsubstituted thienyl. In embodiments, Ring A is unsubstituted phenyl. In embodiments, Ring A is unsubstituted benzothienyl. In embodiments, Ring A is unsubstituted naphthyl. In embodiments, Ring A is unsubstituted benzofuranyl. In embodiments, Ring A is unsubstituted furanyl. In embodiments, Ring A is unsubstituted pyrrolyl.

Ring A may be substituted with one R³⁰. Ring A may be substituted with two optionally different R³⁰ substituents. Ring A may be substituted with three optionally different R³⁰ substituents. Ring A may be substituted with four optionally different R³⁰ substituents. Ring A may be substituted with five optionally different R³⁰ substituents. Ring A may be substituted with six optionally different R³⁰ substituents. Ring A may be substituted with seven optionally different R³⁰ substituents. Ring A may be substituted with eight optionally different R³⁰ substituents. Ring A may be substituted with nine optionally different R³⁰ substituents. Ring A may be substituted with ten optionally different R³⁰ substituents.

In embodiments, L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene. In embodiments, L¹ is a bond.

In embodiments, L¹ is a bond. In embodiments, L¹ is —S(O)₂—. In embodiments, L¹ is —NR⁶—. In embodiments, L¹ is —O—. In embodiments, L¹ is —S—. In embodiments, L¹ is —C(O)—. In embodiments, L¹ is —C(O)NR⁶—. In embodiments, L¹ is —NR⁶C(O)—. In embodiments, L¹ is —NR⁶C(O)NH—. In embodiments, L¹ is —NHC(O)NR⁶—. In embodiments, L¹ is —C(O)O—. In embodiments, L¹ is —OC(O)—. In embodiments, L¹ is —NH—. In embodiments, L¹ is —C(O)NH—. In embodiments, L¹ is —NHC(O)—. In embodiments, L¹ is —NHC(O)NH—. In embodiments, L¹ is —CH₂—. In embodiments, L¹ is —OCH₂—. In embodiments, L¹ is —CH₂O—. In embodiments, L¹ is —CH₂CH₂—. In embodiments, L¹ is —NHCH₂—. In embodiments, L¹ is —CH₂NH—. In embodiments, L¹ is a bond.

In embodiments, L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

In embodiments, L¹ is independently substituted or unsubstituted alkylene (e.g., C₁-C₈, C₁-C₆, C₁-C₄, or C₁-C₂). In embodiments, L¹ is independently substituted alkylene (e.g., C₁-C₈, C₁-C₆, C₁-C₄, or C₁-C₂). In embodiments, L¹ is independently unsubstituted alkylene (e.g., C₁-C₈, C₁-C₆, C₁-C₄, or C₁-C₂). In embodiments, L¹ is independently unsubstituted methylene. In embodiments, L¹ is independently unsubstituted ethylene. In embodiments, L¹ is independently unsubstituted propylene. In embodiments, L¹ is independently unsubstituted isopropylene. In embodiments, L¹ is independently unsubstituted tert-butylene. In embodiments, L¹ is independently substituted or unsubstituted heteroalkylene (e.g., 2 to 8 membered, 2 to 6 membered, 4 to 6 membered, 2 to 3 membered, or 4 to 5 membered). In embodiments, L¹ is independently substituted heteroalkylene (e.g., 2 to 8 membered, 2 to 6 membered, 4 to 6 membered, 2 to 3 membered, or 4 to 5 membered). In embodiments, L¹ is independently unsubstituted heteroalkylene (e.g., 2 to 8 membered, 2 to 6 membered, 4 to 6 membered, 2 to 3 membered, or 4 to 5 membered). In embodiments, L¹ is independently substituted or unsubstituted cycloalkylene (e.g., C₃-C₈, C₃-C₆, C₄-C₆, or C₅-C₆). In embodiments, L¹ is independently substituted cycloalkylene (e.g., C₃-C₈, C₃-C₆, C₄-C₆, or C₅-C₆). In embodiments, L¹ is independently unsubstituted cycloalkylene (e.g., C₃-C₈, C₃-C₆, C₄-C₆, or C₅-C₆). In embodiments, L¹ is independently substituted or unsubstituted heterocycloalkylene (e.g., 3 to 8 membered, 3 to 6 membered, 4 to 6 membered, 4 to 5 membered, or 5 to 6 membered). In embodiments, L¹ is independently substituted heterocycloalkylene (e.g., 3 to 8 membered, 3 to 6 membered, 4 to 6 membered, 4 to 5 membered, or 5 to 6 membered). In embodiments, L¹ is independently unsubstituted heterocycloalkylene (e.g., 3 to 8 membered, 3 to 6 membered, 4 to 6 membered, 4 to 5 membered, or 5 to 6 membered). In embodiments, L¹ is independently substituted or unsubstituted arylene (e.g., C₆-C₁₀ or phenylene). In embodiments, L¹ is independently substituted arylene (e.g., C₆-C₁₀ or phenylene). In embodiments, L¹ is independently unsubstituted arylene (e.g., C₆-C₁₀ or phenylene). In embodiments, L¹ is independently substituted or unsubstituted heteroarylene (e.g., 5 to 10 membered, 5 to 9 membered, or 5 to 6 membered). In embodiments, L¹ is independently substituted heteroarylene (e.g., 5 to 10 membered, 5 to 9 membered, or 5 to 6 membered). In embodiments, L¹ is independently unsubstituted heteroarylene (e.g., 5 to 10 membered, 5 to 9 membered, or 5 to 6 membered).

In embodiments, L¹ is independently

bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, R³⁵-substituted or unsubstituted alkylene (e.g., C₁-C₈, C₁-C₆, C₁-C₄, or C₁-C₂), R³⁵-substituted or unsubstituted heteroalkylene (e.g., 2 to 8 membered, 2 to 6 membered, 4 to 6 membered, 2 to 3 membered, or 4 to 5 membered), R³⁵-substituted or unsubstituted cycloalkylene (e.g., C₃-C₈, C₃-C₆, C₄-C₆, or C₅-C₆), R³⁵-substituted or unsubstituted heterocycloalkylene (e.g., 3 to 8 membered, 3 to 6 membered, 4 to 6 membered, 4 to 5 membered, or 5 to 6 membered), R³⁵-substituted or unsubstituted arylene (e.g., C₆-C₁₀ or phenylene), or R³⁵-substituted or unsubstituted heteroarylene (e.g., 5 to 10 membered, 5 to 9 membered, or 5 to 6 membered). In embodiments, L¹ is independently
bond, —S(O)₂—, —N(R⁴)—, —O—, —S—, —C(O)—, —C(O)N(R⁴)—, —N(R⁴)C(O)—, —N(R⁴)C(O)NH—, —NHC(O)N(R⁴)—, —C(O)O—, —OC(O)—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₃-C₆, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenylene), or unsubstituted heteroarylene (e.g., 5 to 10 membered, 5 to 9 membered, or 5 to 6 membered). In embodiments, L¹ is independently unsubstituted methylene. In embodiments, L¹ is independently unsubstituted ethylene. In embodiments, L¹ is independently methyl-substituted methylene.

R³⁵ is independently oxo,

halogen, —CX³⁵₃, —CHX³⁵₂, —CH₂X³⁵, —OCX³⁵₃, —OCH₂X³⁵, —OCHX³⁵₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³⁶-substituted or unsubstituted alkyl (e.g., C₁-C₈, C₁-C₆, C₁-C₄, or C₁-C₂), R³⁶-substituted or unsubstituted heteroalkyl (e.g., 2 to 8 membered, 2 to 6 membered, 4 to 6 membered, 2 to 3 membered, or 4 to 5 membered), R³⁶-substituted or unsubstituted cycloalkyl (e.g., C₃-C₈, C₃-C₆, C₄-C₆, or C₅-C₆), R³⁶-substituted or unsubstituted heterocycloalkyl (e.g., 3 to 8 membered, 3 to 6 membered, 4 to 6 membered, 4 to 5 membered, or 5 to 6 membered), R³⁶-substituted or unsubstituted aryl (e.g., C₆-C₁₀ or phenyl), or R³⁶-substituted or unsubstituted heteroaryl (e.g., 5 to 10 membered, 5 to 9 membered, or 5 to 6 membered). In embodiments, R³⁵ is independently oxo, halogen, —CX³⁵₃, —CHX³⁵₂, —CH₂X³⁵, —OCX³⁵₃, —OCH₂X³⁵, —OCHX³⁵₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁵ is independently —F, —Cl, —Br, or —I. In embodiments, R³⁵ is independently unsubstituted methyl. In embodiments, R³⁵ is independently unsubstituted ethyl.

R³⁶ is independently oxo,

halogen, —CX³⁶₃, —CHX³⁶₂, —CH₂X³⁶, —OCX³⁶₃, —OCH₂X³⁶, —OCHX³⁶₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³⁷-substituted or unsubstituted alkyl (e.g., C₁-C₈, C₁-C₆, C₁-C₄, or C₁-C₂), R³⁷-substituted or unsubstituted heteroalkyl (e.g., 2 to 8 membered, 2 to 6 membered, 4 to 6 membered, 2 to 3 membered, or 4 to 5 membered), R³⁷-substituted or unsubstituted cycloalkyl (e.g., C₃-C₈, C₃-C₆, C₄-C₆, or C₅-C₆), R³⁷-substituted or unsubstituted heterocycloalkyl (e.g., 3 to 8 membered, 3 to 6 membered, 4 to 6 membered, 4 to 5 membered, or 5 to 6 membered), R³⁷-substituted or unsubstituted aryl (e.g., C₆-C₁₀ or phenyl), or R³⁷-substituted or unsubstituted heteroaryl (e.g., 5 to 10 membered, 5 to 9 membered, or 5 to 6 membered). In embodiments, R³⁶ is independently oxo,
halogen, —CX³⁶₃, —CHX³⁶₂, —CH₂X³⁶, —OCX³⁶₃, —OCH₂X³⁶, —OCHX³⁶₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁶ is independently —F, —Cl, —Br, or —I. In embodiments, R³⁶ is independently unsubstituted methyl. In embodiments, R³⁶ is independently unsubstituted ethyl.

R³⁷ is independently oxo,

halogen, —CX³⁷₃, —CHX³⁷₂, —CH₂X³⁷, —OCX³⁷₃, —OCH₂X³⁷, —OCHX³⁷₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁷ is independently —F, —Cl, —Br, or —I. In embodiments, R³⁷ is independently unsubstituted methyl. In embodiments, R³⁷ is independently unsubstituted ethyl.

In embodiments, R⁴ is independently hydrogen, —CX⁴₃, —CHX⁴₂, —CH₂X⁴, —OCX⁴₃, —OCH₂X⁴, —OCHX⁴₂, —CN, —C(O)R^{4A}, —C(O)OR^{4A}, —C(O)NR^{4A}R^{4B}, —OR^{4A}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁴ is independently hydrogen. In embodiments, R⁴ is independently —CX⁴₃. In embodiments, R⁴ is independently —CHX⁴₂. In embodiments, R⁴ is independently —CH₂X⁴. In embodiments, R⁴ is independently —CN. In embodiments, R⁴ is independently —C(O)R^{4A}. In embodiments, R⁴ is independently —C(O)—OR^{4A}. In embodiments, R⁴ is independently —C(O)NR^{4A}R^{4B}. In embodiments, R⁴ is independently —COOH. In embodiments, R⁴ is independently —CONH₂. In embodiments, R⁴ is independently —CF₃. In embodiments, R⁴ is independently —CHF₂. In embodiments, R⁴ is independently —CH₂F. In embodiments, R⁴ is independently —CH₃. In embodiments, R⁴ is independently —CH₂CH₃. In embodiments, R⁴ is independently —CH₂CH₂CH₃. In embodiments, R⁴ is independently —CH(CH₃)₂. In embodiments, R⁴ is independently —C(CH₃)₃.

In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted isopropyl. In embodiments, R⁴ is independently unsubstituted n-propyl. In embodiments, R⁴ is independently unsubstituted butyl. In embodiments, R⁴ is independently unsubstituted n-butyl. In embodiments, R⁴ is independently unsubstituted t-butyl. In embodiments, R⁴ is independently unsubstituted pentyl. In embodiments, R⁴ is independently unsubstituted n-pentyl. In embodiments, R⁴ is independently unsubstituted hexyl. In embodiments, R⁴ is independently unsubstituted n-hexyl. In embodiments, R⁴ is independently unsubstituted heptyl. In embodiments, R⁴ is independently unsubstituted n-heptyl. In embodiments, R⁴ is independently unsubstituted octyl. In embodiments, R⁴ is independently unsubstituted n-octyl. In embodiments, R⁴ is independently unsubstituted benzyl. In embodiments, R⁴ is independently unsubstituted C₁-C₈ alkyl. In embodiments, R⁴ is independently halo-substituted methyl. In embodiments, R⁴ is independently halo-substituted ethyl. In embodiments, R⁴ is independently halo-substituted isopropyl. In embodiments, R⁴ is independently halo-substituted n-propyl. In embodiments, R⁴ is independently halo-substituted n-butyl. In embodiments, R⁴ is independently halo-substituted t-butyl. In embodiments, R¹ is independently halo-substituted n-pentyl. In embodiments, R⁴ is independently halo-substituted benzyl. In embodiments, R⁴ is independently halo-substituted C₁-C₈ alkyl. In embodiments, R⁴ is independently unsubstituted 2 to 6 membered heteroalkyl. In embodiments, R⁴ is independently unsubstituted 2 to 7 membered heteroalkyl. In embodiments, R⁴ is independently unsubstituted 2 to 8 membered heteroalkyl. In embodiments, R⁴ is independently unsubstituted 2 to 9 membered heteroalkyl. In embodiments, R⁴ is independently unsubstituted 2 to 10 membered heteroalkyl. In embodiments, R⁴ is independently unsubstituted 3 to 10 membered heteroalkyl. In embodiments, R⁴ is independently unsubstituted 4 to 10 membered heteroalkyl. In embodiments, R⁴ is independently unsubstituted 5 to 10 membered heteroalkyl. In embodiments, R⁴ is independently unsubstituted 6 to 10 membered heteroalkyl. In embodiments, R⁴ is independently unsubstituted 7 to 10 membered heteroalkyl. In embodiments, R⁴ is independently unsubstituted 8 to 10 membered heteroalkyl. In embodiments, R⁴ is independently unsubstituted 6 to 10 membered heteroalkyl. In embodiments, R⁴ is independently unsubstituted 7 to 9 membered heteroalkyl.

In embodiments, R⁴ is independently substituted or unsubstituted alkyl (e.g., C₁-C₈, C₁-C₆, C₁-C₄, or C₁-C₂). In embodiments, R⁴ is independently substituted alkyl (e.g., C₁-C₈, C₁-C₆, C₁-C₄, or C₁-C₂). In embodiments, R⁴ is independently unsubstituted alkyl (e.g., C₁-C₈, C₁-C₆, C₁-C₄, or C₁-C₂). In embodiments, R⁴ is independently unsubstituted methyl. In embodiments, R⁴ is independently unsubstituted ethyl. In embodiments, R⁴ is independently unsubstituted propyl. In embodiments, R⁴ is independently unsubstituted isopropyl. In embodiments, R⁴ is independently unsubstituted tert-butyl. In embodiments, R⁴ is independently substituted or unsubstituted heteroalkyl (e.g., 2 to 8 membered, 2 to 6 membered, 4 to 6 membered, 2 to 3 membered, or 4 to 5 membered). In embodiments, R⁴ is independently substituted heteroalkyl (e.g., 2 to 8 membered, 2 to 6 membered, 4 to 6 membered, 2 to 3 membered, or 4 to 5 membered). In embodiments, R⁴ is independently unsubstituted heteroalkyl (e.g., 2 to 8 membered, 2 to 6 membered, 4 to 6 membered, 2 to 3 membered, or 4 to 5 membered). In embodiments, R⁴ is independently substituted or unsubstituted cycloalkyl (e.g., C₃-C₈, C₃-C₆, C₄-C₆, or C₅-C₆). In embodiments, R⁴ is independently substituted cycloalkyl (e.g., C₃-C₈, C₃-C₆, C₄-C₆, or C₅-C₆). In embodiments, R⁴ is independently unsubstituted cycloalkyl (e.g., C₃-C₈, C₃-C₆, C₄-C₆, or C₅-C₆). In embodiments, R⁴ is independently substituted or unsubstituted heterocycloalkyl (e.g., 3 to 8 membered, 3 to 6 membered, 4 to 6 membered, 4 to 5 membered, or 5 to 6 membered). In embodiments, R⁴ is independently substituted heterocycloalkyl (e.g., 3 to 8 membered, 3 to 6 membered, 4 to 6 membered, 4 to 5 membered, or 5 to 6 membered). In embodiments, R⁴ is independently unsubstituted heterocycloalkyl (e.g., 3 to 8 membered, 3 to 6 membered, 4 to 6 membered, 4 to 5 membered, or 5 to 6 membered). In embodiments, R⁴ is independently substituted or unsubstituted aryl (e.g., C₆-C₁₀ or phenyl). In embodiments, R⁴ is independently substituted aryl (e.g., C₆-C₁₀ or phenyl). In embodiments, R⁴ is independently unsubstituted aryl (e.g., C₆-C₁₀ or phenyl). In embodiments, R⁴ is independently substituted or unsubstituted heteroaryl (e.g., 5 to 10 membered, 5 to 9 membered, or 5 to 6 membered). In embodiments, R⁴ is independently substituted heteroaryl (e.g., 5 to 10 membered, 5 to 9 membered, or 5 to 6 membered). In embodiments, R⁴ is independently unsubstituted heteroaryl (e.g., 5 to 10 membered, 5 to 9 membered, or 5 to 6 membered).

In embodiments, R^{4A} is independently hydrogen. In embodiments, R^{4A} is independently —CX^{4A}₃. In embodiments, R^{4A} is independently —CHX^{4A}₂. In embodiments, R^{4A} is independently —CH₂X^{4A}. In embodiments, R^{4A} is independently —CN. In embodiments, R^{4A} is independently —COOH. In embodiments, R^{4A} is independently —CONH₂. In embodiments, X^{4A} is independently —F, —Cl, —Br, or —I.

In embodiments, R^{4A} is independently substituted or unsubstituted alkyl (e.g., C₁-C₈, C₁-C₆, C₁-C₄, or C₁-C₂). In embodiments, R^{4A} is independently substituted alkyl (e.g., C₁-C₈, C₁-C₆, C₁-C₄, or C₁-C₂). In embodiments, R^{4A} is independently unsubstituted alkyl (e.g., C₁-C₈, C₁-C₆, C₁-C₄, or C₁-C₂). In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted isopropyl. In embodiments, R^{4A} is independently unsubstituted tert-butyl. In embodiments, R^{4A} is independently substituted or unsubstituted heteroalkyl (e.g., 2 to 8 membered, 2 to 6 membered, 4 to 6 membered, 2 to 3 membered, or 4 to 5 membered). In embodiments, R^{4A} is independently substituted heteroalkyl (e.g., 2 to 8 membered, 2 to 6 membered, 4 to 6 membered, 2 to 3 membered, or 4 to 5 membered). In embodiments, R^{4A} is independently unsubstituted heteroalkyl (e.g., 2 to 8 membered, 2 to 6 membered, 4 to 6 membered, 2 to 3 membered, or 4 to 5 membered). In embodiments, R^{4A} is independently substituted or unsubstituted cycloalkyl (e.g., C₃-C₈, C₃-C₆, C₄-C₆, or C₅-C₆). In embodiments, R^{4A} is independently substituted cycloalkyl (e.g., C₃-C₈, C₃-C₆, C₄-C₆, or C₅-C₆). In embodiments, R^{4A} is independently unsubstituted cycloalkyl (e.g., C₃-C₈, C₃-C₆, C₄-C₆, or C₅-C₆). In embodiments, R^{4A} is independently substituted or unsubstituted heterocycloalkyl (e.g., 3 to 8 membered, 3 to 6 membered, 4 to 6 membered, 4 to 5 membered, or 5 to 6 membered). In embodiments, R^{4A} is independently substituted heterocycloalkyl (e.g., 3 to 8 membered, 3 to 6 membered, 4 to 6 membered, 4 to 5 membered, or 5 to 6 membered). In embodiments, R^{4A} is independently unsubstituted heterocycloalkyl (e.g., 3 to 8 membered, 3 to 6 membered, 4 to 6 membered, 4 to 5 membered, or 5 to 6 membered). In embodiments, R^{4A} is independently substituted or unsubstituted aryl (e.g., C₆-C₁₀ or phenyl). In embodiments, R^{4A} is independently substituted aryl (e.g., C₆-C₁₀ or phenyl). In embodiments, R^{4A} is independently unsubstituted aryl (e.g., C₆-C₁₀ or phenyl). In embodiments, R^{4A} is independently substituted or unsubstituted heteroaryl (e.g., 5 to 10 membered, 5 to 9 membered, or 5 to 6 membered). In embodiments, R^{4A} is independently substituted heteroaryl (e.g., 5 to 10 membered, 5 to 9 membered, or 5 to 6 membered). In embodiments, R^{4A} is independently unsubstituted heteroaryl (e.g., 5 to 10 membered, 5 to 9 membered, or 5 to 6 membered).

In embodiments, R^{4B} is independently hydrogen. In embodiments, R^{4B} is independently —CX^{4B}₃. In embodiments, R^{4B} is independently —CHX^{4B}₂. In embodiments, R^{4B} is independently —CH₂X^{4B}. In embodiments, R^{4B} is independently —CN. In embodiments, R^{4B} is independently —COOH. In embodiments, R^{4B} is independently —CONH₂. In embodiments, X^{4B} is independently —F, —Cl, —Br, or —I.

In embodiments, R^{4B} is independently substituted or unsubstituted alkyl (e.g., C₁-C₈, C₁-C₆, C₁-C₄, or C₁-C₂). In embodiments, R^{4B} is independently substituted alkyl (e.g., C₁-C₈, C₁-C₆, C₁-C₄, or C₁-C₂). In embodiments, R^{4B} is independently unsubstituted alkyl (e.g., C₁-C₈, C₁-C₆, C₁-C₄, or C₁-C₂). In embodiments, R^{4B} is independently unsubstituted methyl. In embodiments, R^{4B} is independently unsubstituted ethyl. In embodiments, R^{4B} is independently unsubstituted propyl. In embodiments, R^{4B} is independently unsubstituted isopropyl. In embodiments, R^{4B} is independently unsubstituted tert-butyl. In embodiments, R^{4B} is independently substituted or unsubstituted heteroalkyl (e.g., 2 to 8 membered, 2 to 6 membered, 4 to 6 membered, 2 to 3 membered, or 4 to 5 membered). In embodiments, R^{4B} is independently substituted heteroalkyl (e.g., 2 to 8 membered, 2 to 6 membered, 4 to 6 membered, 2 to 3 membered, or 4 to 5 membered). In embodiments, R^{4B} is independently unsubstituted heteroalkyl (e.g., 2 to 8 membered, 2 to 6 membered, 4 to 6 membered, 2 to 3 membered, or 4 to 5 membered). In embodiments, R^{4B} is independently substituted or unsubstituted cycloalkyl (e.g., C₃-C₈, C₃-C₆, C₄-C₆, or C₅-C₆). In embodiments, R^{4B} is independently substituted cycloalkyl (e.g., C₃-C₈, C₃-C₆, C₄-C₆, or C₅-C₆). In embodiments, R^{4B} is independently unsubstituted cycloalkyl (e.g., C₃-C₈, C₃-C₆, C₄-C₆, or C₅-C₆). In embodiments, R^{4B} is independently substituted or unsubstituted heterocycloalkyl (e.g., 3 to 8 membered, 3 to 6 membered, 4 to 6 membered, 4 to 5 membered, or 5 to 6 membered). In embodiments, R^{4B} is independently substituted heterocycloalkyl (e.g., 3 to 8 membered, 3 to 6 membered, 4 to 6 membered, 4 to 5 membered, or 5 to 6 membered). In embodiments, R^{4B} is independently unsubstituted heterocycloalkyl (e.g., 3 to 8 membered, 3 to 6 membered, 4 to 6 membered, 4 to 5 membered, or 5 to 6 membered). In embodiments, R^{4B} is independently substituted or unsubstituted aryl (e.g., C₆-C₁₀ or phenyl). In embodiments, R^{4B} is independently substituted aryl (e.g., C₆-C₁₀ or phenyl). In embodiments, R^{4B} is independently unsubstituted aryl (e.g., C₆-C₁₀ or phenyl). In embodiments, R^{4B} is independently substituted or unsubstituted heteroaryl (e.g., 5 to 10 membered, 5 to 9 membered, or 5 to 6 membered). In embodiments, R^{4B} is independently substituted heteroaryl (e.g., 5 to 10 membered, 5 to 9 membered, or 5 to 6 membered). In embodiments, R^{4B} is independently unsubstituted heteroaryl (e.g., 5 to 10 membered, 5 to 9 membered, or 5 to 6 membered).

In embodiments, R^{4A} and R^{4B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{4A} and R^{4B} substituents bonded to the same nitrogen atom may be joined to form a substituted or unsubstituted heteroaryl (e.g., 5 to 10 membered, 5 to 9 membered, or 5 to 6 membered). In embodiments, R^{4A} and R^{4B} substituents bonded to the same nitrogen atom may be joined to form a substituted heteroaryl (e.g., 5 to 10 membered, 5 to 9 membered, or 5 to 6 membered). In embodiments, R^{4A} and R^{4B} substituents bonded to the same nitrogen atom may be joined to form an unsubstituted heteroaryl (e.g., 5 to 10 membered, 5 to 9 membered, or 5 to 6 membered).

In embodiments, R⁴ is independently

hydrogen, —CX⁴₃, —CHX⁴₂, —CH₂X⁴, —CN, —COOH, —CONH₂, R²⁹-substituted or unsubstituted alkyl (e.g., C₁-C₈, C₁-C₆, C₁-C₄, or C₁-C₂), R²⁹-substituted or unsubstituted heteroalkyl (e.g., 2 to 8 membered, 2 to 6 membered, 4 to 6 membered, 2 to 3 membered, or 4 to 5 membered), R²⁹-substituted or unsubstituted cycloalkyl (e.g., C₃-C₈, C₃-C₆, C₄-C₆, or C₅-C₆), R²⁹-substituted or unsubstituted heterocycloalkyl (e.g., 3 to 8 membered, 3 to 6 membered, 4 to 6 membered, 4 to 5 membered, or 5 to 6 membered), R²⁹-substituted or unsubstituted aryl (e.g., C₆-C₁₀ or phenyl), or R²⁹-substituted or unsubstituted heteroaryl (e.g., 5 to 10 membered, 5 to 9 membered, or 5 to 6 membered). In embodiments, R⁴ is independently
hydrogen, —CX⁴³, —CHX⁴², —CH₂X⁴, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴ is independently —F, —Cl, —Br, or —I. In embodiments, R⁴ is independently hydrogen. In embodiments, R⁴ is independently unsubstituted methyl. In embodiments, R⁴ is independently unsubstituted ethyl.

R²⁹ is independently oxo,

halogen, —CX²⁹₃, —CHX²⁹₂, —CH₂X²⁹, —OCX²⁹₃, —OCH₂X²⁹, —OCHX²⁹², —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³⁰-substituted or unsubstituted alkyl (e.g., C₁-C₈, C₁-C₆, C₁-C₄, or C₁-C₂), R³⁰-substituted or unsubstituted heteroalkyl (e.g., 2 to 8 membered, 2 to 6 membered, 4 to 6 membered, 2 to 3 membered, or 4 to 5 membered), R³⁰-substituted or unsubstituted cycloalkyl (e.g., C₃-C₈, C₃-C₆, C₄-C₆, or C₅-C₆), R³⁰-substituted or unsubstituted heterocycloalkyl (e.g., 3 to 8 membered, 3 to 6 membered, 4 to 6 membered, 4 to 5 membered, or 5 to 6 membered), R³⁰-substituted or unsubstituted aryl (e.g., C₆-C₁₀ or phenyl), or R³⁰-substituted or unsubstituted heteroaryl (e.g., 5 to 10 membered, 5 to 9 membered, or 5 to 6 membered). In embodiments, R²⁹ is independently oxo,
halogen, —CX²⁹₃, —CHX²⁹₂, —CH₂X²⁹, —OCX²⁹³, —OCH₂X²⁹, —OCHX²⁹², —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X²⁹ is independently —F, —Cl, —Br, or —I. In embodiments, R²⁹ is independently unsubstituted methyl. In embodiments, R²⁹ is independently unsubstituted ethyl. In embodiments, R²⁹ is independently unsubstituted phenyl.

R³⁰ is independently oxo,

halogen, —CX³⁰₃, —CHX³⁰₂, —CH₂X³⁰, —OCX³⁰₃, —OCH₂X³⁰, —OCHX³⁰₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³¹-substituted or unsubstituted alkyl (e.g., C₁-C₈, C₁-C₆, C₁-C₄, or C₁-C₂), R³¹-substituted or unsubstituted heteroalkyl (e.g., 2 to 8 membered, 2 to 6 membered, 4 to 6 membered, 2 to 3 membered, or 4 to 5 membered), R³¹-substituted or unsubstituted cycloalkyl (e.g., C₃-C₈, C₃-C₆, C₄-C₆, or C₅-C₆), R³¹-substituted or unsubstituted heterocycloalkyl (e.g., 3 to 8 membered, 3 to 6 membered, 4 to 6 membered, 4 to 5 membered, or 5 to 6 membered), R³¹-substituted or unsubstituted aryl (e.g., C₆-C₁₀ or phenyl), or R³¹-substituted or unsubstituted heteroaryl (e.g., 5 to 10 membered, 5 to 9 membered, or 5 to 6 membered). In embodiments, R³⁰ is independently oxo,
halogen, —CX³⁰₃, —CHX³⁰₂, —CH₂X³⁰, —OCX³⁰₃, —OCH₂X³⁰, —OCHX³⁰₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁰ is independently —F, —Cl, —Br, or —I. In embodiments, R³⁰ is independently unsubstituted methyl. In embodiments, R³⁰ is independently unsubstituted ethyl.

R³¹ is independently oxo,

halogen, —CX³¹₃, —CHX³¹₂, —CH₂X³¹, —OCX³¹₃, —OCH₂X³¹, —OCHX³¹₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³¹ is independently —F, —Cl, —Br, or —I. In embodiments, R³¹ is independently unsubstituted methyl. In embodiments, R³¹ is independently unsubstituted ethyl.

In embodiments, R^{4A} is independently

hydrogen, —CX^{4A}₃, —CHX^{4A}₂, —CH₂X^{4A}, —CN, —COOH, —CONH₂, R^{29A}-substituted or unsubstituted alkyl (e.g., C₁-C₈, C₁-C₆, C₁-C₄, or C₁-C₂), R^{29A}-substituted or unsubstituted heteroalkyl (e.g., 2 to 8 membered, 2 to 6 membered, 4 to 6 membered, 2 to 3 membered, or 4 to 5 membered), R^{29A}-substituted or unsubstituted cycloalkyl (e.g., C₃-C₈, C₃-C₆, C₄-C₆, or C₅-C₆), R^{29A}-substituted or unsubstituted heterocycloalkyl (e.g., 3 to 8 membered, 3 to 6 membered, 4 to 6 membered, 4 to 5 membered, or 5 to 6 membered), R^{29A}-substituted or unsubstituted aryl (e.g., C₆-C₁₀ or phenyl), or R^{29A}-substituted or unsubstituted heteroaryl (e.g., 5 to 10 membered, 5 to 9 membered, or 5 to 6 membered). In embodiments, R^{4A} is independently hydrogen, —CX^{4A}₃, —CHX^{4A}₂, —CH₂X^{4A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4A} is independently —F, —Cl, —Br, or —I. In embodiments, R^{4A} is independently hydrogen. In embodiments, R^{4A} is independently unsubstituted methyl. In embodiments, R^{4A} is independently unsubstituted ethyl.

In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered) or R^{29A}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29A}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{29A} is independently oxo,

halogen, —CX^{29A}₃, —CHX^{29A}₂, —CH₂X^{29A}, —OCX²⁹A₃, —OCH₂X^{29A}, —OCHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{30A}-substituted or unsubstituted alkyl (e.g., C₁-C₈, C₁-C₆, C₁-C₄, or C₁-C₂), R^{30A}-substituted or unsubstituted heteroalkyl (e.g., 2 to 8 membered, 2 to 6 membered, 4 to 6 membered, 2 to 3 membered, or 4 to 5 membered), R^{30A}-substituted or unsubstituted cycloalkyl (e.g., C₃-C₈, C₃-C₆, C₄-C₆, or C₅-C₆), R^{30A}-substituted or unsubstituted heterocycloalkyl (e.g., 3 to 8 membered, 3 to 6 membered, 4 to 6 membered, 4 to 5 membered, or 5 to 6 membered), R^{30A}-substituted or unsubstituted aryl (e.g., C₆-C₁₀ or phenyl), or R^{30A}-substituted or unsubstituted heteroaryl (e.g., 5 to 10 membered, 5 to 9 membered, or 5 to 6 membered). In embodiments, R^{29A} is independently oxo,
halogen, —CX^{29A}₃, —CHX^{29A}₂, —CH₂X^{29A}, —OCX^{29A}₃, —OCH₂X^{29A}, —OCHX^{29A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29A} is independently —F, —Cl, —Br, or —I. In embodiments, R^{29A} is independently unsubstituted methyl. In embodiments, R^{29A} is independently unsubstituted ethyl.

R^{30A} is independently oxo,

halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{31A}-substituted or unsubstituted alkyl (e.g., C₁-C₈, C₁-C₆, C₁-C₄, or C₁-C₂), R^{31A}-substituted or unsubstituted heteroalkyl (e.g., 2 to 8 membered, 2 to 6 membered, 4 to 6 membered, 2 to 3 membered, or 4 to 5 membered), R^{31A}-substituted or unsubstituted cycloalkyl (e.g., C₃-C₈, C₃-C₆, C₄-C₆, or C₅-C₆), R^{31A}-substituted or unsubstituted heterocycloalkyl (e.g., 3 to 8 membered, 3 to 6 membered, 4 to 6 membered, 4 to 5 membered, or 5 to 6 membered), R^{31A}-substituted or unsubstituted aryl (e.g., C₆-C₁₀ or phenyl), or R^{31A}-substituted or unsubstituted heteroaryl (e.g., 5 to 10 membered, 5 to 9 membered, or 5 to 6 membered). In embodiments, R^{30A} is independently oxo,
halogen, —CX^{30A}₃, —CHX^{30A}₂, —CH₂X^{30A}, —OCX^{30A}₃, —OCH₂X^{30A}, —OCHX^{30A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30A} is independently —F, —Cl, —Br, or —I. In embodiments, R^{30A} is independently unsubstituted methyl. In embodiments, R^{30A} is independently unsubstituted ethyl.

R^{31A} is independently oxo,

halogen, —CX^{31A}₃, —CHX^{31A}₂, —CH₂X^{31A}, —OCX^{31A}₃, —OCH₂X^{31A}, —OCHX^{31A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31A} is independently —F, —Cl, —Br, or —I. In embodiments, R^{31A} is independently unsubstituted methyl. In embodiments, R^{31A} is independently unsubstituted ethyl.

In embodiments, R^{4B} is independently

hydrogen, —CX^{4B}₃, —CHX^{4B}₂, —CH₂X^{4B}, —CN, —COOH, —CONH₂, R^{29B}-substituted or unsubstituted alkyl (e.g., C₁-C₈, C₁-C₆, C₁-C₄, or C₁-C₂), R^{29B}-substituted or unsubstituted heteroalkyl (e.g., 2 to 8 membered, 2 to 6 membered, 4 to 6 membered, 2 to 3 membered, or 4 to 5 membered), R^{29B}-substituted or unsubstituted cycloalkyl (e.g., C₃-C₈, C₃-C₆, C₄-C₆, or C₅-C₆), R^{29B}-substituted or unsubstituted heterocycloalkyl (e.g., 3 to 8 membered, 3 to 6 membered, 4 to 6 membered, 4 to 5 membered, or 5 to 6 membered), R^{29B}-substituted or unsubstituted aryl (e.g., C₆-C₁₀ or phenyl), or R^{29B}-substituted or unsubstituted heteroaryl (e.g., 5 to 10 membered, 5 to 9 membered, or 5 to 6 membered). In embodiments, R^{4B} is independently hydrogen, —CX^{4B}₃, —CHX^{4B}₂, —CH₂X^{4B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{4B} is independently —F, —Cl, —Br, or —I. In embodiments, R^{4B} is independently hydrogen. In embodiments, R^{4B} is independently unsubstituted methyl. In embodiments, R^{4B} is independently unsubstituted ethyl.

In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered) or R^{29B}-substituted or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{4A} and R^{4B} substituents bonded to the same nitrogen atom may optionally be joined to form a R^{29B}-substituted or unsubstituted heterocycloalkyl (e.g., 3 to 8 membered, 3 to 6 membered, 4 to 6 membered, 4 to 5 membered, or 5 to 6 membered). In embodiments, R^{4A} and R^{4B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{29B} is independently oxo,

halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{30B}-substituted or unsubstituted alkyl (e.g., C₁-C₈, C₁-C₆, C₁-C₄, or C₁-C₂), R^{30B}-substituted or unsubstituted heteroalkyl (e.g., 2 to 8 membered, 2 to 6 membered, 4 to 6 membered, 2 to 3 membered, or 4 to 5 membered), R^{30B}-substituted or unsubstituted cycloalkyl (e.g., C₃-C₈, C₃-C₆, C₄-C₆, or C₅-C₆), R^{30B}-substituted or unsubstituted heterocycloalkyl (e.g., 3 to 8 membered, 3 to 6 membered, 4 to 6 membered, 4 to 5 membered, or 5 to 6 membered), R^{30B}-substituted or unsubstituted aryl (e.g., C₆-C₁₀ or phenyl), or R^{30B}-substituted or unsubstituted heteroaryl (e.g., 5 to 10 membered, 5 to 9 membered, or 5 to 6 membered). In embodiments, R^{29B} is independently oxo,
halogen, —CX^{29B}₃, —CHX^{29B}₂, —CH₂X^{29B}, —OCX^{29B}₃, —OCH₂X^{29B}, —OCHX^{29B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{29B} is independently —F, —Cl, —Br, or —I. In embodiments, R^{29B} is independently unsubstituted methyl. In embodiments, R^{29B} is independently unsubstituted ethyl.

R^{30B} is independently oxo,

halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{31B}-substituted or unsubstituted alkyl (e.g., C₁-C₈, C₁-C₆, C₁-C₄, or C₁-C₂), R^{31B}-substituted or unsubstituted heteroalkyl (e.g., 2 to 8 membered, 2 to 6 membered, 4 to 6 membered, 2 to 3 membered, or 4 to 5 membered), R^{31B}-substituted or unsubstituted cycloalkyl (e.g., C₃-C₈, C₃-C₆, C₄-C₆, or C₅-C₆), R^{31B}-substituted or unsubstituted heterocycloalkyl (e.g., 3 to 8 membered, 3 to 6 membered, 4 to 6 membered, 4 to 5 membered, or 5 to 6 membered), R^{31B}-substituted or unsubstituted aryl (e.g., C₆-C₁₀ or phenyl), or R^{31B}-substituted or unsubstituted heteroaryl (e.g., 5 to 10 membered, 5 to 9 membered, or 5 to 6 membered). In embodiments, R^{30B} is independently oxo,
halogen, —CX^{30B}₃, —CHX^{30B}₂, —CH₂X^{30B}, —OCX^{30B}₃, —OCH₂X^{30B}, —OCHX^{30B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{30B} is independently —F, —Cl, —Br, or —I. In embodiments, R^{30B} is independently unsubstituted methyl. In embodiments, R^{30B} is independently unsubstituted ethyl.

R^{31B} is independently oxo,

halogen, —CX^{31B}₃, —CHX^{31B}₂, —CH₂X^{31B}, —OCX^{31B}₃, —OCH₂X^{31B}, —OCHX^{31B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{31B} is independently —F, —Cl, —Br, or —I. In embodiments, R^{31B} is independently unsubstituted methyl. In embodiments, R^{31B} is independently unsubstituted ethyl.

In embodiments, L² is —NR⁵— or substituted or unsubstituted heterocycloalkylene including a ring nitrogen bonded directly to E. In embodiments, L² is —NR⁵—.

In embodiments, L² is a bond. In embodiments, L² is —S(O)₂—. In embodiments, L² is —NR⁵—. In embodiments, L² is —O—. In embodiments, L² is —S—. In embodiments, L² is —C(O)—. In embodiments, L² is —C(O)NR⁵—. In embodiments, L² is —NR⁵C(O)—. In embodiments, L² is —NR^{5C}(O)NH—. In embodiments, L² is —NHC(O)NR⁵—. In embodiments, L² is —C(O)O—. In embodiments, L² is —OC(O)—. In embodiments, L² is —NH—. In embodiments, L² is —C(O)NH—. In embodiments, L² is —NHC(O)—. In embodiments, L² is —NHC(O)NH—. In embodiments, L² is —CH₂—. In embodiments, L² is —OCH₂—. In embodiments, L² is —CH₂O—. In embodiments, L² is —NHCH₂—. In embodiments, L² is —CH₂NH—.

In embodiments, L² is a

bond, —S(O)₂—, —NR⁵—, —O—, —S—, —C(O)—, —C(O)NR⁵—, —NR⁴C(O)—, —NR⁵C(O)NH—, —NHC(O)NR⁵—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered).

In embodiments, L² is independently substituted or unsubstituted alkylene (e.g., C₁-C₈, C₁-C₆, C₁-C₄, or C₁-C₂). In embodiments, L² is independently substituted alkylene (e.g., C₁-C₈, C₁-C₆, C₁-C₄, or C₁-C₂). In embodiments, L² is independently unsubstituted alkylene (e.g., C₁-C₈, C₁-C₆, C₁-C₄, or C₁-C₂). In embodiments, L² is independently unsubstituted methylene. In embodiments, L² is independently unsubstituted ethylene. In embodiments, L² is independently unsubstituted propylene. In embodiments, L² is independently unsubstituted isopropylene. In embodiments, L² is independently unsubstituted tert-butylene. In embodiments, L² is independently substituted or unsubstituted heteroalkylene (e.g., 2 to 8 membered, 2 to 6 membered, 4 to 6 membered, 2 to 3 membered, or 4 to 5 membered). In embodiments, L² is independently substituted heteroalkylene (e.g., 2 to 8 membered, 2 to 6 membered, 4 to 6 membered, 2 to 3 membered, or 4 to 5 membered). In embodiments, L² is independently unsubstituted heteroalkylene (e.g., 2 to 8 membered, 2 to 6 membered, 4 to 6 membered, 2 to 3 membered, or 4 to 5 membered). In embodiments, L² is independently substituted or unsubstituted cycloalkylene (e.g., C₃-C₈, C₃-C₆, C₄-C₆, or C₅-C₆). In embodiments, L² is independently substituted cycloalkylene (e.g., C₃-C₈, C₃-C₆, C₄-C₆, or C₅—C₆). In embodiments, L² is independently unsubstituted cycloalkylene (e.g., C₃-C₈, C₃-C₆, C₄-C₆, or C₅-C₆). In embodiments, L² is independently substituted or unsubstituted heterocycloalkylene (e.g., 3 to 8 membered, 3 to 6 membered, 4 to 6 membered, 4 to 5 membered, or 5 to 6 membered). In embodiments, L² is independently substituted heterocycloalkylene (e.g., 3 to 8 membered, 3 to 6 membered, 4 to 6 membered, 4 to 5 membered, or 5 to 6 membered). In embodiments, L² is independently unsubstituted heterocycloalkylene (e.g., 3 to 8 membered, 3 to 6 membered, 4 to 6 membered, 4 to 5 membered, or 5 to 6 membered). In embodiments, L² is independently substituted or unsubstituted arylene (e.g., C₆-C₁₀ or phenylene). In embodiments, L² is independently substituted arylene (e.g., C₆-C₁₀ or phenylene). In embodiments, L² is independently unsubstituted arylene (e.g., C₆-C₁₀ or phenylene). In embodiments, L² is independently substituted or unsubstituted heteroarylene (e.g., 5 to 10 membered, 5 to 9 membered, or 5 to 6 membered). In embodiments, L² is independently substituted heteroarylene (e.g., 5 to 10 membered, 5 to 9 membered, or 5 to 6 membered). In embodiments, L² is independently unsubstituted heteroarylene (e.g., 5 to 10 membered, 5 to 9 membered, or 5 to 6 membered).

In embodiments, L² is independently

bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, R³⁸-substituted or unsubstituted alkylene (e.g., C₁-C₈, C₁-C₆, C₁-C₄, or C₁-C₂), R³⁸-substituted or unsubstituted heteroalkylene (e.g., 2 to 8 membered, 2 to 6 membered, 4 to 6 membered, 2 to 3 membered, or 4 to 5 membered), R³⁸-substituted or unsubstituted cycloalkylene (e.g., C₃-C₈, C₃-C₆, C₄-C₆, or C₅-C₆), R³⁸-substituted or unsubstituted heterocycloalkylene (e.g., 3 to 8 membered, 3 to 6 membered, 4 to 6 membered, 4 to 5 membered, or 5 to 6 membered), R³⁸-substituted or unsubstituted arylene (e.g., C₆-C₁₀ or phenylene), or R³⁸-substituted or unsubstituted heteroarylene (e.g., 5 to 10 membered, 5 to 9 membered, or 5 to 6 membered). In embodiments, L² is independently
bond, —S(O)₂—, —N(R⁵)—, —O—, —S—, —C(O)—, —C(O)N(R⁵)—, —N(R⁵)C(O)—, —N(R⁵)C(O)NH—, —NHC(O)N(R⁵)—, —C(O)O—, —OC(O)—, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₃-C₆, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenylene), or unsubstituted heteroarylene (e.g., 5 to 10 membered, 5 to 9 membered, or 5 to 6 membered). In embodiments, L² is independently unsubstituted methylene. In embodiments, L² is independently unsubstituted ethylene. In embodiments, L² is independently methyl-substituted methylene.

R³⁸ is independently oxo,

halogen, —CX³⁸₃, —CHX³⁸₂, —CH₂X³⁸, —OCX³⁸₃, —OCH₂X³⁸, —OCHX³⁸₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³⁹-substituted or unsubstituted alkyl (e.g., C₁-C₈, C₁-C₆, C₁-C₄, or C₁-C₂), R³⁹-substituted or unsubstituted heteroalkyl (e.g., 2 to 8 membered, 2 to 6 membered, 4 to 6 membered, 2 to 3 membered, or 4 to 5 membered), R³⁹-substituted or unsubstituted cycloalkyl (e.g., C₃-C₈, C₃-C₆, C₄-C₆, or C₅-C₆), R³⁹-substituted or unsubstituted heterocycloalkyl (e.g., 3 to 8 membered, 3 to 6 membered, 4 to 6 membered, 4 to 5 membered, or 5 to 6 membered), R³⁹-substituted or unsubstituted aryl (e.g., C₆-C₁₀ or phenyl), or R³⁹-substituted or unsubstituted heteroaryl (e.g., 5 to 10 membered, 5 to 9 membered, or 5 to 6 membered). In embodiments, R³⁸ is independently oxo,
halogen, —CX³⁸₃, —CHX³⁸₂, —CH₂X³⁸, —OCX³⁸₃, —OCH₂X³⁸, —OCHX³⁸₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁸ is independently —F, —Cl, —Br, or —I. In embodiments, R³⁸ is independently unsubstituted methyl. In embodiments, R³⁸ is independently unsubstituted ethyl.

R³⁹ is independently oxo,

halogen, —CX³⁹₃, —CHX³⁹₂, —CH₂X³⁹, —OCX³⁹₃, —OCH₂X³⁹, —OCHX³⁹₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R⁴⁰-substituted or unsubstituted alkyl (e.g., C₁-C₈, C₁-C₆, C₁-C₄, or C₁-C₂), R⁴⁰-substituted or unsubstituted heteroalkyl (e.g., 2 to 8 membered, 2 to 6 membered, 4 to 6 membered, 2 to 3 membered, or 4 to 5 membered), R⁴⁰-substituted or unsubstituted cycloalkyl (e.g., C₃-C₈, C₃-C₆, C₄-C₆, or C₅-C₆), R⁴⁰-substituted or unsubstituted heterocycloalkyl (e.g., 3 to 8 membered, 3 to 6 membered, 4 to 6 membered, 4 to 5 membered, or 5 to 6 membered), R⁴⁰-substituted or unsubstituted aryl (e.g., C₆-C₁₀ or phenyl), or R⁴⁰-substituted or unsubstituted heteroaryl (e.g., 5 to 10 membered, 5 to 9 membered, or 5 to 6 membered). In embodiments, R³⁹ is independently oxo,
halogen, —CX³⁹₃, —CHX³⁹₂, —CH₂X³⁹, —OCX³⁹₃, —OCH₂X³⁹, —OCHX³⁹₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁹ is independently —F, —Cl, —Br, or —I. In embodiments, R³⁹ is independently unsubstituted methyl. In embodiments, R³⁹ is independently unsubstituted ethyl.

R⁴⁰ is independently oxo,

halogen, —CX⁴⁰₃, —CHX⁴⁰₂, —CH₂X⁴⁰, —OCX⁴⁰₃, —OCH₂X⁴⁰, —OCHX⁴⁰₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁴⁰ is independently —F, —Cl, —Br, or —I. In embodiments, R⁴⁰ is independently unsubstituted methyl. In embodiments, R⁴⁰ is independently unsubstituted ethyl.

In embodiments, R⁵ is hydrogen, substituted or unsubstituted C₁-C₆ alkyl, or substituted or unsubstituted 2 to 6 membered heteroalkyl. In embodiments, R⁵ is hydrogen or unsubstituted C₁-C₃ alkyl. In embodiments, R⁵ is hydrogen, unsubstituted methyl, unsubstituted ethyl, unsubstituted hexyl, or unsubstituted benzyl. In embodiments, R⁵ is hydrogen.

In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted isopropyl. In embodiments, R⁵ is independently unsubstituted n-propyl. In embodiments, R⁵ is independently unsubstituted butyl. In embodiments, R⁵ is independently unsubstituted n-butyl. In embodiments, R⁵ is independently unsubstituted t-butyl. In embodiments, R⁵ is independently unsubstituted pentyl. In embodiments, R⁵ is independently unsubstituted n-pentyl. In embodiments, R⁵ is independently unsubstituted hexyl. In embodiments, R⁵ is independently unsubstituted n-hexyl. In embodiments, R⁵ is independently unsubstituted heptyl. In embodiments, R⁵ is independently unsubstituted n-heptyl. In embodiments, R⁵ is independently unsubstituted octyl. In embodiments, R⁵ is independently unsubstituted n-octyl. In embodiments, R⁵ is independently unsubstituted benzyl. In embodiments, R⁵ is independently unsubstituted C₁-C₈ alkyl. In embodiments, R⁵ is independently halo-substituted methyl. In embodiments, R⁵ is independently halo-substituted ethyl. In embodiments, R⁵ is independently halo-substituted isopropyl. In embodiments, R⁵ is independently halo-substituted n-propyl. In embodiments, R⁵ is independently halo-substituted n-butyl. In embodiments, R⁵ is independently halo-substituted t-butyl. In embodiments, R¹ is independently halo-substituted n-pentyl. In embodiments, R⁵ is independently halo-substituted benzyl. In embodiments, R⁵ is independently halo-substituted C₁-C₈ alkyl. In embodiments, R⁵ is independently unsubstituted 2 to 6 membered heteroalkyl. In embodiments, R⁵ is independently unsubstituted 2 to 7 membered heteroalkyl. In embodiments, R⁵ is independently unsubstituted 2 to 8 membered heteroalkyl. In embodiments, R⁵ is independently unsubstituted 2 to 9 membered heteroalkyl. In embodiments, R⁵ is independently unsubstituted 2 to 10 membered heteroalkyl. In embodiments, R⁵ is independently unsubstituted 3 to 10 membered heteroalkyl. In embodiments, R⁵ is independently unsubstituted 4 to 10 membered heteroalkyl. In embodiments, R⁵ is independently unsubstituted 5 to 10 membered heteroalkyl. In embodiments, R⁵ is independently unsubstituted 6 to 10 membered heteroalkyl. In embodiments, R⁵ is independently unsubstituted 7 to 10 membered heteroalkyl. In embodiments, R⁵ is independently unsubstituted 8 to 10 membered heteroalkyl. In embodiments, R⁵ is independently unsubstituted 6 to 10 membered heteroalkyl. In embodiments, R⁵ is independently unsubstituted 7 to 9 membered heteroalkyl.

In embodiments, R⁵ is independently hydrogen, —CX⁵₃, —CHX⁵₂, —CH₂X⁵, —OCX⁵₃, —OCH₂X⁵, —OCHX⁵₂, —CN, —C(O)R^{5A}, —C(O)OR^{5A}, —C(O)NR^{5A}R^{5B}, —OR^{5A}, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, R⁵ is independently hydrogen. In embodiments, R⁵ is independently —CX⁵₃. In embodiments, R⁵ is independently —CHX⁵₂. In embodiments, R⁵ is independently —CH₂X⁵. In embodiments, R⁵ is independently —CN. In embodiments, R⁵ is independently —C(O)R^{5A}. In embodiments, R⁵ is independently —C(O)—OR^{5A}. In embodiments, R⁵ is independently —C(O)NR^{5A}R^{5B}. In embodiments, R⁵ is independently —COOH. In embodiments, R⁵ is independently —CONH₂. In embodiments, R⁵ is independently —CF₃. In embodiments, R⁵ is independently —CHF₂. In embodiments, R⁵ is independently —CH₂F. In embodiments, R⁵ is independently —CH₃. In embodiments, R⁵ is independently —CH₂CH₃. In embodiments, R⁵ is independently —CH₂CH₂CH₃. In embodiments, R⁵ is independently —CH(CH₃)₂. In embodiments, R⁵ is independently —C(CH₃)₃.

In embodiments, R⁵ is independently substituted or unsubstituted alkyl (e.g., C₁-C₈, C₁-C₆, C₁-C₄, or C₁-C₂). In embodiments, R⁵ is independently substituted alkyl (e.g., C₁-C₈, C₁-C₆, C₁-C₄, or C₁-C₂). In embodiments, R⁵ is independently unsubstituted alkyl (e.g., C₁-C₈, C₁-C₆, C₁-C₄, or C₁-C₂). In embodiments, R⁵ is independently unsubstituted methyl. In embodiments, R⁵ is independently unsubstituted ethyl. In embodiments, R⁵ is independently unsubstituted propyl. In embodiments, R⁵ is independently unsubstituted isopropyl. In embodiments, R⁵ is independently unsubstituted tert-butyl. In embodiments, R⁵ is independently substituted or unsubstituted heteroalkyl (e.g., 2 to 8 membered, 2 to 6 membered, 4 to 6 membered, 2 to 3 membered, or 4 to 5 membered). In embodiments, R⁵ is independently substituted heteroalkyl (e.g., 2 to 8 membered, 2 to 6 membered, 4 to 6 membered, 2 to 3 membered, or 4 to 5 membered). In embodiments, R⁵ is independently unsubstituted heteroalkyl (e.g., 2 to 8 membered, 2 to 6 membered, 4 to 6 membered, 2 to 3 membered, or 4 to 5 membered). In embodiments, R⁵ is independently substituted or unsubstituted cycloalkyl (e.g., C₃-C₈, C₃-C₆, C₄-C₆, or C₅-C₆). In embodiments, R⁵ is independently substituted cycloalkyl (e.g., C₃-C₈, C₃-C₆, C₄-C₆, or C₅-C₆). In embodiments, R⁵ is independently unsubstituted cycloalkyl (e.g., C₃-C₈, C₃-C₆, C₄-C₆, or C₅-C₆). In embodiments, R⁵ is independently substituted or unsubstituted heterocycloalkyl (e.g., 3 to 8 membered, 3 to 6 membered, 4 to 6 membered, 4 to 5 membered, or 5 to 6 membered). In embodiments, R⁵ is independently substituted heterocycloalkyl (e.g., 3 to 8 membered, 3 to 6 membered, 4 to 6 membered, 4 to 5 membered, or 5 to 6 membered). In embodiments, R⁵ is independently unsubstituted heterocycloalkyl (e.g., 3 to 8 membered, 3 to 6 membered, 4 to 6 membered, 4 to 5 membered, or 5 to 6 membered). In embodiments, R⁵ is independently substituted or unsubstituted aryl (e.g., C₆-C₁₀ or phenyl). In embodiments, R⁵ is independently substituted aryl (e.g., C₆-C₁₀ or phenyl). In embodiments, R⁵ is independently unsubstituted aryl (e.g., C₆-C₁₀ or phenyl). In embodiments, R⁵ is independently substituted or unsubstituted heteroaryl (e.g., 5 to 10 membered, 5 to 9 membered, or 5 to 6 membered). In embodiments, R⁵ is independently substituted heteroaryl (e.g., 5 to 10 membered, 5 to 9 membered, or 5 to 6 membered). In embodiments, R⁵ is independently unsubstituted heteroaryl (e.g., 5 to 10 membered, 5 to 9 membered, or 5 to 6 membered).

In embodiments, R^{5A} is independently hydrogen. In embodiments, R^{5A} is independently —CX^{5A}₃. In embodiments, R^{5A} is independently —CHX^{5A}₂. In embodiments, R^{5A} is independently —CH₂X^{5A}. In embodiments, R^{5A} is independently —CN. In embodiments, R^{5A} is independently —COOH. In embodiments, R^{5A} is independently —CONH₂. In embodiments, X^{5A} is independently —F, —Cl, —Br, or —I.

In embodiments, R^{5A} is independently substituted or unsubstituted alkyl (e.g., C₁-C₈, C₁-C₆, C₁-C₄, or C₁-C₂). In embodiments, R^{5A} is independently substituted alkyl (e.g., C₁-C₈, C₁-C₆, C₁-C₄, or C₁-C₂). In embodiments, R^{5A} is independently unsubstituted alkyl (e.g., C₁-C₈, C₁-C₆, C₁-C₄, or C₁-C₂). In embodiments, R^{5A} is independently unsubstituted methyl. In embodiments, R^{5A} is independently unsubstituted ethyl. In embodiments, R^{5A} is independently unsubstituted propyl. In embodiments, R^{5A} is independently unsubstituted isopropyl. In embodiments, R^{5A} is independently unsubstituted tert-butyl. In embodiments, R^{5A} is independently substituted or unsubstituted heteroalkyl (e.g., 2 to 8 membered, 2 to 6 membered, 4 to 6 membered, 2 to 3 membered, or 4 to 5 membered). In embodiments, R^{5A} is independently substituted heteroalkyl (e.g., 2 to 8 membered, 2 to 6 membered, 4 to 6 membered, 2 to 3 membered, or 4 to 5 membered). In embodiments, R^{5A} is independently unsubstituted heteroalkyl (e.g., 2 to 8 membered, 2 to 6 membered, 4 to 6 membered, 2 to 3 membered, or 4 to 5 membered). In embodiments, R^{5A} is independently substituted or unsubstituted cycloalkyl (e.g., C₃-C₈, C₃-C₆, C₄-C₆, or C₅-C₆). In embodiments, R^{5A} is independently substituted cycloalkyl (e.g., C₃-C₈, C₃-C₆, C₄-C₆, or C₅-C₆). In embodiments, R^{5A} is independently unsubstituted cycloalkyl (e.g., C₃-C₈, C₃-C₆, C₄-C₆, or C₅-C₆). In embodiments, R^{5A} is independently substituted or unsubstituted heterocycloalkyl (e.g., 3 to 8 membered, 3 to 6 membered, 4 to 6 membered, 4 to 5 membered, or 5 to 6 membered). In embodiments, R^{5A} is independently substituted heterocycloalkyl (e.g., 3 to 8 membered, 3 to 6 membered, 4 to 6 membered, 4 to 5 membered, or 5 to 6 membered). In embodiments, R^{5A} is independently unsubstituted heterocycloalkyl (e.g., 3 to 8 membered, 3 to 6 membered, 4 to 6 membered, 4 to 5 membered, or 5 to 6 membered). In embodiments, R^{5A} is independently substituted or unsubstituted aryl (e.g., C₆-C₁₀ or phenyl). In embodiments, R^{5A} is independently substituted aryl (e.g., C₆-C₁₀ or phenyl). In embodiments, R^{5A} is independently unsubstituted aryl (e.g., C₆-C₁₀ or phenyl). In embodiments, R^{5A} is independently substituted or unsubstituted heteroaryl (e.g., 5 to 10 membered, 5 to 9 membered, or 5 to 6 membered). In embodiments, R^{5A} is independently substituted heteroaryl (e.g., 5 to 10 membered, 5 to 9 membered, or 5 to 6 membered). In embodiments, R^{5A} is independently unsubstituted heteroaryl (e.g., 5 to 10 membered, 5 to 9 membered, or 5 to 6 membered).

In embodiments, R^{5B} is independently hydrogen. In embodiments, R^{5B} is independently —CX^{5B}₃. In embodiments, R^{5B} is independently —CHX^{5B}₂. In embodiments, R^{5B} is independently —CH₂X^{5B}. In embodiments, R^{5B} is independently —CN. In embodiments, R^{5B} is independently —COOH. In embodiments, R^{5B} is independently —CONH₂. In embodiments, X^{5B} is independently —F, —Cl, —Br, or —I.

In embodiments, R^{5B} is independently substituted or unsubstituted alkyl (e.g., C₁-C₈, C₁-C₆, C₁-C₄, or C₁-C₂). In embodiments, R^{5B} is independently substituted alkyl (e.g., C₁-C₈, C₁-C₆, C₁-C₄, or C₁-C₂). In embodiments, R^{5B} is independently unsubstituted alkyl (e.g., C₁-C₈, C₁-C₆, C₁-C₄, or C₁-C₂). In embodiments, R^{5B} is independently unsubstituted methyl. In embodiments, R^{5B} is independently unsubstituted ethyl. In embodiments, R^{5B} is independently unsubstituted propyl. In embodiments, R^{5B} is independently unsubstituted isopropyl. In embodiments, R^{5B} is independently unsubstituted tert-butyl. In embodiments, R^{5B} is independently substituted or unsubstituted heteroalkyl (e.g., 2 to 8 membered, 2 to 6 membered, 4 to 6 membered, 2 to 3 membered, or 4 to 5 membered). In embodiments, R^{5B} is independently substituted heteroalkyl (e.g., 2 to 8 membered, 2 to 6 membered, 4 to 6 membered, 2 to 3 membered, or 4 to 5 membered). In embodiments, R^{5B} is independently unsubstituted heteroalkyl (e.g., 2 to 8 membered, 2 to 6 membered, 4 to 6 membered, 2 to 3 membered, or 4 to 5 membered). In embodiments, R^{5B} is independently substituted or unsubstituted cycloalkyl (e.g., C₃-C₈, C₃-C₆, C₄-C₆, or C₅-C₆). In embodiments, R^{5B} is independently substituted cycloalkyl (e.g., C₃-C₈, C₃-C₆, C₄-C₆, or C₅-C₆). In embodiments, R^{5B} is independently unsubstituted cycloalkyl (e.g., C₃-C₈, C₃-C₆, C₄-C₆, or C₅-C₆). In embodiments, R^{5B} is independently substituted or unsubstituted heterocycloalkyl (e.g., 3 to 8 membered, 3 to 6 membered, 4 to 6 membered, 4 to 5 membered, or 5 to 6 membered). In embodiments, R^{5B} is independently substituted heterocycloalkyl (e.g., 3 to 8 membered, 3 to 6 membered, 4 to 6 membered, 4 to 5 membered, or 5 to 6 membered). In embodiments, R^{5B} is independently unsubstituted heterocycloalkyl (e.g., 3 to 8 membered, 3 to 6 membered, 4 to 6 membered, 4 to 5 membered, or 5 to 6 membered). In embodiments, R^{5B} is independently substituted or unsubstituted aryl (e.g., C₆-C₁₀ or phenyl). In embodiments, R^{5B} is independently substituted aryl (e.g., C₆-C₁₀ or phenyl). In embodiments, R^{5B} is independently unsubstituted aryl (e.g., C₆-C₁₀ or phenyl). In embodiments, R^{5B} is independently substituted or unsubstituted heteroaryl (e.g., 5 to 10 membered, 5 to 9 membered, or 5 to 6 membered). In embodiments, R^{5B} is independently substituted heteroaryl (e.g., 5 to 10 membered, 5 to 9 membered, or 5 to 6 membered). In embodiments, R^{5B} is independently unsubstituted heteroaryl (e.g., 5 to 10 membered, 5 to 9 membered, or 5 to 6 membered).

In embodiments, R^{5A} and R^{5B} substituents bonded to the same nitrogen atom may be joined to form a substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may be joined to form a substituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may be joined to form an unsubstituted heterocycloalkyl (e.g., 3 to 8 membered, 3 to 6 membered, 4 to 6 membered, 4 to 5 membered, or 5 to 6 membered).

In embodiments, R^{5A} and R^{5B} substituents bonded to the same nitrogen atom may be joined to form a substituted or unsubstituted heteroaryl (e.g., 5 to 10 membered, 5 to 9 membered, or 5 to 6 membered). In embodiments, R^{5A} and R^{5B} substituents bonded to the same nitrogen atom may be joined to form a substituted heteroaryl (e.g., 5 to 10 membered, 5 to 9 membered, or 5 to 6 membered). In embodiments, R^{5A} and R^{5B} substituents bonded to the same nitrogen atom may be joined to form an unsubstituted heteroaryl (e.g., 5 to 10 membered, 5 to 9 membered, or 5 to 6 membered).

In embodiments, R⁵ is independently

hydrogen, —CX⁵₃, —CHX⁵₂, —CH₂X⁵, —CN, —COOH, —CONH₂, R³²-substituted or unsubstituted alkyl (e.g., C₁-C₈, C₁-C₆, C₁-C₄, or C₁-C₂), R³²-substituted or unsubstituted heteroalkyl (e.g., 2 to 8 membered, 2 to 6 membered, 4 to 6 membered, 2 to 3 membered, or 4 to 5 membered), R³²-substituted or unsubstituted cycloalkyl (e.g., C₃-C₈, C₃-C₆, C₄-C₆, or C₅-C₆), R³²-substituted or unsubstituted heterocycloalkyl (e.g., 3 to 8 membered, 3 to 6 membered, 4 to 6 membered, 4 to 5 membered, or 5 to 6 membered), R³²-substituted or unsubstituted aryl (e.g., C₆-C₁₀ or phenyl), or R³²-substituted or unsubstituted heteroaryl (e.g., 5 to 10 membered, 5 to 9 membered, or 5 to 6 membered). In embodiments, R⁵ is independently
hydrogen, —CX⁵₃, —CHX⁵₂, —CH₂X⁵, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X⁵ is independently —F, —Cl, —Br, or —I. In embodiments, R⁵ is independently hydrogen. In embodiments, R⁵ is independently unsubstituted methyl. In embodiments, R⁵ is independently unsubstituted ethyl.

R³² is independently oxo,

halogen, —CX³²₃, —CHX³²₂, —CH₂X³², —OCX³²₃, —OCH₂X³², —OCHX³²₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³³-substituted or unsubstituted alkyl (e.g., C₁-C₈, C₁-C₆, C₁-C₄, or C₁-C₂), R³³-substituted or unsubstituted heteroalkyl (e.g., 2 to 8 membered, 2 to 6 membered, 4 to 6 membered, 2 to 3 membered, or 4 to 5 membered), R³³-substituted or unsubstituted cycloalkyl (e.g., C₃-C₈, C₃-C₆, C₄-C₆, or C₅-C₆), R³³-substituted or unsubstituted heterocycloalkyl (e.g., 3 to 8 membered, 3 to 6 membered, 4 to 6 membered, 4 to 5 membered, or 5 to 6 membered), R³³-substituted or unsubstituted aryl (e.g., C₆-C₁₀ or phenyl), or R³³-substituted or unsubstituted heteroaryl (e.g., 5 to 10 membered, 5 to 9 membered, or 5 to 6 membered). In embodiments, R³² is independently oxo,
halogen, —CX³²₃, —CHX³²₂, —CH₂X³², —OCX³²₃, —OCH₂X³², —OCHX³²₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³² is independently —F, —Cl, —Br, or —I. In embodiments, R³² is independently unsubstituted methyl. In embodiments, R³² is independently unsubstituted ethyl.

R³³ is independently oxo,

halogen, —CX³³₃, —CHX³³₂, —CH₂X³³, —OCX³³₃, —OCH₂X³³, —OCHX³³₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R³⁴-substituted or unsubstituted alkyl (e.g., C₁-C₈, C₁-C₆, C₁-C₄, or C₁-C₂), R³⁴-substituted or unsubstituted heteroalkyl (e.g., 2 to 8 membered, 2 to 6 membered, 4 to 6 membered, 2 to 3 membered, or 4 to 5 membered), R³⁴-substituted or unsubstituted cycloalkyl (e.g., C₃-C₈, C₃-C₆, C₄-C₆, or C₅-C₆), R³⁴-substituted or unsubstituted heterocycloalkyl (e.g., 3 to 8 membered, 3 to 6 membered, 4 to 6 membered, 4 to 5 membered, or 5 to 6 membered), R³⁴-substituted or unsubstituted aryl (e.g., C₆-C₁₀ or phenyl), or R³⁴-substituted or unsubstituted heteroaryl (e.g., 5 to 10 membered, 5 to 9 membered, or 5 to 6 membered). In embodiments, R³³ is independently oxo,
halogen, —CX³³₃, —CHX³³₂, —CH₂X³³, —OCX³³₃, —OCH₂X³³, —OCHX³³₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³³ is independently —F, —Cl, —Br, or —I. In embodiments, R³³ is independently unsubstituted methyl. In embodiments, R³³ is independently unsubstituted ethyl.

R³⁴ is independently oxo,

halogen, —CX³⁴₃, —CHX³⁴₂, —CH₂X³⁴, —OCX³⁴₃, —OCH₂X³⁴, —OCHX³⁴₂, —CN, —OH, —NH₂, —COO H, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X³⁴ is independently —F, —Cl, —Br, or —I. In embodiments, R³⁴ is independently unsubstituted methyl. In embodiments, R³⁴ is independently unsubstituted ethyl.

In embodiments, R^{5A} is independently

hydrogen, —CX^{5A}₃, —CHX^{5A}₂, —CH₂X^{5A}, —CN, —COOH, —CONH₂, R^{32A}-substituted or unsubstituted alkyl (e.g., C₁-C₈, C₁-C₆, C₁-C₄, or C₁-C₂), R^{32A}-substituted or unsubstituted heteroalkyl (e.g., 2 to 8 membered, 2 to 6 membered, 4 to 6 membered, 2 to 3 membered, or 4 to 5 membered), R^{32A}-substituted or unsubstituted cycloalkyl (e.g., C₃-C₈, C₃-C₆, C₄-C₆, or C₅-C₆), R^{32A}-substituted or unsubstituted heterocycloalkyl (e.g., 3 to 8 membered, 3 to 6 membered, 4 to 6 membered, 4 to 5 membered, or 5 to 6 membered), R^{32A}-substituted or unsubstituted aryl (e.g., C₆-C₁₀ or phenyl), or R^{32A}-substituted or unsubstituted heteroaryl (e.g., 5 to 10 membered, 5 to 9 membered, or 5 to 6 membered). In embodiments, R^{5A} is independently hydrogen, —CX^{5A}₃, —CHX^{5A}₂, —CH₂X^{5A}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{5A} is independently —F, —Cl, —Br, or —I. In embodiments, R^{5A} is independently hydrogen. In embodiments, R^{5A} is independently unsubstituted methyl. In embodiments, R^{5A} is independently unsubstituted ethyl.

In embodiments, R^{5A} and R^{5B} substituents bonded to the same nitrogen atom may optionally be joined to form a R^{32A}-substituted or unsubstituted heterocycloalkyl (e.g., 3 to 8 membered, 3 to 6 membered, 4 to 6 membered, 4 to 5 membered, or 5 to 6 membered) or R^{32A}-substituted or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form a R^{32A}-substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{32A} is independently oxo,

halogen, —CX^{32A}₃, —CHX^{32A}₂, —CH₂X³²A, —OCX^{32A}₃, —OCH₂X^{32A}, —OCHX^{32A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{33A}-substituted or unsubstituted alkyl (e.g., C₁-C₈, C₁-C₆, C₁-C₄, or C₁-C₂), R^{33A}-substituted or unsubstituted heteroalkyl (e.g., 2 to 8 membered, 2 to 6 membered, 4 to 6 membered, 2 to 3 membered, or 4 to 5 membered), R^{33A}-substituted or unsubstituted cycloalkyl (e.g., C₃-C₈, C₃-C₆, C₄-C₆, or C₅-C₆), R^{33A}-substituted or unsubstituted heterocycloalkyl (e.g., 3 to 8 membered, 3 to 6 membered, 4 to 6 membered, 4 to 5 membered, or 5 to 6 membered), R^{33A}-substituted or unsubstituted aryl (e.g., C₆-C₁₀ or phenyl), or R^{33A}-substituted or unsubstituted heteroaryl (e.g., 5 to 10 membered, 5 to 9 membered, or 5 to 6 membered). In embodiments, R^{32A} is independently oxo,
halogen, —CX^{32A}₃, —CHX^{32A}₂, —CH₂X^{32A}, —OCX^{32A}₃, —OCH₂X^{32A}, —OCHX^{32A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{32A} is independently —F, —Cl, —Br, or —I. In embodiments, R^{32A} is independently unsubstituted methyl. In embodiments, R^{32A} is independently unsubstituted ethyl.

R^{33A} is independently oxo,

halogen, —CX^{33A}₃, —CHX^{33A}₂, —CH₂X^{33A}, —OCX^{33A}₃, —OCH₂X^{33A}, —OCHX^{33A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{34A}-substituted or unsubstituted alkyl (e.g., C₁-C₈, C₁-C₆, C₁-C₄, or C₁-C₂), R^{34A}-substituted or unsubstituted heteroalkyl (e.g., 2 to 8 membered, 2 to 6 membered, 4 to 6 membered, 2 to 3 membered, or 4 to 5 membered), R^{34A}-substituted or unsubstituted cycloalkyl (e.g., C₃-C₈, C₃-C₆, C₄-C₆, or C₅-C₆), R^{34A}-substituted or unsubstituted heterocycloalkyl (e.g., 3 to 8 membered, 3 to 6 membered, 4 to 6 membered, 4 to 5 membered, or 5 to 6 membered), R^{34A}-substituted or unsubstituted aryl (e.g., C₆-C₁₀ or phenyl), or R^{34A}-substituted or unsubstituted heteroaryl (e.g., 5 to 10 membered, 5 to 9 membered, or 5 to 6 membered). In embodiments, R^{33A} is independently oxo,
halogen, —CX^{33A}₃, —CHX^{33A}₂, —CH₂X^{33A}, —OCX^{33A}₃, —OCH₂X^{33A}, —OCHX^{33A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{33A} is independently —F, —Cl, —Br, or —I. In embodiments, R^{33A} is independently unsubstituted methyl. In embodiments, R^{33A} is independently unsubstituted ethyl.

R^{34A} is independently oxo,

halogen, —CX^{34A}₃, —CHX^{34A}₂, —CH₂X^{34A}, —OCX^{34A}₃, —OCH₂X^{34A}, —OCHX^{34A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{34A} is independently —F, —Cl, —Br, or —I. In embodiments, R^{34A} is independently unsubstituted methyl. In embodiments, R^{34A} is independently unsubstituted ethyl.

In embodiments, R^{5B} is independently

hydrogen, —CX^{5B}₃, —CHX^{5B}₂, —CH₂X^{5B}, —CN, —COOH, —CONH₂, R^{32B}-substituted or unsubstituted alkyl (e.g., C₁-C₈, C₁-C₆, C₁-C₄, or C₁-C₂), R^{32B}-substituted or unsubstituted heteroalkyl (e.g., 2 to 8 membered, 2 to 6 membered, 4 to 6 membered, 2 to 3 membered, or 4 to 5 membered), R^{32B}-substituted or unsubstituted cycloalkyl (e.g., C₃-C₈, C₃-C₆, C₄-C₆, or C₅-C₆), R^{32B}-substituted or unsubstituted heterocycloalkyl (e.g., 3 to 8 membered, 3 to 6 membered, 4 to 6 membered, 4 to 5 membered, or 5 to 6 membered), R^{32B}-substituted or unsubstituted aryl (e.g., C₆-C₁₀ or phenyl), or R^{32B}-substituted or unsubstituted heteroaryl (e.g., 5 to 10 membered, 5 to 9 membered, or 5 to 6 membered). In embodiments, R^{5B} is independently hydrogen, —CX^{5B}₃, —CHX^{5B}₂, —CH₂X^{5B}, —CN, —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{5B} is independently —F, —Cl, —Br, or —I. In embodiments, R^{5B} is independently hydrogen. In embodiments, R^{5B} is independently unsubstituted methyl. In embodiments, R^{5B} is independently unsubstituted ethyl.

In embodiments, R^{5A} and R^{5B} substituents bonded to the same nitrogen atom may optionally be joined to form a R^{32B}-substituted or unsubstituted heterocycloalkyl (e.g., 3 to 8 membered, 3 to 6 membered, 4 to 6 membered, 4 to 5 membered, or 5 to 6 membered) or R^{32B}-substituted or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered) or unsubstituted heteroaryl (e.g., 5 to 10 membered, 5 to 9 membered, or 5 to 6 membered). In embodiments, R^{5A} and R^{5B} substituents bonded to the same nitrogen atom may optionally be joined to form a R^{32B}-substituted or unsubstituted heterocycloalkyl (e.g., 3 to 8 membered, 3 to 6 membered, 4 to 6 membered, 4 to 5 membered, or 5 to 6 membered). In embodiments, R^{5A} and R^{5B} substituents bonded to the same nitrogen atom may optionally be joined to form an unsubstituted heterocycloalkyl (e.g., 3 to 8 membered, 3 to 6 membered, 4 to 6 membered, 4 to 5 membered, or 5 to 6 membered).

R^{32B} is independently oxo,

halogen, —CX^{32B}₃, —CHX^{32B}₂, —CH₂X^{32B}, —OCX^{32B}₃, —OCH₂X^{32B}, —OCHX^{32B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{33B}-substituted or unsubstituted alkyl (e.g., C₁-C₈, C₁-C₆, C₁-C₄, or C₁-C₂), R^{33B}-substituted or unsubstituted heteroalkyl (e.g., 2 to 8 membered, 2 to 6 membered, 4 to 6 membered, 2 to 3 membered, or 4 to 5 membered), R^{33B}-substituted or unsubstituted cycloalkyl (e.g., C₃-C₈, C₃-C₆, C₄-C₆, or C₅-C₆), R^{33B}-substituted or unsubstituted heterocycloalkyl (e.g., 3 to 8 membered, 3 to 6 membered, 4 to 6 membered, 4 to 5 membered, or 5 to 6 membered), R^{33B}-substituted or unsubstituted aryl (e.g., C₆-C₁₀ or phenyl), or R^{33B}-substituted or unsubstituted heteroaryl (e.g., 5 to 10 membered, 5 to 9 membered, or 5 to 6 membered). In embodiments, R^{32B} is independently oxo,
halogen, —CX^{32B}₃, —CHX^{32B}₂, —CH₂X^{32B}, —OCX^{32B}₃, —OCH₂X^{32B}, —OCHX^{32B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{32B} is independently —F, —Cl, —Br, or —I. In embodiments, R^{32B} is independently unsubstituted methyl. In embodiments, R^{32B} is independently unsubstituted ethyl.

R^{33B} is independently oxo,

halogen, —CX^{33B}₃, —CHX^{33B}₂, —CH₂X^{33B}, —OCX^{33B}₃, —OCH₂X^{33B}, —OCHX^{33B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, R^{34B}-substituted or unsubstituted alkyl (e.g., C₁-C₈, C₁-C₆, C₁-C₄, or C₁-C₂), R^{34B}-substituted or unsubstituted heteroalkyl (e.g., 2 to 8 membered, 2 to 6 membered, 4 to 6 membered, 2 to 3 membered, or 4 to 5 membered), R^{34B}-substituted or unsubstituted cycloalkyl (e.g., C₃-C₈, C₃-C₆, C₄-C₆, or C₅-C₆), R^{34B}-substituted or unsubstituted heterocycloalkyl (e.g., 3 to 8 membered, 3 to 6 membered, 4 to 6 membered, 4 to 5 membered, or 5 to 6 membered), R^{34B}-substituted or unsubstituted aryl (e.g., C₆-C₁₀ or phenyl), or R^{34B}-substituted or unsubstituted heteroaryl (e.g., 5 to 10 membered, 5 to 9 membered, or 5 to 6 membered). In embodiments, R^{33B} is independently oxo,
halogen, —CX^{33B}₃, —CHX^{33B}₂, —CH₂X^{33B}, —OCX^{33B}₃, —OCH₂X^{33B}, —OCHX^{33B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{33B} is independently —F, —Cl, —Br, or —I. In embodiments, R^{33B} is independently unsubstituted methyl. In embodiments, R^{33B} is independently unsubstituted ethyl.

R^{34B} is independently oxo,

halogen, —CX^{34B}₃, —CHX^{34B}₂, —CH₂X^{34B}, —OCX^{34B}₃, —OCH₂X^{34B}, —OCHX^{34B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). X^{34B} is independently —F, —Cl, —Br, or —I. In embodiments, R^{34B} is independently unsubstituted methyl. In embodiments, R^{34B} is independently unsubstituted ethyl.

In embodiments, X is —F. In embodiments, X is —Cl. In embodiments, X is —Br. In embodiments, X is —I. In embodiments, X¹ is —F. In embodiments, X¹ is —Cl. In embodiments, X¹ is —Br. In embodiments, X¹ is —I. In embodiments, X² is —F. In embodiments, X² is —Cl. In embodiments, X² is —Br. In embodiments, X² is —I. In embodiments, X⁴ is —F. In embodiments, X⁴ is —Cl. In embodiments, X⁴ is —Br. In embodiments, X⁴ is —I. In embodiments, X⁵ is —F. In embodiments, X⁵ is —Cl. In embodiments, X⁵ is —Br. In embodiments, X⁵ is —I.

In embodiments, n1 is 0. In embodiments, n1 is 1. In embodiments, n1 is 2. In embodiments, n1 is 3. In embodiments, n1 is 4. In embodiments, n2 is 0. In embodiments, n2 is 1. In embodiments, n2 is 2. In embodiments, n2 is 3. In embodiments, n2 is 4. In embodiments, n4 is 0. In embodiments, n4 is 1. In embodiments, n4 is 2. In embodiments, n4 is 3. In embodiments, n4 is 4. In embodiments, n5 is 0. In embodiments, n5 is 1. In embodiments, n5 is 2. In embodiments, n5 is 3. In embodiments, n5 is 4.

In embodiments, m1 is 1. In embodiments, m1 is 2. In embodiments, m2 is 1. In embodiments, m2 is 2. In embodiments, m4 is 1. In embodiments, m4 is 2. In embodiments, m5 is 1. In embodiments, m5 is 2.

In embodiments, v1 is 1. In embodiments, v1 is 2. In embodiments, v2 is 1. In embodiments, v2 is 2. In embodiments, v4 is 1. In embodiments, v4 is 2. In embodiments, v5 is 1. In embodiments, v5 is 2.

In embodiments, E is a covalent cysteine modifier moiety.

In embodiments, E is:

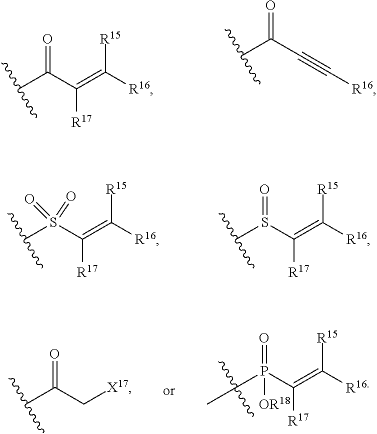

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR¹⁶D, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁷ is independently hydrogen, halogen, CX¹⁷₃, —CHX¹⁷₂, —CH₂X¹⁷, —CN, —SOₙ₁R^{17D}, —SO₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

Each R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl. Each X, X¹⁵, X¹⁶, X¹⁷ and X¹⁸ is independently —F, —Cl, —Br, or —I. The symbols n15, n16, n17, v15, v16, and v17, are independently and integer from 0 to 4. The symbols m15, m16, and m17 are independently and integer between 1 and 2.

In embodiments, E is:

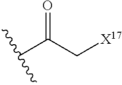

and X¹⁷ is —Cl. In embodiments, E is:

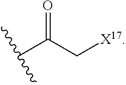

In embodiments, X¹⁷ is —Cl.

In embodiments, E is:

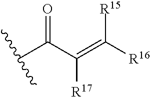

and R¹⁵, R¹⁶, and R¹⁷ are independently hydrogen. In embodiments, E is:

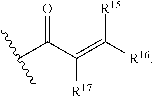

In embodiments, R¹⁵, R¹⁶, and R¹⁷ are independently hydrogen.

In embodiments, E is:

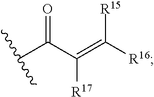

R¹⁵ is independently hydrogen; R¹⁶ is independently hydrogen or —CH₂NR^{16A}R^{16B}; R¹⁷ is independently hydrogen; and R^{16A} and R^{16B} are independently hydrogen or unsubstituted alkyl. In embodiments, E is:

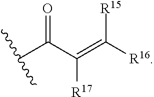

In embodiments, R¹⁵ is independently hydrogen. In embodiments, R¹⁶ is independently hydrogen or —CH₂NR^{16A}R^{16B}. In embodiments, R¹⁷ is independently hydrogen. In embodiments, R^{16A} and R^{16B} are independently hydrogen or unsubstituted alkyl. In embodiments, R^{16A} and R^{16B} are independently unsubstituted methyl.

In embodiments, E is:

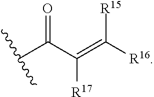

In embodiments, E is:

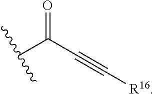

In embodiments, E is:

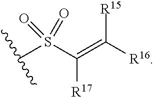

In embodiments, E is:

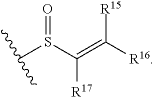

In embodiments, E is:

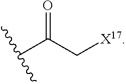

In embodiments, E is:

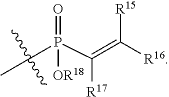

In embodiments, E is:

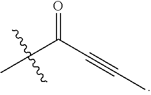 [SMILES: *C(=O)C#CC]

In embodiments, E is:

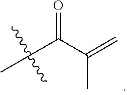 [SMILES: *C(=O)C(=C)C]

In embodiments, E is:

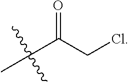 [SMILES: *C(=O)CCl]

X may independently be —F. X may independently be —Cl. X may independently be —Br. X may independently be —I. X¹⁵ may independently be —F. X¹⁵ may independently be —Cl. X¹⁵ may independently be —Br. X¹⁵ may independently be —I. X¹⁶ may independently be —F. X¹⁶ may independently be —Cl. X¹⁶ may independently be —Br. X¹⁶ may independently be —I. X⁷ may independently be —F. X⁷ may independently be —Cl. X¹⁷ may independently be —Br. X¹⁷ may independently be —I. X¹⁸ may independently be —F. X¹⁸ may independently be —Cl. X¹⁸ may independently be —Br. X¹⁸ may independently be —I. n15 may independently be 0. n15 may independently be 1. n15 may independently be 2. n15 may independently be 3. n15 may independently be 4. n16 may independently be 0. n16 may independently be 1. n16 may independently be 2. n16 may independently be 3. n16 may independently be 4. n17 may independently be 0. n17 may independently be 1. n17 may independently be 2. n17 may independently be 3. n17 may independently be 4. v15 may independently be 0. v15 may independently be 1. v15 may independently be 2. v15 may independently be 3. v15 may independently be 4. v16 may independently be 0. v16 may independently be 1. v16 may independently be 2. v16 may independently be 3. v16 may independently be 4. v17 may independently be 0. v17 may independently be 1. v17 may independently be 2. v17 may independently be 3. v17 may independently be 4. m15 may independently be 1. m15 may independently be 2. m16 may independently be 1. m16 may independently be 2. m17 may independently be 1. m17 may independently be 2.

In embodiments, R¹⁵ is hydrogen. In embodiments, R¹⁵ is halogen. In embodiments, R¹⁵ is CX¹⁵₃. In embodiments, R¹⁵ is —CHX¹⁵₂. In embodiments, R¹⁵ is —CH₂X¹⁵. In embodiments, R¹⁵ is —CN. In embodiments, R¹⁵ is —SOₙ₁₅R^{15D}. In embodiments, R¹⁵ is —SOᵥ₁₅NR^{15A}R^{15B}. In embodiments, R¹⁵ is —NHNR^{15A}R^{15B}. In embodiments, R¹⁵ is —ONR^{15A}R^{15B}. In embodiments, R¹⁵ is —NHC═(O)NHNR^{15A}R^{15B}. In embodiments, R¹⁵ is —NHC(O)NR^{15A}R^{15B}. In embodiments, R¹⁵ is —N(O)ₘ₁₅. In embodiments, R¹⁵ is —NR^{15A}R^{15B} In embodiments, R¹⁵ is —C(O)R^{15C}. In embodiments, R¹⁵ is —C(O)—OR^{15C}. In embodiments, R¹⁵ is —C(O)NR^{15A}R^{15B}. In embodiments, R¹⁵ is —OR^{15D}. In embodiments, R¹⁵ is —NR^{15A}SO₂R^{15D}. In embodiments, R¹⁵ is —NR^{15A}C(O)R^{15C}. In embodiments, R¹⁵ is —NR^{15A}C(O)OR^{15C}. In embodiments, R¹⁵ is —NR^{15A}OR^{15C}. In embodiments, R¹⁵ is —OCX¹⁵₃. In embodiments, R¹⁵ is —OCHX¹⁵₂. In embodiments, R¹⁵ is substituted or unsubstituted alkyl. In embodiments, R¹⁵ is substituted or unsubstituted heteroalkyl. In embodiments, R¹⁵ is substituted or unsubstituted cycloalkyl. In embodiments, R¹⁵ is substituted or unsubstituted heterocycloalkyl. In embodiments, R¹⁵ is substituted or unsubstituted aryl. In embodiments, R¹⁵ is substituted or unsubstituted heteroaryl. In embodiments, R¹⁵ is substituted alkyl. In embodiments, R¹⁵ is substituted heteroalkyl. In embodiments, R¹⁵ is substituted cycloalkyl. In embodiments, R¹⁵ is substituted heterocycloalkyl. In embodiments, R¹⁵ is substituted aryl. In embodiments, R¹⁵ is substituted heteroaryl. In embodiments, R¹⁵ is unsubstituted alkyl. In embodiments, R¹⁵ is unsubstituted heteroalkyl. In embodiments, R¹⁵ is unsubstituted cycloalkyl. In embodiments, R¹⁵ is unsubstituted heterocycloalkyl. In embodiments, R¹⁵ is unsubstituted aryl. In embodiments, R¹⁵ is unsubstituted heteroaryl. In embodiments, R¹⁵ is unsubstituted methyl. In embodiments, R¹⁵ is unsubstituted ethyl. In embodiments, R¹⁵ is unsubstituted propyl. In embodiments, R¹⁵ is unsubstituted isopropyl. In embodiments, R¹⁵ is unsubstituted butyl. In embodiments, R¹⁵ is unsubstituted tert-butyl.

In embodiments, R^{15A} is hydrogen. In embodiments, R^{15A} is —CX₃. In embodiments, R^{15A} is —CN. In embodiments, R^{15A} is —COOH. In embodiments, R^{15A} is —CONH₂. In embodiments, R^{15A} is —CHX₂. In embodiments, R^{15A} is —CH₂X. In embodiments, R^{15A} is unsubstituted methyl. In embodiments, R^{15A} is unsubstituted ethyl. In embodiments, R^{15A} is unsubstituted propyl. In embodiments, R^{15A} is unsubstituted isopropyl. In embodiments, R^{15A} is unsubstituted butyl. In embodiments, R^{15A} is unsubstituted tert-butyl.

In embodiments, R^{15B} is hydrogen. In embodiments, R^{15B} is —CX₃. In embodiments, R^{15B} is —CN. In embodiments, R^{15B} is —COOH. In embodiments, R^{15B} is —CONH₂. In embodiments, R^{15B} is —CHX₂. In embodiments, R^{15B} is —CH₂X. In embodiments, R^{15B} is unsubstituted methyl. In embodiments, R^{15B} is unsubstituted ethyl. In embodiments, R^{15B} is unsubstituted propyl. In embodiments, R^{15B} is unsubstituted isopropyl. In embodiments, R^{15B} is unsubstituted butyl. In embodiments, R^{15B} is unsubstituted tert-butyl.

In embodiments, R^{15C} is hydrogen. In embodiments, R^{15C} is —CX₃. In embodiments, R^{15C} is —CN. In embodiments, R^{15C} is —COOH. In embodiments, R^{15C} is —CONH₂. In embodiments, R^{15C} is —CHX₂. In embodiments, R^{15C} is —CH₂X. In embodiments, R^{15C} is unsubstituted methyl. In embodiments, R^{15C} is unsubstituted ethyl. In embodiments, R^{15C} is unsubstituted propyl. In embodiments, R^{15C} is unsubstituted isopropyl. In embodiments, R^{15C} is unsubstituted butyl. In embodiments, R^{15C} is unsubstituted tert-butyl.

In embodiments, R^{15D} is hydrogen. In embodiments, R^{15D} is —CX₃. In embodiments, R^{15D} is —CN. In embodiments, R^{15D} is —COOH. In embodiments, R^{15D} is —CONH₂. In embodiments, R^{15D} is —CHX₂. In embodiments, R^{15D} is —CH₂X. In embodiments, R^{15D} is unsubstituted methyl. In embodiments, R^{15D} is unsubstituted ethyl. In embodiments, R^{15D} is unsubstituted propyl. In embodiments, R^{15D} is unsubstituted isopropyl. In embodiments, R^{15D} is unsubstituted butyl. In embodiments, R^{15D} is unsubstituted tert-butyl.

In embodiments, R¹⁵ is independently hydrogen, oxo,

halogen, —CX¹⁵₃, —CHX¹⁵₂, —OCH₂X¹⁵, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁵₃, —OCHX¹⁵₂, R⁷²-substituted or unsubstituted alkyl, R⁷²-substituted or unsubstituted heteroalkyl, R⁷²-substituted or unsubstituted cycloalkyl, R⁷²-substituted or unsubstituted heterocycloalkyl, R⁷²-substituted or unsubstituted aryl, or R⁷²-substituted or unsubstituted heteroaryl. X¹⁵ is halogen. In embodiments, X¹⁵ is F.

R⁷² is independently oxo,

halogen, —CX⁷²₃, —CHX⁷²₂, —OCH₂X⁷², —OCHX⁷²₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷²₃, —OCHX⁷²₂, R⁷³-substituted or unsubstituted alkyl, R⁷³-substituted or unsubstituted heteroalkyl, R⁷³-substituted or unsubstituted cycloalkyl, R⁷³-substituted or unsubstituted heterocycloalkyl, R⁷³-substituted or unsubstituted aryl, or R⁷³-substituted or unsubstituted heteroaryl. X⁷² is halogen. In embodiments, X⁷² is F.

R⁷³ is independently oxo,

halogen, —CX⁷³₃, —CHX⁷³₂, —OCH₂X⁷³, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷³₃, —OCHX⁷³₂, R⁷⁴-substituted or unsubstituted alkyl, R⁷⁴-substituted or unsubstituted heteroalkyl, R⁷⁴-substituted or unsubstituted cycloalkyl, R⁷⁴-substituted or unsubstituted heterocycloalkyl, R⁷⁴-substituted or unsubstituted aryl, or R⁷⁴-substituted or unsubstituted heteroaryl. X⁷³ is halogen. In embodiments, X⁷³ is F.

In embodiments, R^{15A} is independently hydrogen, oxo,

halogen, —CX^{15A}₃, —CHX^{15A}₂, —OCH₂X^{15A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15A}₃, —OCHX^{15A}₂, R^{72A}-substituted or unsubstituted alkyl, R^{72A}-substituted or unsubstituted heteroalkyl, R^{72A}-substituted or unsubstituted cycloalkyl, R^{72A}-substituted or unsubstituted heterocycloalkyl, R^{72A}-substituted or unsubstituted aryl, or R^{72A}-substituted or unsubstituted heteroaryl. X^{15A} is halogen. In embodiments, X^{15A} is F.

R^{72A} is independently oxo,

halogen, —CX^{72A}₃, —CHX^{72A}₂, —OCH₂X^{72A}, —OCHX^{72A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72A}₃, —OCHX^{72A}₂, R^{73A}-substituted or unsubstituted alkyl, R^{73A}-substituted or unsubstituted heteroalkyl, R^{73A}-substituted or unsubstituted cycloalkyl, R^{73A}-substituted or unsubstituted heterocycloalkyl, R^{73A}-substituted or unsubstituted aryl, or R^{73A}-substituted or unsubstituted heteroaryl. X^{72A} is halogen. In embodiments, X^{72A} is F.

R^{73A} is independently oxo,

halogen, —CX^{73A}₃, —CHX^{73A}₂, —OCH₂X^{73A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73A}₃, —OCHX^{73A}₂, R^{74A}-substituted or unsubstituted alkyl, R^{74A}-substituted or unsubstituted heteroalkyl, R^{74A}-substituted or unsubstituted cycloalkyl, R^{74A}-substituted or unsubstituted heterocycloalkyl, R^{74A}-substituted or unsubstituted aryl, or R^{74A}-substituted or unsubstituted heteroaryl. X^{73A} is halogen. In embodiments, X^{73A} is F.

In embodiments, R^{15B} is independently hydrogen, oxo,

halogen, —CX^{15B}₃, —CHX^{15B}₂, —OCH₂X^{15B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15B}₃, —OCHX^{15B}₂, R^{72B}-substituted or unsubstituted alkyl, R^{72B}-substituted or unsubstituted heteroalkyl, R^{72B}-substituted or unsubstituted cycloalkyl, R^{72B}-substituted or unsubstituted heterocycloalkyl, R^{72B}-substituted or unsubstituted aryl, or R^{72B}-substituted or unsubstituted heteroaryl. X^{15B} is halogen. In embodiments, X^{15B} is F.

R^{72B} is independently oxo,

halogen, —CX^{72B}₃, —CHX^{72B}₂, —OCH₂X^{72B}, —OCHX^{72B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72B}₃, —OCHX^{72B}₂, R^{73B}-substituted or unsubstituted alkyl, R^{73B}-substituted or unsubstituted heteroalkyl, R^{73B}-substituted or unsubstituted cycloalkyl, R^{73B}-substituted or unsubstituted heterocycloalkyl, R^{73B}-substituted or unsubstituted aryl, or R^{73B}-substituted or unsubstituted heteroaryl. X^{72B} is halogen. In embodiments, X^{72B} is F.

R^{73B} is independently oxo,

halogen, —CX^{73B}₃, —CHX^{73B}₂, —OCH₂X^{73B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73B}₃, —OCHX^{73B}₂, R^{74B}-substituted or unsubstituted alkyl, R^{74B}-substituted or unsubstituted heteroalkyl, R^{74B}-substituted or unsubstituted cycloalkyl, R^{74B}-substituted or unsubstituted heterocycloalkyl, R^{74B}-substituted or unsubstituted aryl, or R^{74B}-substituted or unsubstituted heteroaryl. X^{73B} is halogen. In embodiments, X^{73B} is F.

In embodiments, R^{15C} is independently hydrogen, oxo,

halogen, —CX^{15C}₃, —CHX^{15C}₂, —OCH₂X^{15C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15C}₃, —OCHX^{15C}₂, R^{72C}-substituted or unsubstituted alkyl, R^{72C}-substituted or unsubstituted heteroalkyl, R^{72C}-substituted or unsubstituted cycloalkyl, R^{72C}-substituted or unsubstituted heterocycloalkyl, R^{72C}-substituted or unsubstituted aryl, or R^{72C}-substituted or unsubstituted heteroaryl. X^{15C} is halogen. In embodiments, X^{15C} is F.

R^{72C} is independently oxo,

halogen, —CX^{72C}₃, —CHX^{72C}₂, —OCH₂X^{72C}, —OCHX^{72C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72C}₃, —OCHX^{72C}₂, R^{73C}-substituted or unsubstituted alkyl, R^{73C}-substituted or unsubstituted heteroalkyl, R^{73C}-substituted or unsubstituted cycloalkyl, R^{73C}-substituted or unsubstituted heterocycloalkyl, R^{73C}-substituted or unsubstituted aryl, or R^{73C}-substituted or unsubstituted heteroaryl. X^{72C} is halogen. In embodiments, X^{72C} is F.

R^{73C} is independently oxo,

halogen, —CX^{73C}₃, —CHX^{73C}₂, —OCH₂X^{73C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73C}₃, —OCHX^{73C}₂, R^{74C}-substituted or unsubstituted alkyl, R^{74C}-substituted or unsubstituted heteroalkyl, R^{74C}-substituted or unsubstituted cycloalkyl, R^{74C}-substituted or unsubstituted heterocycloalkyl, R^{74C}-substituted or unsubstituted aryl, or R^{74C}-substituted or unsubstituted heteroaryl. X^{73C} is halogen. In embodiments, X^{73C} is F.

In embodiments, R^{15D} is independently hydrogen, oxo,

halogen, —CX^{15D}₃, —CHX^{15D}₂, —OCH₂X^{15D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{15D}₃, —OCHX^{15D}₂, R^{72D}-substituted or unsubstituted alkyl, R^{72D}-substituted or unsubstituted heteroalkyl, R^{72D}-substituted or unsubstituted cycloalkyl, R^{72D}-substituted or unsubstituted heterocycloalkyl, R^{72D}-substituted or unsubstituted aryl, or R^{72D}-substituted or unsubstituted heteroaryl. X^{15D} is halogen. In embodiments, X^{15D} is F.

R^{72D} is independently oxo,

halogen, —CX^{72D}₃, —CHX^{72D}₂, —OCH₂X^{72D}, —OCHX^{72D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{72D}₃, —OCHX^{72D}₂, R^{73D}-substituted or unsubstituted alkyl, R^{73D}-substituted or unsubstituted heteroalkyl, R^{73D}-substituted or unsubstituted cycloalkyl, R^{73D}-substituted or unsubstituted heterocycloalkyl, R^{73D}-substituted or unsubstituted aryl, or R^{73D}-substituted or unsubstituted heteroaryl. X^{72D} is halogen. In embodiments, X^{72D} is F.

R^{73D} is independently oxo,

halogen, —CX^{73D}₃, —CHX^{73D}₂, —OCH₂X^{73D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{73D}₃, —OCHX^{73D}₂, R^{74D}-substituted or unsubstituted alkyl, R^{74D}-substituted or unsubstituted heteroalkyl, R^{74D}-substituted or unsubstituted cycloalkyl, R^{74D}-substituted or unsubstituted heterocycloalkyl, R^{74D}-substituted or unsubstituted aryl, or R^{74D}-substituted or unsubstituted heteroaryl. X^{73D} is halogen. In embodiments, X^{73D} is F.

In embodiments, R¹⁶ is hydrogen. In embodiments, R¹⁶ is halogen. In embodiments, R¹⁶ is CX¹⁶₃. In embodiments, R¹⁶ is —CHX¹⁶₂. In embodiments, R¹⁶ is —CH₂X¹⁶. In embodiments, R¹⁶ is —CN. In embodiments, R¹⁶ is —SOₙ₁₆R^{16D}. In embodiments, R¹⁶ is —SOᵥ₁₆NR^{16A}R^{16B}. In embodiments, R¹⁶ is —NHNR^{16A}R^{16B}. In embodiments, R¹⁶ is —ONR^{16A}R^{16B}. In embodiments, R¹⁶ is —NHC═(O)NHNR^{16A}R^{16B}. In embodiments, R¹⁶ is —NHC(O)NR^{16A}R^{16B}. In embodiments, R¹⁶ is —N(O)ₘ₁₆. In embodiments, R¹⁶ is —NR^{16A}R^{16B}. In embodiments, R¹⁶ is —C(O)R^{16C}. In embodiments, R¹⁶ is —C(O)—OR^{16C}. In embodiments, R¹⁶ is —C(O)NR^{16A}R^{16B}. In embodiments, R¹⁶ is —OR^{16D}. In embodiments, R¹⁶ is —NR^{16A}SO₂R^{16D}. In embodiments, R¹⁶ is —NR^{16A}C(O)R^{16C}. In embodiments, R¹⁶ is —NR^{16A}C(O)OR^{16C}. In embodiments, R¹⁶ is —NR^{16A}OR^{16C}. In embodiments, R¹⁶ is —OCX¹⁶₃. In embodiments, R¹⁶ is —OCHX¹⁶₂. In embodiments, R¹⁶ is substituted or unsubstituted alkyl. In embodiments, R¹⁶ is substituted or unsubstituted heteroalkyl. In embodiments, R¹⁶ is substituted or unsubstituted cycloalkyl. In embodiments, R¹⁶ is substituted or unsubstituted heterocycloalkyl. In embodiments, R¹⁶ is substituted or unsubstituted aryl. In embodiments, R¹⁶ is substituted or unsubstituted heteroaryl. In embodiments, R¹⁶ is substituted alkyl. In embodiments, R¹⁶ is substituted heteroalkyl. In embodiments, R¹⁶ is substituted cycloalkyl. In embodiments, R¹⁶ is substituted heterocycloalkyl. In embodiments, R¹⁶ is substituted aryl. In embodiments, R¹⁶ is substituted heteroaryl. In embodiments, R¹⁶ is unsubstituted alkyl. In embodiments, R¹⁶ is unsubstituted heteroalkyl. In embodiments, R¹⁶ is unsubstituted cycloalkyl. In embodiments, R¹⁶ is unsubstituted heterocycloalkyl. In embodiments, R¹⁶ is unsubstituted aryl. In embodiments, R¹⁶ is unsubstituted heteroaryl. In embodiments, R¹⁶ is unsubstituted methyl. In embodiments, R¹⁶ is unsubstituted ethyl. In embodiments, R¹⁶ is unsubstituted propyl. In embodiments, R¹⁶ is unsubstituted isopropyl. In embodiments, R¹⁶ is unsubstituted butyl. In embodiments, R¹⁶ is unsubstituted tert-butyl.

In embodiments, R^{16A} is hydrogen. In embodiments, R^{16A} is —CX₃. In embodiments, R^{16A} is —CN. In embodiments, R^{16A} is —COOH. In embodiments, R^{16A} is —CONH₂. In embodiments, R^{16A} is —CHX₂. In embodiments, R^{16A} is —CH₂X. In embodiments, R^{16A} is unsubstituted methyl. In embodiments, R^{16A} is unsubstituted ethyl. In embodiments, R^{16A} is unsubstituted propyl. In embodiments, R^{16A} is unsubstituted isopropyl. In embodiments, R^{16A} is unsubstituted butyl. In embodiments, R^{16A} is unsubstituted tert-butyl.

In embodiments, R^{16B} is hydrogen. In embodiments, R^{16B} is —CX₃. In embodiments, R^{16B} is —CN. In embodiments, R^{16B} is —COOH. In embodiments, R^{16B} is —CONH₂. In embodiments, R^{16B} is —CHX₂. In embodiments, R^{16B} is —CH₂X. In embodiments, R^{16B} is unsubstituted methyl. In embodiments, R^{16B} is unsubstituted ethyl. In embodiments, R^{16B} is unsubstituted propyl. In embodiments, R^{16B} is unsubstituted isopropyl. In embodiments, R^{16B} is unsubstituted butyl. In embodiments, R^{16B} is unsubstituted tert-butyl.

In embodiments, R^{16C} is hydrogen. In embodiments, R^{16C} is —CX₃. In embodiments, R^{16C} is —CN. In embodiments, R^{16C} is —COOH. In embodiments, R^{16C} is —CONH₂. In embodiments, R^{16C} is —CHX₂. In embodiments, R^{16C} is —CH₂X. In embodiments, R^{16C} is unsubstituted methyl. In embodiments, R^{16C} is unsubstituted ethyl. In embodiments, R^{16C} is unsubstituted propyl. In embodiments, R^{16C} is unsubstituted isopropyl. In embodiments, R^{16C} is unsubstituted butyl. In embodiments, R^{16C} is unsubstituted tert-butyl.

In embodiments, R^{16D} is hydrogen. In embodiments, R^{16D} is —CX₃. In embodiments, R^{16D} is —CN. In embodiments, R^{16D} is —COOH. In embodiments, R^{16D} is —CONH₂. In embodiments, R^{16D} is —CHX₂. In embodiments, R^{16D} is —CH₂X. In embodiments, R^{16D} is unsubstituted methyl. In embodiments, R^{16D} is unsubstituted ethyl. In embodiments, R^{16D} is unsubstituted propyl. In embodiments, R^{16D} is unsubstituted isopropyl. In embodiments, R^{16D} is unsubstituted butyl. In embodiments, R^{16D} is unsubstituted tert-butyl.

In embodiments, R¹⁶ is independently hydrogen, oxo,

halogen, —CX¹⁶₃, —CHX¹⁶₂, —OCH₂X¹⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁶₃, —OCHX¹⁶₂, R⁷⁵-substituted or unsubstituted alkyl, R⁷⁵-substituted or unsubstituted heteroalkyl, R⁷⁵-substituted or unsubstituted cycloalkyl, R⁷⁵-substituted or unsubstituted heterocycloalkyl, R⁷⁵-substituted or unsubstituted aryl, or R⁷⁵-substituted or unsubstituted heteroaryl. X¹⁶ is halogen. In embodiments, X¹⁶ is F.

R⁷⁵ is independently oxo,

halogen, —CX⁷⁵₃, —CHX⁷⁵₂, —OCH₂X⁷⁵, —OCHX⁷⁵₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁵₃, —OCHX⁷⁵₂, R⁷⁶-substituted or unsubstituted alkyl, R⁷⁶-substituted or unsubstituted heteroalkyl, R⁷⁶-substituted or unsubstituted cycloalkyl, R⁷⁶-substituted or unsubstituted heterocycloalkyl, R⁷⁶-substituted or unsubstituted aryl, or R⁷⁶-substituted or unsubstituted heteroaryl. X⁷⁵ is halogen. In embodiments, X⁷⁵ is F.

R⁷⁶ is independently oxo,

halogen, —CX⁷⁶₃, —CHX⁷⁶₂, —OCH₂X⁷⁶, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁶₃, —OCHX⁷⁶₂, R⁷⁷-substituted or unsubstituted alkyl, R⁷⁷-substituted or unsubstituted heteroalkyl, R⁷⁷-substituted or unsubstituted cycloalkyl, R⁷⁷-substituted or unsubstituted heterocycloalkyl, R⁷⁷-substituted or unsubstituted aryl, or R⁷⁷-substituted or unsubstituted heteroaryl. X⁷⁶ is halogen. In embodiments, X⁷⁶ is F.

In embodiments, R^{16A} is independently hydrogen, oxo,

halogen, —CX^{16A}₃, —CHX^{16A}₂, —OCH₂X^{16A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16A}₃, —OCHX^{16A}₂, R^{75A}-substituted or unsubstituted alkyl, R^{75A}-substituted or unsubstituted heteroalkyl, R^{75A}-substituted or unsubstituted cycloalkyl, R^{75A}-substituted or unsubstituted heterocycloalkyl, R^{75A}-substituted or unsubstituted aryl, or R^{75A}-substituted or unsubstituted heteroaryl. X^{16A} is halogen. In embodiments, X^{16A} is F.

R^{75A} is independently oxo,

halogen, —CX^{75A}₃, —CHX^{75A}₂, —OCH₂X^{75A}, —OCHX^{75A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75A}₃, —OCHX^{75A}₂, R^{76A}-substituted or unsubstituted alkyl, R^{76A}-substituted or unsubstituted heteroalkyl, R^{76A}-substituted or unsubstituted cycloalkyl, R^{76A}-substituted or unsubstituted heterocycloalkyl, R^{76A}-substituted or unsubstituted aryl, or R^{76A}-substituted or unsubstituted heteroaryl. X^{75A} is halogen. In embodiments, X^{75A} is F.

R^{76A} is independently oxo,

halogen, —CX^{76A}₃, —CHX^{76A}₂, —OCH₂X^{76A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76A}₃, —OCHX^{76A}₂, R^{77A}-substituted or unsubstituted alkyl, R^{77A}-substituted or unsubstituted heteroalkyl, R^{77A}-substituted or unsubstituted cycloalkyl, R^{77A}-substituted or unsubstituted heterocycloalkyl, R^{77A}-substituted or unsubstituted aryl, or R^{77A}-substituted or unsubstituted heteroaryl. X^{76A} is halogen. In embodiments, X^{76A} is F.

In embodiments, R^{16B} is independently hydrogen, oxo,

halogen, —CX^{16B}₃, —CHX^{16B}₂, —OCH₂X^{16B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16B}₃, —OCHX^{16B}₂, R^{75B}-substituted or unsubstituted alkyl, R^{75B}-substituted or unsubstituted heteroalkyl, R^{75B}-substituted or unsubstituted cycloalkyl, R^{75B}-substituted or unsubstituted heterocycloalkyl, R^{75B}-substituted or unsubstituted aryl, or R^{75B}-substituted or unsubstituted heteroaryl. X^{16B} is halogen. In embodiments, X^{16B} is F.

R^{75B} is independently oxo,

halogen, —CX^{75B}₃, —CHX^{75B}₂, —OCH₂X^{75B}, —OCHX^{75B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75B}₃, —OCHX^{75B}₂, R^{76B}-substituted or unsubstituted alkyl, R^{76B}-substituted or unsubstituted heteroalkyl, R^{76B}-substituted or unsubstituted cycloalkyl, R^{76B}-substituted or unsubstituted heterocycloalkyl, R^{76B}-substituted or unsubstituted aryl, or R^{76B}-substituted or unsubstituted heteroaryl. X^{75B} is halogen. In embodiments, X^{75B} is F.

R^{76B} is independently oxo,

halogen, —CX^{76B}₃, —CHX^{76B}₂, —OCH₂X^{76B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76B}₃, —OCHX^{76B}₂, R^{77B}-substituted or unsubstituted alkyl, R^{77B}-substituted or unsubstituted heteroalkyl, R^{77B}-substituted or unsubstituted cycloalkyl, R^{77B}-substituted or unsubstituted heterocycloalkyl, R^{77B}-substituted or unsubstituted aryl, or R^{77B}-substituted or unsubstituted heteroaryl. X^{76B} is halogen. In embodiments, X^{76B} is F.

In embodiments, R^{16C} is independently hydrogen, oxo,

halogen, —CX^{16C}₃, —CHX^{16C}₂, —OCH₂X^{16C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16C}₃, —OCHX^{16C}₂, R^{75C}-substituted or unsubstituted alkyl, R^{75C}-substituted or unsubstituted heteroalkyl, R^{75C}-substituted or unsubstituted cycloalkyl, R^{75C}-substituted or unsubstituted heterocycloalkyl, R^{75C}-substituted or unsubstituted aryl, or R^{75C}-substituted or unsubstituted heteroaryl. X^{16C} is halogen. In embodiments, X^{16C} is F.

R^{75C} is independently oxo,

halogen, —CX^{75C}₃, —CHX^{75C}₂, —OCH₂X^{75C}, —OCHX^{75C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75C}₃, —OCHX^{75C}₂, R^{76C}-substituted or unsubstituted alkyl, R^{76C}-substituted or unsubstituted heteroalkyl, R^{76C}-substituted or unsubstituted cycloalkyl, R^{76C}-substituted or unsubstituted heterocycloalkyl, R^{76C}-substituted or unsubstituted aryl, or R^{76C}-substituted or unsubstituted heteroaryl. X^{75C} is halogen. In embodiments, X^{75C} is F.

R^{76C} is independently oxo,

halogen, —CX^{76C}₃, —CHX^{76C}₂, —OCH₂X^{76C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76C}₃, —OCHX^{76C}₂, R^{77C}-substituted or unsubstituted alkyl, R^{77C}-substituted or unsubstituted heteroalkyl, R^{77C}-substituted or unsubstituted cycloalkyl, R^{77C}-substituted or unsubstituted heterocycloalkyl, R^{77C}-substituted or unsubstituted aryl, or R^{77C}-substituted or unsubstituted heteroaryl. X^{76C} is halogen. In embodiments, X^{76C} is F.

In embodiments, R^{16D} is independently hydrogen, oxo,

halogen, —CX^{16D}₃, —CHX^{16D}₂, —OCH₂X^{16D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{16D}₃, —OCHX^{16D}₂, R^{75D}-substituted or unsubstituted alkyl, R^{75D}-substituted or unsubstituted heteroalkyl, R^{75D}-substituted or unsubstituted cycloalkyl, R^{75D}-substituted or unsubstituted heterocycloalkyl, R^{75D}-substituted or unsubstituted aryl, or R^{75D}-substituted or unsubstituted heteroaryl. X^{16D} is halogen. In embodiments, X^{16D} is F.

R^{75D} is independently oxo,

halogen, —CX^{75D}₃, —CHX^{75D}₂, —OCH₂X^{75D}, —OCHX^{75D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{75D}₃, —OCHX^{75D}₂, R^{76D}-substituted or unsubstituted alkyl, R^{76D}-substituted or unsubstituted heteroalkyl, R^{76D}-substituted or unsubstituted cycloalkyl, R^{76D}-substituted or unsubstituted heterocycloalkyl, R^{76D}-substituted or unsubstituted aryl, or R^{76D}-substituted or unsubstituted heteroaryl. X^{75D} is halogen. In embodiments, X^{75D} is F.

R^{76D} is independently oxo,

halogen, —CX^{76D}₃, —CHX^{76D}₂, —OCH₂X^{76D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{76D}₃, —OCHX^{76D}₂, R^{77D}-substituted or unsubstituted alkyl, R^{77D}-substituted or unsubstituted heteroalkyl, R^{77D}-substituted or unsubstituted cycloalkyl, R^{77D}-substituted or unsubstituted heterocycloalkyl, R^{77D}-substituted or unsubstituted aryl, or R^{77D}-substituted or unsubstituted heteroaryl. X^{76D} is halogen. In embodiments, X^{76D} is F.

In embodiments, R¹⁷ is hydrogen. In embodiments, R¹⁷ is halogen. In embodiments, R¹⁷ is CX¹⁷₃. In embodiments, R¹⁷ is —CHX¹⁷₂. In embodiments, R¹⁷ is —CH₂X¹⁷. In embodiments, R¹⁷ is —CN. In embodiments, R¹⁷ is —SOₙ₁₇R^{17D}. In embodiments, R¹⁷ is —SOᵥ₁NR^{17A}R^{17B}. In embodiments, R¹⁷ is —NHNR^{17A}R^{17B}. In embodiments, R¹⁷ is —ONR^{17A}R^{17B}. In embodiments, R¹⁷ is —NHC═(O)NHNR^{17A}R^{17B}. In embodiments, R¹⁷ is —NHC(O)NR^{17A}R^{17B}. In embodiments, R¹⁷ is —N(O)ₘ₁₇. In embodiments, R¹⁷ is —NR^{17A}R^{17B}In embodiments, R¹⁷ is —C(O)R^{17C}. In embodiments, R¹⁷ is —C(O)—OR^{17C}. In embodiments, R¹⁷ is —C(O)NR^{17A}R^{17B}. In embodiments, R¹⁷ is —OR^{17D}. In embodiments, R¹⁷ is —NR^{17A}SO₂R^{17D}. In embodiments, R¹⁷ is —NR^{17A}C(O)R^{17C}. In embodiments, R¹⁷ is —NR^{17A}C(O)OR^{17C}. In embodiments, R¹⁷ is —NR^{17A}OR^{17C}. In embodiments, R¹⁷ is —OCX¹⁷₃. In embodiments, R¹⁷ is —OCHX¹⁷₂. In embodiments, R¹⁷ is substituted or unsubstituted alkyl. In embodiments, R¹⁷ is substituted or unsubstituted heteroalkyl. In embodiments, R¹⁷ is substituted or unsubstituted cycloalkyl. In embodiments, R¹⁷ is substituted or unsubstituted heterocycloalkyl. In embodiments, R¹⁷ is substituted or unsubstituted aryl. In embodiments, R¹⁷ is substituted or unsubstituted heteroaryl. In embodiments, R¹⁷ is substituted alkyl. In embodiments, R¹⁷ is substituted heteroalkyl. In embodiments, R¹⁷ is substituted cycloalkyl. In embodiments, R¹⁷ is substituted heterocycloalkyl. In embodiments, R¹⁷ is substituted aryl. In embodiments, R¹⁷ is substituted heteroaryl. In embodiments, R¹⁷ is unsubstituted alkyl. In embodiments, R¹⁷ is unsubstituted heteroalkyl. In embodiments, R¹⁷ is unsubstituted cycloalkyl. In embodiments, R¹⁷ is unsubstituted heterocycloalkyl. In embodiments, R¹⁷ is unsubstituted aryl. In embodiments, R¹⁷ is unsubstituted heteroaryl. In embodiments, R¹⁷ is unsubstituted methyl. In embodiments, R¹⁷ is unsubstituted ethyl. In embodiments, R¹⁷ is unsubstituted propyl. In embodiments, R¹⁷ is unsubstituted isopropyl. In embodiments, R¹⁷ is unsubstituted butyl. In embodiments, R¹⁷ is unsubstituted tert-butyl.

In embodiments, R^{17A} is hydrogen. In embodiments, R^{17A} is —CX₃. In embodiments, R^{17A} is —CN. In embodiments, R^{17A} is —COOH. In embodiments, R^{17A} is —CONH₂. In embodiments, R^{17A} is —CHX₂. In embodiments, R^{17A} is —CH₂X. In embodiments, R^{17A} is unsubstituted methyl. In embodiments, R^{17A} is unsubstituted ethyl. In embodiments, R^{17A} is unsubstituted propyl. In embodiments, R^{17A} is unsubstituted isopropyl. In embodiments, R^{17A} is unsubstituted butyl. In embodiments, R^{17A} is unsubstituted tert-butyl.

In embodiments, R^{17B} is hydrogen. In embodiments, R^{17B} is —CX₃. In embodiments, R^{17B} is —CN. In embodiments, R^{17B} is —COOH. In embodiments, R^{17B} is —CONH₂. In embodiments, R^{17B} is —CHX₂. In embodiments, R^{17B} is —CH₂X. In embodiments, R^{17B} is unsubstituted methyl. In embodiments, R^{17B} is unsubstituted ethyl. In embodiments, R^{17B} is unsubstituted propyl. In embodiments, R^{17B} is unsubstituted isopropyl. In embodiments, R^{17B} is unsubstituted butyl. In embodiments, R^{17B} is unsubstituted tert-butyl.

In embodiments, R^{17C} is hydrogen. In embodiments, R^{17C} is —CX₃. In embodiments, R^{17C} is —CN. In embodiments, R^{17C} is —COOH. In embodiments, R^{17C} is —CONH₂. In embodiments, R^{17C} is —CHX₂. In embodiments, R^{17C} is —CH₂X. In embodiments, R^{17C} is unsubstituted methyl. In embodiments, R^{17C} is unsubstituted ethyl. In embodiments, R^{17C} is unsubstituted propyl. In embodiments, R^{17C} is unsubstituted isopropyl. In embodiments, R^{17C} is unsubstituted butyl. In embodiments, R^{17C} is unsubstituted tert-butyl.

In embodiments, R^{17D} is hydrogen. In embodiments, R^{17D} is —CX₃. In embodiments, R^{17D} is —CN. In embodiments, R^{17D} is —COOH. In embodiments, R^{17D} is —CONH₂. In embodiments, R^{17D} is —CHX₂. In embodiments, R^{17D} is —CH₂X. In embodiments, R^{17D} is unsubstituted methyl. In embodiments, R^{17D} is unsubstituted ethyl. In embodiments, R^{17D} is unsubstituted propyl. In embodiments, R^{17D} is unsubstituted isopropyl. In embodiments, R^{17D} is unsubstituted butyl. In embodiments, R^{17D} is unsubstituted tert-butyl.

In embodiments, R¹⁷ is independently hydrogen, oxo,

halogen, —CX¹⁷₃, —CHX¹⁷₂, —OCH₂X¹⁷, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁷₃, —OCHX¹⁷₂, R⁷⁸-substituted or unsubstituted alkyl, R⁷⁸-substituted or unsubstituted heteroalkyl, R⁷⁸-substituted or unsubstituted cycloalkyl, R⁷⁸-substituted or unsubstituted heterocycloalkyl, R⁷⁸-substituted or unsubstituted aryl, or R⁷⁸-substituted or unsubstituted heteroaryl. X¹⁷ is halogen. In embodiments, X¹⁷ is F.

R⁷⁸ is independently oxo,

halogen, —CX⁷⁸₃, —CHX⁷⁸₂, —OCH₂X⁷⁸, —OCHX⁷⁸₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁸₃, —OCHX⁷⁸₂, R⁷⁹-substituted or unsubstituted alkyl, R⁷⁹-substituted or unsubstituted heteroalkyl, R⁷⁹-substituted or unsubstituted cycloalkyl, R⁷⁹-substituted or unsubstituted heterocycloalkyl, R⁷⁹-substituted or unsubstituted aryl, or R⁷⁹-substituted or unsubstituted heteroaryl. X⁷⁸ is halogen. In embodiments, X⁷⁸ is F.

R⁷⁹ is independently oxo,

halogen, —CX⁷⁹₃, —CHX⁷⁹₂, —OCH₂X⁷⁹, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁹₃, —OCHX⁷⁹₂, R⁸⁰-substituted or unsubstituted alkyl, R⁸⁰-substituted or unsubstituted heteroalkyl, R⁸⁰-substituted or unsubstituted cycloalkyl, R⁸⁰-substituted or unsubstituted heterocycloalkyl, R⁸⁰-substituted or unsubstituted aryl, or R⁸⁰-substituted or unsubstituted heteroaryl. X⁷⁹ is halogen. In embodiments, X⁷⁹ is F.

In embodiments, R^{17A} is independently hydrogen, oxo,

halogen, —CX^{17A}₃, —CHX^{17A}₂, —OCH₂X^{17A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17A}₃, —OCHX^{17A}₂, R^{78A}-substituted or unsubstituted alkyl, R^{78A}-substituted or unsubstituted heteroalkyl, R^{78A}-substituted or unsubstituted cycloalkyl, R^{78A}-substituted or unsubstituted heterocycloalkyl, R^{78A}-substituted or unsubstituted aryl, or R^{78A}-substituted or unsubstituted heteroaryl. X^{17A} is halogen. In embodiments, X^{17A} is F.

R^{78A} is independently oxo,

halogen, —CX^{78A}₃, —CHX^{78A}₂, —OCH₂X^{78A}, —OCHX^{78A}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78A}₃, —OCHX^{78A}₂, R^{79A}-substituted or unsubstituted alkyl, R^{79A}-substituted or unsubstituted heteroalkyl, R^{79A}-substituted or unsubstituted cycloalkyl, R^{79A}-substituted or unsubstituted heterocycloalkyl, R^{79A}-substituted or unsubstituted aryl, or R^{79A}-substituted or unsubstituted heteroaryl. X^{78A} is halogen. In embodiments, X^{78A} is F.

R^{79A} is independently oxo,

halogen, —CX^{79A}₃, —CHX^{79A}₂, —OCH₂X^{79A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79A}₃, —OCHX^{79A}₂, R^{80A}-substituted or unsubstituted alkyl, R^{80A}-substituted or unsubstituted heteroalkyl, R^{80A}-substituted or unsubstituted cycloalkyl, R^{80A}-substituted or unsubstituted heterocycloalkyl, R^{80A}-substituted or unsubstituted aryl, or R^{80A}-substituted or unsubstituted heteroaryl. X^{79A} is halogen. In embodiments, X^{79A} is F.

In embodiments, R^{17B} is independently hydrogen, oxo,

halogen, —CX^{17B}₃, —CHX^{17B}₂, —OCH₂X^{17B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17B}₃, —OCHX^{17B}₂, R^{78B}-substituted or unsubstituted alkyl, R^{78B}-substituted or unsubstituted heteroalkyl, R^{78B}-substituted or unsubstituted cycloalkyl, R^{78B}-substituted or unsubstituted heterocycloalkyl, R^{78B}-substituted or unsubstituted aryl, or R^{78B}-substituted or unsubstituted heteroaryl. X^{17B} is halogen. In embodiments, X^{17B} is F.

R^{78B} is independently oxo,

halogen, —CX^{78B}₃, —CHX^{78B}₂, —OCH₂X^{78B}, —OCHX^{78B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78B}₃, —OCHX^{78B}₂, R^{79B}-substituted or unsubstituted alkyl, R^{79B}-substituted or unsubstituted heteroalkyl, R^{79B}-substituted or unsubstituted cycloalkyl, R^{79B}-substituted or unsubstituted heterocycloalkyl, R^{79B}-substituted or unsubstituted aryl, or R^{79B}-substituted or unsubstituted heteroaryl. X^{78B} is halogen. In embodiments, X^{78B} is F.

R^{79B} is independently oxo,

halogen, —CX^{79B}₃, —CHX^{79B}₂, —OCH₂X^{79B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79B}₃, —OCHX^{79B}₂, R^{80B}-substituted or unsubstituted alkyl, R^{80B}-substituted or unsubstituted heteroalkyl, R^{80B}-substituted or unsubstituted cycloalkyl, R^{80B}-substituted or unsubstituted heterocycloalkyl, R^{80B}-substituted or unsubstituted aryl, or R^{80B}-substituted or unsubstituted heteroaryl. X^{79B} is halogen. In embodiments, X^{79B} is F.

In embodiments, R^{17C} is independently hydrogen, oxo,

halogen, —CX^{17C}₃, —CHX^{17C}₂, —OCH₂X^{17C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17C}₃, —OCHX^{17C}₂, R^{78C}-substituted or unsubstituted alkyl, R^{78C}-substituted or unsubstituted heteroalkyl, R^{78C}-substituted or unsubstituted cycloalkyl, R^{78C}-substituted or unsubstituted heterocycloalkyl, R^{78C}-substituted or unsubstituted aryl, or R^{78C}-substituted or unsubstituted heteroaryl. X^{17C} is halogen. In embodiments, X^{17C} is F.

R^{78C} is independently oxo,

halogen, —CX^{78C}₃, —CHX^{78C}₂, —OCH₂X^{78C}, —OCHX⁷⁸C₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁷⁸_{C3}, —OCHX^{78C}₂, R^{79C}-substituted or unsubstituted alkyl, R^{79C}-substituted or unsubstituted heteroalkyl, R^{79C}-substituted or unsubstituted cycloalkyl, R^{79C}-substituted or unsubstituted heterocycloalkyl, R^{79C}-substituted or unsubstituted aryl, or R^{79C}-substituted or unsubstituted heteroaryl. X^{78C} is halogen. In embodiments, X^{78C} is F.

R^{79C} is independently oxo,

halogen, —CX^{79C}₃, —CHX^{79C}₂, —OCH₂X^{79C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79C}₃, —OCHX^{79C}₂, R^{80C}-substituted or unsubstituted alkyl, R^{80C}-substituted or unsubstituted heteroalkyl, R^{80C}-substituted or unsubstituted cycloalkyl, R^{80C}-substituted or unsubstituted heterocycloalkyl, R^{80C}-substituted or unsubstituted aryl, or R^{80C}-substituted or unsubstituted heteroaryl. X^{79C} is halogen. In embodiments, X^{79C} is F.

In embodiments, R^{17D} is independently hydrogen, oxo,

halogen, —CX^{17D}₃, —CHX^{17D}₂, —OCH₂X^{17D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{17D}₃, —OCHX^{17D}₂, R^{78D}-substituted or unsubstituted alkyl, R^{78D}-substituted or unsubstituted heteroalkyl, R^{78D}-substituted or unsubstituted cycloalkyl, R^{78D}-substituted or unsubstituted heterocycloalkyl, R^{78D}-substituted or unsubstituted aryl, or R^{78D}-substituted or unsubstituted heteroaryl. X^{17D} is halogen. In embodiments, X^{17D} is F.

R^{78D} is independently oxo,

halogen, —CX^{78D}₃, —CHX^{78D}₂, —OCH₂X^{78D}, —OCHX^{78D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{78D}₃, —OCHX^{78D}₂, R^{79D}-substituted or unsubstituted alkyl, R^{79D}-substituted or unsubstituted heteroalkyl, R^{79D}-substituted or unsubstituted cycloalkyl, R^{79D}-substituted or unsubstituted heterocycloalkyl, R^{79D}-substituted or unsubstituted aryl, or R^{79D}-substituted or unsubstituted heteroaryl. X^{78D} is halogen. In embodiments, X^{78D} is F.

R^{79D} is independently oxo,

halogen, —CX^{79D}₃, —CHX^{79D}₂, —OCH₂X^{79D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{79D}₃, —OCHX^{79D}₂, R^{80D}-substituted or unsubstituted alkyl, R^{80D}-substituted or unsubstituted heteroalkyl, R^{80D}-substituted or unsubstituted cycloalkyl, R^{80D}-substituted or unsubstituted heterocycloalkyl, R^{80D}-substituted or unsubstituted aryl, or R^{80D}-substituted or unsubstituted heteroaryl. X^{79D} is halogen. In embodiments, X^{79D} is F.

In embodiments, R¹⁸ is hydrogen. In embodiments, R¹⁸ is halogen. In embodiments, R¹⁸ is CX¹⁸₃. In embodiments, R¹⁸ is —CHX¹⁸₂. In embodiments, R¹⁸ is —CH₂X¹⁸. In embodiments, R¹⁸ is —CN. In embodiments, R¹⁸ is —SOₙ₁₈R^{18D}. In embodiments, R¹⁸ is —SOᵥ₁₈NR^{18A}R^{18B}. In embodiments, R¹⁸ is —NHNR^{18A}R^{18B}. In embodiments, R¹⁸ is —ONR^{18A}R^{18B}. In embodiments, R¹⁸ is —NHC═(O)NHNR^{18A}R^{18B}. In embodiments, R¹⁸ is —NHC(O)NR^{18A}R^{18B}. In embodiments, R¹⁸ is —N(O)ₘ₁₈. In embodiments, R¹⁸ is —NR^{18A}R^{18B}. In embodiments, R¹⁸ is —C(O)R^{18C}. In embodiments, R¹⁸ is —C(O)—OR^{18C}. In embodiments, R¹⁸ is —C(O)NR^{18A}R^{18B}. In embodiments, R¹⁸ is —OR^{18D}. In embodiments, R¹⁸ is —NR^{18A}SO₂R^{18D}. In embodiments, R¹⁸ is —NR^{18A}C(O)R^{18C}. In embodiments, R¹⁸ is —NR^{18A}C(O)OR^{18C}. In embodiments, R¹⁸ is —NR^{18A}OR^{18C}. In embodiments, R¹⁸ is —OCX¹⁸₃. In embodiments, R¹⁸ is —OCHX¹⁸₂. In embodiments, R¹⁸ is substituted or unsubstituted alkyl. In embodiments, R¹⁸ is substituted or unsubstituted heteroalkyl. In embodiments, R¹⁸ is substituted or unsubstituted cycloalkyl. In embodiments, R¹⁸ is substituted or unsubstituted heterocycloalkyl. In embodiments, R¹⁸ is substituted or unsubstituted aryl. In embodiments, R¹⁸ is substituted or unsubstituted heteroaryl. In embodiments, R¹⁸ is substituted alkyl. In embodiments, R¹⁸ is substituted heteroalkyl. In embodiments, R¹⁸ is substituted cycloalkyl. In embodiments, R¹⁸ is substituted heterocycloalkyl. In embodiments, R¹⁸ is substituted aryl. In embodiments, R¹⁸ is substituted heteroaryl. In embodiments, R¹⁸ is unsubstituted alkyl. In embodiments, R¹⁸ is unsubstituted heteroalkyl. In embodiments, R¹⁸ is unsubstituted cycloalkyl. In embodiments, R¹⁸ is unsubstituted heterocycloalkyl. In embodiments, R¹⁸ is unsubstituted aryl. In embodiments, R¹⁸ is unsubstituted heteroaryl. In embodiments, R¹⁸ is unsubstituted methyl. In embodiments, R¹⁸ is unsubstituted ethyl. In embodiments, R¹⁸ is unsubstituted propyl. In embodiments, R¹⁸ is unsubstituted isopropyl. In embodiments, R¹⁸ is unsubstituted butyl. In embodiments, R¹⁸ is unsubstituted tert-butyl.

In embodiments, R^{18A} is hydrogen. In embodiments, R^{18A} is —CX₃. In embodiments, R^{18A} is —CN. In embodiments, R^{18A} is —COOH. In embodiments, R^{18A} is —CONH₂. In embodiments, R^{18A} is —CHX₂. In embodiments, R^{18A} is —CH₂X. In embodiments, R^{18A} is unsubstituted methyl. In embodiments, R^{18A} is unsubstituted ethyl. In embodiments, R^{18A} is unsubstituted propyl. In embodiments, R^{18A} is unsubstituted isopropyl. In embodiments, R^{18A} is unsubstituted butyl. In embodiments, R^{18A} is unsubstituted tert-butyl.

In embodiments, R^{18B} is hydrogen. In embodiments, R^{18B} is —CX₃. In embodiments, R^{18B} is —CN. In embodiments, R^{18B} is —COOH. In embodiments, R^{18B} is —CONH₂. In embodiments, R^{18B} is —CHX₂. In embodiments, R^{18B} is —CH₂X. In embodiments, R^{18B} is unsubstituted methyl. In embodiments, R^{18B} is unsubstituted ethyl. In embodiments, R^{18B} is unsubstituted propyl. In embodiments, R^{18B} is unsubstituted isopropyl. In embodiments, R^{18B} is unsubstituted butyl. In embodiments, R^{18B} is unsubstituted tert-butyl.

In embodiments, R^{18C} is hydrogen. In embodiments, R^{18C} is —CX₃. In embodiments, R^{18C} is —CN. In embodiments, R^{18C} is —COOH. In embodiments, R^{18C} is —CONH₂. In embodiments, R^{18C} is —CHX₂. In embodiments, R^{18C} is —CH₂X. In embodiments, R^{18C} is unsubstituted methyl. In embodiments, R^{18C} is unsubstituted ethyl. In embodiments, R^{18C} is unsubstituted propyl. In embodiments, R^{18C} is unsubstituted isopropyl. In embodiments, R^{18C} is unsubstituted butyl. In embodiments, R^{18C} is unsubstituted tert-butyl.

In embodiments, R^{18D} is hydrogen. In embodiments, R^{18D} is —CX₃. In embodiments, R^{18D} is —CN. In embodiments, R^{18D} is —COOH. In embodiments, R^{18D} is —CONH₂. In embodiments, R^{18D} is —CHX₂. In embodiments, R^{18D} is —CH₂X. In embodiments, R^{18D} is unsubstituted methyl. In embodiments, R^{18D} is unsubstituted ethyl. In embodiments, R^{18D} is unsubstituted propyl. In embodiments, R^{18D} is unsubstituted isopropyl. In embodiments, R^{18D} is unsubstituted butyl. In embodiments, R^{18D} is unsubstituted tert-butyl.

In embodiments, R¹⁸ is independently hydrogen, oxo,

halogen, —CX¹⁸₃, —CHX¹⁸₂, —OCH₂X¹⁸, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX¹⁸₃, —OCHX¹⁸₂, R⁸¹-substituted or unsubstituted alkyl, R⁸¹-substituted or unsubstituted heteroalkyl, R⁸¹-substituted or unsubstituted cycloalkyl, R⁸¹-substituted or unsubstituted heterocycloalkyl, R⁸¹-substituted or unsubstituted aryl, or R⁸¹-substituted or unsubstituted heteroaryl. X¹⁸ is halogen. In embodiments, X¹⁸ is F.

R⁸¹ is independently oxo,

halogen, —CX⁸¹₃, —CHX⁸¹₂, —OCH₂X⁸¹, —OCHX⁸¹₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸¹₃, —OCHX⁸¹₂, R⁸²-substituted or unsubstituted alkyl, R⁸²-substituted or unsubstituted heteroalkyl, R⁸²-substituted or unsubstituted cycloalkyl, R⁸²-substituted or unsubstituted heterocycloalkyl, R⁸²-substituted or unsubstituted aryl, or R⁸²-substituted or unsubstituted heteroaryl. X⁸¹ is halogen. In embodiments, X⁸¹ is F.

R⁸² is independently oxo,

halogen, —CX⁸²₃, —CHX⁸²₂, —OCH₂X⁸², —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸²₃, —OCHX⁸²₂, R⁸³-substituted or unsubstituted alkyl, R⁸³-substituted or unsubstituted heteroalkyl, R⁸³-substituted or unsubstituted cycloalkyl, R⁸³-substituted or unsubstituted heterocycloalkyl, R⁸³-substituted or unsubstituted aryl, or R⁸³-substituted or unsubstituted heteroaryl. X⁸² is halogen. In embodiments, X⁸² is F.

In embodiments, R^{18A} is independently hydrogen, oxo,

halogen, —CX^{18A}₃, —CHX^{18A}₂, —OCH₂X^{18A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18A}₃, —OCHX^{18A}₂, R^{81A}-substituted or unsubstituted alkyl, R^{81A}-substituted or unsubstituted heteroalkyl, R^{81A}-substituted or unsubstituted cycloalkyl, R^{81A}-substituted or unsubstituted heterocycloalkyl, R^{81A}-substituted or unsubstituted aryl, or R^{81A}-substituted or unsubstituted heteroaryl. X^{18A} is halogen. In embodiments, X^{18A} is F.

R^{81A} is independently oxo,

halogen, —CX^{81A}₃, —CHX^{81A}₂, —OCH₂X^{81A}, —OCHX^{81A}₂, —CN, —OH, —NH₂, —COOH, —CONH₂, —N O₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81A}₃, —OCHX^{81A}₂, R^{82A}-substituted or unsubstituted alkyl, R^{82A}-substituted or unsubstituted heteroalkyl, R^{82A}-substituted or unsubstituted cycloalkyl, R^{82A}-substituted or unsubstituted heterocycloalkyl, R^{82A}-substituted or unsubstituted aryl, or R^{82A}-substituted or unsubstituted heteroaryl. X^{81A} is halogen. In embodiments, X^{81A} is F.

R^{82A} is independently oxo,

halogen, —CX^{82A}₃, —CHX^{82A}₂, —OCH₂X^{82A}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82A}₃, —OCHX^{82A}₂, R^{83A}-substituted or unsubstituted alkyl, R^{83A}-substituted or unsubstituted heteroalkyl, R^{83A}-substituted or unsubstituted cycloalkyl, R^{83A}-substituted or unsubstituted heterocycloalkyl, R^{83A}-substituted or unsubstituted aryl, or R^{83A}-substituted or unsubstituted heteroaryl. X^{82A} is halogen. In embodiments, X^{82A} is F.

In embodiments, R^{18B} is independently hydrogen, oxo,

halogen, —CX^{18B}₃, —CHX^{18B}₂, —OCH₂X^{18B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18B}₃, —OCHX^{18B}₂, R^{81B}-substituted or unsubstituted alkyl, R^{81B}-substituted or unsubstituted heteroalkyl, R^{81B}-substituted or unsubstituted cycloalkyl, R^{81B}-substituted or unsubstituted heterocycloalkyl, R^{81B}-substituted or unsubstituted aryl, or R^{81B}-substituted or unsubstituted heteroaryl. X^{18B} is halogen. In embodiments, X^{18B} is F.

R^{81B} is independently oxo,

halogen, —CX^{81B}₃, —CHX^{81B}₂, —OCH₂X⁸¹_{B}, —OCHX^{81B}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81B}₃, —OCHX^{81B}₂, R^{82B}-substituted or unsubstituted alkyl, R^{82B}-substituted or unsubstituted heteroalkyl, R^{82B}-substituted or unsubstituted cycloalkyl, R^{82B}-substituted or unsubstituted heterocycloalkyl, R^{82B}-substituted or unsubstituted aryl, or R^{82B}-substituted or unsubstituted heteroaryl. X^{81B} is halogen. In embodiments, X^{81B} is F.

R^{82B} is independently oxo,

halogen, —CX^{82B}₃, —CHX^{82B}₂, —OCH₂X^{82B}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82B}₃, —OCHX^{82B}₂, R^{83B}-substituted or unsubstituted alkyl, R^{83B}-substituted or unsubstituted heteroalkyl, R^{83B}-substituted or unsubstituted cycloalkyl, R^{83B}-substituted or unsubstituted heterocycloalkyl, R^{83B}-substituted or unsubstituted aryl, or R^{83B}-substituted or unsubstituted heteroaryl. X^{82B} is halogen. In embodiments, X^{82B} is F.

In embodiments, R^{18C} is independently hydrogen, oxo,

halogen, —CX^{18C}₃, —CHX^{18C}₂, —OCH₂X^{18C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18C}₃, —OCHX^{18C}₂, R^{81C}-substituted or unsubstituted alkyl, R^{81C}-substituted or unsubstituted heteroalkyl, R^{81C}-substituted or unsubstituted cycloalkyl, R^{81C}-substituted or unsubstituted heterocycloalkyl, R^{81C}-substituted or unsubstituted aryl, or R^{81C}-substituted or unsubstituted heteroaryl. X^{18C} is halogen. In embodiments, X^{18C} is F.

R^{81C} is independently oxo,

halogen, —CX^{81C}₃, —CHX^{81C}₂, —OCH₂X^{81C}, —OCHX^{81C}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX⁸¹C₃, —OCHX⁸¹C₂, R^{82C}-substituted or unsubstituted alkyl, R^{82C}-substituted or unsubstituted heteroalkyl, R^{82C}-substituted or unsubstituted cycloalkyl, R^{82C}-substituted or unsubstituted heterocycloalkyl, R^{82C}-substituted or unsubstituted aryl, or R^{82C}-substituted or unsubstituted heteroaryl. X^{81C} is halogen. In embodiments, X^{81C} is F.

R^{82C} is independently oxo,

halogen, —CX^{82C}₃, —CHX^{82C}₂, —OCH₂X^{82C}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82C}₃, —OCHX^{82C}₂, R^{83C}-substituted or unsubstituted alkyl, R^{83C}-substituted or unsubstituted heteroalkyl, R^{83C}-substituted or unsubstituted cycloalkyl, R^{83C}-substituted or unsubstituted heterocycloalkyl, R^{83C}-substituted or unsubstituted aryl, or R^{83C}-substituted or unsubstituted heteroaryl. X^{82C} is halogen. In embodiments, X^{82C} is F.

In embodiments, R^{18D} is independently hydrogen, oxo,

halogen, —CX^{18D}₃, —CHX^{18D}₂, —OCH₂X^{18D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{18D}₃, —OCHX^{18D}₂, R^{81D}-substituted or unsubstituted alkyl, R^{81D}-substituted or unsubstituted heteroalkyl, R^{81D}-substituted or unsubstituted cycloalkyl, R^{81D}-substituted or unsubstituted heterocycloalkyl, R^{81D}-substituted or unsubstituted aryl, or R^{81D}-substituted or unsubstituted heteroaryl. X^{18D} is halogen. In embodiments, X^{18D} is F.

R^{81D} is independently oxo,

halogen, —CX^{81D}₃, —CHX^{81D}₂, —OCH₂X^{81D}, —OCHX^{81D}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{81D}₃, —OCHX^{81D}₂, R^{82D}-substituted or unsubstituted alkyl, R^{82D}-substituted or unsubstituted heteroalkyl, R^{82D}-substituted or unsubstituted cycloalkyl, R^{82D}-substituted or unsubstituted heterocycloalkyl, R^{82D}-substituted or unsubstituted aryl, or R^{82D}-substituted or unsubstituted heteroaryl. X^{81D} is halogen. In embodiments, X^{81D} is F.

R^{82D} is independently oxo,

halogen, —CX^{82D}₃, —CHX^{82D}₂, —OCH₂X^{82D}, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCX^{82D}₃, —OCHX^{82D}₂, R^{83D}-substituted or unsubstituted alkyl, R^{83D}-substituted or unsubstituted heteroalkyl, R^{83D}-substituted or unsubstituted cycloalkyl, R^{83D}-substituted or unsubstituted heterocycloalkyl, R^{83D}-substituted or unsubstituted aryl, or R^{83D}-substituted or unsubstituted heteroaryl. X^{82D} is halogen. In embodiments, X^{82D} is F.

R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently hydrogen, oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently oxo,

halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. In embodiments, R⁷⁴, R⁷⁷, R⁸⁰, R⁸³, R^{74A}, R^{77A}, R^{80A}, R^{83A}, R^{74B}, R^{77B}, R^{80B}, R^{83B}, R^{74C}, R^{77C}, R^{80C}, R^{83C}, R^{74D}, R^{77D}, R^{80D}, R^{83D}, R⁸⁶, R⁸⁹, R⁹², and R⁹⁸ are independently oxo,
halogen, —CF₃, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCHF₂, unsubstituted C₁-C₈ alkyl, unsubstituted 2 to 8 membered heteroalkyl, unsubstituted C₃-C₈ cycloalkyl, unsubstituted 3 to 6 membered heterocycloalkyl, unsubstituted phenyl, or unsubstituted 5 to 6 membered heteroaryl.

In embodiments, R¹⁵, R¹⁶, R¹⁷, and R¹⁸ are hydrogen.

In embodiments, E is:

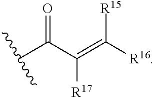

In embodiments, E is:

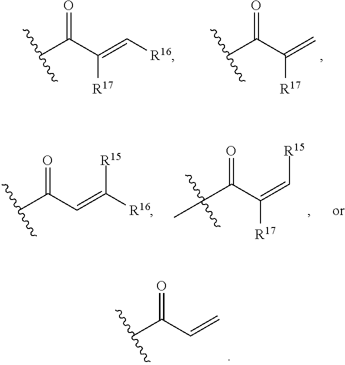

In embodiments, E is:

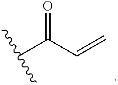 [SMILES: *C(=O)C=C]

In embodiments, E is:

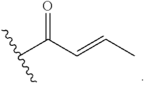 [SMILES: *C(=O)/C=C/C]

In embodiments, E is:

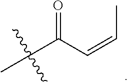 [SMILES: *C(=O)/C=C\C]

In embodiments, E is:

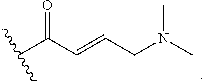 [SMILES: *C(=O)/C=C/CN(C)C]

In embodiments, E is:

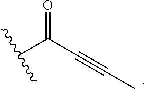 [SMILES: *C(=O)C#CC]

In some embodiments, a compound as described herein may include multiple instances of R¹ or R², and/or other variables. In such embodiments, each variable may optional be different and be appropriately labeled to distinguish each group for greater clarity. For example, where each R¹ and/or R², is different, they may be referred to, for example, as R^{1.1}, R^{1.2}, R^{1.3}, R^{1.4}, R^{1.5}, R^{2.1}, R^{2.2}, R^{2.3}, or R^{2.4}, respectively, wherein the definition of R¹ is assumed by R^{1.1}, R^{1.2}, R^{1.3}, R^{1.4}, R^{1.5}; and/or R² is assumed by R^{2.1}, R^{2.2}, R^{2.3}, R^{2.4}. The variables used within a definition of R¹ and/or R², and/or other variables that appear at multiple instances and are different may similarly be appropriately labeled to distinguish each group for greater clarity. In some embodiments, the compound is a compound described herein (e.g., in an aspect, embodiment, example, claim, table, scheme, drawing, or figure).

In embodiments, unless otherwise indicated, a compound described herein is a racemic mixture of all stereoisomers. In embodiments, unless otherwise indicated, a compound described herein is a racemic mixture of all enantiomers. In embodiments, unless otherwise indicated, a compound described herein is a racemic mixture of two opposite stereoisomers. In embodiments, unless otherwise indicated, a compound described herein is a racemic mixture of two opposite enantiomers. In embodiments, unless otherwise indicated, a compound described herein is a single stereoisomer. In embodiments, unless otherwise indicated, a compound described herein is a single enantiomer. In embodiments, the compound is a compound described herein (e.g., in an aspect, embodiment, example, figure, table, scheme, or claim).

In an aspect is provided a Serine/threonine-protein phosphatase 2A regulatory subunit A alpha isoform (PPP2R1A) modulator (i.e., the PPP2R1A modulator). In embodiments, the Serine/threonine-protein phosphatase 2A regulatory subunit A alpha isoform (PPP2R1A) modulator (i.e., the PPP2R1A modulator) increases protein phosphatase 2A activity (e.g., may in addition inhibit the AKT/protein kinase B signaling, optionally through increasing PP2A activity). In embodiments, the PPP2R1A modulator is a compound described herein. In embodiments, the PPP2R1A modulator is an oligonucleotide (e.g., DNA, RNA, shRNA, or siRNA), protein (e.g., antibody, anti-PPP2R1A antibody, anti-PPP2R1A antibody fragment), withaferin A, or compound (e.g., compound described herein). In embodiments, the PPP2R1A modulator contacts one or more amino acids of the protein phosphatase 2A (PP2A) complex (e.g., human), the one or more amino acids corresponding to H340, S343, C377, E379, Q339, and K416 in human protein phosphatase 2A regulatory subunit A alpha isoform (PPP2R1A) (e.g., SEQ ID NO:4); corresponding to N264, Q272, D290, and M245 in human protein phosphatase 2A catalytic subunit alpha isoform (PPP2CA) (e.g., SEQ ID NO:6); and corresponding to F118, E117, and P113 in human protein phosphatase 2A regulatory subunit gamma isoform (PPP2R5C) (e.g., SEQ ID NO:5). In embodiments, the PPP2R1A modulator contacts one or more amino acids corresponding to H340, S343, C377, E379, Q339, and K416 of SEQ ID NO:4.

In embodiments, the PPP2R1A modulator covalently binds to an amino acid corresponding to C377 SEQ ID NO:4. In embodiments, the PPP2R1A modulator contacts an amino acid corresponding to H340 SEQ ID NO:4. In embodiments, the PPP2R1A modulator contacts an amino acid corresponding to S343 SEQ ID NO:4. In embodiments, the PPP2R1A modulator contacts an amino acid corresponding to E379 SEQ ID NO:4. In embodiments, the PPP2R1A modulator contacts an amino acid corresponding to Q339 SEQ ID NO:4. In embodiments, the PPP2R1A modulator contacts an amino acid corresponding to K416 SEQ ID NO:4. In embodiments, the PPP2R1A modulator contacts one or more amino acids corresponding to N264, Q272, D290, and M245 SEQ ID NO:6. In embodiments, the PPP2R1A modulator contacts an amino acid corresponding to N264 SEQ ID NO:6. In embodiments, the PPP2R1A modulator contacts an amino acid corresponding to Q272 SEQ ID NO:6. In embodiments, the PPP2R1A modulator contacts an amino acid corresponding to D290 SEQ ID NO:6. In embodiments, the PPP2R1A modulator contacts an amino acid corresponding to M245 SEQ ID NO:6. In embodiments, the PPP2R1A modulator contacts one or more amino acids corresponding to F118, E117, and P113 SEQ ID NO:5. In embodiments, the PPP2R1A modulator contacts an amino acid corresponding to F118 SEQ ID NO:5. In embodiments, the PPP2R1A modulator contacts an amino acid corresponding to E117 SEQ ID NO:5. In embodiments, the PPP2R1A modulator contacts an amino acid corresponding to P113 SEQ ID NO:5. In embodiments, the PPP2R1A modulator stabilizes the interaction of one of more proteins corresponding to the human regulatory subunit A alpha isoform (PPP2R1A), the human catalytic subunit alpha isoform (PPP2CA), and the human regulatory subunit gamma isoform (PPP2R5C) of the human protein phosphatase 2A (PP2A) complex. In embodiments, the PPP2R1A modulator modulates the interaction of one of more proteins corresponding to the human regulatory subunit A alpha isoform (PPP2R1A), the human catalytic subunit alpha isoform (PPP2CA), and the human regulatory subunit gamma isoform (PPP2R5C) of the human protein phosphatase 2A (PP2A) complex (e.g., resulting in an increase in the level of phosphatase activity).

In embodiments, the compound has the formula:

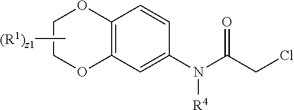

R¹, z1, and R⁴ are as described herein.

In embodiments, the compound has the formula:

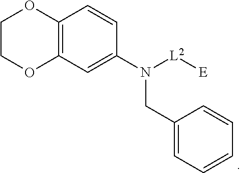

L² and E are as described herein.

In embodiments, the compound has the formula:

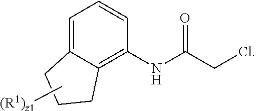

R¹ and z1 are as described herein.

In embodiments, the compound has the formula:

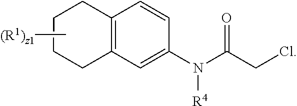

R¹, R⁴, and z1 are as described herein.

In embodiments, the compound has the formula:

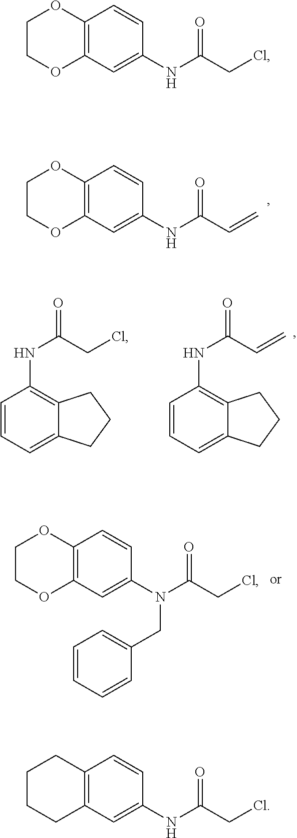

In embodiments, the compound has the formula:

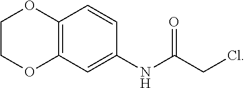 [SMILES: O=C(CCl)Nc1ccc2c(c1)OCCO2]

In embodiments, the compound has the formula:

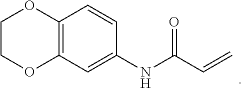

In embodiments, the compound has the formula:

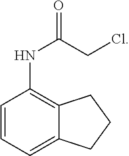 [SMILES: O=C(CCl)Nc1cccc2c1CCC2]

In embodiments, the compound has the formula:

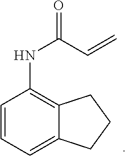

In embodiments, the compound has the formula:

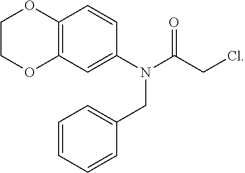 [SMILES: O=C(CCl)N(Cc1ccccc1)c1ccc2c(c1)OCCO2]

In embodiments, the compound has the formula:

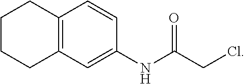 [SMILES: O=C(CCl)Nc1ccc2c(c1)CCCC2]

In embodiments, the compound has the formula:

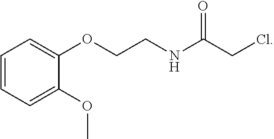 [SMILES: COc1ccccc1OCCNC(=O)CCl]

In embodiments, the compound has the formula:

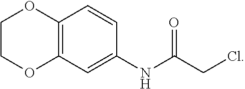 [SMILES: O=C(CCl)Nc1ccc2c(c1)OCCO2]

In embodiments, the compound has the formula:

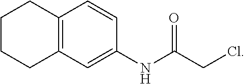 [SMILES: O=C(CCl)Nc1ccc2c(c1)CCCC2]

In embodiments, the compound has the formula:

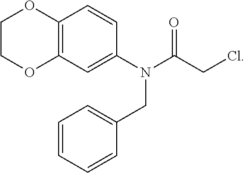 [SMILES: O=C(CCl)N(Cc1ccccc1)c1ccc2c(c1)OCCO2]

In embodiments, the compound has the formula:

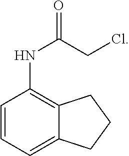 [SMILES: O=C(CCl)Nc1cccc2c1CCC2]

In embodiments, the compound is withaferin A.

### III. Pharmaceutical Compositions

In an aspect is provided a pharmaceutical composition including a Serine/threonine-protein phosphatase 2A 65 kDa regulatory subunit A alpha isoform (PPP2R1A) modulator and a pharmaceutically acceptable excipient. In embodiments, the PPP2R1A modulator is a compound described herein. In embodiments, the PPP2R1A modulator is an oligonucleotide (e.g., DNA, RNA, or siRNA), antisense nucleic acid, protein (e.g., antibody, anti-PPP2R1A antibody, anti-PPP2R1A binding antibody fragment), Withaferin A, or compound (e.g., compound described herein). In embodiments, the PPP2R1A modulator is included in a therapeutically effective amount.

In an aspect is provided a pharmaceutical composition including a compound described herein, or pharmaceutically acceptable salt thereof, and a pharmaceutically acceptable excipient.

In embodiments of the pharmaceutical compositions, the compound, or pharmaceutically acceptable salt thereof, is included in a therapeutically effective amount.

In embodiments of the pharmaceutical compositions, the pharmaceutical composition includes a second agent (e.g. therapeutic agent). In embodiments of the pharmaceutical compositions, the pharmaceutical composition includes a second agent (e.g. therapeutic agent) in a therapeutically effective amount. In embodiments of the pharmaceutical compositions, the second agent is an agent for treating cancer. In embodiments, the second agent is an anti-cancer agent. In embodiments, the second agent is a chemotherapeutic.

### IV. Methods of Treatment

In an aspect is provided a method of treating cancer, said method including administering to a subject in need thereof an effective amount of a serine/threonine-protein phosphatase 2A 65 kDa regulatory subunit A alpha isoform (PPP2R1A) modulator. In embodiments, the PPP2R1A modulator is a compound described herein. In embodiments, the PPP2R1A modulator is an oligonucleotide (e.g., DNA, RNA, or siRNA), antisense nucleic acid, protein (e.g., antibody, anti-PPP2R1A antibody, anti-PPP2R1A antibody fragment), withaferin A, or compound (e.g., compound described herein). In embodiments, the PPP2R1A modulator is included in a therapeutically effective amount.

In an aspect is provided a method of treating cancer including administering to a subject in need thereof an effective amount of a compound described herein. In embodiments, the cancer is gynecological cancer. In embodiments, the cancer is uterine cancer. In embodiments, the cancer is ovarian cancer. In embodiments, the cancer is endometrial cancer. In embodiments, the cancer is vulvar cancer. In embodiments, the cancer is colon cancer. In embodiments, the cancer is breast cancer. In embodiments, the cancer is estrogen receptor positive breast cancer. In embodiments, the cancer is estrogen receptor (ER) negative breast cancer. In embodiments, the cancer is tamoxifen resistant breast cancer. In embodiments, the cancer is HER2 negative breast cancer. In embodiments, the cancer is HER2 positive breast cancer. In embodiments, the cancer is low grade (well differentiated) breast cancer. In embodiments, the cancer is intermediate grade (moderately differentiated) breast cancer. In embodiments, the cancer is high grade (poorly differentiated) breast cancer. In embodiments, the cancer is stage 0 breast cancer. In embodiments, the cancer is stage I breast cancer. In embodiments, the cancer is stage II breast cancer. In embodiments, the cancer is stage III breast cancer. In embodiments, the cancer is stage IV breast cancer. In embodiments, the cancer is triple negative breast cancer. In embodiments, the cancer is glioblastoma. In embodiments, the cancer is pancreatic cancer. In embodiments, the cancer is prostate cancer.

In embodiments, the cancer is metastatic cancer. In embodiments, the cancer is associated with a cysteine containing protein. In embodiments, the cancer expresses a protein containing a cysteine. In embodiments, the compound contacts a cysteine containing protein (e.g., covalently binds to a cysteine containing protein). In embodiments, the compound contacts a cysteine containing protein (e.g., covalently binds to a cysteine containing protein). In embodiments, the compound covalently reacts with a cysteine containing protein.

In an aspect is provided a method of treating cancer, said method including administering to a subject in need thereof an effective amount of a cysteine modulator (e.g., a compound described herein). As used herein, a cysteine modulator is a compound which modulates (e.g., increases or decreases) the level activity of a protein relative to the level or activity of the protin in the absence of the modulator. In embodiments, the cancer is associated with a cysteine containing protein. In embodiments, the cancer expresses a protein containing a cysteine. In embodiments, the compound contacts a cysteine containing protein (e.g., covalently binds to a cysteine containing protein). In embodiments, the compound contacts a cysteine containing protein (e.g., covalently binds to a cysteine containing protein). In embodiments, the compound covalently reacts with a cysteine containing protein.

In an aspect is provided a method of treating cancer associated with the AKT/protein kinase B signaling pathway (e.g., AKT protein activity), said method including administering to a subject in need thereof an effective amount of a serine/threonine-protein phosphatase 2A regulatory subunit A alpha isoform (PPP2R1A) modulator. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A antibody fragment), withaferin A, or compound (e.g., compound described herein). In embodiments, the PPP2R1A modulator is included in a therapeutically effective amount.

In an aspect is provided a method of treating a disease associated with protein phosphatase 2A (PP2A) activity (e.g., increasing or activating PP2A phosphatase activity) including administering to a subject in need thereof an effective amount of a a serine/threonine-protein phosphatase 2A regulatory subunit A alpha isoform (PPP2R1A) modulator. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A antibody fragment), withaferin A, or compound (e.g., compound described herein). In embodiments, the PPP2R1A modulator is included in a therapeutically effective amount.

In an aspect is provided a method of treating a disease, including activation of protein phosphatase 2A (PP2A) complex by administering to a subject in need thereof an effective amount of a a serine/threonine-protein phosphatase 2A regulatory subunit A alpha isoform (PPP2R1A) modulator. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A antibody fragment), withaferin A, or compound (e.g., compound described herein). In embodiments, the PPP2R1A modulator is included in a therapeutically effective amount.

In embodiments, the method includes administering a second agent (e.g. therapeutic agent). In embodiments, the method includes administering a second agent (e.g. therapeutic agent) in a therapeutically effective amount. In embodiments, the second agent is an agent for treating cancer. In embodiments, the second agent is an anti-cancer agent. In embodiments, the second agent is a chemotherapeutic.

### V. Methods of Modulation

In an aspect is provided a method of modulating a serine/threonine-protein phosphatase 2A 65 kDa regulatory subunit A alpha isoform (PPP2R1A) protein, the method including contacting the Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator (e.g., a compound described herein). In embodiments, modulating is changing the physical state of the PPP2R1A protein (e.g., covalently modifying the protein). In embodiments, modulating is changing the physical state of the PPP2R1A protein (e.g., covalently modifying the protein) which activates PP2A. In embodiments, modulating includes the activation of PP2A (e.g. and inhibition of AKT signaling).

In an aspect is provided a method of modulating a serine/threonine-protein phosphatase 2A 65 kDa regulatory subunit A alpha isoform (PPP2R1A) protein, the method including contacting the Serine/threonine-protein phosphatase 2A 65 kDa regulatory subunit A alpha isoform (PPP2R1A) protein with an effective amount of a compound described herein.

In an aspect is provided a method of activating a tumor suppressor protein phosphatase 2A (PP2A), the method including contacting a Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator (e.g., a compound described herein).

In an aspect is provided a method of modulating PPP2R1A including contacting the PPP2R1A with a PPP2R1A modulator. In embodiments, the PPP2R1A is a human PPP2R1A. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A binding antibody fragment), or compound (e.g., compound described herein). In embodiments, the PPP2R1A modulator is provided in a therapeutically effective amount.

In embodiments, the PPP2R1A modulator contacts one or more amino acids corresponding to E379, H340, S343, C377, C198, Q339, and K416 of human PPP2R1A. In embodiments, the PPP2R1A modulator covalently binds an amino acid corresponding to C377 in human PPP2R1A. In embodiments, the PPP2R1A modulator covalently binds an amino acid corresponding to C198 in human PPP2R1A. In embodiments, the PPP2R1A modulator contacts an amino acid corresponding to E379, H340, S343, C377, Q339, K416, and H340 of human PPP2R1A. In embodiments, the PPP2R1A modulator contacts an amino acids corresponding to E379 of human PPP2R1A. In embodiments, the PPP2R1A modulator contacts an amino acids corresponding to H340 of human PPP2R1A. In embodiments, the PPP2R1A modulator contacts an amino acids corresponding to S343 of human PPP2R1A. In embodiments, the PPP2R1A modulator contacts an amino acids corresponding to C377 of human PPP2R1A. In embodiments, the PPP2R1A modulator contacts an amino acids corresponding to Q339 of human PPP2R1A. In embodiments, the PPP2R1A modulator contacts an amino acids corresponding to K416 of human PPP2R1A. In embodiments, the PPP2R1A modulator contacts an amino acids corresponding to H340 of human PPP2R1A.

In embodiments, the PPP2R1A modulator contacts one or more amino acids corresponding to E379, H340, S343, C377, C198, Q339, and K416 of SEQ ID NO:4. In embodiments, the PPP2R1A modulator covalently binds an amino acid corresponding to C377 in SEQ ID NO:4. In embodiments, the PPP2R1A modulator covalently binds an amino acid corresponding to C198 in SEQ ID NO:4. In embodiments, the PPP2R1A modulator contacts an amino acid corresponding to E379, H340, S343, C377, Q339, K416, and H340 of SEQ ID NO:4. In embodiments, the PPP2R1A modulator contacts an amino acids corresponding to E379 of SEQ ID NO:4. In embodiments, the PPP2R1A modulator contacts an amino acids corresponding to H340 of SEQ ID NO:4. In embodiments, the PPP2R1A modulator contacts an amino acids corresponding to S343 of SEQ ID NO:4. In embodiments, the PPP2R1A modulator contacts an amino acids corresponding to C377 of SEQ ID NO:4. In embodiments, the PPP2R1A modulator contacts an amino acids corresponding to Q339 of SEQ ID NO:4. In embodiments, the PPP2R1A modulator contacts an amino acids corresponding to K416 of SEQ ID NO:4. In embodiments, the PPP2R1A modulator contacts an amino acids corresponding to H340 of SEQ ID NO:4.

Where the compound covalently binds to the PPP2R1A a PPP2R1A protein (e.g., human PPP2R1A) covalently bonded to a PPP2R1A modulator is formed (also referred to herein as a “PPP2R1A-compound adduct”), as described below. In embodiments, the resulting covalent bond is reversible. Where the resulting covalent bond is reversible, the bonding reverses upon denaturation of the protein. Thus, in embodiments, the reversibility of a covalent bond between the compound and the PPP2R1A upon denaturation of the PPP2R1A avoids or decreases autoimmune response in a subject subsequent to administration of the compound (relative to irreversibility). Moreover, in embodiments, the reversibility of a covalent bond between the compound and the PPP2R1A upon denaturation of the PPP2R1A avoids or decreases the toxicity (e.g. liver toxicity) of the compound in a subject (relative to irreversibility).

In an aspect is provided a method of modulating protein phosphatase 2A (PP2A) activity, the method including contacting a Serine/threonine-protein phosphatase 2A 65 kDa regulatory subunit A alpha isoform (PPP2R1A) protein included in the PP2CA, with an effective amount of a Serine/threonine-protein phosphatase 2A 65 kDa regulatory subunit A alpha isoform (PPP2R1A) modulator. In embodiments, modulating is changing the physical state of the PPP2R1A protein (e.g., covalently modifying the protein). In embodiments, modulating includes the increasing the activity (e.g., protein phosphatase activity) of PP2A (e.g. and inhibition of AKT signaling).

In an aspect is provided a method of modulating PP2CA activity, the method including contacting the Serine/threonine-protein phosphatase 2A 65 kDa regulatory subunit A alpha isoform (PPP2R1A) protein of the PP2CA with an effective amount of a compound described herein.

In an aspect is provided a method of modulating PP2CA activity including contacting the PPP2R1A included in the PP2CA with a PPP2R1A modulator. In embodiments, the PPP2R1A modulator contact PPP2R1A protein when not associated with the PP2CA complex (e.g., followed by formation of the PP2CA complex with the PPP2R1A protein). In embodiments, the PPP2R1A modulator contact PPP2R1A protein when associated with the PP2CA complex (e.g., following formation of the PP2CA complex with the PPP2R1A protein). In embodiments, the PPP2R1A is a human PPP2R1A. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A binding antibody fragment), or compound (e.g., compound described herein). In embodiments, the PPP2R1A modulator is provided in a therapeutically effective amount.

In embodiments, the PPP2R1A modulator contacts one or more amino acids corresponding to E379, H340, S343, C377, C198, Q339, and K416 of human PPP2R1A. In embodiments, the PPP2R1A modulator covalently binds an amino acid corresponding to C377 in human PPP2R1A. In embodiments, the PPP2R1A modulator covalently binds an amino acid corresponding to C198 in human PPP2R1A. In embodiments, the PPP2R1A modulator contacts an amino acid corresponding to E379, H340, S343, C377, C198, Q339, and K416 of human PPP2R1A. In embodiments, the PPP2R1A modulator contacts an amino acids corresponding to E379 of human PPP2R1A. In embodiments, the PPP2R1A modulator contacts an amino acids corresponding to H340 of human PPP2R1A. In embodiments, the PPP2R1A modulator contacts an amino acids corresponding to S343 of human PPP2R1A. In embodiments, the PPP2R1A modulator contacts an amino acids corresponding to C377 of human PPP2R1A. In embodiments, the PPP2R1A modulator contacts an amino acids corresponding to C198 of human PPP2R1A. In embodiments, the PPP2R1A modulator contacts an amino acids corresponding to Q339 of human PPP2R1A. In embodiments, the PPP2R1A modulator contacts an amino acids corresponding to K416 of human PPP2R1A. In embodiments, the PPP2R1A modulator contacts an amino acids corresponding to H340 of human PPP2R1A.

In embodiments, the PPP2R1A modulator contacts one or more amino acids corresponding to E379, H340, S343, C377, C198, Q339, and K416 of SEQ ID NO:4. In embodiments, the PPP2R1A modulator covalently binds an amino acid corresponding to C377 in SEQ ID NO:4. In embodiments, the PPP2R1A modulator covalently binds an amino acid corresponding to C198 in SEQ ID NO:4. In embodiments, the PPP2R1A modulator contacts an amino acid corresponding to E379, H340, S343, C377, C198, Q339, and K416 of SEQ ID NO:4. In embodiments, the PPP2R1A modulator contacts an amino acids corresponding to E379 of SEQ ID NO:4. In embodiments, the PPP2R1A modulator contacts an amino acids corresponding to H340 of SEQ ID NO:4. In embodiments, the PPP2R1A modulator contacts an amino acids corresponding to S343 of SEQ ID NO:4. In embodiments, the PPP2R1A modulator contacts an amino acids corresponding to C377 of SEQ ID NO:4. In embodiments, the PPP2R1A modulator contacts an amino acids corresponding to C198 of SEQ ID NO:4. In embodiments, the PPP2R1A modulator contacts an amino acids corresponding to Q339 of SEQ ID NO:4. In embodiments, the PPP2R1A modulator contacts an amino acids corresponding to K416 of SEQ ID NO:4. In embodiments, the PPP2R1A modulator contacts an amino acids corresponding to H340 of SEQ ID NO:4.

In embodiments, the modulation is irreversible. In embodiments, the modulation is reversible. In embodiments, the compound covalently binds to the PPP2R1A protein.

In embodiments, PPP2R1A modulator binding to PPP2R1A increases activity of PP2A (e.g., PPP2CA) activity. In embodiments, PPP2R1A modulator binding to PPP2R1A increases activity of PP2A (e.g., PPP2CA) phosphatase activity. In embodiments, PPP2R1A modulator binding to PPP2R1A increases PP2A (e.g., PPP2CA) binding to another protein. In embodiments, PPP2R1A modulator binding to PPP2R1A increases PP2A (e.g., PPP2CA) de-phosphorylation of a protein. In embodiments, PPP2R1A modulator binding to PPP2R1A increases PP2A (e.g., PPP2CA) de-phosphorylation of Akt. In embodiments, PPP2R1A modulator binding to PPP2R1A increases Akt de-phosphorylation. In embodiments, PPP2R1A modulator binding to PPP2R1A increases PPP2R1A binding to PP2A (e.g., PPP2CA). In embodiments, PPP2R1A modulator binding to PPP2R1A decreases cell division. In embodiments, PPP2R1A modulator binding to PPP2R1A decreases the rate of cell division. In embodiments, PPP2R1A modulator binding to PPP2R1A decreases cell survival. In embodiments, PPP2R1A modulator binding to PPP2R1A decreases cell migration. In embodiments, PPP2R1A modulator binding to PPP2R1A decreases actin cytoskeleton polymerization. In embodiments, PPP2R1A modulator binding to PPP2R1A decreases actin cytoskeleton stabilization. In embodiments, PPP2R1A modulator binding to PPP2R1A decreases epithelial-mesenchymal transition. In embodiments, PPP2R1A modulator binding to PPP2R1A decreases promotion of the epithelial-mesenchymal transition. In embodiments, the PPP2R1A modulator binding to PPP2R1A decreases angiogenesis.

### VI. PPP2R1A Protein

In an aspect is provided a PPP2R1A protein covalently bonded to a PPP2R1A modulator (a PPP2R1A protein-PPP2R1A modulator complex). In embodiments, the PPP2R1A is a human PPP2R1A. In embodiments, the PPP2R1A modulator is a compound described herein. In embodiments, the PPP2R1A modulator is an oligonucleotide (e.g., DNA, RNA, or siRNA), protein (e.g., antibody, anti-PPP2R1A antibody, anti-PPP2R1A binding antibody fragment), or compound (e.g., compound described herein). In embodiments, the PPP2R1A modulator is provided in a therapeutically effective amount.

In embodiments, the PPP2R1A modulator contacts one or more amino acids corresponding to E379, H340, S343, C377, C198, Q339, and K416 of SEQ ID NO:4. In embodiments, the PPP2R1A modulator covalently binds an amino acid corresponding to C377 in SEQ ID NO:4. In embodiments, the PPP2R1A modulator contacts an amino acid corresponding to E379, H340, S343, C377, C198, Q339, and K416 of SEQ ID NO:4. In embodiments, the PPP2R1A modulator contacts an amino acids corresponding to E379 of SEQ ID NO:4. In embodiments, the PPP2R1A modulator contacts an amino acids corresponding to H340 of SEQ ID NO:4. In embodiments, the PPP2R1A modulator contacts an amino acids corresponding to S343 of SEQ ID NO:4. In embodiments, the PPP2R1A modulator contacts an amino acids corresponding to C377 of SEQ ID NO:4. In embodiments, the PPP2R1A modulator contacts an amino acids corresponding to Q339 of SEQ ID NO:4. In embodiments, the PPP2R1A modulator contacts an amino acids corresponding to K416 of SEQ ID NO:4. In embodiments, the PPP2R1A modulator contacts an amino acids corresponding to H340 of SEQ ID NO:4.

In an aspect is provided a PPP2R1A protein covalently bonded to a compound described herein. In embodiments, compound is covalently bonded to the amino acid corresponding to C377 of human PPP2R1A. In embodiments, compound is covalently bonded to the amino acid corresponding to C198 of human PPP2R1A.

In an aspect is provided a PPP2R1A protein covalently bonded to a compound described herein. In embodiments, compound is covalently bonded to the amino acid corresponding to C377 of SEQ ID NO:4. In embodiments, compound is covalently bonded to the amino acid corresponding to C198 of SEQ ID NO:4.

In embodiments, the PPP2R1A modulator contacts one or more amino acids corresponding to M245; N264, Q272, and D290 of human PPP2CA. In embodiments, the PPP2R1A modulator contacts an amino acids corresponding to M245 of human PPP2CA. In embodiments, the PPP2R1A modulator contacts an amino acids corresponding to N264 of human PPP2CA. In embodiments, the PPP2R1A modulator contacts an amino acids corresponding to Q272 of human PPP2CA. In embodiments, the PPP2R1A modulator contacts an amino acids corresponding to D290 of human PPP2CA.

In embodiments, the PPP2R1A modulator contacts one or more amino acids corresponding to M245; N264, Q272, and D290 of SEQ ID NO:6. In embodiments, the PPP2R1A modulator contacts an amino acids corresponding to M245 of SEQ ID NO:6. In embodiments, the PPP2R1A modulator contacts an amino acids corresponding to N264 of SEQ ID NO:6. In embodiments, the PPP2R1A modulator contacts an amino acids corresponding to Q272 of SEQ ID NO:6. In embodiments, the PPP2R1A modulator contacts an amino acids corresponding to D290 of SEQ ID NO:6.

In embodiments, the PPP2R1A modulator contacts one or more amino acids corresponding to E117, F118, and P113 of human PPP2R5C. In embodiments, the PPP2R1A modulator contacts an amino acids corresponding to E117 of human PPP2R5C. In embodiments, the PPP2R1A modulator contacts an amino acids corresponding to F118 of human PPP2R5C. In embodiments, the PPP2R1A modulator contacts an amino acids corresponding to P113 of human PPP2R5C.

In embodiments, the PPP2R1A modulator contacts one or more amino acids corresponding to E117, F118, and P113 of SEQ ID NO:5. In embodiments, the PPP2R1A modulator contacts an amino acids corresponding to E117 of SEQ ID NO:5. In embodiments, the PPP2R1A modulator contacts an amino acids corresponding to F118 of SEQ ID NO:5. In embodiments, the PPP2R1A modulator contacts an amino acids corresponding to P113 of SEQ ID NO:5.

In an aspect is provided a PPP2R1A protein covalently bonded to a compound described herein. In embodiments, compound is covalently bonded to the amino acid corresponding to C377 of human PPP2R1A (e.g., SEQ ID NO:4). In embodiments, compound is covalently bonded to the amino acid corresponding to C198 of human PPP2R1A (e.g., SEQ ID NO:4). In embodiments, the compound is bonded to a cysteine residue of the PPP2R1A protein. In embodiments, the compound is covalently bonded to a cysteine residue of the PPP2R1A protein. In embodiments, the compound is reversibly covalently bonded to a cysteine residue of the PPP2R1A protein. In embodiments, the compound is irreversibly covalently bonded to a cysteine residue of the PPP2R1A protein. In embodiments, the cysteine residue corresponds to C377 of human PPP2R1A (e.g., SEQ ID NO:4).

In an embodiment, the PPP2R1A protein is covalently bonded (e.g., reversibly or irreversibly) to a portion of a compound described herein (e.g., portion of a PPP2R1A modulator or portion of a compound described herein). In an embodiment, the PPP2R1A protein is covalently bonded (e.g., reversibly or irreversibly) to a monovalent variant of a compound described herein (e.g., monovalent variant of a PPP2R1A compound described herein). In embodiments, a monovalent variant of a compound (e.g., a compound described herein) is formed via a reaction with an electrophilic moiety and a cysteine. As a non-limiting example, a monovalent variant of a compound described herein may be represented as:

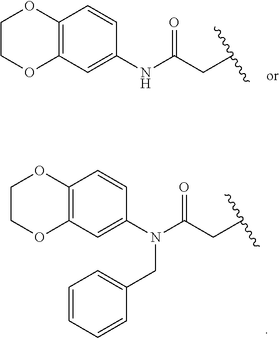 [SMILES: *CC(=O)Nc1ccc2c(c1)OCCO2 ; *CC(=O)N(Cc1ccccc1)c1ccc2c(c1)OCCO2]

In an aspect is provided a PPP2R1A protein (e.g., human PPP2R1A) covalently bonded to a PPP2R1A modulator (e.g., PPP2R1A modulator, compound described herein, or a portion of a compound described herein).

In embodiments, the PPP2R1A protein (e.g., human PPP2R1A) is covalently bonded to a PPP2R1A modulator (e.g., compound described herein or a portion of a compound described herein). In embodiments, the PPP2R1A protein (e.g., human PPP2R1A) is irreversibly covalently bonded to a PPP2R1A modulator (e.g., compound described herein or a portion of a compound described herein). In embodiments, the PPP2R1A protein (e.g., human PPP2R1A) is reversibly covalently bonded to a PPP2R1A modulator (e.g., compound described herein or a portion of a compound described herein). In embodiments, the PPP2R1A protein (e.g., human PPP2R1A) is covalently bonded to a portion of a PPP2R1A modulator (e.g., compound described herein). In embodiments, the PPP2R1A protein (e.g., human PPP2R1A) is irreversibly covalently bonded to a portion of a PPP2R1A modulator (e.g., compound described herein). In embodiments, the PPP2R1A protein (e.g., human PPP2R1A) is reversibly covalently bonded to a portion of a PPP2R1A modulator (e.g., compound described herein). In embodiments, the PPP2R1A modulator (e.g., compound described herein) is bonded to a cysteine residue (e.g., Cys377 of SEQ ID NO:4 or cysteine corresponding to Cys377 of SEQ ID NO:4) of the PPP2R1A protein (e.g., human PPP2R1A). In embodiments, the portion of a PPP2R1A modulator (e.g., compound described herein) is bonded to a cysteine residue (e.g., Cys377 of SEQ ID NO:4 or cysteine corresponding to Cys377 of SEQ ID NO:4) of the PPP2R1A protein (e.g., human PPP2R1A).

In embodiments, the PPP2R1A protein covalently bonded to a PPP2R1A modulator or compound described herein is the product of a reaction between the PPP2R1A protein and a PPP2R1A modulator or compound described herein. It will be understood that the covalently bonded PPP2R1A protein and PPP2R1A modulator (e.g., compound described herein) are the remnants of the reactant PPP2R1A protein and PPP2R1A modulator or compound, wherein each reactant now participates in the covalent bond between the PPP2R1A protein and PPP2R1A modulator or compound. In embodiments of the covalently bonded PPP2R1A protein and compound described herein, the remnant of the E substituent is a linker including a covalent bond between the PPP2R1A protein and the remainder of the compound described herein. It will be understood by a person of ordinary skill in the art that when a PPP2R1A protein is covalently bonded to a PPP2R1A modulator (e.g., compound described herein), the PPP2R1A modulator (e.g., compound described herein) forms a remnant of the pre-reacted PPP2R1A modulator (e.g., compound described herein) wherein a bond connects the remnant of the PPP2R1A modulator (e.g., compound described herein) to the remnant of the PPP2R1A protein (e.g., cysteine sulfur, sulfur of amino acid corresponding to C377 of human PPP2R1A, sulfur of C377 of human PPP2R1A (e.g., SEQ ID NO:4)). The remnant of the PPP2R1A modulator (compound described herein) may also be called a portion of the PPP2R1A modulator. In embodiments, the remnant of the E substituent is a linker selected from a

bond, —S(O)₂—, —NH—, —O—, —S—, —C(O)—, —C(O)NH—, —NHC(O)—, —NHC(O)NH—, —NHC(O)NH—, —C(O)O—, —OC(O)—, —CH₂NH—, substituted (e.g., substituted with a substituent group, a size-limited substituent group, or lower substituent group) or unsubstituted alkylene (e.g., C₁-C₈, C₁-C₆, C₁-C₄, or C₁-C₂), substituted (e.g., substituted with a substituent group, a size-limited substituent group, or lower substituent group) or unsubstituted heteroalkylene (e.g., 2 to 8 membered, 2 to 6 membered, 4 to 6 membered, 2 to 3 membered, or 4 to 5 membered), substituted (e.g., substituted with a substituent group, a size-limited substituent group, or lower substituent group) or unsubstituted cycloalkylene (e.g., C₃-C₈, C₃-C₆, C₄-C₆, or C₅-C₆), substituted (e.g., substituted with a substituent group, a size-limited substituent group, or lower substituent group) or unsubstituted heterocycloalkylene (e.g., 3 to 8 membered, 3 to 6 membered, 4 to 6 membered, 4 to 5 membered, or 5 to 6 membered), substituted (e.g., substituted with a substituent group, a size-limited substituent group, or lower substituent group) or unsubstituted arylene (e.g., C₆-C₁₀ or phenyl), or substituted (e.g., substituted with a substituent group, a size-limited substituent group, or lower substituent group) or unsubstituted heteroarylene (e.g., 5 to 10 membered, 5 to 9 membered, or 5 to 6 membered). As a non-limiting example, the PPP2R1A protein covalently bonded to a PPP2R1A modulator may have the formula:

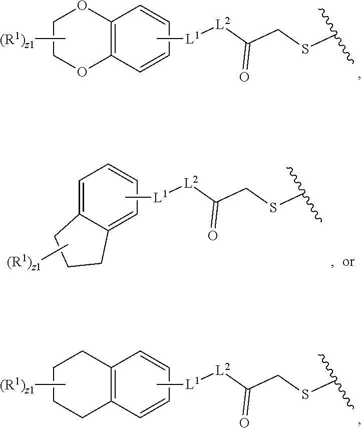

wherein S is the sulfur of a PPP2R1A protein cysteine (e.g., corresponding to C377 or C198 of human PPP2R1A (e.g., SEQ ID NO:4)), which is bonded to the remainder of the PPP2R1A protein and wherein R¹, L¹, L², and z1 are as described herein. As a non-limiting example, the PPP2R1A protein covalently bonded to a PPP2R1A modulator may have the formula:

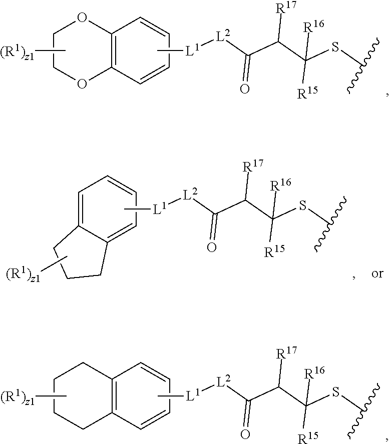

wherein S is the sulfur of a PPP2R1A protein cysteine (e.g., corresponding to C377 or C198 of human PPP2R1A (e.g., SEQ ID NO:4)), which is bonded to the remainder of the PPP2R1A protein and wherein R¹, R¹⁵, R¹⁶, R¹⁷, L¹, L², and z1 are as described herein.

As a non-limiting example, the PPP2R1A protein covalently bonded to a PPP2R1A modulator may have the formula:

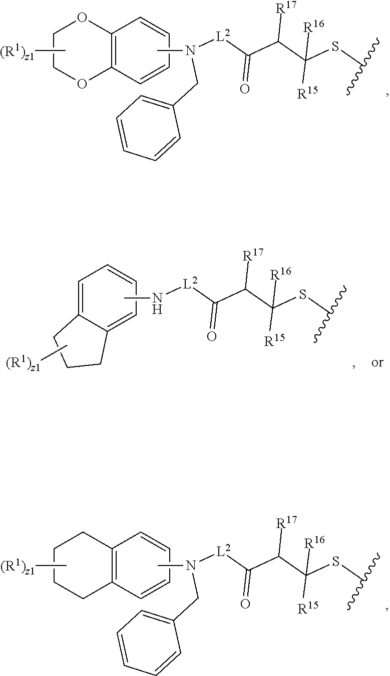

wherein S is the sulfur of a PPP2R1A protein cysteine (e.g., corresponding to C377 or C198 of human PPP2R1A (e.g., SEQ ID NO:4)), which is bonded to the remainder of the PPP2R1A protein and wherein R¹, R¹⁵, R¹⁶, R¹⁷, L¹, L², and z1 are as described herein.

As a non-limiting example, the PPP2R1A protein covalently bonded to a PPP2R1A modulator may have the formula:

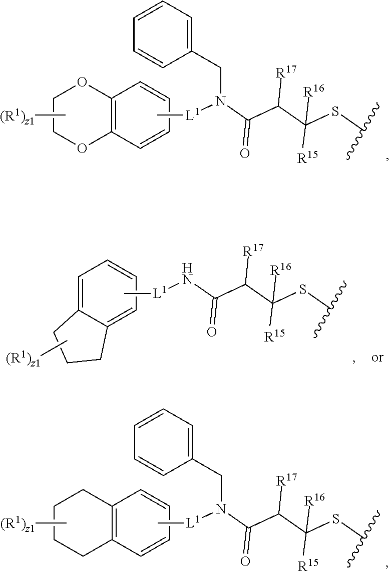

wherein S is the sulfur of a PPP2R1A protein cysteine (e.g., corresponding to C377 or C198 of human PPP2R1A (e.g., SEQ ID NO:4)), which is bonded to the remainder of the PPP2R1A protein and wherein R¹, R¹⁵, R¹⁶, R¹⁷, L¹, L², and z1 are as described herein.

In an aspect is provided a serine/threonine-protein phosphatase 2A 65 kDa regulatory subunit A alpha isoform (PPP2R1A) protein covalently bonded to a PPP2R1A modulator.

In an aspect is provided a serine/threonine-protein phosphatase 2A 65 kDa regulatory subunit A alpha isoform (PPP2R1A) protein covalently bonded to a compound described herein.

### VII. Embodiments

### Embodiment P1

A compound having the formula:

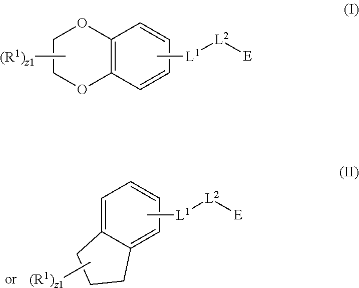

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}C(O)R^{1C}, —C(O)—OR^{1C}, —C(O)NR^{1A}R^{1B}, —OR^{1D}, —NR^{1A}SO₂R^{1D}—NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴², —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2.

### Embodiment P2

The compound of embodiment P1 having the formula:

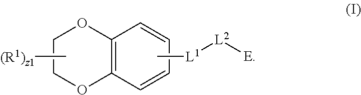

### Embodiment P3

The compound of embodiment P1 having the formula:

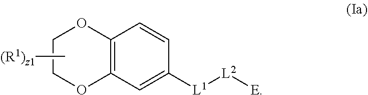

### Embodiment P4

The compound of embodiment P2 or P3 having the formula:

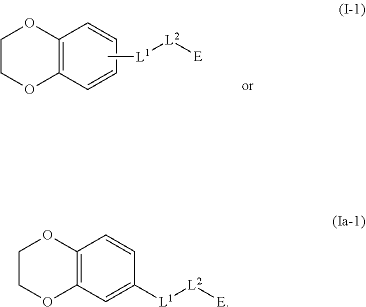

### Embodiment P5

The compound of embodiment P1 having the formula:

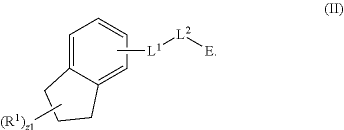

### Embodiment P6

The compound of embodiment P1 having the formula:

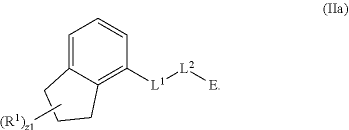

### Embodiment P7

The compound of embodiment P5 or P6 having the formula:

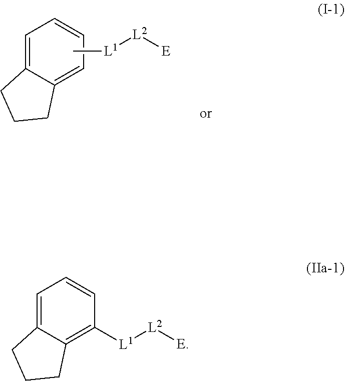

### Embodiment P8

The compound of one of embodiments P1 to P7, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment P9

The compound of one of embodiments P1 to P7, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ cycloalkyl, or substituted or unsubstituted 5 to 12 membered heteroaryl.

### Embodiment P10

The compound of one of embodiments P1 to P7, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment P11

The compound of embodiment P1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment P12

The compound of one of embodiments P1 to P11, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment P13

The compound of one of embodiments P1 to P11, wherein L¹ is a bond.

### Embodiment P14

The compound of one of embodiments P1 to P13, wherein L² is —NR⁵— or substituted or unsubstituted heterocycloalkylene comprising a ring nitrogen bonded directly to E.

### Embodiment P15

The compound of one of embodiments P1 to P13, wherein L² is —NR⁵—.

### Embodiment P16

The compound of embodiment P15, wherein R⁵ is hydrogen, substituted or unsubstituted C₁-C₆ alkyl, or substituted or unsubstituted 2 to 6 membered heteroalkyl.

### Embodiment P17

The compound of embodiment P15, wherein R⁵ is hydrogen or unsubstituted C₁-C₃ alkyl.

### Embodiment P18

The compound of embodiment P15, wherein R⁵ is hydrogen, unsubstituted methyl, unsubstituted ethyl, unsubstituted hexyl, or unsubstituted benzyl.

### Embodiment P19

The compound of embodiment P15, wherein R⁵ is hydrogen.

### Embodiment P20

The compound of one of embodiments P1 to P19, wherein E is a covalent cysteine modifier moiety.

### Embodiment P21

The compound of one of embodiments P1 to P19, wherein E is:

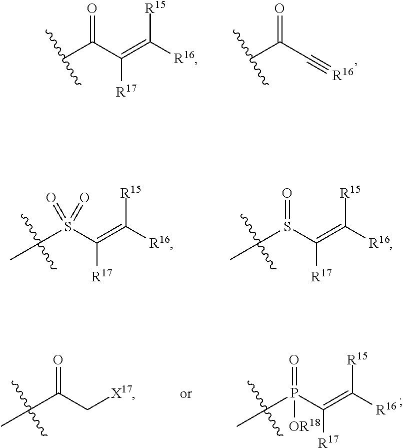

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —R^{16A}C(O)R^{16C}, —NR^{16A}C(O)OR^{16C}—NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —CX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment P22

The compound of embodiment P21, wherein R¹⁵, R¹⁶, R¹⁷, and R¹⁸ are hydrogen.

### Embodiment 23

The compound of one of embodiments P21 to P22, wherein E is:

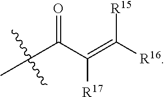

### Embodiment P24

The compound of one of embodiments P21 to P22, wherein E is:

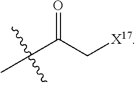

### Embodiment P25

A pharmaceutical composition comprising a Serine/threonine-protein phosphatase 2A 65 kDa regulatory subunit A alpha isoform (PPP2R1A) modulator and a pharmaceutically acceptable excipient.

### Embodiment P26

A pharmaceutical composition comprising the compound of any one of embodiments P1 to P23 and a pharmaceutically acceptable excipient.

### Embodiment P27

A method of modulating Serine/threonine-protein phosphatase 2A 65 kDa regulatory subunit A alpha isoform (PPP2R1A) protein, said method comprising contacting the Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator.

### Embodiment P28

The method of embodiment P27, wherein the Serine/threonine-protein phosphatase 2A 65 kDa regulatory subunit A alpha isoform (PPP2R1A) modulator is an siRNA, antibody, or compound.

### Embodiment P29

The method of embodiment P27, wherein the Serine/threonine-protein phosphatase 2A 65 kDa regulatory subunit A alpha isoform (PPP2R1A) modulator contacts one or more amino acids corresponding to Q339, S343, E379, K416, H340 of human PPP2RR1A; N264, Q272, M245, and D290 of human PPP2CA; or E117, and P113 and F118 of human PPP2R5C.

### Embodiment P30

A method of modulating Serine/threonine-protein phosphatase 2A 65 kDa regulatory subunit A alpha isoform (PPP2R1A) protein, said method comprising contacting the Serine/threonine-protein phosphatase 2A 65 kDa regulatory subunit A alpha isoform (PPP2R1A) protein with an effective amount of a compound of one of embodiments 1 to 23.

### Embodiment P31

The method of embodiment P30, wherein the compound is covalently bonded to the amino acid corresponding to C377 of human Serine/threonine-protein phosphatase 2A 65 kDa regulatory subunit A alpha isoform (PPP2R1A).

### Embodiment P32

The method of embodiment P30, wherein the compound contacts one or more amino acids corresponding to Q339, S343, E379, K416, H340 of human Serine/threonine-protein phosphatase 2A 65 kDa regulatory subunit A alpha isoform (PPP2R1A).

### Embodiment P33

A method of treating cancer, said method comprising administering to a subject in need thereof an effective amount of a Serine/threonine-protein phosphatase 2A 65 kDa regulatory subunit A alpha isoform (PPP2R1A) modulator.

### Embodiment P34

A method of treating cancer, said method comprising administering to a subject in need thereof an effective amount of a compound of one of embodiments 1 to 23.

### Embodiment P35

The method of one of embodiments P33 to P34, wherein the cancer is breast cancer.

### Embodiment P36

The method of one of embodiments P33 to P34, wherein the cancer is triple negative breast cancer.

### Embodiment P37

A Serine/threonine-protein phosphatase 2A 65 kDa regulatory subunit A alpha isoform (PPP2R1A) protein covalently bonded to a compound of one of embodiments P1 to P23 through the reacted residue of said electrophilic moiety.

### Embodiment P38

The Serine/threonine-protein phosphatase 2A 65 kDa regulatory subunit A alpha isoform (PPP2R1A) protein of embodiment P37, wherein the compound is bonded to a cysteine residue of the protein.

### Embodiment P39

The Serine/threonine-protein phosphatase 2A 65 kDa regulatory subunit A alpha isoform (PPP2R1A) protein of embodiment P37, covalently bonded to a portion of a compound of one of embodiments P1 to P23.

### Embodiment P40

The Serine/threonine-protein phosphatase 2A 65 kDa regulatory subunit A alpha isoform (PPP2R1A) protein of embodiment P37, irreversibly covalently bonded to a portion of a compound of one of embodiments P1 to P23.

### Embodiment P41

The Serine/threonine-protein phosphatase 2A 65 kDa regulatory subunit A alpha isoform (PPP2R1A) protein of one of embodiments P37 to P40, wherein the compound or portion of the compound is covalently bonded to an amino acid corresponding to C377 of human Serine/threonine-protein phosphatase 2A 65 kDa regulatory subunit A alpha isoform (PPP2R1A).

### Embodiment P42

A method of increasing protein phosphatase 2A (PP2A) activity, said method comprising contacting the PP2A protein complex with an effective amount of a Serine/threonine-protein phosphatase 2A 65 kDa regulatory subunit A alpha isoform (PPP2R1A) modulator.

### Embodiment P43

The method of embodiment P42, wherein the Serine/threonine-protein phosphatase 2A 65 kDa regulatory subunit A alpha isoform (PPP2R1A) modulator is an siRNA, antibody, or compound.

### Embodiment P44

The method of embodiment P42, wherein the Serine/threonine-protein phosphatase 2A 65 kDa regulatory subunit A alpha isoform (PPP2R1A) modulator contacts one or more amino acids corresponding to Q339, S343, E379, K416, H340 of human PPP2RR1A; N264, Q272, M245, and D290 of human PPP2CA; or E117, and P113 and F118 of human PPP2R5C.

### Embodiment P45

A method of modulating protein phosphatase 2A (PP2A) activity, said method comprising contacting the protein phosphatase 2A (PP2A) protein complex with an effective amount of a compound of one of embodiments P1 to P23.

### Embodiment P46

The method of embodiment P45, wherein the compound is covalently bonded to the amino acid corresponding to C377 of human Serine/threonine-protein phosphatase 2A 65 kDa regulatory subunit A alpha isoform (PPP2R1A).

### Embodiment P47

The method of embodiment P45, wherein the compound contacts one or more amino acids corresponding to Q339, S343, E379, K416, H340 of human Serine/threonine-protein phosphatase 2A 65 kDa regulatory subunit A alpha isoform (PPP2R1A).

### VIII. Embodiments

### Embodiment 1

A method of treating cancer, said method comprising administering to a subject in need thereof an effective amount of a compound having the formula:

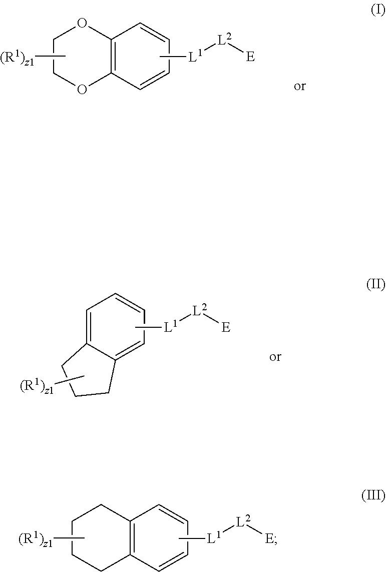

wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵², —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and m1, m4, m5, v1, v4, and v5 are independently an integer from 1 to 2.

### Embodiment 2

The method of embodiment 1 having the formula:

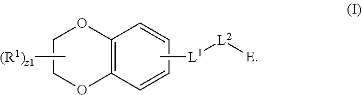

### Embodiment 3

The method of embodiment 1 having the formula:

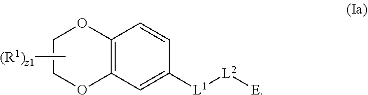

### Embodiment 4

The method of embodiment 2 or 3 having the formula:

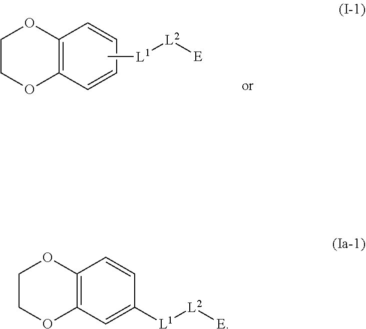

### Embodiment 5

The method of embodiment 1 having the formula:

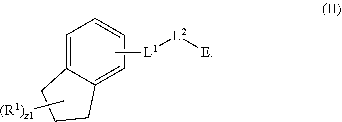

### Embodiment 6

The method of embodiment 1 having the formula:

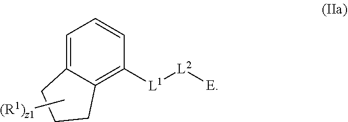

### Embodiment 7

The method of embodiment 5 or 6 having the formula:

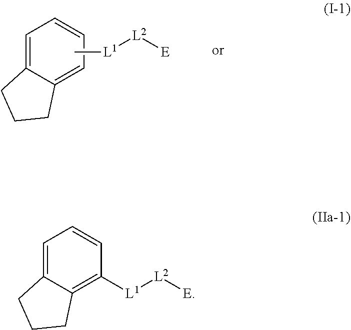

### Embodiment 8

The method of one of embodiments 1 to 7, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 9

The method of one of embodiments 1 to 7, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ cycloalkyl, or substituted or unsubstituted 5 to 12 membered heteroaryl.

### Embodiment 10

The method of one of embodiments 1 to 7, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 11

The method of embodiment 1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 12

The method of one of embodiments 1 to 11, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 13

The method of one of embodiments 1 to 11, wherein L¹ is a bond.

### Embodiment 14

The method of one of embodiments 1 to 13, wherein L² is —NR⁵— or substituted or unsubstituted heterocycloalkylene comprising a ring nitrogen bonded directly to E.

### Embodiment 15

The method of one of embodiments 1 to 13, wherein L² is —NR⁵—.

### Embodiment 16

The method of embodiment 15, wherein R⁵ is hydrogen, substituted or unsubstituted C₁-C₆ alkyl, or substituted or unsubstituted 2 to 6 membered heteroalkyl.

### Embodiment 17

The method of embodiment 15, wherein R⁵ is hydrogen or unsubstituted C₁-C₃ alkyl.

### Embodiment 18

The method of embodiment 15, wherein R⁵ is hydrogen, unsubstituted methyl, unsubstituted ethyl, unsubstituted hexyl, or unsubstituted benzyl.

### Embodiment 19

The method of embodiment 15, wherein R⁵ is hydrogen.

### Embodiment 20

The method of one of embodiments 1 to 19, wherein E is a covalent cysteine modifier moiety.

### Embodiment 21

The method of one of embodiments 1 to 19, wherein E is:

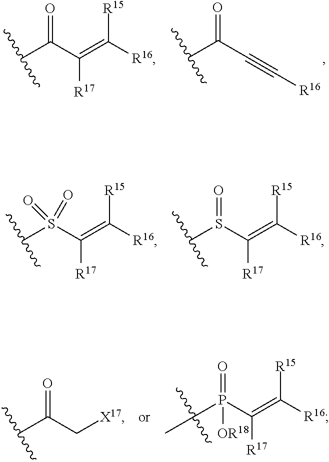

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and m15, m16, and m17 are independently and integer from 1 to 2.

### Embodiment 22

The method of embodiment 21, wherein R¹⁵, R¹⁶, R¹⁷, and R¹⁸ are hydrogen.

### Embodiment 23

The method of one of embodiments 21 to 22, wherein E is:

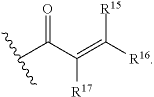

### Embodiment 24

The method of one of embodiments 21 to 22, wherein E is:

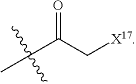

### Embodiment 25

The method of embodiment 1, wherein the compound has the formula:

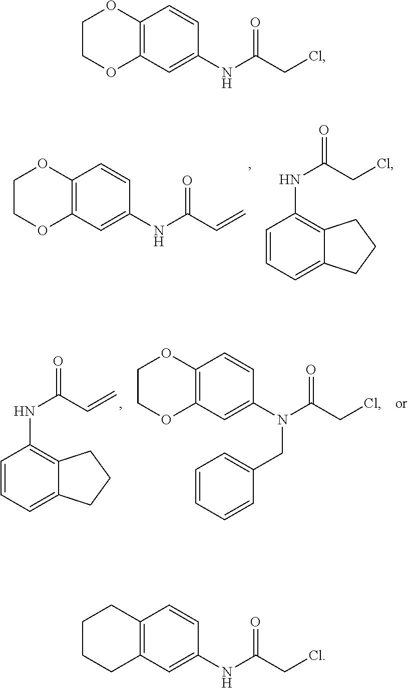

### Embodiment 26

The method of embodiment 1, wherein the compound has the formula:

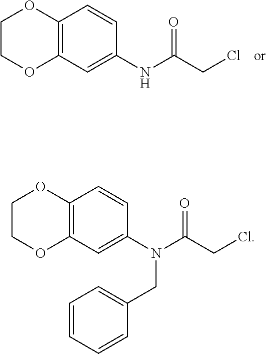 [SMILES: O=C(CCl)Nc1ccc2c(c1)OCCO2 ; O=C(CCl)N(Cc1ccccc1)c1ccc2c(c1)OCCO2]

### Embodiment 27

The method of one of embodiments 1 to 26, wherein the cancer is breast cancer.

### Embodiment 28

The method of one of embodiments 1 to 26, wherein the cancer is triple negative breast cancer.

### Embodiment 29

The use of a compound for the preparation of a medicament for the treatment of cancer, wherein the compound has the formula:

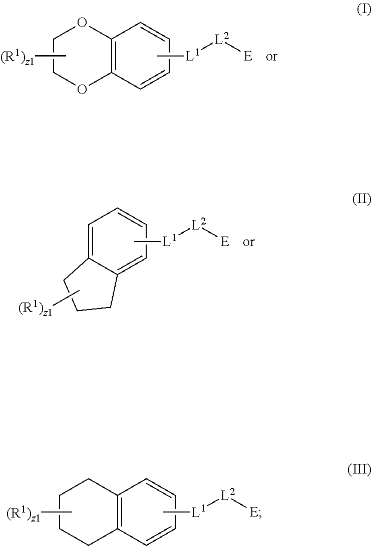

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}—NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2.

### Embodiment 30

The compound of embodiment 29 having the formula:

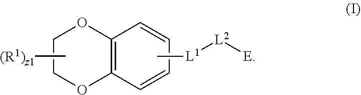

### Embodiment 31

The compound of embodiment 29 having the formula:

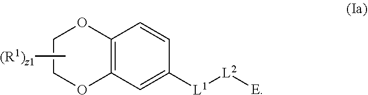

### Embodiment 32

The compound of embodiments 30 or 31 having the formula:

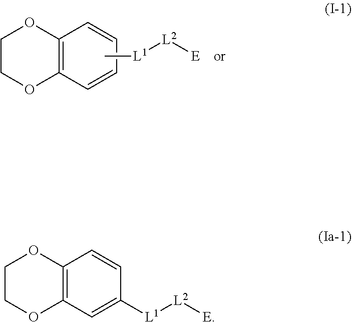

### Embodiment 33

The compound of embodiment 29 having the formula:

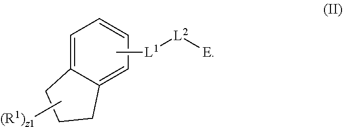

### Embodiment 34

The compound of embodiment 29 having the formula:

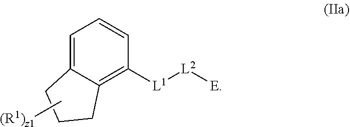

### Embodiment 35

The compound of embodiment 33 or 34 having the formula:

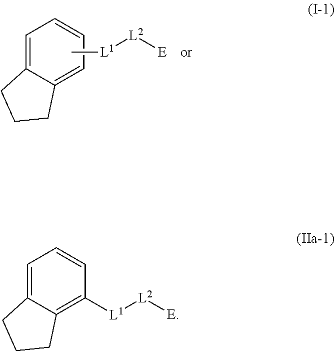

### Embodiment 36

The compound of one of embodiments 29 to 35, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 37

The compound of one of embodiments 29 to 35, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ cycloalkyl, or substituted or unsubstituted 5 to 12 membered heteroaryl.

### Embodiment 38

The compound of one of embodiments 29 to 35, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 39

The compound of embodiment 29, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 40

The compound of one of embodiments 29 to 39, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 41

The compound of one of embodiments 29 to 39, wherein L¹ is a bond.

### Embodiment 42

The compound of one of embodiments 29 to 41, wherein L² is —NR⁵— or substituted or unsubstituted heterocycloalkylene comprising a ring nitrogen bonded directly to E.

### Embodiment 43

The compound of one of embodiments 29 to 41, wherein L² is —NR⁵—.

### Embodiment 44

The compound of embodiment 43, wherein R⁵ is hydrogen, substituted or unsubstituted C₁-C₆ alkyl, or substituted or unsubstituted 2 to 6 membered heteroalkyl.

### Embodiment 45

The compound of embodiment 43, wherein R⁵ is hydrogen or unsubstituted C₁-C₃ alkyl.

### Embodiment 46

The compound of embodiment 43, wherein R⁵ is hydrogen, unsubstituted methyl, unsubstituted ethyl, unsubstituted hexyl, or unsubstituted benzyl.

### Embodiment 47

The compound of embodiment 43, wherein R⁵ is hydrogen.

### Embodiment 48

The compound of one of embodiments 29 to 47, wherein E is a covalent cysteine modifier moiety.

### Embodiment 49

The compound of one of embodiments 29 to 47, wherein E is:

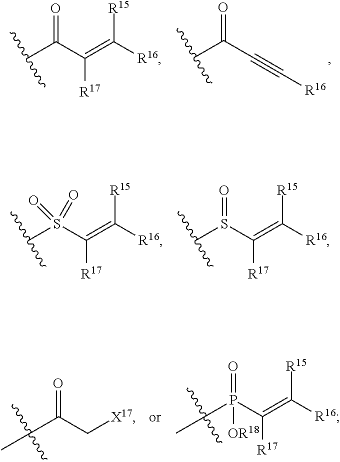

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{1C}, —C(O)—OR^{1C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}—NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment 50

The compound of embodiment 49, wherein R¹⁵, R¹⁶, R¹⁷, and R¹⁸ are hydrogen.

### Embodiment 51

The compound of one of embodiments 49 to 50, wherein E is:

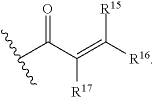

### Embodiment 52

The compound of one of embodiments 49 to 50, wherein E is:

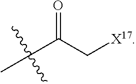

### Embodiment 53

The compound of embodiment 29, wherein the compound has the formula:

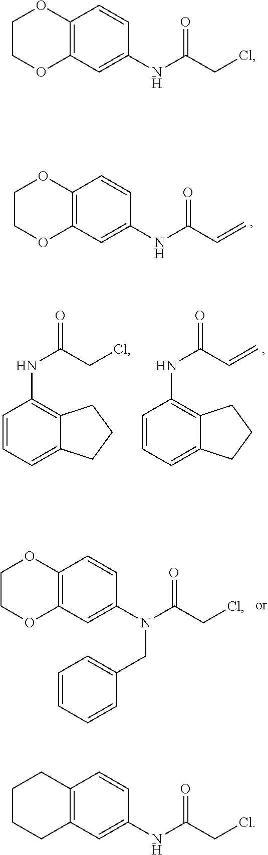

### Embodiment 54

The compound of embodiment 29, wherein the compound has the formula:

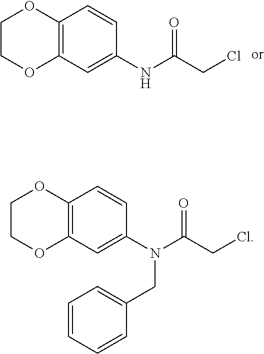 [SMILES: O=C(CCl)Nc1ccc2c(c1)OCCO2 ; "or" ; O=C(CCl)N(Cc1ccccc1)c1ccc2c(c1)OCCO2]

### Embodiment 55

A pharmaceutical composition comprising a Serine/threonine-protein phosphatase 2A 65 kDa regulatory subunit A alpha isoform (PPP2R1A) modulator and a pharmaceutically acceptable excipient.

### Embodiment 56

The pharmaceutical composition of embodiment 55, wherein the Serine/threonine-protein phosphatase 2A 65 kDa regulatory subunit A alpha isoform (PPP2R1A) modulator is a compound having the formula:

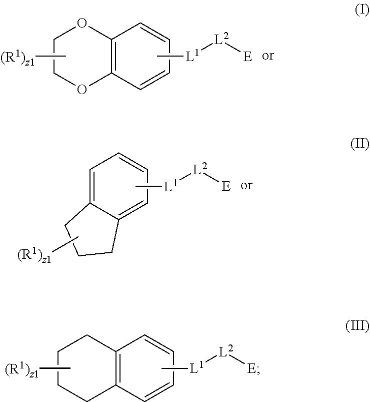

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵³, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and m1, m4, m5, v1, v4, and v5 are independently an integer from 1 to 2.

### Embodiment 57

A method of modulating a Serine/threonine-protein phosphatase 2A 65 kDa regulatory subunit A alpha isoform (PPP2R1A) protein, said method comprising contacting the PPP2R1A protein with an effective amount of a Serine/threonine-protein phosphatase 2A 65 kDa regulatory subunit A alpha isoform (PPP2R1A) modulator.

### Embodiment 58

A method of activating a tumor suppressor protein phosphatase 2A (PP2A), said method comprising contacting a Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator.

### Embodiment 59

The method of embodiments 57 or 58, wherein the Serine/threonine-protein phosphatase 2A 65 kDa regulatory subunit A alpha isoform (PPP2R1A) modulator is an antisense nucleic acid, antibody, or compound.

### Embodiment 60

The method of embodiments 57 or 58, wherein the Serine/threonine-protein phosphatase 2A 65 kDa regulatory subunit A alpha isoform (PPP2R1A) modulator contacts one or more amino acids corresponding to Q339, S343, E379, K416, H340 of SEQ ID NO:4; N264, Q272, M245, and D290 of SEQ ID NO:6; or E117, and P113 and F118 of human SEQ ID NO:5.

### Embodiment 61

The method of embodiments 57 or 58, wherein the Serine/threonine-protein phosphatase 2A 65 kDa regulatory subunit A alpha isoform (PPP2R1A) modulator is a compound having the formula:

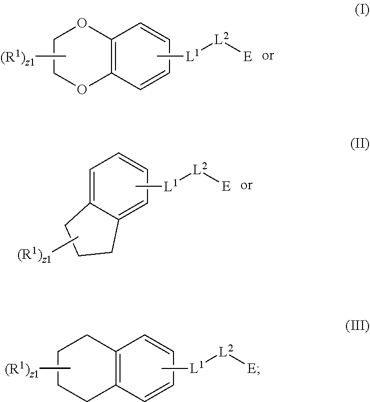

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2.

### Embodiment 62

The method of embodiment 61, wherein the compound is covalently bonded to an amino acid corresponding to C377 of SEQ ID NO:4.

### Embodiment 63

The method of embodiment 61, wherein the compound contacts one or more amino acids corresponding to Q339, S343, E379, K416, H340 of SEQ ID NO:4.

### Embodiment 64

A Serine/threonine-protein phosphatase 2A 65 kDa regulatory subunit A alpha isoform (PPP2R1A) protein covalently bonded to a compound having the formula:

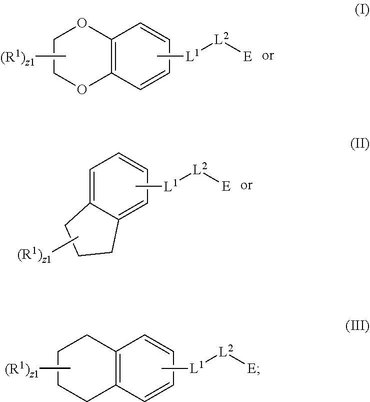

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2;

wherein the PPP2R1A protein is covalently bonded through a reacted residue of said electrophilic moiety.

### Embodiment 65

The Serine/threonine-protein phosphatase 2A 65 kDa regulatory subunit A alpha isoform (PPP2R1A) protein of embodiment 64, wherein the compound is bonded to a cysteine residue of the protein.

### Embodiment 66

The Serine/threonine-protein phosphatase 2A 65 kDa regulatory subunit A alpha isoform (PPP2R1A) protein of embodiment 64, irreversibly covalently bonded to said compound.

### Embodiment 67

The Serine/threonine-protein phosphatase 2A 65 kDa regulatory subunit A alpha isoform (PPP2R1A) protein of one of embodiments 64 to 66, wherein the compound or portion of the compound is covalently bonded to an amino acid corresponding to C377 of SEQ ID NO:4.

### Embodiment 68

A method of increasing protein phosphatase 2A (PP2A) activity, said method comprising contacting a PP2A protein complex with an effective amount of a Serine/threonine-protein phosphatase 2A 65 kDa regulatory subunit A alpha isoform (PPP2R1A) modulator.

### Embodiment 69

The method of embodiment 68, wherein the Serine/threonine-protein phosphatase 2A 65 kDa regulatory subunit A alpha isoform (PPP2R1A) modulator is an antisense nucleic acid, antibody, or compound.

### Embodiment 70

The method of embodiment 68, wherein the Serine/threonine-protein phosphatase 2A 65 kDa regulatory subunit A alpha isoform (PPP2R1A) modulator contacts one or more amino acids corresponding to Q339, S343, E379, K416, H340 of SEQ ID NO:4; N264, Q272, M245, and D290 of SEQ ID NO:6; or E117, and P113 and F118 of SEQ ID NO: 5.

### Embodiment 71

The method of one of embodiments 68 to 70 wherein the Serine/threonine-protein phosphatase 2A 65 kDa regulatory subunit A alpha isoform (PPP2R1A) modulator is a compound having the formula:

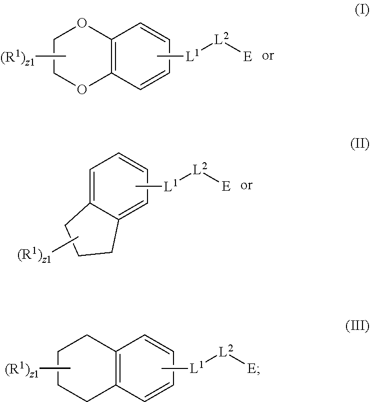

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴², —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2.

### Embodiment 72

The method of embodiment 71, wherein the compound is covalently bonded to an amino acid corresponding to C377 of human SEQ ID NO:4.

### Embodiment 73

The method of embodiment 71, wherein the compound contacts one or more amino acids corresponding to Q339, S343, E379, K416, H340 of SEQ ID NO:4.

### Embodiment 74

A compound having the formula:

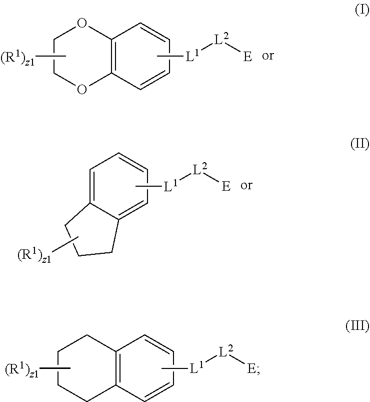

wherein,

R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;

z1 is an integer from 0 to 7;

L¹ is a

bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L² is a

bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

E is an electrophilic moiety;

Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;

each X, X¹, X⁴, and X⁵ is independently —F, —Cl, —Br, or —I;

n1, n4, and n5 are independently an integer from 0 to 4; and

m1, m4, m5, v1, v4, and v5 are independently an integer from 1 to 2.

### Embodiment 75

The compound of embodiment 74 having the formula:

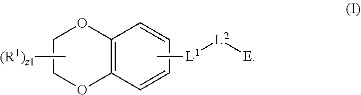

### Embodiment 76

The compound of embodiment 74 having the formula:

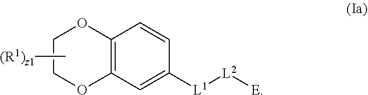

### Embodiment 77

The compound of embodiment 75 or 76 having the formula:

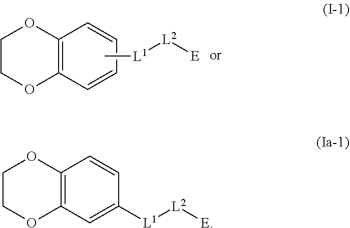

### Embodiment 78

The compound of embodiment 74 having the formula: L L2

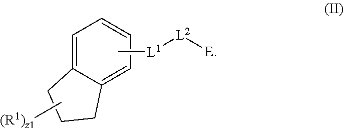

### Embodiment 79

The compound of embodiment 74 having the formula:

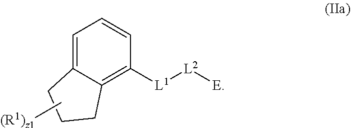

### Embodiment 80

The compound of embodiment 78 or 79 having the formula:

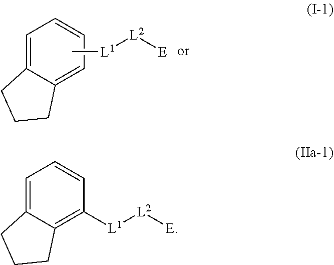

### Embodiment 81

The compound of one of embodiments 74 to 80, wherein R¹ is independently halogen, —CX¹₃, —CHX¹₂, —CH₂X¹, —OCX¹₃, —OCH₂X¹, —OCHX¹₂, —CN, —SR^{1D}, —NR^{1A}R^{1B}, —C(O)R^{1C}, —C(O)OR^{1C}, —C(O)NR^{1A}R^{1B}, —OR^{1D}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### Embodiment 82

The compound of one of embodiments 74 to 80, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ cycloalkyl, or substituted or unsubstituted 5 to 12 membered heteroaryl.

### Embodiment 83

The compound of one of embodiments 74 to 80, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

### Embodiment 84

The compound of embodiment 74, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

### Embodiment 85

The compound of one of embodiments 74 to 84, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to 6 membered heteroarylene.

### Embodiment 86

The compound of one of embodiments 74 to 84, wherein L¹ is a bond.

### Embodiment 87

The compound of one of embodiments 74 to 86, wherein L² is —NR⁵— or substituted or unsubstituted heterocycloalkylene comprising a ring nitrogen bonded directly to E.

### Embodiment 88

The compound of one of embodiments 74 to 86, wherein L² is —NR⁵—.

### Embodiment 89

The compound of embodiment 88, wherein R⁵ is hydrogen, substituted or unsubstituted C₁-C₆ alkyl, or substituted or unsubstituted 2 to 6 membered heteroalkyl.

### Embodiment 90

The compound of embodiment 88, wherein R⁵ is hydrogen or unsubstituted C₁-C₃ alkyl.

### Embodiment 91

The compound of embodiment 88, wherein R⁵ is hydrogen, unsubstituted methyl, unsubstituted ethyl, unsubstituted hexyl, or unsubstituted benzyl.

### Embodiment 92

The compound of embodiment 88, wherein R⁵ is hydrogen.

### Embodiment 93

The compound of one of embodiments 74 to 92, wherein E is a covalent cysteine modifier moiety.

### Embodiment 94

The compound of one of embodiments 74 to 92, wherein E is:

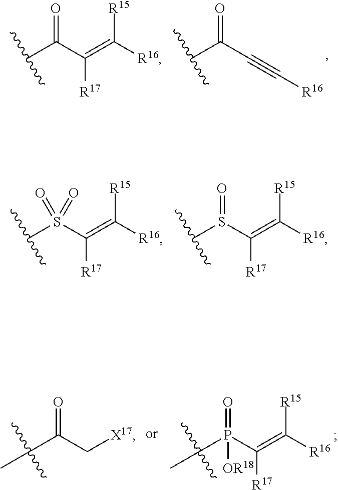

R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}, —NR^{15A}C(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;

R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;

each X, X¹⁵, X¹⁶, X¹⁷ and X¹⁸ is independently —F, —Cl, —Br, or —I;

n15, n16, n17, v15, v16, and v17, are independently an integer from 0 to 4; and

m15, m16, and m17 are independently and integer from 1 to 2.

### Embodiment 95

The compound of embodiment 94, wherein R¹⁵, R¹⁶, R¹⁷, and R¹⁸ are hydrogen.

### Embodiment 96

The compound of one of embodiments 94 to 95, wherein E is:

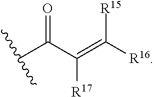

### Embodiment 97

The compound of one of embodiments 94 to 95, wherein E is:

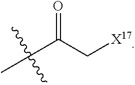

### Embodiment 98

The compound of embodiment 74, wherein the compound has the formula:

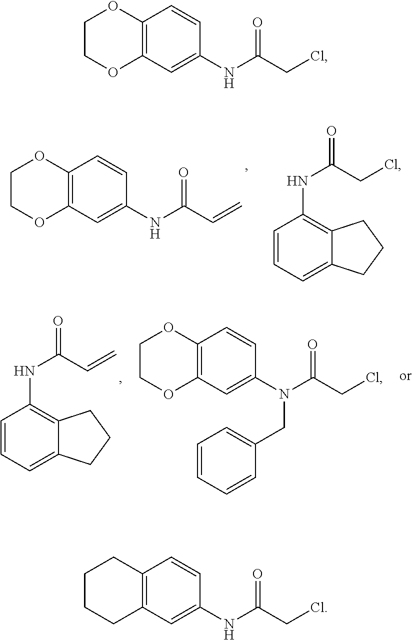

### Embodiment 99

The compound of embodiment 74, wherein the compound has the formula:

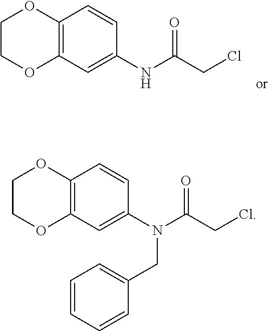 [SMILES: O=C(CCl)Nc1ccc2c(c1)OCCO2 ; O=C(CCl)N(Cc1ccccc1)c1ccc2c(c1)OCCO2]

### EXAMPLES

While there are countless covalently-acting natural products that have been shown to possess anti-cancer activity, the direct protein targets for most of these natural products are not well understood. Furthermore, many of these natural products are oftentimes difficult to synthesize or isolate, hindering their development as drugs. Identifying potential druggable hotspots targeted by covalently-acting anti-cancer natural products may enable pharmacological interrogation of these sites by more synthetically tractable compounds. Here, we used isotopic tandem orthogonal proteolysis-enabled activity-based protein profiling (isoTOP-ABPP) to map proteome-wide targets of withaferin A, a covalently-acting natural product with anti-cancer activity, directly in breast cancer proteomes. We showed that withaferin A targets C377 on the regulatory subunit PPP2R1A of the tumor suppressor protein phosphatase 2A (PP2A) complex leading to activation of PP2A, inactivation of AKT signaling, and impairments in breast cancer cell proliferation. Covalent ligand screening in breast cancer cells revealed compounds (e.g., a cysteine-reactive chloroacetamide) that modify the same cysteine on PPP2R1A. Further optimization of this covalent ligand led to the generation of JNS 1-40 that selectively targets C377 of PPP2R1A to also activate PP2A and recapitulate the signaling and pathogenic impairments observed with withaferin A in breast cancer cells. Our study highlights the utility of using chemoproteomic strategies for mapping druggable hotspots targeted by complex natural products and subsequently interrogating these sites with more synthetically tractable covalent ligands for potential cancer therapy.

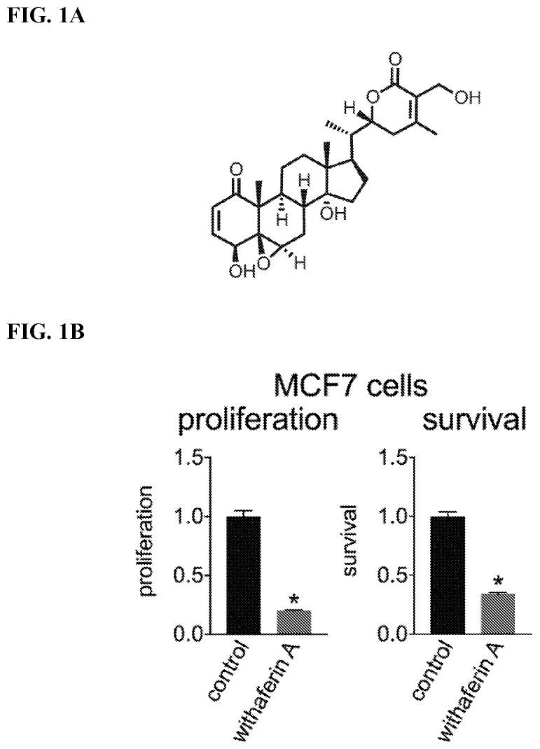

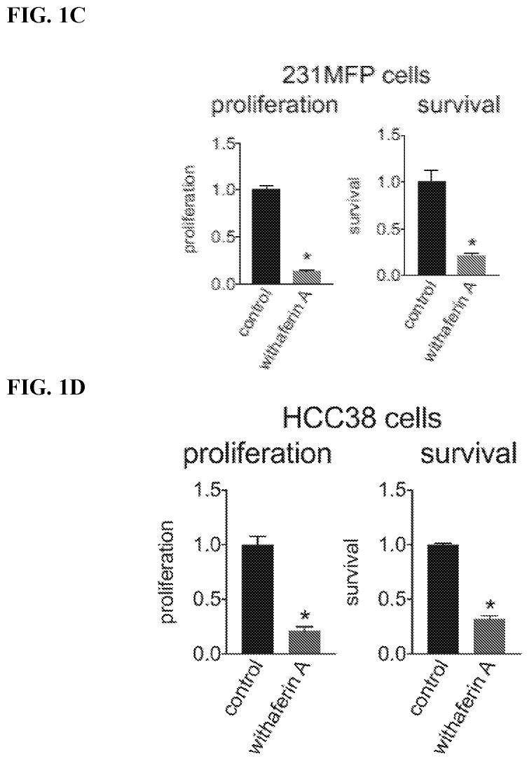

Isotopic tandem orthogonal proteolysis-enabled activity-based protein profiling (isoTOP-ABPP) has arisen as a complementary chemoproteomic approach for target discovery of covalently-acting small-molecules. IsoTOP-ABPP uses reactivity-based chemical probes to map proteome-wide reactive, functional, and ligandable hotspots directly in complex proteomes. When used in a competitive manner, covalently-acting small-molecules can be competed against the binding of reactivity-based probes directly in complex proteomes to map its proteome-wide reactivity and targets (2-5). This type of competitive isoTOP-ABPP strategy can be performed with the original parent molecule without having to synthesize analogs or derivatize the molecule. The advantage of identifying the direct targets and druggable hotspots targeted by covalently-acting anti-cancer natural products is that these targets can subsequently be deconvoluted to identify the specific target(s) responsible for the specific bioactivity; the identified targets can then be further pharmacologically interrogated with different chemical scaffolds for drug discovery efforts. This method contrasts with having to perform medicinal chemistry efforts on natural product scaffolds that are oftentimes synthetically challenging, with readouts based on their bioactivity rather than affinity to specific protein targets. Furthermore, identifying the nucleophilic amino acid hotspots targeted by reactive natural products also enables covalent ligand discovery against these sites, towards developing more potent and selective covalent inhibitors against these targets, which may also be more synthetically accessible compared to the oftentimes more complex structures of natural products. Recent studies by Backus et al. have shown that covalent ligand discovery can be used to identify selective lead ligands against unique nucleophilic druggable hotspots in proteins (5). Here, we have used the isoTOP-ABPP platform to couple target identification of a covalently-acting anti-cancer natural product with covalent ligand screening to identify a lead ligand that selectively interacts with the same target. For this study, we have chosen to investigate the proteome-wide reactivity and targets of the natural product withaferin A, a steroidal lactone from the Ayurvedic plant Withania somnifera (6-9). Withaferin A bears a Michael acceptor that may react with cysteine nucleophilic side chains in protein targets (FIG. 1A). Previous studies have shown that withaferin A binds to functional cysteines in targets such as vimentin and NF-KB which have been attributed to its anti-cancer and anti-inflammatory activities, respectively (10, 11). However, these studies either used a derivatized form of withaferin A, which could have missed targets that did not interact with this derivatized form, or performed studies with specific proteins. Thus, withaferin A may potentially possess additional targets that may be responsible for its anti-cancer activity. We have determined that withaferin A targets a particular cysteine on a regulatory subunit of the tumor suppressor protein phosphatase 2A (PP2A) to activate PP2A activity and inactivate multiple oncogenic signaling pathways which likely contributes to impairments in breast cancer pathogenicity and metabolism. We have also identified a lead covalent ligand that selectively targets this same site to recapitulate the effects observed with withaferin A. We show that withaferin A impairs breast cancer pathogenicity, potentially through reacting with multiple cysteines across multiple protein targets, including C377 on a regulatory subunit of PP2A. We show that withaferin A, through targeting this specific site, may activate the tumor-suppressor PP2A to dephosphorylate and inactivate AKT and impair glycolytic and lipid metabolism and cellular energy, which may be a mechanism through which withaferin A exerts its anti-cancer activity. We also demonstrate how covalent ligands such as DKM 2-90 and JNS 1-40, targets the same site as withaferin A and recapitulates the phenotypic, signaling, and metabolic effects.

### Example 1: General Methods

A. Materials

Chemicals and Reagents:

Unless otherwise specified, chemicals and reagents were purchased and used without further purification. Heavy and light TEV-biotin tags were synthesized using the procedures described in “Nat Protoc 2(6), 1414-1425 (2007)” and “Nature 468(7325), 790-795 (2010)”, both by Weerapana E, et. al.

Cell Culture:

The 231MFP cells were generated from explanted tumor xenografts of MDA-MB-231 cells. These cells have been previously characterized as a more aggressive variant of the MDA-MB-231 cells. HCC38, MCF7, and MCF10A cells were obtained from the American Type Culture Collection. The 231MFP cells were cultured in L15 medium containing 10% FBS, supplemented with 1% glutamine (200 mM stock), and maintained at 37° C. with 0% CO₂. Both HCC38 and MCF7 cells were cultured in RPMI medium containing 10% FBS, supplemented with 1% glutamine (200 mM stock), and maintained at 37° C. with 5% CO₂. The MCF10A cells were cultured in DMEM/F12K media containing 5% horse serum, supplemented with 1% glutamine (200 mM stock), 20 ng/mL EGF, 100 ng/mL cholera toxin, 10 ng/mL insulin, and 500 ng/mL hydrocortisone and maintained at 37° C. at 5% CO₂. Withaferin A, DKM 2-90, JNS 1-40, acrylamide or chloroacetamide compounds were dissolved in DMSO as compound stock solutions, and the final DMSO content in cells was about 0.1 vol %.

Purification of PPP2R1A and PPP2R2A Subunits.

Wild-type mammalian expression plasmids with C-terminal FLAG tag were purchased from Origene (PPP2R1A: RC200056; PPP2R2A, MR207137). The PPP2R1A C337A mutant was generated with Agilent QuickChange Lightning site-directed mutagenesis kit according to manufacturer's instructions. HEK293T cells (ATCC CRL-11268) were grown to 60% confluency in DMEM (Corning) supplemented with 10% FBS (Corning) and 2 mM L-glutamine (Life Technologies) and maintained at 37° C. with 5% CO₂. Immediately prior to transfection, media was replaced with DMEM+5% FBS. Each plate was transfected with 20 μg of overexpression plasmid with 100 μg PEI (Sigma). After 48 hrs cells were collected in TBS, lysed by sonication, and batch bound with anti-DYKDDDDK (SEQ ID NO: 1) resin (GenScript) for 1 hr. Lysate and resin was loaded onto a gravity flow column and washed, followed by elution with 250 ng/uL 3×FLAG peptide (ApexBio A6001). Purity and concentration were verified by PAGE, UV/Spectroscopy, and BCA assay.

In Vitro PP2A Activity Assay.

Recombinant PPP2CA (40 nM, Origene TP301334) was combined with pulled-down WT or mutant PPP2R1A (50 nM) as well as PPP2R2A (50 nM) and incubated with 10 μM withaferin A, JS 1-40, or vehicle for 30 min at RT in TBS. Activity was assayed by addition of 60 μM Thr phosphopeptide (KRpTIRR, Millipore, 12-219) at 37° C. for 25 min, and free phosphate was detected colorimetrically by malachite green kit (Cayman 10009325) per manufacturer's instructions.

PPP2R1A Knockdown Studies.

PPP2R1A was transiently knocked down with siRNA using previously described methods (Benjamin et al., 2014). siRNA for a scrambled RNA oligonucleotide control and pooled RNA oligonucleotides targeting PPP2R1A were purchased from Dharmacon.

B. Analytical and Purification Methods for Preparing Acrylamide or Chloroacetamide Compounds

High resolution mass spectroscopy was performed using positive or negative electrospray ionization (+ESI or −ESI). Nuclear magnetic resonance was run on a Bruker AVB 400 MHz, AVQ 400 MHz, or AV 600 MHz instrument. Silica gel flash column chromatography was used for purification of compounds described herein.

C. Cellular Phenotype Studies

Cell survival and proliferation assays were performed using Hoechst 33342 dye according to the protocol described in “Cell Chem Biol 23(5), 567-578 (2016)” by Louie S. M., et al. Cells were seeded into 96-well plates (40,000 for survival and 20,000 for proliferation) in a volume of 150 μl and allowed to adhere overnight. Cells were treated with an additional 50 μL of media containing 1:250 dilution of 1000× compound stock in DMSO. Medium was removed from each well and 100 μl of staining solution containing 10% formalin and Hoechst 33342 dye was added to each well and incubated for 15 min in the dark at room temperature. After incubation, staining solution was removed and wells were washed with PBS before imaging. Studies with HCC38 cells were also performed as above but were seeded with 20,000 cells for survival and 10,000 cells for proliferation.

D. Western Blotting

Antibodies to vinculin, phospho-Akt (Ser473), and Akt were obtained from a commercial source and proteomes were blotted per recommended manufacturer's' procedure. Cells were lysed in lysis buffer (containing the following: 20 mM Tris pH 7.5, 150 mM NaCl, 1 mM EDTA, 1 mM EGTA, 1% Triton X-100, 2.5 mM pyrophosphate, 50 mM NaF, 5 mM (3-glycero-phosphate, 1 mM Na₃VO₄, 50 nM calyculin A, and protease inhibitors). Lysate was incubated on a rotator at 4° C. for 30 min, and insoluble material was removed via centrifugation at a max speed for 10 minutes. Proteins were resolved by SDS/PAGE and transferred to nitrocellulose membranes using the iBlot system. Blots were blocked with 5% nonfat milk in Tris-buffered saline containing Tween 20 (TBST) solution for 1 hour at room temperature, washed in TBST, and probed with primary antibody diluted in a manufacturer recommended diluent overnight at 4° C. Following washes with TBST, the blots were incubated in the dark with secondary antibodies purchased from Rockland and used at 1:10 000 dilution in 5% nonfat milk in TBST at room temperature. Blots were visualized using an Odyssey Li-Cor scanner after additional washes.

E. IsoTOP-ABPP Studies

IsoTOP-ABPP studies were performed using the methods described in “Nature 534(7608), 570-574 (2016)” by Backus K. M., et al. and “Nature 468(7325), 790-795 (2010)” by Weerapana E., et al. Proteome samples diluted in PBS were treated with Withaferin A or vehicle for 30 minutes at 37° C. Then, IAyne (iodoacetamide-alkyne) labeling was performed for 1 hour at room temperature. CuAAC was used by sequential addition of tris(2-carboxyethyl)phosphine (1 mM), tris[(1-benzyl-1H-1,2,3-triazol-4-yl)methyl]amine (34 μM), copper (II) sulfate (1 mM), and biotin-linker-azide where the linker was functionalized with a TEV protease recognition sequence along with an isotopically light or heavy valine for treatment of control or treated proteome, respectively. After click reactions, proteomes were precipitated by centrifugation at 6500×g, washed in ice-cold methanol, combined in a 1:1 control/treated ratio, washed again, then denatured and re-solubilized by heating in 1.2% SDS/PBS to 80° C. for 5 minutes. Insoluble components were precipitated by centrifugation at 6500×g and soluble proteome was diluted in 0.2% SDS/PBS (5 ml). Labeled proteins were bound to avidin-agarose beads (170 μl re-suspended beads/sample) while rotating overnight at 4° C. Bead-linked proteins were enriched by washing three times each in PBS and water, then re-suspended in urea/PBS (6 M), reduced in TCEP (1 mM), alkylated with iodoacetamide (18 mM), then washed and re-suspended in urea/PBS (2 M), and finally trypsinized overnight with 0.5 μg/μl sequencing grade trypsin. Tryptic peptides were eluted off. Beads were washed in PBS (3 times) and water (3 times), washed in TEV buffer solution (water, TEV buffer, 100 μM dithiothreitol), and re-suspended in buffer with Ac-TEV protease and then incubated overnight. Peptides were diluted in water, acidified with formic acid (1.2 M), and prepared for analysis.

F. Mass Analysis of Peptides Prepared from Method E

Peptides from all proteomic experiments were pressure-loaded onto an Agilent 600 series RP-HPLC system equipped with a capillary tubing (250 mm) packed with Aqua C18 reverse-phase resin (4 cm). The system was previously equilibrated with a gradient (0% B to 100% B over 10 minutes, 100% B for 5 minutes, then 0% B for 5 minutes; buffer A: 95:5 water:acetonitrile, 0.1% formic acid; buffer B: 80:20 acetonitrile:water, 0.1% formic acid). The samples were then attached using a MicroTee PEEK 360 μm fitting to a laser pulled column (13 cm) packed with Aqua C18 reverse-phase resin (10 cm) and strong-cation exchange resin (3 cm) for isoTOP-ABPP studies. Samples were analyzed using an Q Exactive Plus mass spectrometer using a 5-step Multidimensional Protein Identification Technology (MudPIT) program. The system was run using a gradient (5% B to 55% B; buffer A: 95:5 water:acetonitrile, 0.1% formic acid; buffer B: 80:20 acetonitrile:water, 0.1% formic acid), along with a salt bump feeding aqueous ammonium acetate (500 mM) at an increment of 0%, 25%, 50%, 80%, to 100%. Data was collected in data-dependent acquisition mode with dynamic exclusion enabled (60 sec). One full MS scan (400-1800 m/z) (MS1 scan) was conducted, followed by MS2 scan (ITMS) (15 times) of the most abundant ions. Heated capillary temperature was set to 200° C. and the nanospray voltage was set to 2.75 kV.

Data was extracted in the form of MS1 and MS2 files using Raw Extractor 1.9.9.2 and searched against the Uniprot mouse database using ProLuCID search methodology in IP2 v.3, described in “J Proteomics 129:16-24 (2015)” by Xu T, et al. Cysteine residues were searched with a static modification for carboxyaminomethylation (+m/z 57.02146), up to two differential modifications for methionine oxidation, and either the light or heavy TEV tags (+m/z 464.28596 or +m/z 470.29977, respectively). Peptides were required to have at least one tryptic end and to contain the TEV modification. ProLUCID data was filtered through DTASelect to achieve a peptide false-positive rate below 1%.

Only those probe-modified peptides that were evident in two out of three biological replicates were interpreted for their isotopic light to heavy ratios. MS 1 peak shapes were confirmed to be of good quality for interpreted peptides. Targets of covalently-acting molecules are defined here as targets that showed >4 light to heavy ratios across all three biological replicates.

G. Gel-Based ABPP

Gel-based ABPP methods were performed using the methods described in “Chem Biol 22(10), 1394-1405 (2015)” by Medina-Cleghorn D., et al. Recombinant pure human proteins were purchased from Origene. Pure proteins were pre-treated with DMSO, Withaferin A, or acrylamide or chloroacetamide compounds (e.g., DKM 2-90 or JNS 1-40) for 30 minutes at 37° C. in an incubation volume of PBS (50 μL), and were subsequently treated with IAyne (10 μM final concentration) for 30 minutes at room temperature. CuAAC was performed to append rhodamine-azide onto IAyne probe-labeled proteins. The samples were separated by SDS/PAGE and scanned using a ChemiDoc MP. Inhibition of target labeling was assessed by densitometry using ImageStudio Light software.

H. Metabolomic Profiling

Metabolomic profiling was performed using the method described in “Cell Chem Biol 23(5), 567-578 (2016)” by Louie S. M., et al. For metabolomic profiling, cells (2 million) were harvested per replicate and flash-frozen. For polar metabolites, cell pellets were extracted in a mixture of acetonitrile/methanol/water (40:40:20), with inclusion of D3-15N-serine (10 nM) as an internal standard. Insoluble debris was separated via centrifugation at 13,000 rpm for 10 minutes. For nonpolar metabolites, metabolomes were extracted in 2:1 chloroform:methanol (3 ml) and PBS (1 ml) with inclusion of dodecylglycerol (10 nmol) and pentadecanoic acid (10 nmol) as internal standards. Organic and aqueous layers were separated by centrifugation at 1000×g for 5 minutes; the organic layer was collected, dried under a stream of nitrogen, and then dissolved in chloroform (120 μl). An aliquot of the nonpolar or polar extracts were then injected into an Agilent 6460 or 6430 QQQ-LC/MS/MS system. Separation of polar metabolites was achieved using normal-phase chromatography with a Luna NH2 column (5 mm) using a mobile phase (Buffer A: acetonitrile; Buffer B, 95:5 water/acetonitrile; with a modifier of 0.1% formic acid or 0.2% ammonium hydroxide) along with ammonium acetate (50 mM) for positive and negative ionization mode, respectively. The following gradient was used for each run: 0% B for 5 minutes (flow rate: 0.2 mL/min), 0% B to 100% B (linear) over 15 min (flow rate: 0.7 mL/min), then followed by an isocratic gradient of 100% B for 5 minutes (flow rate: 0.7 mL/min) and then 0% B for 5 minutes (flow rate of 0.7 mL/min). For nonpolar metabolites, metabolomes were separated using reverse-phase chromatography with a Luna C5 column (50 mm×4.6 mm with 5 μm diameter particles). Mobile phase A consisted of 95:5 ratio of water/methanol and mobile phase B consisted of 2-propanol, methanol, and water in a 60:35:5 ratio. Solvent modifiers of 0.1% formic acid, 5 mM ammonium formate, and 0.1% ammonium hydroxide were used to assist ion formation as well as to improve the LC resolution in both positive and negative ionization modes, respectively. The flow rate for each run started at 0.1 ml/min for 5 minutes to alleviate backpressure associated with injecting chloroform. The gradient started at 0% B and increased linearly to 100% B over the course of 45 min with a flow rate of 0.4 ml/min, followed by an isocratic gradient of 100% B for 17 minutes at 0.5 ml/min before equilibrating for 8 minutes at 0% B with a flow rate of 0.5 ml/min.

MS analysis was performed with an electrospray ionization (ESI) source on an Agilent 6430 or 6460 QQQ LC-MS/MS system. The capillary voltage was set to 3.0 kV, and the fragmentor voltage was set to 100 V. The drying gas temperature was 350° C., the drying gas flow rate was 10 L/min, and the nebulizer pressure was 35 psi. Metabolites were identified by SRM of the transition from precursor to product ions at associated optimized collision energies, and retention times were described in “Cell Chem Biol 23(5), 567-578 (2016)” by Louie S. M., et al. and “Proc Natl Acad Sci USA 110(37), 14912-14917 (2013)” by Benjamin D. I., et al. Metabolites were quantified by integrating the area under the curve, and then normalized to internal standard values. Metabolite levels are expressed as relative abundances as compared to controls.

### Example 2: General Procedures for Preparing Acrylamide or Chloroacetamide Compounds

A. Preparation of Acrylamide Compounds

A solution of an amine (1 eq., 0.2 mM) in dichloromethane was prepared and then cooled to 0° C. Acryloyl chloride (1.2 eq.) was added to the prepared solution followed by addition of triethylamine (1.2 eq.). The resulting solution was warmed to room temperature and stirred overnight. The final solution was then washed with brine. After removing the solvent, the crude product was purified to afford the corresponding acrylamide. In some cases, further purification may be needed, for example, recrystallization.

B. Preparation of Chloroacetamide Compounds

A solution of an amine (1 eq., 0.2 mM) in dichloromethane was prepared, and then cooled to 0° C. Chloroacetyl chloride (1.2 eq.) was added to the prepared solution followed by addition of triethylamine (1.2 eq.). The resulting solution was warmed to room temperature and stirred overnight. The final solution was then washed with brine. After removing the solvent, the crude product was purified to afford the corresponding acrylamide. In some cases, further purification may be needed, for example, recrystallization.

### Example 3: Preparation of 2-Chloro-N-(2,3-dihydrobenzo[β][1,4]dioxin-6-yl)acetamide

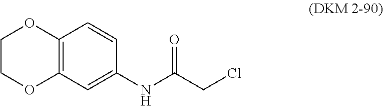 [SMILES: O=C(CCl)Nc1ccc2c(c1)OCCO2]

The procedure B of Example 2 was repeated with 1,4-benzodioxan-6-amine (1.51 g, 10 mmol); product was obtained after silica gel chromatography (40% ethyl acetate in hexanes) in 70% yield as an off-white solid (1.59 g). ¹H NMR (400 MHz, CDCl₃): δ 8.11 (s, 1H), 7.18 (d, J=2.4 Hz, 1H), 6.92 (dd, J=2.4, 8.7 Hz, 1H), 6.83 (d, J=8.7 Hz, 1H), 4.25 (s, 4H), 4.17 (s, 2H). ¹³C NMR (100 MHz, CDCl₃): δ 163.8, 143.7, 141.3, 130.4, 117.5, 114.0, 110.2, 64.5, 64.4, 43.0. HRMS (+ESI): calculated for C₁₀H₁₁ClNO₃ (M+1): 228.0422; found: 228.0421.

### Example 4: Preparation of 2-Chloro-1-(indolin-1-yl)ethan-1-one

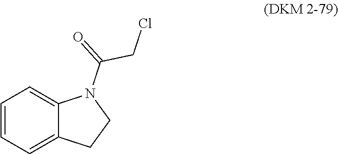 [SMILES: O=C(CCl)N1CCc2ccccc21]

The procedure B of Example 2 was repeated with indoline (331 mg, 2.8 mmol) to provide the desired product as a pale brown solid (278 mg, 51%). ¹H NMR (400 MHz, CDCl₃): δ 8.17 (d, J=8.0 Hz, 1H), 7.20-7.16 (m, 2H), 7.04 (t, J=7.4 Hz, 1H), 4.09 (s, 2H), 4.05 (t, J=8.4 Hz, 2H), 3.17 (t, J=8.4 Hz, 2H). ¹³C NMR (100 MHz, CDCl₃): δ 164.0, 142.4, 131.3, 127.6, 124.7, 124.5, 117.1, 47.7, 43.02, 28.1. HRMS (+ESI): calculated for C₁₀H₁₁ClNO (M+1): 196.0524; found: 196.0523.

### Example 5: Preparation of N-(4-Benzoylphenyl)-2-chloroacetamide

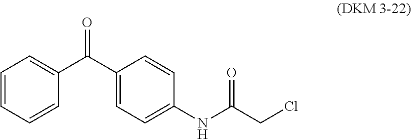 [SMILES: O=C(CCl)Nc1ccc(C(=O)c2ccccc2)cc1]

The procedure B of Example 2 was repeated with 4-aminobenzophenone (590 mg, 3.0 mmol) to provide the desired product as a light brown solid (679 mg, 83%). ¹H NMR (400 MHz, CDCl₃): δ 8.48 (s, 1H), 7.85-7.83 (m, 2H), 7.78-7.76 (m, 2H), 7.71-7.68 (m, 2H), 7.61-7.57 (m, 1H), 7.50-7.46 (m, 2H), 4.22 (s, 2H). ¹³C NMR (100 MHz, CDCl₃): δ 195.7, 164.2, 140., 137.7, 134.1, 132.5, 131.7, 130.0, 128.5, 119.3, 43.0. HRMS (−ESI): calculated for C₁₅H₁₁NO₂Cl (M-1): 272.0484; found: 272.0482.

### Example 6: Preparation of Ethyl 4-(2-chloroacetamido)benzoate

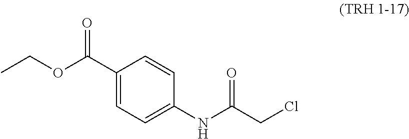 [SMILES: CCOC(=O)c1ccc(NC(=O)CCl)cc1]

The procedure B of Example 2 was repeated with benzocaine (498 mg, 3.0 mmol) to provide the desired product as a white solid (494 mg, 68%). ¹H NMR (400 MHz, CDCl₃): δ 8.67 (s, 1H), 7.98 (d, J=8.0 Hz, 2H), 7.62 (d, J=8.0 Hz, 2H), 4.33 (q, J=8.0 Hz, 2H), 4.15 (s, 2H), 1.34 (t, J=6.0 Hz, 3H). ¹³C NMR (100 MHz, CDCl₃): δ 166.1, 164.5, 141.0, 130.7, 126.7, 119.3, 61.1, 43.0, 14.3. HRMS (−ESI): calculated for C₁₁H₁₁NO₃Cl (M-1): 240.0433; found: 240.0430.

### Example 7: Preparation of 2-Chloro-N-(4-(trifluoromethyl)phenyl)acetamide

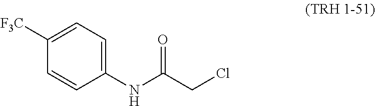 [SMILES: O=C(CCl)Nc1ccc(C(F)(F)F)cc1]

The procedure B of Example 2 was repeated with 4-(trifluoromethyl)aniline (346 mg, 2.0 mmol) to provide the desired product as a white solid (309 mg, 61%). ¹H NMR (400 MHz, MeOD): δ 7.77 (d, J=8.3 Hz, 2H), 7.61 (d, J=8.3 Hz, 2H), 4.20 (s, 2H). ¹³C NMR (100 MHz, MeOD): δ 167.7, 162.4, 142.9, 127.14, 127.10, 127.06, 127.02, 124.3, 120.9, 44.0. HRMS (−ESI): calculated for C9H6NOC1F3 (M-1): 236.0095; found: 236.0094.

### Example 8: Preparation of N,N-diphenylacrylamide

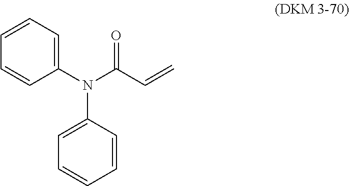 [SMILES: C=CC(=O)N(c1ccccc1)c1ccccc1]

A solution of diphenylamine (347 mg, 2.1 mmol) in dichloromethane (10 mL) was cooled to 0° C. Acryloyl chloride (222 mg, 2.5 mmol) was added to the solution followed by addition of triethylamine (279 mg, 2.8 mmol). The solution was allowed to warm to room temperature and stirred overnight. The resulting solution was washed with brine and citric acid. After removing the solvent, the crude material was purified to afford the desired product as a dark yellow oil (112 mg, 24%). ¹H NMR (400 MHz, CDCl₃): δ 7.43-7.28 (m, 10H), 6.52 (dd, J=2.0, 16.8 Hz, 1H), 6.25 (dd, J=10.2, 16.8 Hz, 1H), 5.67 (dd, J=1.8, 10.2 Hz, 1H). ¹³C NMR (100 MHz, CDCl₃): δ 165.8, 142.6, 129.7, 129.3, 128.5, 127.0. HRMS (+ESI): Calculated for C₁₅H₁₃NONa (M+Na+): 246.0889; found: 246.0887.

### Example 9: N-(3′,5′-dichloro-[1,1′-biphenyl]-4-yl)acrylamide

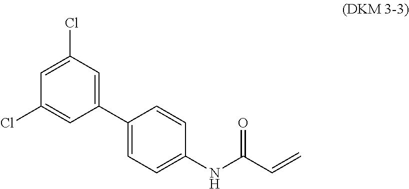 [SMILES: C=CC(=O)Nc1ccc(-c2cc(Cl)cc(Cl)c2)cc1]

The procedure A of Example 2 was repeated with 4-amino-3,5-dichlorobiphenyl (717 mg, 3.0 mmol) to provide the product. After further recrystallization from toluene, the desired product was obtained as a white solid (203 mg, 23%). ¹H NMR (600 MHz, MeOD): δ 7.77 (d, J=8.6 Hz, 2H), 7.59 (d, J=8.6 Hz, 2H), 7.56 (d, J=1.7 Hz, 2H), 7.37 (t, J=1.7 Hz, 1H), 6.46 (dd, J=9.9, 17.0 Hz, 1H), 6.39 (dd, J=1.7, 17.0 Hz, 1H), 5.80 (dd, J=1.7, 9.9 Hz, 1H). ¹³C NMR (150 MHz, MeOD): δ 166.2, 145.2, 140.4, 136.5, 135.2, 132.4, 128.5, 128.0, 127.7, 126.2, 121. HRMS (−ESI): calculated for C₁₅H₁₀NOCl₂ (M-1): 290.0145; found: 290.0143.

### Example 10: Preparation of N-(4-phenoxyphenyl)acrylamide

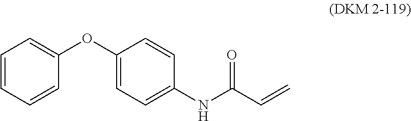 [SMILES: C=CC(=O)Nc1ccc(Oc2ccccc2)cc1]

The procedure A of Example 2 was repeated with 4-phenoxyaniline (571 mg, 3.1 mmol) to provide the desired product as a white solid (512 mg, 69%). ¹H NMR (400 MHz, CDCl₃): δ 8.17 (s, 1H), 7.55 (d, J=8.9 Hz, 2H), 7.33-7.29 (m, 2H), 7.08 (t, J=7.4 Hz, 1H), 6.98-6.94 (m, 4H), 6.42 (dd, J=1.4, 16.9 Hz, 1H), 6.31 (dd, J=10.0, 16.9 Hz, 1H), 5.73 (dd, J=1.4, 10.0 Hz, 1H). ¹³C NMR (100 MHz, CDCl₃): δ 16.0, 157.5, 153.8, 13.4, 131.2, 129., 12.8, 123.3, 122.1, 119.6, 118.6. HRMS (+ESI): calculated for C₁₅H₁₄NO₂ (M+1): 240.1019; found: 240.1015.

### Example 11: Preparation of N-(2,3-dihydrobenzo[b][1,4]dioxin-6-yl)acrylamide

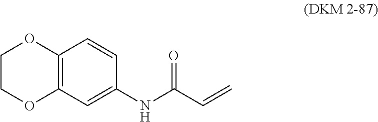

The procedure A of Example 2 was repeated with 1,4-benzodioxan-6-amine (462 mg, 3.1 mmol) to provide the desired product as a light yellow solid (239 mg, 38%). ¹H NMR (400 MHz, DMSO-d₆): δ 9.97 (s, 1H), 7.33 (d, J=2.4 Hz, 1H), 7.03 (dd, J=2.4, 8.7 Hz, 1H), 6.79 (d, J=8.7 Hz, 1H), 6.38 (dd, J=10.0, 17.0, 1H), 6.22 (dd, J=2.1, 17.0 Hz, 1H), 5.71 (dd, J=2.1, 10.0 Hz, 1H), 4.23-4.18 (m, 4H). ¹³C NMR (100 MHz, DMSO-d₆): δ 162.7, 142.9, 139.5, 132.7, 131.9, 126.4, 116.8, 112.5, 108.4, 64.2, 63.9. HRMS (+ESI): calculated for C₁₁H₁₂NO₃ (M+1): 206.0812; found: 206.0807.

### Example 12: Preparation of N-((tetrahydrofuran-2-yl)methyl)acrylamide

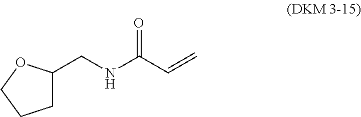

The procedure A of Example 2 was repeated with tetrahydrofurfurylamine (294 mg, 2.9 mmol) to provide the desired product as a pale yellow oil (246 mg, 55%). ¹H NMR (400 MHz, CDCl₃): 6.48 (s, 1H), 6.20 (dd, J=1.7, 17.0 Hz, 1H), 6.07 (dd, J=10.1, 17.0 Hz, 1H), 5.54 (dd, J=1.7, 10.1 Hz, 1H), 3.96-3.90 (m, 1H), 3.80-3.75 (m, 1H), 3.70-3.64 (m, 1H), 3.58-3.52 (m, 1H), 3.17-3.11 (m, 1H), 1.95-1.87 (m, 1H), 1.86-1.78 (m, 2H), 1.53-1.44 (m, 1H). ¹³C NMR (100 MHz, CDCl₃): δ 165.7, 130.8, 126.3, 77.7, 68.0, 43.2, 28.7, 25.7. HRMS (+ESI): calculated for C8H14NO2 (M+): 156.1019; found: 156.1017.

### Example 13: Preparation of N-(4-benzoylphenyl)acrylamide

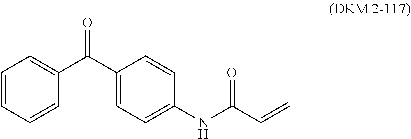 [SMILES: C=CC(=O)Nc1ccc(C(=O)c2ccccc2)cc1]

The procedure A of Example 2 was repeated with 4-aminobenzophenone (587 mg, 3.0 mmol) to provide the desired product as a yellow solid (275 mg, 37%). ¹H NMR (400 MHz, CDCl₃): δ 8.77 (s, 1H), 7.80-7.73 (m, 6H), 7.57 (tt, J=1.5, 7.4 Hz, 1H), 7.46 (t, J=7.6 Hz, 2H), 6.46 (dd, J=1.6 16.9 Hz, 1H), 6.37 (dd, J=9.9, 16.9 Hz, 1H), 5.75 (dd, J=1.6, 9.9 Hz, 1H). ¹³C NMR (100 MHz, CDCl₃): δ 196.3, 164.4, 142.3, 137.8, 133.0, 132.5, 131.7, 131.0, 130.0, 128.8, 128.4, 119.3. HRMS (+ESI): calculated for C₁₆H₁₄NO₂ (M+1): 252.1019; found: 252.1014.

### Example 14: Preparation of N-([1,1′-biphenyl]-4-ylmethyl)acrylamide

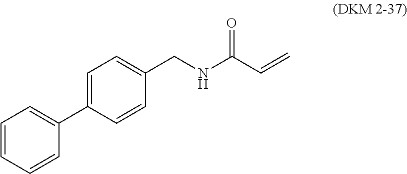 [SMILES: C=CC(=O)NCc1ccc(-c2ccccc2)cc1]

The procedure A of Example 2 was repeated with 4-phenylbenzylamine (552 mg, 3.0 mmol) to provide the desired product as an off-white solid (73 mg, 10%). ¹H NMR (400 MHz, CDCl₃): δ 7.58-7.55 (m, 4H), 7.44 (t, J=7.5 Hz, 2H), 7.38-7.33 (m, 3H), 6.35 (dd, J=1.3, 17.0 Hz, 1H), 6.13 (dd, J=10.3, 17.0 Hz, 1H), 6.01 (s, 1H), 5.68 (dd, J=1.3, 10.3 Hz, 1H), 4.56 (d, J=5.8 Hz, 2H). ¹³C NMR (100 MHz, CDCl₃): δ 165.5, 140.77, 140.73, 137.2, 130.7, 128.9, 128.5, 127.6, 127.5, 127.2, 127.1, 43.5. HRMS (+ESI): calculated for C₁₆H₁₆NO (M+1): 238.1226; found: 238.1224.

### Example 14: Preparation of 2-Chloro-N-(4-phenylbutan-2-yl)acetamide

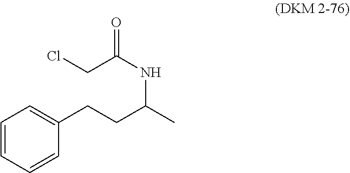

The procedure B of Example 2 was repeated with 1-methyl-3-phenylpropylamine (614 mg, 4.1 mmol) to provide the desired product as a white solid (662 mg, 81%). ¹H NMR (400 MHz, CDCl₃): δ 7.34-7.31 (m, 2H), 7.24-7.21 (m, 3H), 6.55 (d, J=7.4 Hz, 1H), 4.15-4.07 (m, 1H), 4.04 (s, 2H), 2.70 (t, J=8.2 Hz, 2H), 1.89-1.83 (m, 2H), 1.26 (d, J=6.4 Hz, 3H). ¹³C NMR (100 MHz, CDCl₃): δ 165.1, 141.3, 128.4, 128.2, 125.9, 45.7, 42.7, 381, 32.3, 20.7. HRMS (+ESI): calculated for C₁₂H₁₇ClNO (M+1): 226.0993; found: 226.0992.

### Example 15: Preparation of 2-chloro-N-(4-fluorobenzyl)acetamide

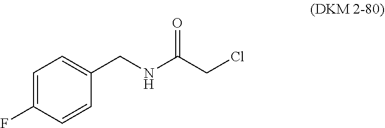 [SMILES: O=C(CCl)NCc1ccc(F)cc1]

The procedure B of Example 2 was repeated with 4-fluorobenzylamine (369 mg, 2.9 mmol) to provide the desired product as a white solid (452 mg, 77%). ¹H NMR (400 MHz, CDCl₃): δ 7.28-7.24 (m, 2H), 7.05-7.01 (m, 2H), 6.97 (s, 1H), 4.45 (d, J=5.6 Hz, 2H), 4.09 (s, 2H). ¹³C NMR (100 MHz, CDCl₃): δ 166.1, 163.6, 161.2, 133.20, 133.17, 129.64, 129.56, 115.9, 115.7, 43.2, 42.7. HRMS (−ESI): calculated for C₉H₈NOClF (M-1): 200.0284; found: 200.0284.

### Example 16: Preparation of N-(benzo[d][1,3]dioxol-5-yl)acrylamide

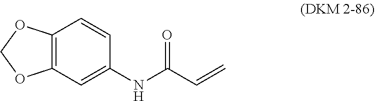

The procedure A of Example 2 was repeated with 3,4-(methylenedioxy)aniline (486 mg, 2.9 mmol) to provide the desired product as a white solid (438 mg, 68%). ¹H NMR (400 MHz, (CD₃)₂SO): δ 10.05 (s, 1H), 7.39 (d, J=2.0 Hz, 1H), 7.02 (dd, J=2.0, 8.4 Hz, 1H), 6.87 (d, J=8.4 Hz, 1H), 6.38 (dd, J=10.1, 17.0 Hz, 1H), 6.22 (dd, J=2.1, 17.0 Hz, 1H), 5.99 (s, 2H), 5.72 (dd, J=2.1, 10.1 Hz, 1H). ¹³C NMR (100 MHz, (CD₃)₂SO): δ 162.8, 147.0, 143.1, 133.4, 131.8, 126.5, 112.1, 108.1, 101.4, 101.0. HRMS (+ESI): calculated for C₁₀H₁₀NO₃(M+1): 192.0655; found: 192.0651.

### Example 17: Preparation of 2-chloro-N-(2,3-dihydro-1H-inden-4-yl)acetamide

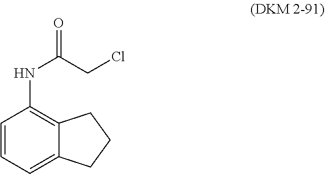 [SMILES: O=C(CCl)Nc1cccc2c1CCC2]

The procedure B of Example 2 was repeated with 4-aminoindan (372 mg, 2.8 mmol) to provide the desired product was obtained as an off-white solid (289 mg, 49%). ¹H NMR (400 MHz, CDCl₃): δ 8.19 (s, 1H), 7.74 (d, J=8.4 Hz, 1H), 7.15 (t, J=7.8 Hz, 1H), 7.05 (d, J=7.6 Hz, 1H), 4.16 (s, 2H), 2.94 (t, J=7.6 Hz, 2H), 2.82 (t, J=7.4 Hz, 2H), 2.10 (quint, J=7.5 Hz, 2H). ¹³C NMR (100 MHz, CDCl₃): δ 163.8, 145.5, 134.5, 132.8, 127.3, 121.6, 118.5, 43.1, 33.2, 29.8, 24.8. HRMS (+ESI): calculated for C₁₁H₁₃ClNO (M+1): 210.0680; found: 210.0680.

### Example 18: Preparation of 2-Chloro-N-(2-chlorobenzyl)acetamide

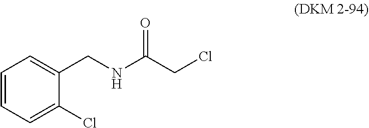 [SMILES: O=C(CCl)NCc1ccccc1Cl]

The procedure B of Example 2 was repeated with 2-chlorobenzylamine (432 mg, 3.1 mmol) to provide the desired product as a white solid (443 mg, 67%). ¹H NMR (400 MHz, CDCl₃): δ 7.36-7.18 (m, 5H), 4.51 (d, J=6.4 Hz, 2H), 4.01 (s, 2H). ¹³C NMR (100 MHz, CDCl₃): δ 166.1, 134.7, 133.5 129.8, 129.5, 129.1, 127.1, 42.5, 41.6. HRMS (−ESI): Calculated for C₉H₈NOCl₂(M-1): 215.9988; found: 215.9988.

### Example 19: Preparation of N-(4′-cyano-[1,1′-biphenyl]-4-yl)acrylamide

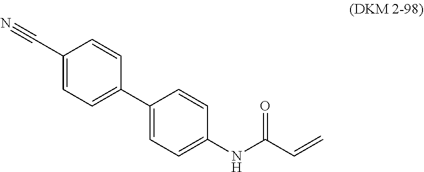 [SMILES: C=CC(=O)Nc1ccc(-c2ccc(C#N)cc2)cc1]

The procedure A of Example 2 was repeated with 4-(4-aminophenyl)benzonitrile (387 mg, 2.0 mmol) to provide the desire product as a yellow solid (348 mg, 70%). ¹H NMR (600 MHz, (D₃C)₂CO): 9.52 (s, 1H), 7.90-7.89 (m, 2H), 7.87-7.86 (m, 2H), 7.84-7.82 (m, 2H), 7.73-7.71 (m, 2H), 6.49 (dd, J=10.0, 16.9 Hz, 1H), 6.39 (dd, J=2.0, 16.9 Hz, 1H), 5.76 (dd, J=2.0, 10.0 Hz, 1H). ¹³C NMR (150 MHz, (D₃C)₂CO): δ 164.3, 145.7, 140.9, 134.8, 133.6, 132.7, 128.5, 128.2, 127.6, 120.8, 119.5, 111.3. HRMS (−ESI): calculated for C₁₆H₁₁N₂O (M-1): 247.0877; found: 247.0875.

### Example 20: Preparation of N-(4-(methylthio)phenyl)acrylamide

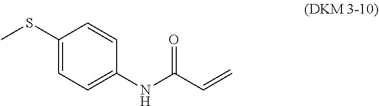

The procedure A of Example 2 was repeated with 4-(methylthio)aniline (405 mg, 2.9 mmol) to provide the desired product as a clear oil (362 mg, 64%). ¹H NMR (400 MHz, MeOD): δ 7.59-7.56 (m, 2H), 7.26-7.22 (m, 2H), 6.42 (dd, J=9.6, 17.0 Hz, 1H), 6.34 (dd, J=2.3, 17.0 Hz, 1H), 5.75 (dd, J=2.3, 9.6 Hz, 1H), 2.45 (s, 3H). ¹³C NMR (100 MHz, MeOD): δ 166.0, 137.2, 135.4, 132.4, 128.6, 127.7, 121.9, 16.4. HRMS (+ESI): Calculated for C₁₀H₁₂NOS (M+1): 194.0634; found: 194.0631.

### Example 21: Preparation of N-(4′-ethyl-[1,1′-biphenyl]-4-yl)acrylamide

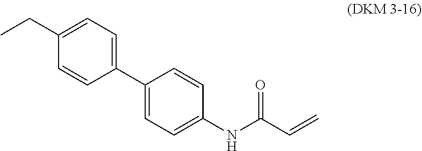 [SMILES: C=CC(=O)Nc1ccc(-c2ccc(CC)cc2)cc1]

The procedure A of Example 2 was repeated with 4-amino-4-ethylbiphenyl (386 mg, 2.0 mmol) to provide the desired product as a white solid (164 mg, 65%). ¹H NMR (400 MHz, DMSO-d₆): δ 7.82 (d, J=8.2 Hz, 2H), 7.62-7.59 (m, 2H), 7.58-7.54 (m, 2H), 7.29 (d, J=8.2 Hz, 2H), 6.47 (dd, J=9.9, 16.9 Hz, 1H), 6.36 (dd, J=2.2, 16.9 Hz, 1H), 5.72 (dd, J=2.2, 9.9 Hz, 1H), 2.67 (q, J=7.6 Hz, 2H), 1.24 (t, J=7.6 Hz, 3H). ¹³C NMR (100 MHz, DMSO-d₆): δ 164.1, 144.0, 139.5, 13.9, 137.1, 132.9, 129.3, 127.9, 127.4, 127.2, 120.7, 29.2, 16.2. HRMS (+ESI): Calculated for C₁₇H₁₈NO (M+1): 252.1383; found: 252.1379.

### Example 22: Preparation of N,N-diphenylacrylamide (Replicate)

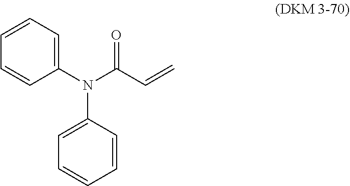 [SMILES: C=CC(=O)N(c1ccccc1)c1ccccc1]

A solution of diphenylamine (347 mg, 2.1 mmol) in DCM (10 mL) was cooled to 0° C. To the solution was added acryloyl chloride (222 mg, 2.5 mmol) followed by triethylamine (279 mg, 2.8 mmol). The solution was allowed to warm to room temperature and stirred overnight. The solution was washed with brine and citric acid. After purification, the desired product was obtained as a dark yellow oil (112 mg, 24%). ¹H NMR (400 MHz, CDCl₃): δ 7.43-7.28 (m, 10H), 6.52 (dd, J=2.0, 16.8 Hz, 1H), 6.25 (dd, J=10.2, 16.8 Hz, 1H), 5.67 (dd, J=1.8, 10.2 Hz, 1H). ¹³C NMR (100 MHz, CDCl₃): δ 165.8, 142.6, 129.7, 129.3, 128.5, 127.0. HRMS (+ESI): Calculated for C₁₅H₁₃NONa (M+Na+): 246.0889; found: 246.0887.

### Example 23: Preparation of 2-Chloro-N-(4-phenoxyphenyl)acetamide

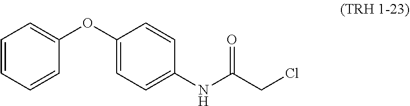 [SMILES: O=C(CCl)Nc1ccc(Oc2ccccc2)cc1]

The procedure A of Example 2 was repeated with 4-phenoxyaniline (370 mg, 2.0 mmol) to provide the desired product as a white solid (315 mg, 46%). ¹H NMR (400 MHz, CDCl₃): δ 8.42 (s, 1H), 7.52-7.48 (m, 2H), 7.35-7.31 (m, 2H), 7.10 (t, J=7.3 Hz, 1H), 7.01-6.98 (m, 4H), 4.17 (s, 2H). ¹³C NMR (100 MHz, CDCl₃): δ 164.2, 157.2, 154.4, 132.1, 129.8, 123.4, 122.2, 119.4, 118.7, 42.9. HRMS (−ESI): Calculated for C₁₄H₁₁NO₂Cl (M-1): 260.0484; found: 260.0482.

### Example 24: N-(4-(trifluoromethyl)phenyl)acrylamide

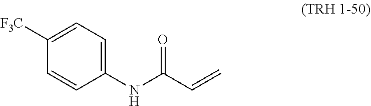

The procedure A of Example 2 was repeated with 4-(trifluoromethyl)aniline (328 mg, 2.0 mmol) to provide the desired product as a white solid (239 mg, 55%). ¹H NMR (400 MHz, MeOD): δ 7.78 (d, J=8.3 Hz, 2H), 7.55 (d, J=8.6 Hz, 2H), 6.44-6.32 (m, 2H), 5.75 (dd, J=8.4, 2.8 Hz, 1H). ¹³C NMR (100 MHz, MeOD): δ 166.3, 143.3, 132.1, 128.6, 127.04, 127.00, 126.97, 126.93, 126.6, 124.3, 120.9. HRMS (−ESI): Calculated for C₁₀H₇NOF₃ (M-1): 214.0485; found: 214.0484.

### Example 25: Preparation of 2-Chloro-N-(2-methylbenzyl)acetamide

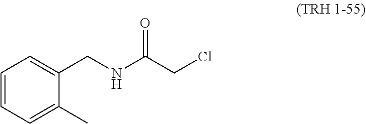

The procedure A of Example 2 was repeated with 2-methylbenzylamine (239 mg, 2.0 mmol) to provide the desired product as a white solid (191 mg, 64%) after recrystallization. ¹H NMR (400 MHz, CDCl₃): δ 7.25-7.19 (m, 4H), 6.85 (s, 1H), 4.46 (d, J=5.6 Hz, 2H), 4.04 (s, 2H), 2.33 (s, 3H). ¹³C NMR (100 MHz, CDCl₃): δ 165.8, 136.4, 135.0, 130.6, 128.4, 128.0, 126.3, 42.6, 42.0, 19.0. HRMS (−ESI): Calculated for C₁₀H₁₁NOCl (M-1): 196.0535; found: 196.0534.

### Example 26: Preparation of N-benzylacrylamide

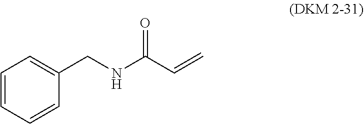

The procedure A of Example 2 was repeated with benzylamine (334 mg, 3.1 mmol) to provide the desired product as a white solid (376 mg, 75%). ¹H NMR (400 MHz, CDCl₃): δ 7.28-7.18 (m, 6H), 6.19-6.16 (m, 2H), 5.53 (dd, J=4.6, 7.3 Hz, 1H), 4.36 (d, J=5.9 Hz, 2H). ¹³C NMR (100 MHz, CDCl₃): δ 165.8, 138.1, 130.8, 128.6, 127.7, 127.3, 126.5, 43.5. HRMS (+ESI): Calculated for C₁₀H₁₂NO (M+1): 162.0913; found: 162.0912.

### Example 27: N-(4-phenylbutan-2-yl)acrylamide

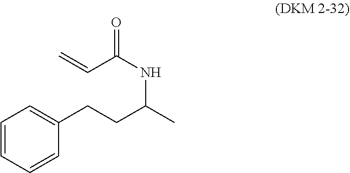

The procedure A of Example 2 was repeated with 1-methyl-3-phenylpropylamine (606 mg, 4.0 mmol) to provide the desired product as a clear oil (735 mg, 89%). ¹H NMR (400 MHz, CDCl₃): δ 7.32-7.29 (m, 2H), 7.23-7.20 (m, 3H), 6.84 (d, J=8.4 Hz, 1H), 6.36-6.24 (m, 2H), 5.64 (dd, J=2.8, 9.2 Hz, 1H), 4.21-4.14 (m, 1H), 2.70 (t, J=7.8 Hz, 2H), 1.93-1.77 (m, 2H), 1.24 (d, J=6.4 Hz, 3H). ¹³C NMR (100 MHz, CDCl₃): δ 165.1, 141.7, 131.3, 128.3, 128.2, 125.80, 125.77, 45.1, 38.4, 32.5, 20.8. HRMS (+ESI): Calculated for C₁₃H₁₈NO (M+1): 204.1383; found: 204.1380.

### Example 28: Preparation of N-(4-methoxybenzyl)acrylamide

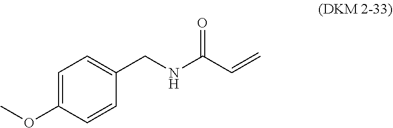

The procedure A of Example 2 was repeated with 4-methoxybenzylamine (424 mg, 3.1 mmol) to provide the desired product as a clear oil (343 mg, 60%). ¹H NMR (400 MHz, CDCl₃): δ 7.14 (d, J=8.8 Hz, 2H), 6.85 (s, 1H), 6.79 (d, J=8.4 Hz, 2H), 6.24-6.14 (m, 2H), 5.56 (dd, J=2.0, 9.6 Hz, 1H), 4.33 (d, J=5.6 Hz, 2H), 3.73 (s, 3H). ¹³C NMR (100 MHz, CDCl₃): δ 165.6, 158.9, 130.9, 130.3, 129.1, 126.4, 113.9, 55.2, 42.9. HRMS (+ESI): Calculated for C₁₁H₁₄NO₂ (M+1): 192.1019; found: 192.1017.

### Example 29: Preparation of N-(4-fluorobenzyl)acrylamide

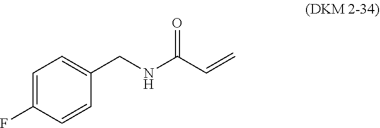

The procedure A of Example 2 was repeated with 4-fluorobenzylamine (368 mg, 2.9 mmol) to provide the desire product as an off-white solid (276 mg, 52%). ¹H NMR (400 MHz, CDCl₃): δ 7.24-7.19 (m, 2H), 6.97 (t, J=8.5 Hz, 2H), 6.42 (s, 1H), 6.27 (d, J=17.0 Hz, 1H), 6.12 (dd, J=17.0, 10.2 Hz, 1H), 5.63 (d, J=10.2 Hz, 1H), 4.42 (d, J=5.8 Hz, 2H). ¹³C NMR (100 MHz, CDCl₃): δ 165.7, 163.5, 134.0, 130.6, 129.6, 129.5, 127.0, 115.7, 115.5, 43.0. HRMS (+ESI): Calculated for C₁₀H₁₁NOF (M+1): 180.0819; found: 180.0818.

### Example 30: Preparation of Ethyl 4-acryloylpiperazine-1-carboxylate

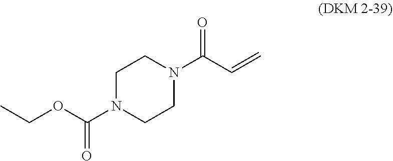

The procedure A of Example 2 was repeated with ethyl 1-piperazinecarboxylate (477 mg, 3.0 mmol) to provide the desired product as a yellow oil (372 mg, 58%). ¹H NMR (400 MHz, CDCl₃): δ 6.46 (dd, J=10.5, 16.8 Hz, 1H), 6.18 (dd, J=1.9, 16.8 Hz), 5.60 (dd, J=1.9, 10.5 Hz), 4.03 (q, J=7.1 Hz, 2H), 3.54 (s, 2H), 3.44 (s, 2H), 3.39-3.36 (m, 4H), 1.15 (t, J=7.1 Hz, 3H). ¹³C NMR (100 MHz, CDCl₃): δ 165.3, 155.1, 128.2, 127.1, 61.5, 45.4, 43.6, 43.3, 41.5, 14.5. HRMS (+ESI): Calculated for C₁₀H₁₇N₂O₃ (M+1): 213.1234; found: 213.1232.

### Example 31: Preparation of N-(2,5-difluorophenyl)acrylamide

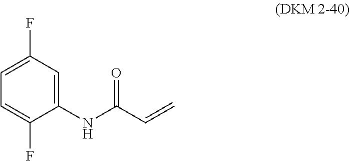

The procedure A of Example 2 was repeated with 2,5-difluoroaniline (369 mg, 2.9 mmol) to provide the desired product as a white solid (141 mg, 27%). ¹H NMR (400 MHz, (CD₃)₂CO): δ 9.26 (s, 1H), 8.29-8.24 (m, 1H), 7.24-7.18 (m, 1H), 6.90-6.84 (m, 1H), 6.67 (dd, J=10.2, 16.9 Hz, 1H), 6.41 (dd, J=1.9, 16.9 Hz, 1H), 5.79 (dd, J=1.9, 10.2 Hz, 1H). ¹³C NMR (100 MHz, (CD₃)₂CO): δ 164.6, 160.4, 151.0, 148.7, 132.0, 128.9, 128.8, 128.5, 116.7, 116.6, 116.5, 116.4, 111.1, 111.0, 110.8, 110.7, 110.0, 109.7. HRMS (+ESI): Calculated for C₉H₈F₂NO (M+1): 184.0568; found: 184.0567.

### Example 32: Preparation of N-phenethylacrylamide

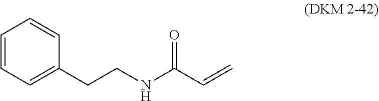

The procedure A of Example 2 was repeated with phenylethylamine (367 mg, 3.0 mmol) to provide the desired product as a yellow oil (450 mg, 85%). ¹H NMR (400 MHz, CDCl₃): δ 7.30-7.18 (m, 5H), 6.63 (s, 1H), 6.25 (dd, J=1.8, 17.0 Hz, 1H), 6.13 (dd, J=10.0, 17.0 Hz 1H), 5.59 (dd, J=1.6, 10.0 Hz, 1H), 3.56 (q, J=6.8 Hz, 2H), 2.85 (t, J=7.3 Hz, 2H). ¹³C NMR (100 MHz, CDCl₃): δ 165.8, 138.8, 131.0, 128.7, 128.6, 126.4, 126.1, 40.8, 35.6. HRMS (+ESI): Calculated for C₁₁H₁₄NO (M=1): 176.1070; found: 176.1068.

### Example 33: Preparation of N-(4-bromobenzyl)acrylamide

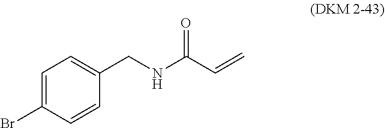

The procedure A of Example 2 was repeated with 4-bromobenzylamine (535 mg, 2.9 mmol) to provide the desired product as a white solid (407 mg, 59%). ¹H NMR (400 MHz, CDCl₃): δ 7.37 (d, J=8.4 Hz, 2H), 7.07 (d, J=8.4 Hz, 2H), 7.00 (s, 1H), 6.24-6.10 (m, 2H), 5.59 (dd, J=2.0, 9.7 Hz, 1H), 4.32 (d, J=6.0 Hz, 2H). ¹³C NMR (100 MHz, CDCl₃): δ 165.9, 137.2, 131.7, 130.6, 129.4, 126.9, 121.2, 42.8. HRMS (+ESI): Calculated for C₁₀H₁₁BrNO (M=1): 240.0019; found: 240.0016.

### Example 34: Preparation of N-(3,5-dimethylbenzyl)acrylamide

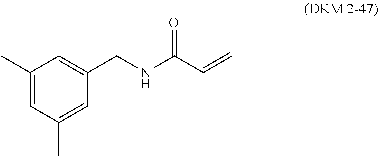

The procedure A of Example 2 was repeated with 3,5-dimethylbenzylamine (257 mg, 1.9 mmol) to provide the desired product as a white solid (276 mg, 77%). ¹H NMR (400 MHz, CDCl₃): δ 6.89-6.87 (m, 4H), 6.26 (dd, J=2.1, 17.0 Hz, 1H), 6.18 (dd, J=9.7, 17.0 Hz, 1H) 5.59 (dd, J=2.1, 9.7 Hz, 1H), 4.35 (d, J=6.0 Hz, 2H), 2.28 (s, 6H). ¹³C NMR (100 MHz, CDCl₃): δ 165.6, 138.1, 138.0, 130.9, 129.0, 126.3, 125.6, 43.4, 12.2. HRMS (+ESI): Calculated for C₁₂H₁₆NO (M+1): 190.1226; found: 190.1225.

### Example 35: Preparation of 1-(pyrrolidin-1-yl)prop-2-en-1-one

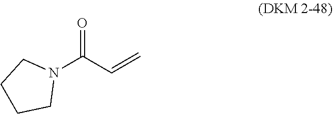

The procedure A of Example 2 was repeated with pyrrolidine (223 mg, 3.1 mmol) to provide the desired product as a pale yellow oil (148 mg, 38%). ¹H NMR (400 MHz, CDCl₃): δ 6.40 (dd, J=10.0, 16.8 Hz, 1H), 6.29 (dd, J=2.4, 16.8 Hz, 1H), 5.60 (dd, J=2.4, 10.0 Hz, 1H), 3.48 (t, J=6.8 Hz, 4H), 1.91 (quint, J=6.7 Hz, 2H), 1.82 (quint, J=6.7 Hz, 2H). ¹³C NMR (100 MHz, CDCl₃): δ 164.4, 128.8, 127.2, 46.6, 45.9, 26.1, 24.3. HRMS (+ESI): Calculated for C₇H₁₂NO (M+1): 126.0913; found: 126.0912.

### Example 36: Preparation of 1-morpholinoprop-2-en-1-one

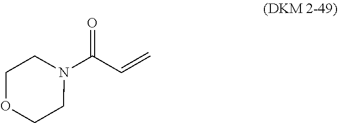

The procedure A of Example 2 was repeated with morpholine (273 mg, 3.1 mmol) to provide the desired product as a yellow oil (205 mg, 46%). ¹H NMR (400 MHz, CDCl₃): δ 6.45 (dd, J=10.5, 16.8 Hz, 1H), 6.20 (dd, J=1.9, 16.8 Hz, 1H), 5.61 (dd, J=1.9, 10.5 Hz, 1H), 5.38 (s, 6H), 3.46 (s, 2H). ¹³C NMR (100 MHz, CDCl₃): δ 165.3, 128.1, 126.9, 66.6, 46.0, 42.1. HRMS (+ESI): Calculated for C₇H₁₂NO₂ (M+1): 142.0863; found: 142.0861.

### Example 37: Preparation of N-(3-phenylpropyl)acrylamide

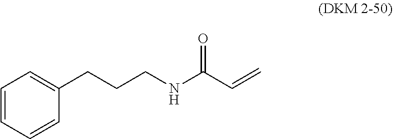

The procedure A of Example 2 was repeated with 3-phenyl-1-propylamine (275 mg, 2.0 mmol) to provide the desired product as a yellow oil (223 mg, 58%). ¹H NMR (400 MHz, CDCl₃): δ 7.29-7.25 (m, 2H), 7.20-7.16 (m, 3H), 6.99 (s, 1H), 6.29-6.17 (m, 2H), 5.59 (dd, J=2.6, 9.0 Hz, 1H), 3.34 (q, J=6.7 Hz, 2H), 2.65 (t, J=7.6 Hz, 2H), 1.87 (quint, J=7.4 Hz, 2H). ¹³C NMR (100 MHz, CDCl₃): δ 166.0, 141.4, 131.1, 128.33, 128.26, 125.9, 39.2, 33.2, 31.0. HRMS (+ESI): Calculated for C₁₂H₁₆NO (M+1): 190.1226; found: 190.1225.

### Example 38: Preparation of N-(2-(2-methoxyphenoxy)ethyl)acrylamide

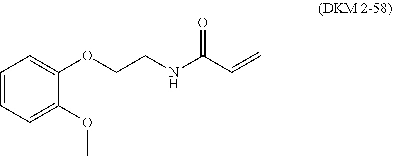 [SMILES: C=CC(=O)NCCOc1ccccc1OC]

The procedure A of Example 2 was repeated with 2-(2-methoxyphenoxy)ethanamine (509 mg, 3.0 mmol) to provide the desired product as a yellow oil (470 mg, 70%). ¹H NMR (400 MHz, CDCl₃): δ 6.95-6.84 (m, 4H), 6.77 (s, 1H), 6.26 (d, J=17.1 Hz, 1H), 6.11 (dd, J=10.2, 17.1 Hz, 1H), 5.59 (d, J=10.2 Hz, 1H), 4.07 (t, J=5.2 Hz, 2H), 3.79 (s, 3H), 3.69 (q, J=5.4 Hz, 2H). ¹³C NMR (100 MHz, CDCl₃): δ 165.7, 149.6, 147.7, 130.8, 126.4, 122.1, 121.0, 114.8, 111.8, 68.5, 55.7, 38.9. HRMS (+ESI): Calculated for C₁₂H₁₅NO₃Na (M+Na+): 244.0944; found: 244.0940.

### Example 39: Preparation of N-([1,1′-biphenyl]-2-ylmethyl)acrylamide

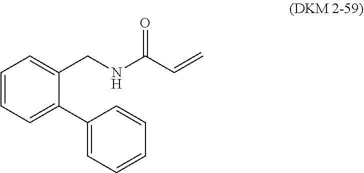 [SMILES: C=CC(=O)NCc1ccccc1-c1ccccc1]

The procedure A of Example 2 was repeated with 2-phenylbenzylamine (202 mg, 1.1 mmol) to provide the desired product as a yellow oil (184 mg, 70%). ¹H NMR (400 MHz, CDCl₃): δ 7.41-7.22 (m, 9H), 6.16 (dd, J=1.2, 17.2 Hz, 1H), 6.03-5.97 (m, 2H), 5.55 (dd, J=1.2, 10.0 Hz, 1H), 4.44 (d, J=5.6 Hz, 2H). ¹³C NMR (100 MHz, CDCl₃): δ 165.3, 141.6, 140.6, 135.2, 1306, 130.2, 129.0, 128.7, 128.4, 127.8, 127.4, 127.3, 126.4, 41.4. HRMS (+ESI): Calculated for C₁₆H₁₆NO (M+1): 238.1226; found: 238.1223.

### Example 40: Preparation of N-(2-chlorobenzyl)acrylamide

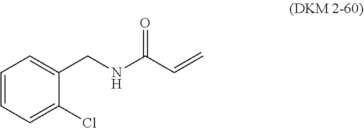

The procedure A of Example 2 was repeated with 2-chlorobenzylamine (406 mg, 2.9 mmol) to provide the desired product as a white solid (162 mg, 34%). ¹H NMR (400 MHz, CDCl₃): δ 7.34-30 (m, 2H), 7.20-7.16 (m, 2H), 6.84 (s, 1H), 6.25 (dd, J=2.0, 17.0 Hz, 1H), 6.16 (dd, J=9.7, 17.0 Hz, 2H), 5.60 (dd, J=2.0, 9.7 Hz, 1H), 4.52 (d, J=6.1 Hz, 2H). ¹³C NMR (100 MHz, CDCl₃): δ 165.9, 135.5, 133.5, 130.6, 129.8, 129.5, 128.8, 127.1, 126.8, 41.4. HRMS (+ESI): Calculated for C₁₀H₁₁ClNO (M+1): 196.0524; found: 196.0521.

### Example 41: Preparation of N-(2-nitrobenzyl)acrylamide

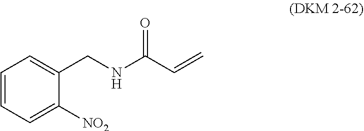

The procedure A of Example 2 was repeated with 2-nitrobenzylamine hydrochloride (406 mg, 2.9 mmol) with an extra equivalent of trimethylamine to provide the desired product as a yellow solid (255 mg, 42%). ¹H NMR (400 MHz, CDCl₃): δ 7.98 (dd, J=1.1, 8.2 Hz, 1H), 7.58-7.52 (m, 2H), 7.41-7.37 (m, 1H), 7.03 (s, 1H), 6.22 (dd, J=2.0, 17.0 Hz, 1H), 6.14 (dd, J=9.7, 17.0 Hz, 1H), 5.59 (dd, J=2.0, 9.7 Hz, 1H), 4.68 (d, J=6.4 Hz, 2H). ¹³C NMR (100 MHz, CDCl₃): δ 165.8, 148.2, 134.1, 133.6, 131.9, 130.4, 128.7, 127.1, 125.1, 41.2. HRMS (+ESI): Calculated for C₁₀H₁₁N₂O₃ (M+1): 207.0764; found: 207.0760.

### Example 42: Preparation of N-(2,3-dihydro-1H-inden-4-yl)acrylamide

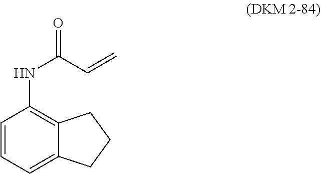

The procedure A of Example 2 was repeated with 4-aminoindan (402 mg, 3.0 mmol) to provide the desired product as a white solid (332 mg, 59%). ¹H NMR (400 MHz, CDCl₃): δ 7.72 (d, J=7.5 Hz, 1H), 7.54 (s, 1H), 7.10 (t, J=7.7 Hz, 1H), 7.01 (d, J=7.2 Hz, 1H), 6.40-6.26 (m, 2H), 5.69 (dd, J=1.9, 9.7 Hz, 1H), 2.91 (t, J=7.4 Hz, 2H), 2.78 (t, J=7.4 Hz, 2H), 2.05 (quint, J=7.4 Hz, 2H). ¹³C NMR (100 MHz, CDCl₃): 163.5, 145.3, 134.4, 133.6, 131.2, 127.5, 127.2, 12.0, 19.2, 33.2, 30.1, 24.8. HRMS (+ESI): Calculated: 188.1070 (C₁₂H₁₄NO). Observed: 188.1069.

### Example 43: Preparation of Ethyl 4-acrylamidobenzoate

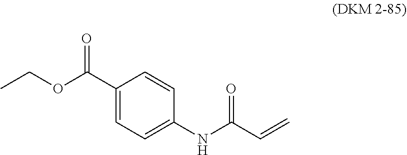 [SMILES: C=CC(=O)Nc1ccc(C(=O)OCC)cc1]

The procedure A of Example 2 was repeated with benzocaine (486 mg, 2.9 mmol) to provide the product as a white solid (438 mg, 68%). ¹H NMR (400 MHz, CDCl₃): δ 9.39 (s, 1H), 7.95 (d, J=8.7 Hz, 2H), 7.74 (d, J=8.7 Hz, 2H), 6.43-6.41 (m, 2H), 5.71 (dd, J=4.7, 6.9 Hz, 2H), 4.31 (q, J=7.1 Hz, 2H), 1.33 (s, J=7.1 Hz, 3H). ¹³C NMR (100 MHz, CDCl₃): δ 166.5, 164.6, 142.5, 131.0, 130.6, 128.4, 125.7, 119.4, 61.0, 14.2. HRMS (−ESI): Calculated: 218.0823 (C₁₂H₁₂NO₃). Observed: 218.0822.

### Example 44: Preparation of N-benzyl-N-methylacrylamide

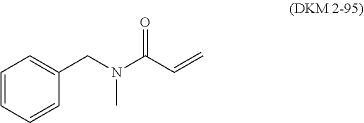

The procedure A of Example 2 was repeated with N-methylbenzylamine (350 mg, 2.9 mmol) to provide the product as a clear oil (304 mg, 60%). ¹H NMR (˜48:52 rotamer ratio, asterisks denote minor peaks, 400 MHz, CDCl₃): δ 7.34-7.23 (m, 4H), 7.16 (s, 1H), 7.14* (s, 1H), 6.61 (dd, J=10.4, 16.8 Hz, 1H), 6.57* (dd, J=10.4, 16.8 Hz, 1H), 6.38 (dd, J=1.9, 16.8 Hz, 1H), 6.36* (dd, J=1.9, 16.8 Hz, 1H), 5.71 (dd, J=1.9, 10.4 Hz, 1H), 5.64* (dd, J=1.9, 10.4 Hz), 4.63 (s, 2H), 4.56* (s, 2H), 2.98* (s, 3H), 2.96 (s, 3H). ¹³C NMR (100 MHz, CDCl₃): δ 167.0, 166.4, 137.1, 136.5, 128.8, 128.5, 128.2, 128.0, 17.62, 127.59, 127.3, 126.3, 53.3, 51.0, 34.8, 34.0. HRMS (+ESI): Calculated: 176.1070 (C₁₁H₁₄NO). Observed: 176.1070.

### Example 45: Preparation of 1-(4-phenylpiperidin-1-yl)prop-2-en-1-one

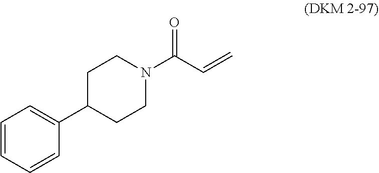 [SMILES: C=CC(=O)N1CCC(c2ccccc2)CC1]

The procedure A of Example 2 was repeated with 4-phenylpiperidine (331 mg, 2.1 mmol) to provide the product as a yellow oil (379 mg, 86%). ¹H NMR (400 MHz, CDCl₃): δ 7.32-7.28 (m, 2H), 7.22-7.17 (m, 3H), 6.62 (dd, J=10.6, 16.8 Hz, 1H), 6.30 (dd, J=1.9, 16.8 Hz, 1H), 5.68 (dd, J=1.9, 10.6, Hz, 1H), 4.82 (d, J=12.9 Hz, 1H), 4.11 (d, J=13.2 Hz, 1H), 3.15 (t, J=8.5 Hz, 1H), 2.78-2.67 (m, 2H), 1.90 (d, J=12.9 Hz, 2H), 1.64 (quint, J=12.3 Hz, 2H). ¹³C NMR (100 MHz, CDCl₃): 165.3, 145.0, 128.5, 127.8, 127.4, 126.6, 126.4, 46.4, 42.7, 33.9, 32.7. HRMS (+ESI): Calculated: 216.1383 (C₁₄H₁₈NO). Observed: 216.1383.

### Example 46: Preparation of N-(2-morpholinoethyl)acrylamide

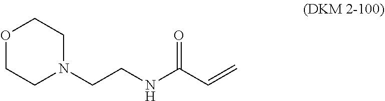

The procedure A of Example 2 was repeated with 2-morpholinoethylamine (580 mg, 3.0 mmol) to provide the product as a white solid (184 mg, 33%). ¹H NMR (400 MHz, CDCl₃): δ 6.39 (s, 1H), 6.21 (dd, J=1.7, 17.0 Hz, 1H), 6.08 (dd, J=10.1, 17.0 Hz, 1H), 5.56 (dd, J=1.7, 10.1 Hz, 1H), 3.63 (t, J=4.6 Hz, 4H), 3.36 (q, J=6.2 Hz, 2H), 2.45 (t, J=6.2 Hz, 2H), 2.40-2.38 (m, 4H). ¹³C NMR (100 MHz, CDCl₃): δ 165.5, 130.9, 126.2, 66.9, 57.0, 53.3, 35.7. HRMS (+ESI): Calculated: 185.1285 (C₉H₁₇N₂O₂). Observed: 185.1280.

### Example 47: Preparation of 1-(indolin-1-yl)prop-2-en-1-one

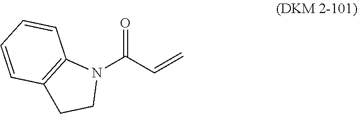

The procedure A of Example 2 was repeated with indoline (580 mg, 3.0 mmol) to provide the product as a green solid (285 mg, 56%). ¹H NMR (400 MHz, CDCl₃): δ 8.30 (d, J=7.7 Hz, 1H), 7.22-7.17 (m, 2H), 7.03 (t, J=7.9 Hz, 1H), 6.60-6.48 (m, 2H), 5.79 (dd, J=2.6, 9.5 Hz, 1H), 4.15 (t, J=8.5 Hz, 2H), 3.20 (t, J=8.1, 2H). ¹³C NMR (100 MHz, CDCl₃): δ 163.6, 142.6, 131.5, 129.0, 128.6, 127.2, 124.4, 123.8, 117.2, 47.8, 27.7. HRMS (+ESI): Calculated: 174.0913 (C₁₁H₁₂NO). Observed: 174.0911.

### Example 48: Preparation of N-butylacrylamide

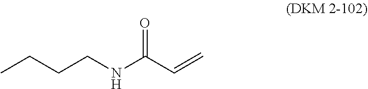

The procedure A of Example 2 was repeated with butylamine (223 mg, 3.0 mmol) to provide the product was obtained as a clear oil (237 mg, 61%). ¹H NMR (400 MHz, (CDCl₃): δ 6.81 (s, 1H), 6.21-6.10 (m, 2H), 5.52 (dd, J=3.6, 8.3 Hz, 1H), 3.26-3.21 (m, 2H), 1.48-1.41 (m, 2H), 1.33-1.23 (m, 2H), 0.84 (t, J=7.3 Hz, 3H). ¹³C NMR (100 MHz, CDCl₃): δ 166.0, 131.2, 125.6, 39.3, 31.5, 20.1, 13.7. HRMS (+ESI): Calculated: 128.1070 (C₇H₁₄NO). Observed: 128.1068.

### Example 49: Preparation of N-(3-methoxypropyl)acrylamide

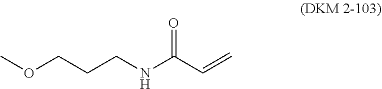

The procedure A of Example 2 was repeated with 3-methoxypropylamine (274 mg, 3.1 mmol) to provide the product as a clear oil (236 mg, 54%). ¹H NMR (400 MHz, CDCl₃): δ 6.84 (s, 1H), 6.15 (dd, J=2.0, 17.0 Hz. 1H), 6.07 (dd, J=9.8, 17.0 Hz, 1H), 5.51 (dd, J=2.0, 9.8 Hz, 1H), 3.39 (t, J=5.9 Hz, 2H), 3.33 (q, J=6.3 Hz, 2H), 3.25 (s, 3H), 1.72 (quint, J=6.3 Hz, 2H). ¹³C NMR (100 MHz, CDCl₃): δ 165.8, 131.2, 125.7, 71.3, 58.7, 37.7, 29.0. HRMS (+ESI): Calculated: 144.1019 (C₇H₁₄NO₂). Observed: 144.1017.

### Example 50: Preparation of N-cyclohexylacrylamide

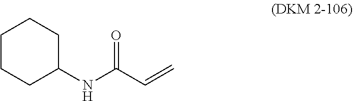

The procedure A of Example 2 was repeated with cyclohexylamine (292 mg, 2.9 mmol) to provide the product as a white solid (313 mg, 86%). ¹H NMR (400 MHz, (CDCl₃): δ 6.55 (d, J=6.7 Hz, 1H), 6.21-6.09 (m, 2H), 5.51 (dd, J=2.5, 9.1 Hz, 1H), 3.79-3.70 (m, 1H), 1.86-1.82 (m, 2H), 1.67-1.63 (m, 2H), 1.56-1.52 (m, 1H), 1.28-1.21 (m, 2H), 1.16-1.05 (m, 3H). ¹³C NMR (100 MHz, CDCl₃): δ 164.8, 131.5, 125.7, 48.3, 32.9, 25.5, 24.9. HRMS (+ESI): Calculated: 154.1226 (C₉H₁₆NO). Observed: 154.1224.

### Example 51: Preparation of N-(4-chlorophenyl)acrylamide

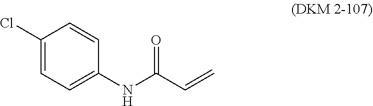

The procedure A of Example 2 was repeated with 4-chloroaniline (386 mg, 3.0 mmol) to provide the product was obtained after silica gel chromatography (0% to 40% ethyl acetate in hexanes) followed by recrystallization from toluene in 31% yield as a white solid (168 mg). ¹H NMR (400 MHz, (CD₃)₂CO): δ 9.47 (s, 1H), 7.77-7.74 (m, 2H), 7.35-7.31 (m, 2H), 6.43 (dd, J=9.6, 16.9 Hz, 1H), 6.35 (dd, J=2.5, 16.9 Hz, 1H), 5.73 (dd, J=2.5, 9.6 Hz, 1H). ¹³C NMR (100 MHz, (CD₃)₂CO): δ 164.1, 139.0, 132.5, 129.5, 128., 127.5, 121.7. HRMS (−ESI): Calculated: 180.0222 (C₉H₇NOCl). Observed: 180.0221.

### Example 52: Preparation of N-cyclopentylacrylamide

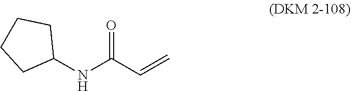

The procedure A of Example 2 was repeated with cyclopentylamine (257 mg, 3.0 mmol) to provide the product as a colorless oil (229 mg, 55%). ¹H NMR (400 MHz, (CDCl₃): δ 6.70 (s, 1H), 6.21-6.10 (m, 2H), 5.51 (dd, J=3.5, 8.5 Hz, 1H), 5.53-5.50 (sex, J=7.1 Hz, 1H), 1.94-1.86 (m, 2H), 1.65-1.46 (m, 4H), 1.41-1.32 (m, 2H). ¹³C NMR (100 MHz, CDCl₃): δ 165.4, 131.3, 125.7, 51.1, 32.9, 23.8. HRMS (+ESI): Calculated: 140.1070 (C₈H₁₄NO). Observed: 140.1067.

### Example 53: Preparation of 1-(4-methoxypiperidin-1-yl)prop-2-en-1-one

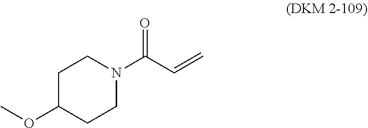

The procedure A of Example 2 was repeated with 4-methoxypiperidine (461 mg, 3.0 mmol) to provide the product as a pale yellow oil (386 mg, 75%). ¹H NMR (400 MHz, (CDCl₃): δ 6.45 (dd, J=10.6, 16.8 Hz, 1H), 6.09 (dd, J=2.0, 16.8 Hz, 1H), 5.51 (dd, J=2.0, 10.6 Hz, 1H), 3.80-3.74 (m, 1H), 3.65-3.58 (m, 1H), 3.33-3.17 (m, 6H), 1.74-1.67 (m, 2H), 1.47-1.39 (m, 2H). ¹³C NMR (100 MHz, CDCl₃): δ 165.1, 127.6, 127.2, 75.0, 55.5, 42.7, 38.9, 31.1, 29.9. HRMS (+ESI): Calculated: 170.1176 (C₉H₁₆NO₂). Observed: 170.1176.

### Example 54: Preparation of N-(3,4-dimethoxybenzyl)acrylamide

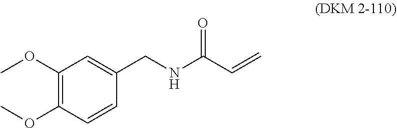 [SMILES: C=CC(=O)NCc1ccc(OC)c(OC)c1]

The procedure A of Example 2 was repeated with 3,4-dimethoxybenzylamine (497 mg, 3.0 mmol) to provide the product as a white solid (425 mg, 65%). ¹H NMR (400 MHz, CDCl₃): δ 7.07 (s, 1H), 6.70-6.64 (m, 3H), 6.18-6.08 (m, 2H), 5.50 (dd, J=3.1, 8.8 Hz, 1H), 4.26 (d, J=5.8 Hz, 2H), 3.70 (d, J=7.8 Hz, 6H). ¹³C NMR (400 MHz, CDCl₃): δ 165.5, 148.7, 148.0, 130.73, 130.67, 126.2, 119.9, 110.98, 110.96, 55.64, 55.55, 43.12. HRMS (+ESI): Calculated: 222.1125 (C₁₂H₁₆NO₃). Observed: 222.1121.

### Example 55: Preparation of Tert-butyl 4-acryloylpiperazine-1-carboxylate

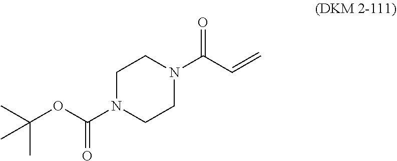 [SMILES: C=CC(=O)N1CCN(C(=O)OC(C)(C)C)CC1]

The procedure A of Example 2 was repeated with 1-boc-piperazine (552 mg, 3.0 mmol) to provide the product as a pale yellow oil (534 mg, 75%). ¹H NMR (400 MHz, CDCl₃): δ 6.48 (dd, J=10.5, 16.8 Hz, 1H), 6.20 (dd, J=1.8, 16.8 Hz, 1H), 5.60 (dd, J=1.8, 10.5 Hz, 1H), 3.55 (s, 2H), 3.44 (s, 2H), 3.36-3.34 (m, 4H), 1.37 (s, 9H). ¹³C NMR (100 MHz, CDCl₃): δ 165.4, 154.4, 128.2, 127.2, 80.2, 45.5, 41.7, 28.3. HRMS (+ESI): Calculated: 241.1547 (C₁₂H₂₁N₂O₃). Observed: 241.1543.

### Example 56: Preparation of N-(2-phenoxyethyl)acrylamide

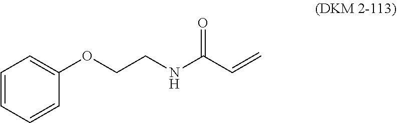

The procedure A of Example 2 was repeated with 2-phenoxyethylamine (279 mg, 2.0 mmol) to provide the product as a white solid (239 mg, 61%). ¹H NMR (400 MHz, CDCl₃): δ 7.31-7.25 (m, 2H), 6.98-6.94 (m, 1H), 6.90-6.87 (m, 2H), 6.58 (s, 1H), 6.31 (dd, J=1.6, 17.0 Hz, 1H), 6.17 (dd, J=10.2, 17.0 Hz, 1H), 5.64 (dd, J=1.6, 10.2 Hz, 1H), 4.05 (t, J=5.2 Hz, 2H), 3.73 (q, J=5.4 Hz, 2H). ¹³C NMR (100 MHz, CDCl₃): δ 165.9, 158.4, 130.7, 129.6, 126.7, 121.2, 114.4, 66.5, 39.1. HRMS (+ESI): Calculated: 192.1019 (C₁₁H₁₄NO₂). Observed: 192.1016.

### Example 57: Preparation of N,N-dicyclohexylacrylamide

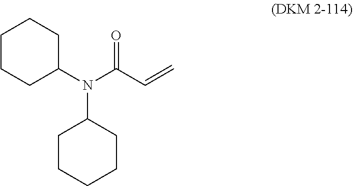 [SMILES: C=CC(=O)N(C1CCCCC1)C1CCCCC1]

The procedure A of Example 2 was repeated with dicyclohexylamine (537 mg, 3.0 mmol) to provide the product as a white solid (382 mg, 55%). ¹H NMR (400 MHz, CDCl₃): δ 6.49 (dd, J=10.6, 16.8 Hz, 1H), 6.11 (dd, J=1.9, 16.8 Hz, 1H), 5.49 (dd, J=2.0, 10.6 Hz, 1H), 3.45 (s, 1H), 3.22 (s, 1H), 2.22 (s, 2H), 1.74-1.49 (m, 12H), 1.22-1.07 (m, 6H). ¹³C NMR (100 MHz, CDCl₃): δ 166.2, 130.9, 125.5, 57.5, 55.6, 31.6, 30.1, 26.4, 26.0, 25.3. HRMS (+ESI): Calculated: 236.2009 (C₁₅H₂₆NO). Observed: 236.2004.

### Example 58: Preparation of N-(4-(trifluoromethyl)benzyl)acrylamide

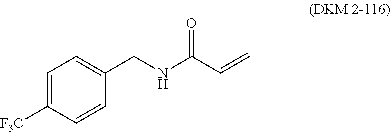 [SMILES: C=CC(=O)NCc1ccc(C(F)(F)F)cc1]

The procedure A of Example 2 was repeated with 4-(trifluoromethyl)benzylamine (516 mg, 2.9 mmol) to provide the product as a white solid (165 mg, 24%). ¹H NMR (600 MHz, CDCl₃): δ 7.53 (d, J=8.0 Hz, 2H), 7.35 (d, J=8.0 Hz, 2H), 6.58 (s, 1H), 6.28 (dd, J=1.5, 17.0 Hz, 1H), 6.14 (dd, J=10.1, 17.0 Hz, 1H), 5.64 (dd, J=1.5, 10.1 Hz, 1H), 4.50 (d, J=6.0 Hz, 2H). ¹³C NMR (150 MHz, CDCl₃): δ 165.9, 142.3, 130.5, 130.0, 129.7, 128.0, 127.3, 125.73, 125.69, 12566, 125.62, 43.1. HRMS (−ESI): Calculated: 228.0642 (C₁₁H₉NOF₃). Observed: 228.0641.

### Example 59: Preparation of Ethyl 1-acryloylpiperidine-4-carboxylate

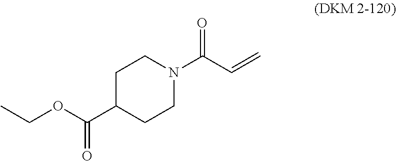

The procedure A of Example 2 was repeated with ethyl isonipecotate (459 mg, 2.9 mmol) to provide the product as a pale yellow liquid (440 mg, 71%). ¹H NMR (400 MHz, CDCl₃): δ 6.40 (dd, J=10.6, 16.8 Hz, 1H), 6.04 (dd, J=2.0, 16.8 Hz, 1H), 5.47 (dd, J=2.0, 10.6 Hz, 1H), 4.23 (d, J=13.2 1H), 3.93 (q, J=7.1 Hz, 2H), 3.76 (d, J=14.0 Hz, 1H), 2.99 (t, J=11.8 Hz, 1H), 2.70 (t, J=11.5 Hz, 1H), 2.37 (tt, J=4.1, 10.7 Hz, 1H), 1.77-1.73 (m, 2H), 1.51-1.42 (m, 2H), 1.05 (t, J=7.1 Hz, 3H). ¹³C NMR (100 MHz, CDCl₃): δ 173.7, 165.0, 127.5, 127.2, 60., 44.7, 41.0, 40.5, 28.2, 27.4, 13.8. HRMS (+ESI): Calculated: 212.1281 (C₁₁H₁₈NO₃). Observed: 212.1276.

### Example 60: Preparation of N-benzhydrylacrylamide

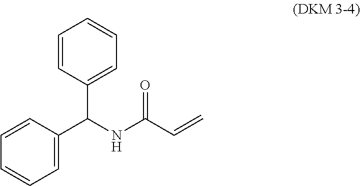 [SMILES: C=CC(=O)NC(c1ccccc1)c1ccccc1]

The procedure A of Example 2 was repeated with benzhydrylamine (459 mg, 3.0 mmol) to provide the product as a white solid (110 mg, 15%) after recrystallization from toluene. ¹H NMR (400 MHz, (CD₃)₂CO): δ 7.35-7.23 (m, 10H), 6.45 (dd, J=10.2, 17.0 Hz, 1H), 6.36-6.34 (m, 1H), 6.25 (dd, J=2.2, 17.0 Hz, 1H), 5.61 (dd, J=2.2, 10.2 Hz, 1H). ¹³C NMR (100 MHz, (CD₃)₂CO): δ 164.84, 164.76, 143.51, 143.48, 132.51, 132.47, 129.4, 128.5, 1280, 126.3, 57.5, 57.4. HRMS (+ESI): Calculated: 238.1226 (C₁₆H₁₆NO). Observed: 238.1222.

### Example 61: Preparation of 1-(4-phenylpiperazin-1-yl)prop-2-en-1-one

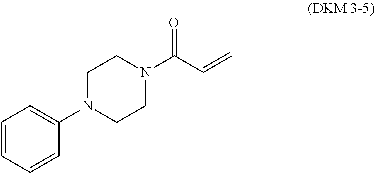 [SMILES: C=CC(=O)N1CCN(c2ccccc2)CC1]

The procedure A of Example 2 was repeated with 1-phenylpiperazine (479 mg, 3.0 mmol) to provide the product as a yellow oil (555 mg, 87%). ¹H NMR (400 MHz, CDCl₃): 7.30-7.25 (m, 2H), 6.92-6.87 (m, 3H), 6.60 (dd, J=10.5, 16.8 Hz 1H), 6.33 (dd, J=2.0, 16.8 Hz, 1H), 5.72 (dd, J=2.0, 10.5 Hz, 1H), 3.81 (s, 2H), 3.66 (s, 2H), 3.14 (t, J=5.2 Hz, 4H). ¹³C NMR (100 MHz, CDCl₃): δ 165.0, 150.6, 18.9, 127.8, 127.1, 120.2, 116.3, 49.4, 48.9, 45.3, 41.5. HRMS (+ESI): Calculated: 217.1335 (C₁₃H₁₇N₂O). Observed: 217.1332.

### Example 62: Preparation of N-(4-acetylphenyl)acrylamide

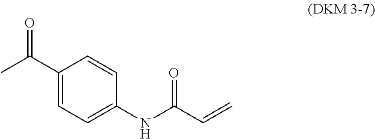

The procedure A of Example 2 was repeated with 4-aminoacetophenone (398 mg, 2.9 mmol) to provide the product as a white solid (253 mg, 45%). ¹H NMR (400 MHz, CDCl₃): δ 8.40 (s, 1H), 7.92 (d, J=8.7 Hz, 2H), 7.73 (d, J=8.7 Hz, 2H), 6.46 (dd, J=1.3, 16.9 Hz, 1H), 6.34 (dd, J=10.1, 16.9 Hz, 1H), 5.79 (dd, J=1.3, 10.1 Hz, 1H), 2.57 (s, 3H). ¹³C NMR (100 MHz, CDCl₃): δ 197.5, 164.1, 142.5, 133.0, 130.9, 129.9, 128.9, 119.4, 26.6. HRMS (+ESI): Calculated: 190.0863 (C₁₁H₁₂NO₂). Observed: 190.0858.

### Example 63: Preparation of 1-(4-methylpiperidin-1-yl)prop-2-en-1-one

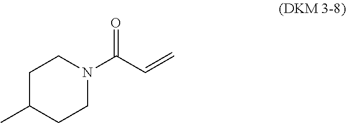

The procedure A of Example 2 was repeated with 4-methylpiperidine (295 mg, 3.0 mmol) to provide the product as a yellow oil (385 mg, 84%). ¹H NMR (400 MHz, CDCl₃): δ 6.51 (dd, J=10.6, 16.5 Hz, 1H), 6.16 (dd, J=2.0, 16.5 Hz, 1H), 5.57 (dd, J=2.0, 10.6 Hz, 1H), 4.53 (d, J=13.1 Hz, 1H), 3.88 (d, J=13.3 Hz, 1H), 2.99-2.92 (m, 1H), 2.55 (td, J=2.1, 12.8 Hz, 1H), 1.62 (d, J=13.1 Hz, 2H), 1.57-1.49 (m, 1H), 1.10-0.98 (m, 2H), 0.87 (d, J=6.5 Hz, 3H). ¹³C NMR (100 MHz, CDCl₃): δ 165.2, 128.0, 127.0, 46.2, 42.4, 34.7, 33.7, 31.1, 21.7. HRMS (+ESI): Calculated: 154.1226 (C₉H₁₆NO). Observed: 154.1224.

### Example 64: Preparation of N-(2,2-diethoxyethyl)acrylamide

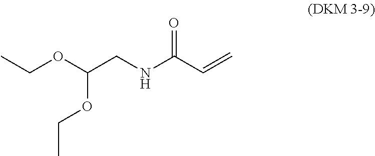

The procedure A of Example 2 was repeated with aminoacetaldehyde diethyl acetal (402 mg, 3.0 mmol) to provide the product as a clear oil (313 mg, 75%). ¹H NMR (400 MHz, CDCl₃): 6.25-6.19 (m, 2H), 6.09 (dd, J=10.1, 17.0 Hz, 1H), 5.56 (dd, J=1.7, 10.1 Hz, 1H), 4.48 (t, J=5.3 Hz, 1H), 3.64 (dq, J=7.1, 9.4 Hz, 2H), 3.47 (dq, J=7.1, 9.4 Hz, 2H), 3.38 (t, J=5.6 Hz, 2H), 1.13 (t, J=7.1 Hz, 6H). ¹³C NMR (100 MHz, CDCl₃): δ 165.7, 130.6, 126.4, 100.6, 62.8, 42.0, 15.2. HRMS (+ESI): Calculated: 188.1281 (C₉H₁₈NO₃). Observed: 188.1278.

### Example 65: Preparation of 1-acryloylpiperidine-4-carbonitrile

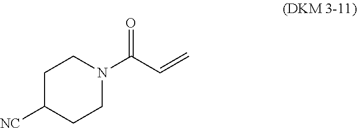

The procedure A of Example 2 was repeated with piperidine-4-carbonitrile (329 mg, 3.0 mmol) to provide the product as a colorless oil (234 mg, 48%). ¹H NMR (400 MHz, CDCl₃): 6.49 (dd, J=10.6, 16.8 Hz, 1H), 6.19 (d, J=1.9, 16.8 Hz, 1H), 5.64 (dd, J=1.9, 10.6 Hz, 1H), 3.77-3.46 (m, 4H), 2.88-2.82 (sept, J=3.9 Hz, 1H), 1.90-1.73 (m, 4H). ¹³C NMR (100 MHz, CDCl₃): δ 165.4, 128.3, 127.3, 120.8, 43.8, 39.9, 29.1, 28.1, 26.3. HRMS (+ESI): Calculated: 165.1022 (C₉H₁₃N₂O). Observed: 165.1020.

### Example 66: Preparation of N-(3-(methylthio)propyl)acrylamide

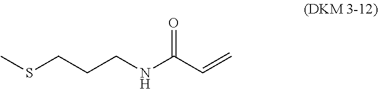

The procedure A of Example 2 was repeated with 3-(methylthio)propylamine (313 mg, 3.0 mmol) to provide the product as a colorless oil (328 mg, 69%). ¹H NMR (400 MHz, CDCl₃): δ 6.79 (s, 1H), 6.19 (dd, J=2.2, 17.0 Hz, 1H), 6.11 (dd, J=9.6, 17.0 Hz, 1H), 5.55 (dd, J=2.2, 9.6 Hz, 1H), 3.35 (q, J=6.5 Hz, 2H), 2.47 (t, J=7.2 Hz, 2H), 2.02 (s, 3H), 1.78 (quint, J=7.0 Hz, 2H). ¹³C NMR (100 MHz, CDCl₃): δ 165.9, 131.0, 126.1, 38.6, 31.6, 28.6, 15.4. HRMS (+ESI): Calculated: 160.0791 (C₇H₁₄NOS). Observed: 160.0788.

### Example 67: Preparation of N-(cyclohexylmethyl)acrylamide

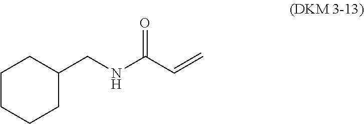

The procedure A of Example 2 was repeated with cyclohexanemethylamine (331 mg, 2.9 mmol) to provide the product as a pale yellow solid (330 mg, 67%). ¹H NMR (400 MHz, CDCl₃): 6.51 (s, 1H), 6.22 (dd, J=2.5, 17.0 Hz, 1H) 6.15 (dd, J=9.3, 17.0 Hz, 1H), 5.55 (dd, J=2.5, 9.3 Hz, 1H), 3.11 (t, J=6.5 Hz, 2H), 1.70-1.58 (m, 5H), 1.51-1.40 (m, 1H), 1.22-1.04 (m, 3H), 0.93-0.83 (m, 2H). ¹³C NMR (100 MHz, CDCl₃): δ 165.9, 131.2, 125.9, 45.9, 38.0, 30.9, 26.4, 25.8. HRMS (+ESI): Calculated: 168.1383 (C₁₀H₁₈NO). Observed: 168.1380.

### Example 68: Preparation of 1-(4-(4-acetylphenyl)piperazin-1-yl)prop-2-en-1-one

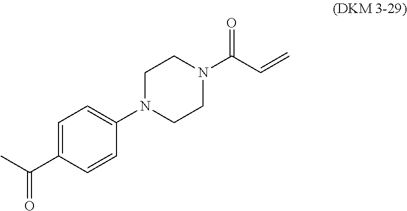 [SMILES: C=CC(=O)N1CCN(c2ccc(C(C)=O)cc2)CC1]

The procedure A of Example 2 was repeated with 4′-piperazinoacetophenone (607 mg, 3.0 mmol) to provide the product as a yellow solid (496 mg, 65%). ¹H NMR (400 MHz, CDCl₃): δ 7.79 (d, J=9.0 Hz, 2H), 6.78 (d, J=9.0 Hz, 2H), 6.54 (dd, J=10.5, 16.8 Hz, 1H), 6.25 (dd, J=1.9, 16.8 Hz, 1H), 5.66 (dd, J=1.9, 10.5 Hz, 1H), 3.75 (s, 2H), 3.66 (s, 2H), 3.31-3.29 (m, 4H), 2.42 (s, 3H). ¹³C NMR (100 MHz, CDCl₃): δ 196.3, 165.2, 153.4, 130.2, 128.3, 127.9, 127.0, 113.5, 47.3, 47.0, 45.0, 41.2, 26.0. HRMS (+ESI): Calculated: 259.1441 (C₁₅H₁₉N₂O₂). Observed: 259.1436.

### Example 69: Preparation of N-(4-(4-chlorophenoxy)phenyl)acrylamide

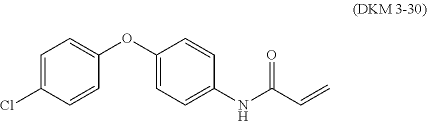 [SMILES: C=CC(=O)Nc1ccc(Oc2ccc(Cl)cc2)cc1]

The procedure A of Example 2 was repeated with 4-(4-chlorophenoxy)aniline (440 mg, 2.0 mmol) to provide the product as a white solid (180 mg, 33%). ¹H NMR (400 MHz, CDCl₃): δ 8.00 (s, 1H), 7.56 (d, J=8.9 Hz, 2H), 7.29-7.25 (m, 2H), 6.96-6.88 (m, 4H), 6.43 (dd, J=1.4, 16.9 Hz, 1H), 6.30 (dd, J=10.1, 16.9 Hz, 1H), 5.75 (dd, J=1.4, 10.1 Hz, 1H). ¹³C NMR (100 MHz, CDCl₃): δ 163.9, 156.2, 153.4, 133.7, 131.2, 129.8, 128.2, 128.0, 122.1, 119.8, 119.7. HRMS (+ESI): Calculated: 272.0484 (C₁₅H₁₁NO₂C1). Observed: 272.0479.

### Example 70: Preparation of N-(4-fluorophenyl)acrylamide

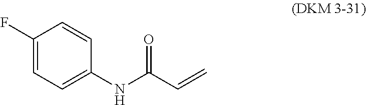

The procedure A of Example 2 was repeated with 4-fluoroaniline (239 mg, 2.2 mmol), product as a white solid (56 mg, 16%). ¹H NMR (600 MHz, MeOD): δ 7.64-7.60 (m, 2H), 7.07-7.03 (m, 2H), 6.41 (dd, J=9.8, 17.0 Hz, 1H), 6.35 (dd, J=2.1, 17.0 Hz, 1H), 5.76 (dd, J=2.1, 9.8 Hz, 1H). ¹³C NMR (150 MHz, MeOD): δ 166.0, 161.56, 160.0, 135.93, 135.91, 132.3, 127.8, 123.2, 123.1, 116.4, 116.2. HRMS (−ESI): Calculated: 164.0517 (C₉H₇NOC). Observed: 164.0517.

### Example 71: Preparation of N-(sec-butyl)acrylamide

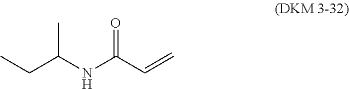

The procedure A of Example 2 was repeated with sec-butylamine (222 mg, 3.0 mmol) to provide the product as a yellow oil (287 mg, 74%). ¹H NMR (400 MHz, CDCl₃): δ 6.56 (d, J=5.6 Hz, 1H), 6.17 (s, 1H), 6.16 (d, J=3.5 Hz, 1H), 5.51 (dd, J=4.3, 7.6 Hz, 1H), 3.93-3.83 (m, 1H), 1.47-1.36 (m, 2H), 1.06 (d, J=6.6 Hz, 3H), 0.82 (t, J=7.5 Hz, 3H). ¹³C NMR (100 MHz, CDCl₃): δ 165.2, 131.4, 125.6, 46.6, 29.5, 20.2, 10.4. HRMS (+ESI): Calculated: 128.1070 (C₇H₁₄NO). Observed: 128.1069.

### Example 71: Preparation of 1-(4-(4-methoxyphenyl)piperazin-1-yl)prop-2-en-1-one

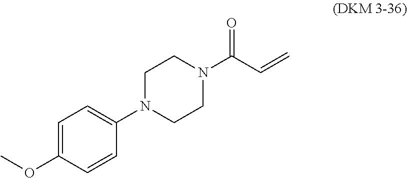 [SMILES: C=CC(=O)N1CCN(c2ccc(OC)cc2)CC1]

The procedure A of Example 2 was repeated with 1-(4-methoxyphenyl)piperazine (388 mg, 2.0 mmol) to provide the product as a white solid (143 mg, 29%). ¹H NMR (400 MHz, CDCl₃): δ 6.87-6.79 (m, 4H), 6.57 (dd, J=10.5, 16.8 Hz, 1H), 6.28 (dd, J=1.9, 16.8 Hz, 1H), 5.68 (dd, J=1.9, 10.5 Hz, 1H), 3.79 (s, 2H), 3.72 (s, 3H), 3.66 (s, 2H), 3.01 (t, J=5.1 Hz, 4H). ¹³C NMR (100 MHz, CDCl₃): δ 165.2, 154.3, 145.1, 128.0, 127.3, 118.8, 114.4, 55.4, 51.3, 50.7, 45.8, 41.9. HRMS (+ESI): Calculated: 247.1441 (C₁₄H₁₉N₂O₂). Observed: 247.1443.

### Example 72: Preparation of N-tritylacrylamide

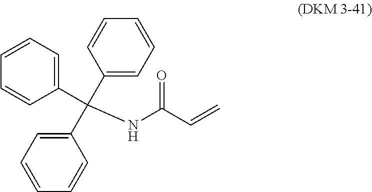 [SMILES: C=CC(=O)NC(c1ccccc1)(c1ccccc1)c1ccccc1]

The procedure A of Example 2 was repeated with triphenylmethylamine (386 mg, 1.5 mmol) to provide the product as a white solid (346 mg, 74%). ¹H NMR (400 MHz, CDCl₃): δ 7.38-7.27 (m, 15H), 6.83 (s, 1H), 6.28-6.26 (m, 2H), 5.66 (dd, J=3.9, 7.2 Hz, 1H). ¹³C NMR (100 MHz, CDCl₃): δ 164.6, 144.6, 131.5, 128.8, 128.1, 127.2, 127.1, 70.7. HRMS (+ESI): Calculated: 314.1539 (C₂₂H₂₀NO). Observed: 314.1542.

### Example 73: Preparation of (E)-N-(3,7-dimethylocta-2,6-dien-1-yl)acrylamide

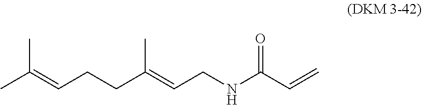

The procedure A of Example 2 was repeated with geranylamine (462 mg, 3.0 mmol) to provide the product as a colorless oil (141 mg, 23%). ¹H NMR (400 MHz, CDCl₃): δ 6.25 (dd, J=1.5, 17.0 Hz, 1H), 6.09 (dd, J=10.2, 17.0 Hz, 1H), 5.83 (s, 1H), 5.59 (dd, J=1.5, 10.2 Hz), 5.22-5.18 (m, 1H), 5.07-5.03 (m, 1H), 3.90 (t, J=6.2 Hz, 2H), 2.09-2.03 (m, 2H), 2.00-1.97 (m, 2H), 1.65 (s, 6H), 1.57 (s, 3H). ¹³C NMR (100 MHz, CDCl₃): δ 165.5, 140.2, 131.8, 131.0, 126.2, 123.9, 119.7, 39.6, 37.6, 265, 25.8, 17.8, 16.4. HRMS (+ESI): Calculated: 208.1696 (C₁₃H₂₂NO). Observed: 208.1697.

### Example 74: Preparation of N-(benzo[d][1,3]dioxol-5-ylmethyl)acrylamide

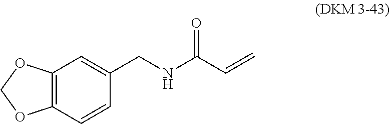

The procedure A of Example 2 was repeated with piperonylamine (312 mg, 2.1 mmol) to provide the product as a white solid (315 mg, 74). ¹H NMR (400 MHz, CDCl₃): δ 6.78 (s, 1H), 6.71 (s, 1H), 6.68 (s, 2H), 6.22 (dd, J=1.9, 17.0 Hz, 1H), 6.13 (dd, J=9.9, 17.0 Hz, 1H), 5.87 (s, 2H), 5.58 (dd, J=1.9, 9.9 Hz, 1H), 4.30 (d, J=5.8 Hz, 2H). ¹³C NMR (100 MHz, CDCl₃): δ 165.7, 147.8, 146.9, 132.0, 130.8, 126.6, 121.1, 108.4, 108.2, 101.0, 43.4. HRMS (+ESI): Calculated: 206.0812 (C₁₁H₁₂NO₃). Observed: 206.0808.

### Example 75: Preparation of N-decylacrylamide

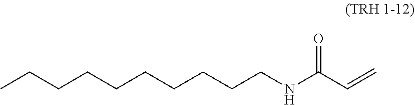

The procedure A of Example 2 was repeated with decylamine (479 mg, 3.0 mmol) to provide the product as a white solid (163 mg, 26%). ¹H NMR (400 MHz, CDCl₃): δ 6.54 (s, 1H), 6.21 (dd, J=2.0, 16.9 Hz, 1H) 6.13 (dd, J=9.7, 16.9 Hz, 1H), 5.55 (dd, J=2.0, 9.7 Hz, 1H), 3.25 (q, J=6.7 Hz, 2H), 1.50-1.45 (m, 2H), 1.29-1.20 (m, 14H), 0.83 (t, J=6.7 Hz, 3H). ¹³C NMR (100 MHz, CDCl₃): δ 165.8, 131.2, 125.9, 71.9, 39.7, 31.9, 29.6, 29.6, 29.38, 29.35, 27.0, 22.7, 14.1. HRMS (+ESI): Calculated: 212.2009 (C₁₃H₂₆NO). Observed: 212.2009.

### Example 76: Preparation of N-(2,4-dimethoxybenzyl)acrylamide

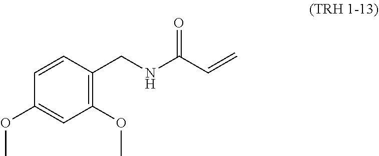 [SMILES: C=CC(=O)NCc1ccc(OC)cc1OC]

The procedure A of Example 2 was repeated with 2,4-dimethoxybenzylamine (514 mg, 3.0 mmol) to provide the product as a white solid (73 mg, 11%). ¹H NMR (400 MHz, CDCl₃): δ 7.17 (d, J=8.1 Hz, 1H), 6.43-6.39 (m, 2H), 6.26-6.22 (m, 2H), 6.07 (dd, J=10.7, 17.0 Hz, 1H), 5.57 (dd, J=1.4, 10.7 Hz, 1H), 4.41 (d, J=5.8 Hz, 2H), 3.79 (s, 3H), 3.77 (s, 3H). ¹³C NMR (100 MHz, CDCl₃): δ 165.2, 160.6, 158.6, 131.1, 130.7, 126.2, 118.7, 104.0, 98.6, 55.5, 55.4, 39.0. HRMS (+ESI): Calculated: 222.1125 (C₁₂H₁₆NO₃). Observed: 222.1124.

### Example 77: Preparation of N-Phenylacrylamide

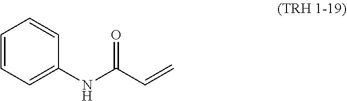

The procedure A of Example 2 was repeated with aniline (277 mg, 3.0 mmol) to provide the product as a white solid (200 mg, 46%). ¹H NMR (400 MHz, CDCl₃): δ 8.59 (s, 1H), 7.63 (d, J=7.9 Hz, 2H), 7.30 (t, J=7.9 Hz, 2H), 7.11 (t, J=7.4 Hz, 1H), 6.44-6.33 (m, 2H), 5.70 (dd, J=2.8, 8.9 Hz, 1H). ¹³C NMR (100 MHz, CDCl₃): δ 164.3, 138.0, 131.4, 129.0, 127.7, 124.6, 120.5. HRMS (+ESI): Calculated: 148.0757 (C₉H₁₀NO). Observed: 148.0754.

### Example 78: Preparation of N-(1-phenylethyl)acrylamide

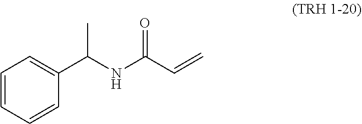

The procedure A of Example 2 was repeated with 1-phenylethan-1-amine (387 mg, 3.0 mmol) to provide the product as a white solid (315 mg, 46%). ¹H NMR (400 MHz, CDCl₃): δ 7.61 (d, J=7.8 Hz, 1H) 7.37-7.24 (m, 5H), 6.33-6.24 (m, 2H), 5.57 (dd, J=4.8, 7.9 Hz, 1H), 5.20 (quint, J=7.2 Hz, 1H), 1.49 (d, J=7.0 Hz, 3H). ¹³C NMR (100 MHz, CDCl₃): δ 165.0, 143.4, 131.1, 128.4, 126.9, 126.0, 126.0, 48.7, 21.8. HRMS (+ESI): Calculated: 176.1070 (C₁₁H₁₄NO). Observed: 176.1067.

### Example 79: Preparation of 1-(2-ethylpiperidin-1-yl)prop-2-en-1-one

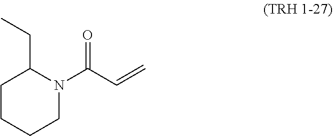

The procedure A of Example 2 was repeated with 2-ethylpiperidine (238 mg, 2.0 mmol) to provide the product as a white solid (253 mg, 72%). ¹H NMR (400 MHz, CDCl₃): δ 6.41 (dd, J=10.6, 16.7 Hz, 1H), 6.03 (d, J=16.4 Hz, 1H), 5.43 (dd, J=2.0, 10.6 Hz, 1H), 4.54-4.34 (m, 1H), 3.77-3.58 (m, 1H), 2.93-2.42 (m, 1H), 1.61-1.06 (m, 8H), 0.66 (t, J=7.5 Hz, 3H). ¹³C NMR (100 MHz, CDCl₃): δ 165.9, 130.0, 129.1, 128.4, 126.6, 54.4, 49.6, 41.1, 36.5, 28.8, 27.5, 26.2, 25.2, 23.0, 22.1, 18.8, 10.4. HRMS (+ESI): Calculated: 168.1383 (C₁₀H₁₈NO). Observed: 168.1380.

### Example 80: Preparation of N-(4-methoxyphenyl)acrylamide

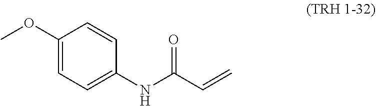

The procedure A of Example 2 was repeated with p-anisidine (258 mg, 2.0 mmol), product was obtained after silica gel chromatography (10% to 50% ethyl acetate in hexanes) in 58% yield as a white solid (216 mg). ¹H NMR (400 MHz, CDCl₃): δ 8.94 (s, 1H), 7.48 (d, J=9.1 Hz, 2H), 6.78 (d, J=9.1 Hz, 2H), 6.34 (d, J=5.6 Hz, 2H), 5.61 (t, J=5.9 Hz, 1H), 3.73 (s, 3H). ¹³C NMR (100 MHz, CDCl₃): δ 164.3, 156.4, 131.4, 131.1, 127., 122.3, 114.0, 55.4. HRMS (+ESI): Calculated: 178.0863 (C₁₀H₁₂O₂N). Observed: 178.0859.

### Example 81: Preparation of N-(2-methylbenzyl)acrylamide

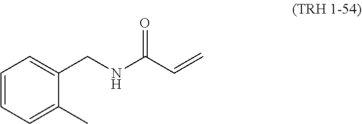

The procedure A of Example 2 was repeated with 2-methylbenzylamine (240 mg, 2.0 mmol) to provide the product as a white solid (257 mg, 73%). ¹H NMR (400 MHz, CDCl₃): δ 7.26-7.12 (m, 4H), 6.66 (s, 1H), 6.24-6.12 (m, 2H), 5.57 (dd, J=9.5, 2.2 Hz, 1H), 4.39 (d, J=5.4 Hz, 2H), 2.27 (s, 3H). ¹³C NMR (100 MHz, CDCl₃): δ 165.6, 136.3, 135.7, 130.7, 130.4, 128.4, 127.6, 126.4, 126.1, 41.6, 19.0. HRMS (+ESI): Calculated: 176.1070 (C₁₁H₁₄NO). Observed: 176.1067.

### Example 82: Preparation of Ethyl 4-(2-chloroacetyl)piperazine-1-carboxylate

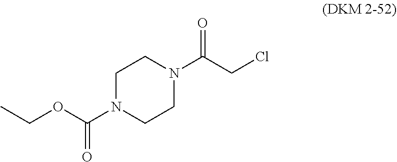

The procedure A of Example 2 was repeated with ethyl 1-piperazinecarboxylate (477 mg, 3.0 mmol) to provide the product as a pale yellow oil (569 mg, 80%). ¹H NMR (400 MHz, CDCl₃): δ 4.04-3.99 (m, 4H), 3.48-3.34 (m, 8H), 1.14 (t, J=7.1 Hz, 3H). ¹³C NMR (100 MHz, CDCl₃): δ 165.1, 155.0, 61.5, 45.8, 43.3, 43.0, 41.7, 40.7, 14.4. HRMS (+SI): Calculated: 235.0844 (C₉H₁₆ClN₂O₃). Observed: 235.0842.

### Example 83: Preparation of N-benzyl-2-chloroacetamide

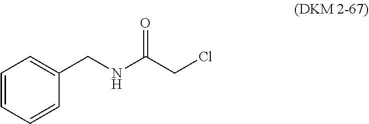 [SMILES: O=C(CCl)NCc1ccccc1]

The procedure B of Example 2 was repeated with benzylamine (430 mg, 3.1 mmol) to provide the product as a white solid (416 mg, 70%). ¹H NMR (400 MHz, CDCl₃): δ 7.40-7.31 (m, 5H), 7.08 (s, is), 4.50 (d, J=5.8 Hz, 2H), 4.09 (s, 2H). ¹³C NMR (100 MHz, CDCl₃): δ 166.0, 137.4, 128.8, 127.8, 43.8, 42.6. HRMS (−ESI): Calculated: 182.0378 (C₉H₉NOCl). Observed: 182.0378.

### Example 84: Preparation of 2-Chloro-1-(pyrrolidin-1-yl)ethan-1-one

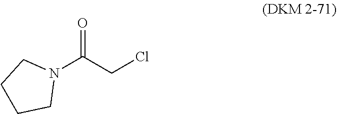

The procedure B of Example 2 was repeated with pyrrolidine (511 mg, 3.0 mmol) to provide the product as a clear oil (368 mg, 83%)¹H NMR (400 MHz, CDCl₃): δ 3.94 (s, 2H), 3.41 (quint, J=7.2 Hz, 4H), 1.91 (quint, J=6.3 Hz, 2H), 1.80 (quint, J=6.6 Hz, 2H). ¹³C NMR (100 MHz, CDCl₃): δ 164.7, 46.5, 46.3, 42.1, 26.1, 24.1. HRMS (+ESI): Calculated: 170.0343 (C₆H₁₀ClNNaO). Observed: 170.0343.

### Example 85: Preparation of 2-Chloro-N-decylacetamide

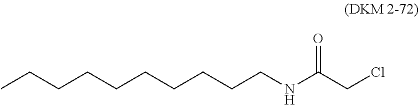

The procedure B of Example 2 was repeated with decylamine (472 mg, 3.0 mmol) to provide the product as a white solid (555 mg, 81%). ¹H NMR (400 MHz, CDCl₃): δ 6.71 (s, 1H), 3.97 (s, 2H), 3.22 (q, J=6.8 Hz, 2H), 1.51-1.44 (m, 2H), 1.24-1.19 (m, 14H), 0.81 (t, J=6.8 Hz, 3H). ¹³C NMR (100 MHz, CDCl₃): δ 165.8, 42.7, 39.9, 31.9, 29.5, 29.29, 29.27, 29.22, 26.8, 22.6, 14.1. HRMS (+ESI): Calculated: 234.1619 (C₁₂H₂₅ClNO). Observed: 234.1618.

### Example 86: Preparation of 2-chloro-N-(4-methoxybenzyl)acetamide

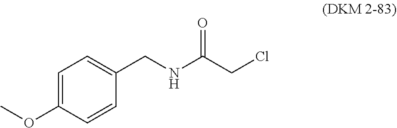

The procedure B of Example 2 was repeated with 4-methoxybenzylamine (430 mg, 3.1 mmol) to provide the product as an off-white solid (369 mg, 55%). ¹H NMR (400 MHz, CDCl₃): δ 7.20 (d, J=8.6 Hz, 2H), 6.91 (s, 1H), 6.86 (d, J=8.6 Hz, 2H), 4.40 (d, J=5.7 Hz, 2H), 4.05 (s, 2H), 3.78 (s, 3H). ¹³C NMR (100 MHz, CDCl₃): δ 165.9, 159.2, 129.4, 129.2, 114.2, 55.3, 43.4, 42.7. HRMS (+ESI): Calculated: 214.0629 (C₁₀H₁₃ClNO₂). Observed: 214.0627.

### Example 87: Preparation of 2-chloro-N-(3,4-dimethoxybenzyl)acetamide

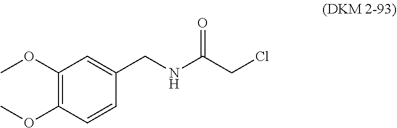 [SMILES: COc1ccc(CNC(=O)CCl)cc1OC]

The procedure B of Example 2 was repeated with 3,4-dimethoxybenzylamine (517 mg, 3.1 mmol) to provide the product as an off-white solid (416 mg, 55%). ¹H NMR (400 MHz, CDCl₃): δ 6.97 (s, 1H), 6.77 (m, 3H), 4.35 (d, J=5.8 Hz, 2H), 4.01 (s, 2H), 3.81 (s, 3H), 3.80 (s, 3H). ¹³C NMR (100 MHz, CDCl₃): δ 165.8, 149.0, 148.5, 129.8, 120.1, 111.13, 111.07, 55.83, 55.79, 43.6, 42.5. HRMS (+ESI): Calculated: 266.0554 (C₁₁H₁₄NO₃ClNa). Observed: 266.0553.

### Example 88: Preparation of 2-Chloro-N-methyl-N-propylacetamide

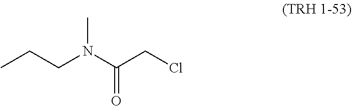

The procedure B of Example 2 was repeated with N-methylpropylamine (147 mg, 2.0 mmol) to provide the product as a white solid (191 mg, 64%). ¹H NMR (˜46:54 rotamer ratio, asterisks denote minor peaks, 400 MHz, CDCl₃): δ 4.03* (s, 2H), 4.02 (s, 2H), 3.28* (t, J=7.4 Hz, 2H), 3.23 (t, J=7.5 Hz, 2H), 3.00 (s, 3H), 2.88* (s, 3H), 1.64-1.56* (m, 2H), 1.53-1.46 (m, 2H), 0.87* (t, J=7.5 Hz, 3H), 0.83 (t, J=7.5 Hz, 3H). ¹³C NMR (asterisks denote minor rotamer peaks, 100 MHz, CDCl₃): δ 166.4, 166.3*, 51.9*, 49.8, 41.5, 40.9*, 35.6, 33.6*, 21.6*, 20.1, 11.1, 11.0*. HRMS (+ESI): Calculated: 150.0680 (C₆H₁₃NOCl). Observed: 150.0678.

### Example 88A: N-benzyl-2-chloro-N-(2,3-dihydrobenzo[b][1,4]dioxin-6-yl)acetamide (JNS 1-40)

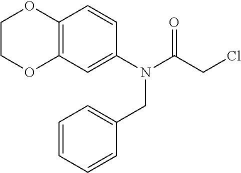 [SMILES: O=C(CCl)N(Cc1ccccc1)c1ccc2c(c1)OCCO2]

A solution of DKM 2-90 (1 g, 4.39 mmol) and sodium hydride (0.7 g 60% dispersion in mineral oil, 17.56 mmol, 4 eq.) in THF (50 mL) was allowed to stir at 0° C. for 15 min, after which benzyl bromide (2.1 mL, 17.56 mmol, 4 eq.) was added. After 3 hr at 0° C., the reaction was quenched with NaHCO₃ and diluted with EtOAc for extraction. The organic layer was subsequently washed with brine and dried over MgSO₄. The crude product was purified by silica gel chromatography (30% ethyl acetate in hexanes) to obtain the desired product in 55% yield as an off-white solid (770 mg). ¹H NMR (400 MHz, CDCl₃): δ 7.26 (m, 5H), 6.79 (d, J=8.6 Hz, 1H), 6.56 (d, 2.5 Hz, 1H), 6.45 (dd, J=8.5 Hz, 2.5 Hz, 1H), 4.84 (s, 2H), 4.25 (s, 4H), 3.90 (s, 2H). ¹³C NMR (100 MHz, CDCl₃): δ 166.4, 144.0, 143.9, 136.7, 134.0, 129.0, 128.5, 127.7, 121.31, 118.0, 117.1, 64.3, 53.8, 42.2. HRMS (+ESI): Calculated: 318.0891 (C₁₇H₁₇ClNO₃). Observed: 318.0898.

### Example 88B: 2-Chloro-N-(5,6,7,8-tetrahydronaphthalen-2-yl)acetamide (JNS 1-37)

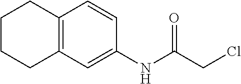 [SMILES: O=C(CCl)Nc1ccc2c(c1)CCCC2]

Following General Procedure B starting from 1,2,3,4-tetrahydronaphthalen-2-amine (1.472 g, 10.0 mmol) product was obtained after silica gel chromatography (30% ethyl acetate in hexanes) in 98% yield as an off-white solid (2.2 g). ¹H NMR (400 MHz, CDCl₃): δ 8.17 (s, 1H), 7.23 (m, 2H), 7.03 (d, J=8.1 Hz, 1H), 4.12 (s, 2H), 4.55 (s, 4H), 1.78 (s, 4H). ¹³C NMR (100 MHz, CDCl₃): δ 170.9, 163.8, 137.8, 134.3, 134.0, 129.4, 120.6, 117.6, 60.2, 53.4, 42.9, 30.7, 29.4, 28.8, 23.0, 20.8, 14.1. HRMS (+ESI): Calculated: 224.0837 (C₁₂H₁₅ClNO). Observed: 224.0835.

### Example 89: Anti-Cancer Activity of Withaferin A in Breast Cancer Cells

The anti-cancer activity of withaferin A was tested across several breast cancer cell lines including the receptor-positive MCF7 cells and triple-negative breast cancer (TNBC) cells 231MFP and HCC38 devoid in estrogen, progesterone, and HER2 receptors.). Structure of withaferin A was shown in FIG. 1A. Withaferin A (10 μM) impairs cell proliferation and serum-free cell survival after 48 h in MCF7, 231MFP, and HCC38 cells, shown in FIG. 1B.

Data are presented as mean±sem, n=5. Significance is shown as *p<0.05 compared to vehicle-treated controls. Consistent with previous studies, withaferin A was shown to impair serum-free cell survival and proliferation in MCF7, 231MFP, and HCC38 breast cancer cells (FIGS. 1B-1D). We show that withaferin A impairs 231MFP cell proliferation in a dose-dependent manner with a 50% effective concentration (EC50) of 7.5 μM.

### Example 90: Mapping Withaferin A Targets with IsoTOP-ABPP

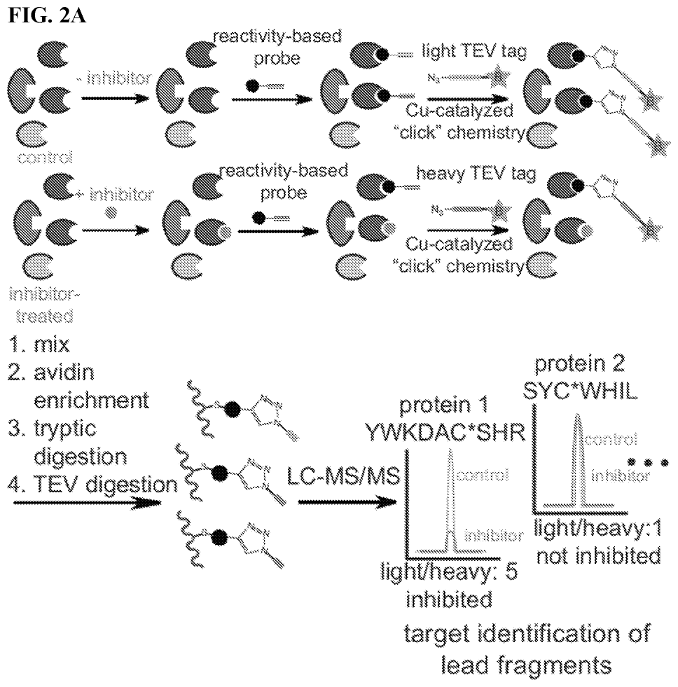

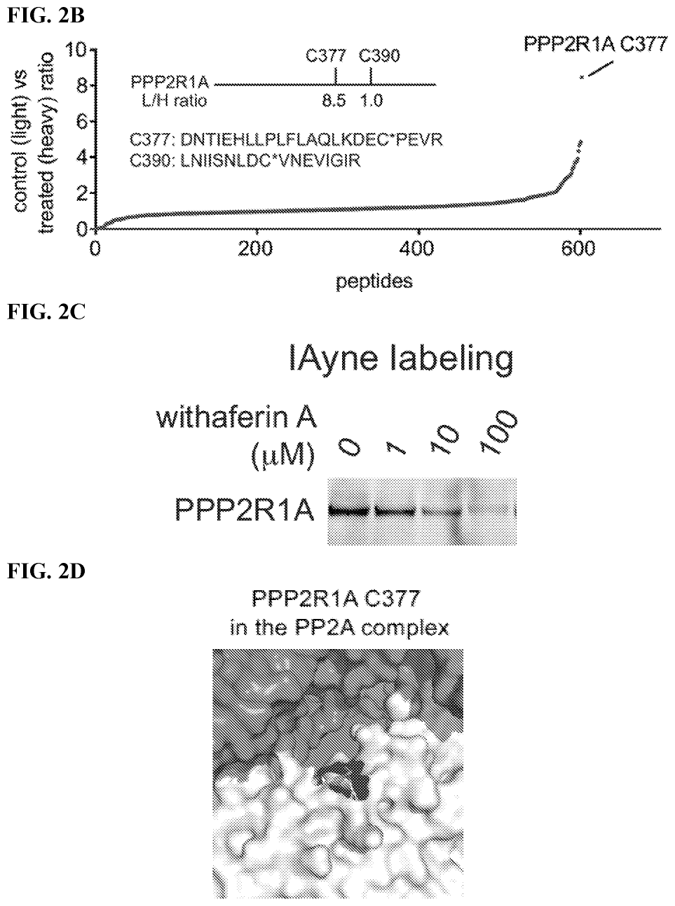

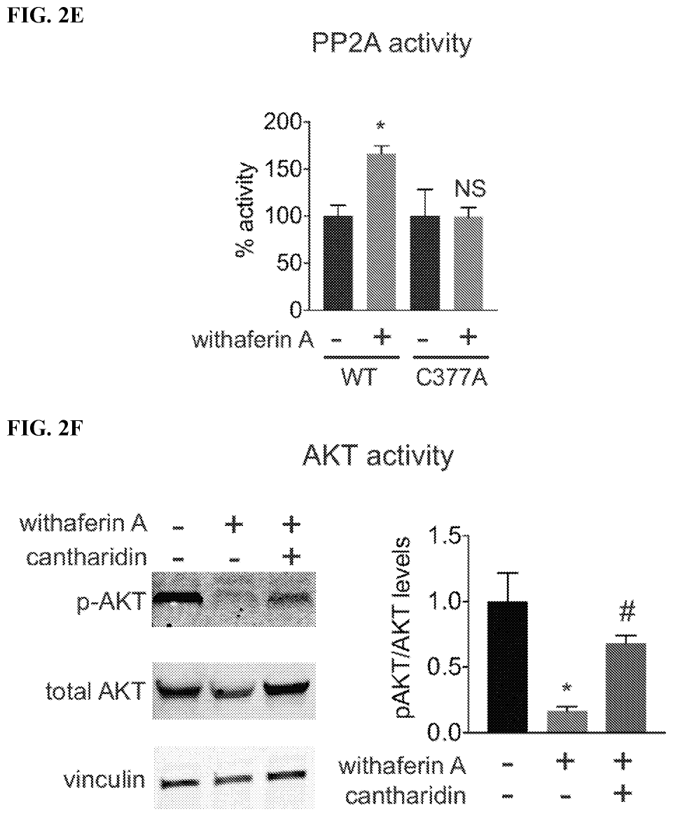

The isoTOP-ABPP studies were performed to map proteome-wide cysteine-reactivity and targets of withaferin A by competing withaferin A against binding of a broad cysteine-reactive iodoacetamide-alkyne (IAyne) probe in 231MFP breast cancer cell proteomes. The Method E of Example 1 was followed. Withaferin A bears a Michael acceptor that is potentially cysteine-reactive. In FIG. 2A, the cysteine-reactivity of withaferin A was mapped by pre-incubating withaferin A (10 μM) for 30 min in 231MFP breast cancer cell proteomes, prior to labeling with the cysteine-reactive iodoacetamide-alkyne (IAyne) probe (100 μM, 30 min). Probe labeled proteins were then tagged with an isotopically light (for control) or heavy (for withaferin A-treated) biotin-azide tag bearing a TEV protease recognition site by CuAAC. Control and treated proteomes were then mixed in a 1:1 ratio, and enrichment and isolation of probe-modified typtic peptides for quantitative proteomic methods and light to heavy peptide ratios were quantified. Competitive isoTOP-ABPP analysis of withaferin A cysteine-reactivity in 231MFP breast cancer cell proteomes was performed (FIG. 2B). Light to heavy ratios of ˜1 indicate peptides that were labeled by IAyne, but not bound by withaferin A. Light to heavy ratios of >10 was designated as targets that were bound by withaferin A. Through this analysis, we identified C377 of PPP2R1A, a regulatory subunit of PP2A, as target that showed a light to heavy ratio >5 across all three biological replicates (FIG. 2B). We also confirmed that C377 of PPP2R1A was the primary in situ target of withaferin A in 231MFP cells showing an isotopically light to heavy ratio of 4.0.

Previous studies have uncovered other targets of withaferin A, including C328 on vimentin as well as some cysteines on Keapi (Bargagna-Mohan et al., 2007; Heyninck et al., 2016). In our study, we identified C328 on vimentin as a site of IAyne labeling, but this site was not a target of withaferin A, as evidenced by a light to heavy ratio of 1.0 from in vitro treatment of 231MFP breast cancer cell proteomes with withaferin A (10 μM) as well as a lack of competition observed between withaferin A and IAyne labeling of pure human vimentin by gel-based ABPP. While we did not observe IAyne labeled KEAP1 peptides in our isoTOP-ABPP studies, we also showed no competition between withaferin A and IAyne labeling of pure human KEAP1. These results do not negate the possibility that withaferin A may still interact with these targets under other conditions, but suggest that these proteins are likely not the primary targets of withaferin A in 231MFP breast cancer cells. We thus focused on further investigating the role of withaferin A interactions with PPP2R1A and its influence on PP2A activity and breast cancer pathogenicity.

### Example 91: Withaferin A Interactions with PPP2R1A

Withaferin A targeting of PPP2R1A, a regulatory subunit of the protein phosphatase 2A (PP2A) was tested (FIG. 2B). PP2A is a tumor suppressor which dephosphorylates and inactivates oncogenic signaling pathways such as AKT and there has been considerable interest in developing direct or indirect activators of PP2A for cancer therapy (13). While the IAyne probe labeled both C377 and C390 on PPP2R1A, it was shown that the C377 was the specific target of withaferin A on PPP2R1A (FIG. 2B). To confirm the PPP2R1A as a target of withaferin A, the competition of withaferin A against IAyne labeling of pure human PPP2R1A protein using gel-based ABPP methods was performed (FIG. 2C), following the Method G of Example 1. In these gel-based studies, we used a lower concentration of IAyne than our isoTOP-ABPP studies, which may explain why we observe full competition of withaferin A against IAyne labeling. C377 sits at an interface between three predominant subunits in the PP2A complex based on previously solved crystal structures of the PP2A heterotrimeric holoenzyme complex (FIG. 2D) (14). We postulated that withaferin A activated PP2A activity through targeting C377 on PPP2R1A to impair 231MFP breast cancer cell proliferation. Consistent with this premise, we showed that withaferin A activated PP2A activity in a reconstituted in vitro biochemical assay with purified human wild-type PPP2R1A protein and the regulatory and catalytic subunits PPP2R2A and PPP2CA, respectively, but not with the PPP2R1A C377A mutant protein (FIG. 2E). Treatment of 231MFP cells with withaferin A also reduced phosphorylated AKT levels and this effect was rescued by co-treatment with the PP2A-selective inhibitor cantharidin (FIG. 2F). Further confirming that targeting of PPP2R1A is involved in withaferin A effects, PPP2R1A knockdown with short interfering RNA (siPPP2R1A) significantly attenuated the anti-proliferative effects observed with withaferin A treatment in 231MFP breast cancer cells. The lack of complete attenuation of withaferin A-induced anti-proliferative effects in siPPP2R1A cells may be due to residual PPP2R1A protein expression in the knockdown cells or the contribution of additional withaferin A targets to the anti-proliferative effects. Nonetheless, our data indicate that withaferin A targeting of C377 of PPP2R1A and activation of PP2A activity is in-part involved in the observed anti-proliferative effects.

### Example 92: Screening Cysteine-Reactive Fragment Libraries to Reveal PPP2R1A Ligands

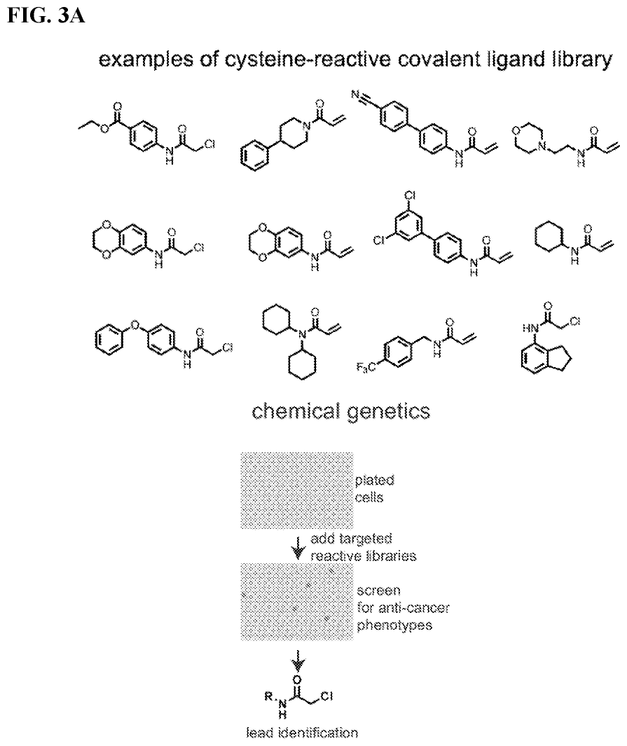

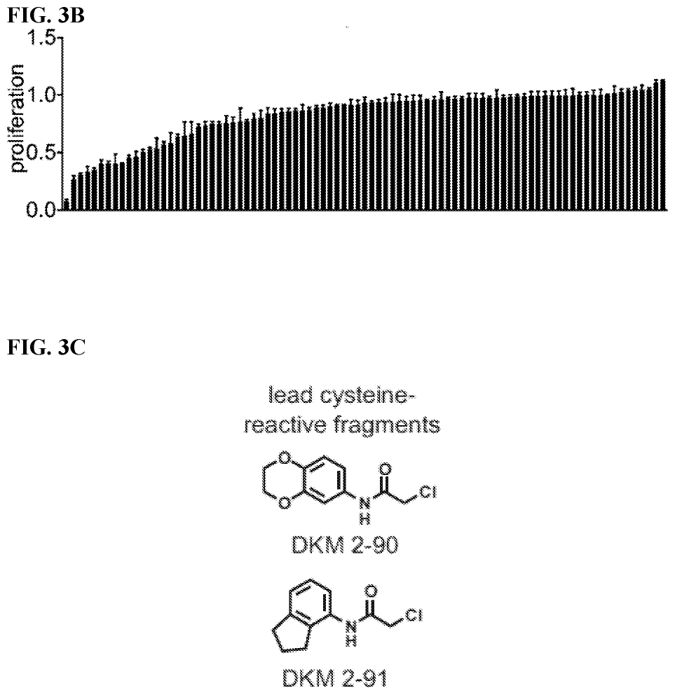

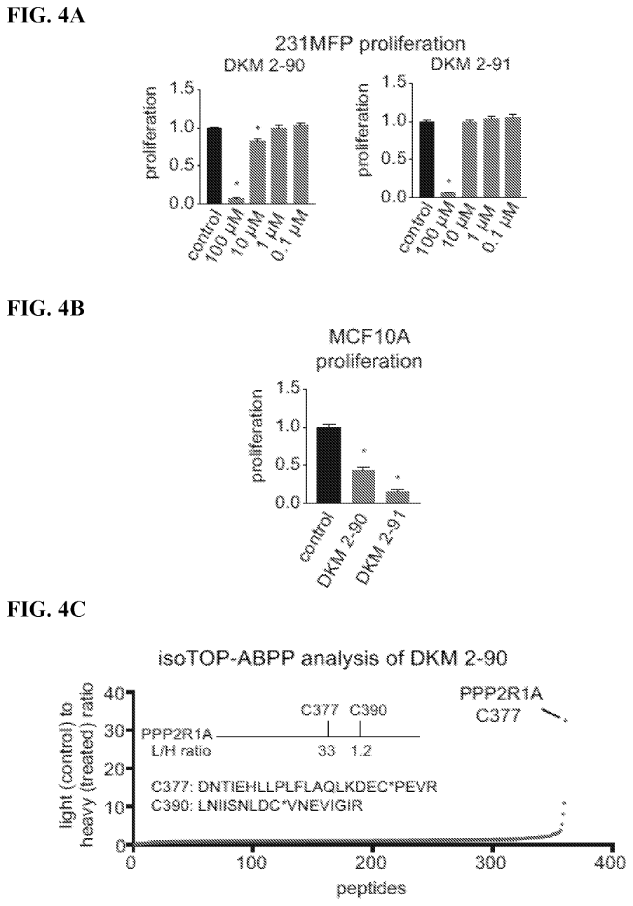

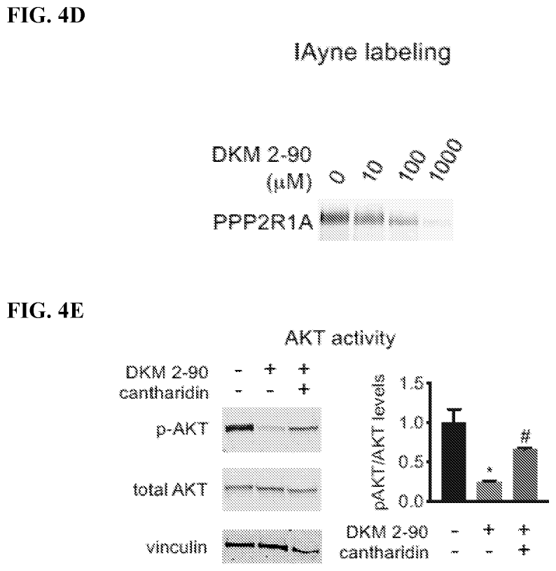

To identify potential covalent ligands against C377 of PPP2R1A, a library of cysteine-reactive small-molecule fragments were screened in 231MFP breast cancer cells to identify any compounds that recapitulated the phenotypes of withaferin A in impairing 231MFP cell proliferation (FIGS. 3A, 3B). The two lead compounds that arose from this screen were the chloroacetamides DKM 2-90 and DKM 2-91 (FIG. 3C, 4A). These two compounds containing cysteine-reactive fragments were tested in MCF 10A non-transformed mammary epithelial cells and showed that DKM 2-90 was less toxic than DKM 2-91 to these cells (FIG. 4B). Based on this result, it was decided to characterize the targets of DKM 2-90.

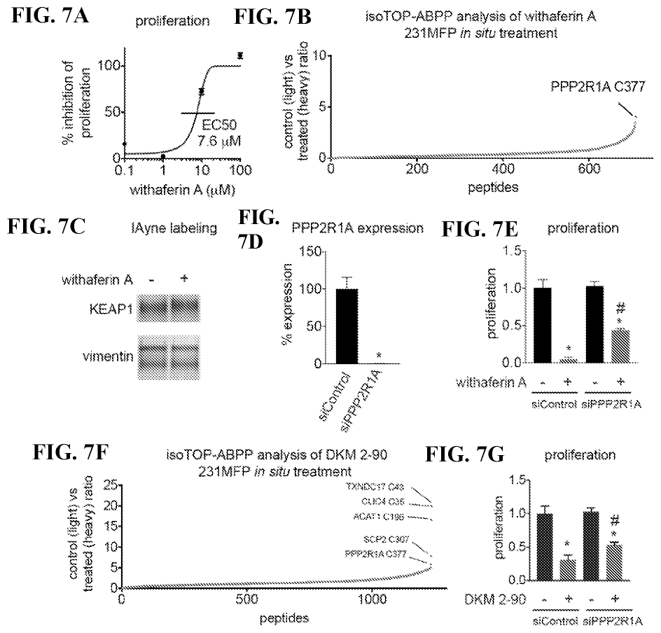

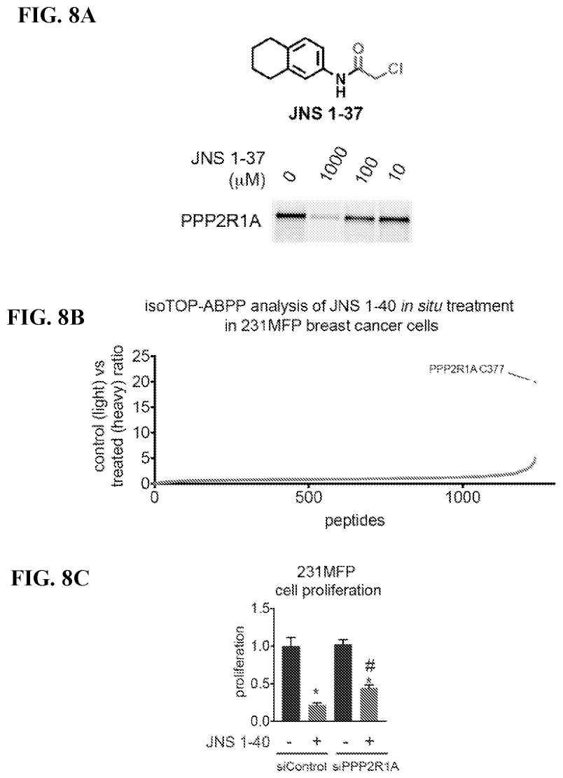

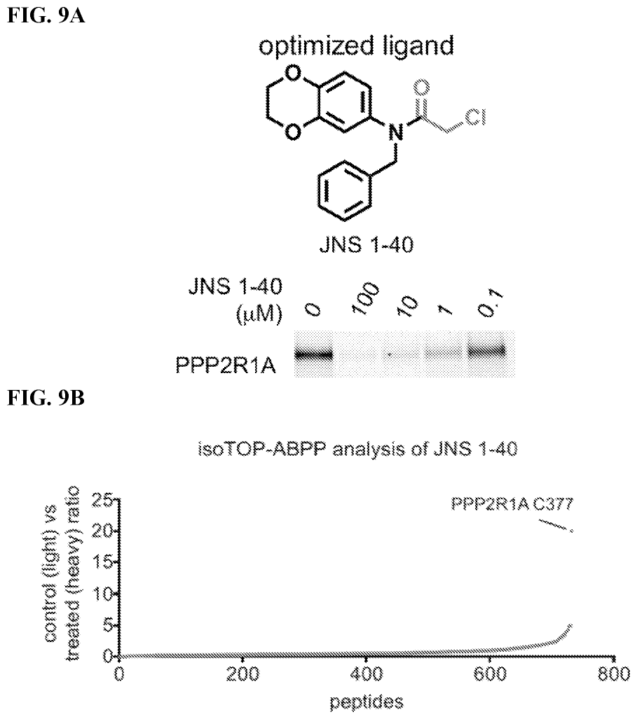

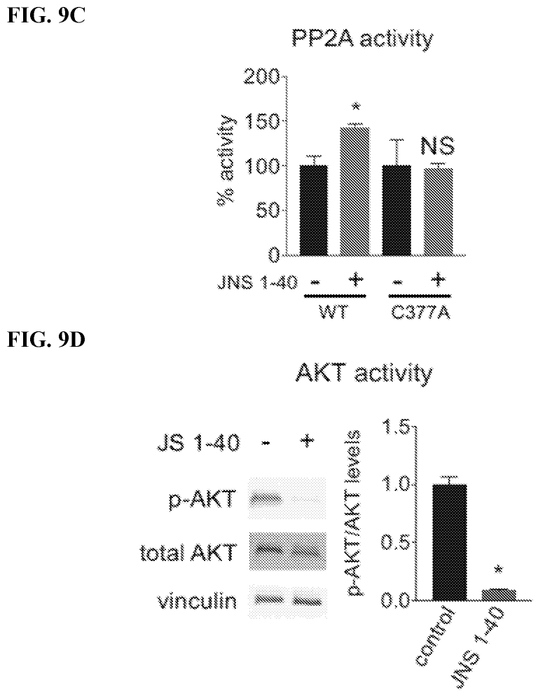

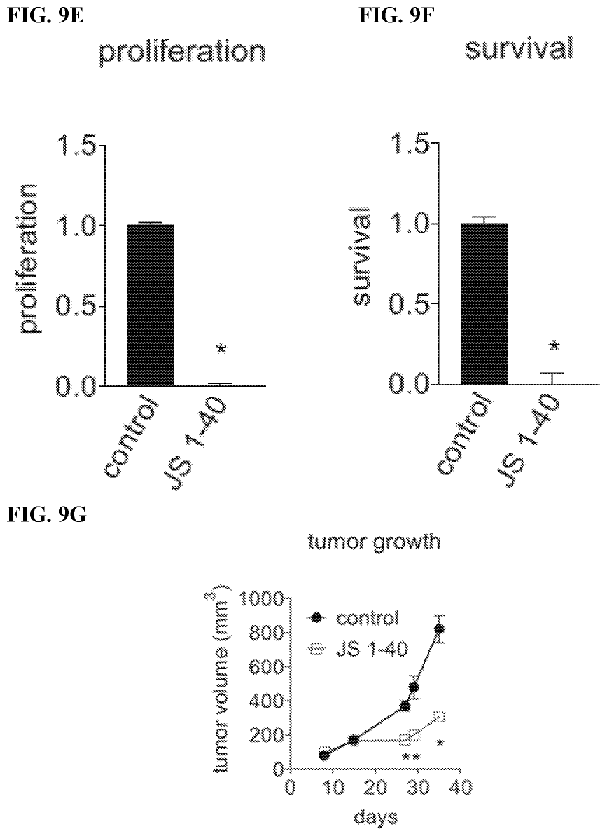

The competitive isoTOP-ABPP experiments were performed to identify the targets of DKM 2-90 through competition of this lead fragment against IAyne labeling of 231MFP proteomes. It was found that DKM 2-90 quite selectively targeted C377 of PPP2R1A, the same target as withaferin A (FIG. 4C). It was further confirmed by the gel-based ABPP methods; therefore confirming the competition of DKM 2-90 against IAyne labeling of pure human PPP2R1A protein (FIG. 4D). IsoTOP-ABPP analysis of DKM 2-90 treatment in 231MFP cells in situ also showed targeting of C377 of PPP2R1A with an isotopically light to heavy ratio of 5.9. However, four additional targets were also evident that showed an isotopically light to heavy ratio >5, including TXNDC17 C43, CLIC4 C35, ACAT1 C196, and SCP2 C307 (FIG. 7F). Nonetheless, DKM 2-90 showed remarkable overall selectivity with only 5 total sites showing >5 ratio out of >1000 cysteines profiled. Despite additional targets of DKM 2-90, we still observed an attenuation of DKM 2-90-mediated anti-proliferative effects in siPPP2R1A 231MFP cells compared to DKM 2-90-treated siControl cells.

JNS 1-40: An Optimized Covalent Ligand Targeting C377 of PPP2R1A:

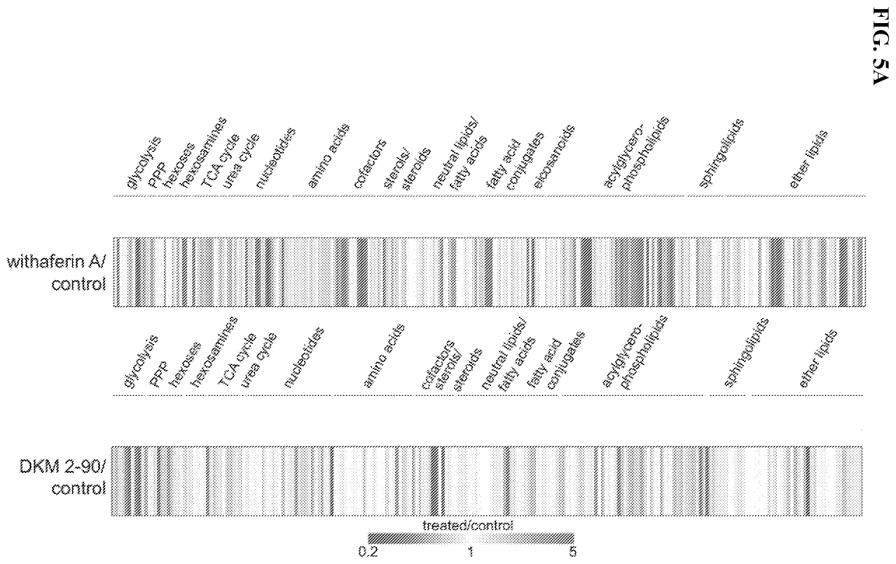

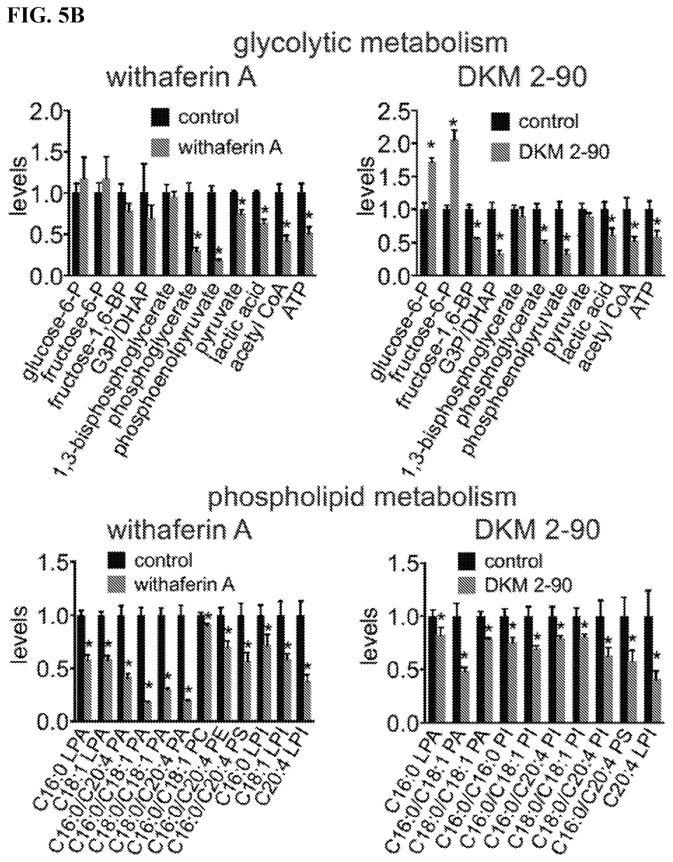

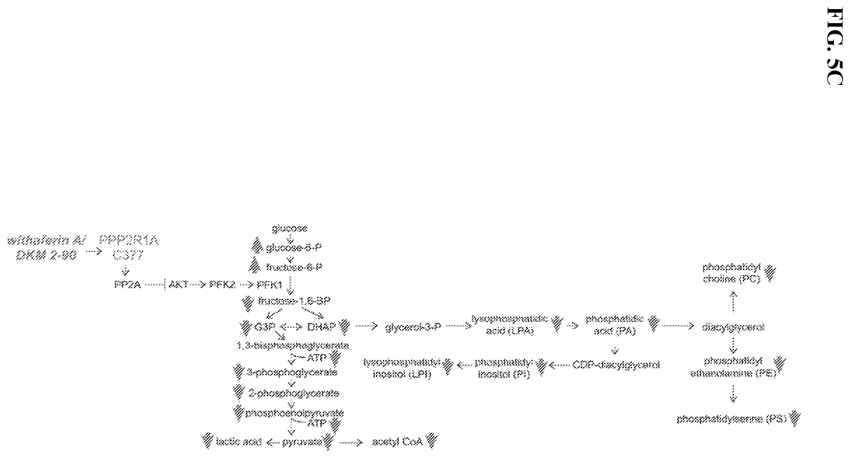

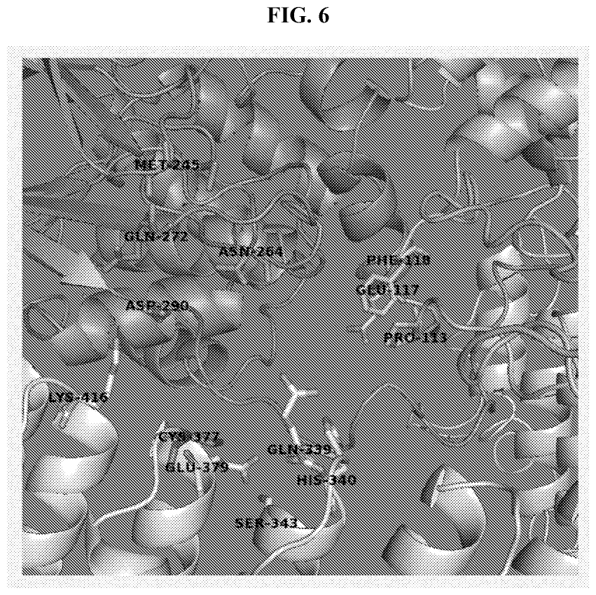

We next sought to optimize the potency of DKM 2-90. We found that replacing the benzodioxan ring with a tetralin with JNS 1-37 dramatically reduced potency with an IC50 value of 300 μM compared to 10 μM with DKM 2-90. Adding an N-benzyl group to DKM 2-90 with JNS 1-40 improved potency towards PPP2R1A by 16-fold with an IC50 of 630 nM. We thus moved forward with further characterization of JNS 1-40. Both in vitro and ex situ isoTOP-ABPP analysis showed that JNS 1-40 selectively targets C377 of PPP2R1A in both 231MFP complex proteome and cells, and is the only target exhibiting an isotopically light to heavy ratio >5 (FIG. 5B; Fig. S2B). Much like withaferin A, we showed that JNS 1-40 activated PP2A activity in vitro with purified PP2A complex proteins with wild-type PPP2R1A, but not with the PPP2R1A C377A mutant protein (FIG. 5C). Similarly, JNS 1-40 treatment in 231MFP cells significantly reduced phosphorylated AKT levels and impaired proliferation and survival (FIG. 5D). We also showed that the anti-proliferative effects observed with JNS 1-40 are attenuated in siPPP2R1A 231MFP cells compared to siControl cells (Fig. S2C). Daily treatment of mice with JNS 1-40 (50 mg/kg ip) in vivo initiated 15 days after 231MFP tumor xenograft injection significantly attenuated tumor growth (FIG. 5G). Daily treatment with JNS 1-40 for >30 days did not cause any overt toxicity or body weight loss, suggesting that this compound is well tolerated in vivo.

### Example 93: Withaferin A and DKM 2-90 Effects Upon Breast Cancer Metabolism

AKT signaling is known to activate glycolytic metabolism and the “Warburg effect” that fuels cancer pathogenicity through various mechanisms, including phosphorylation of phosphofructokinase 2 (PFK2) to generate fructose-2,6-bisphosphate which can allosterically activate PFK1 to stimulate glycolysis (15). It was hypothesized that withaferin A- and DKM 2-90-mediated impairment in AKT signaling could lead to defects in glycolytic metabolism, potentially downstream of PFK1. Following the Method H of Example 1, the metabolomic profiling was performed on withaferin A and DKM 2-90 treated 231MFP breast cancer cells using single-reaction monitoring (SRM)-based liquid chromatography-mass spectrometry (LC-MS/MS) to measure the relative levels of ˜280 metabolites encompassing glycolysis, pentose phosphate pathway (PPP), other hexose pathways, hexosamines, tricarboxylic acid (TCA) cycle, urea cycle, nucleotides, amino acids, cofactors, sterols and steroids, neutral lipids, fatty acids, fatty acid conjugates, eicosanoids, acylglycerophospholipids, sphingolipids, and ether lipids (FIG. 5A). Consistent with the hypothesis, it was shown that the levels of several glycolytic metabolites downstream of PFK1, including phosphoglycerate, phosphoenolpyruvate, as well as glycolytic end-products lactic acid and acetyl CoA were all reduced upon treatment with both withaferin A and DKM 2-90 (FIG. 5B). Reductions in the levels of ATP with both withaferin A and DKM 2-90 treatment were observed, indicating impaired energetics (FIG. 5B). It is known that glycolytic metabolism also feeds into key signaling and structural lipids through conversion of dihydroxyacetone phosphate (DHAP) to glycerol-3-phosphate (glycerol-3-P) and subsequent acylation steps to generate lysophosphatidic acid (LPA), phosphatidic acid (PA), and other phospholipid species. It was shown that both withaferin A and DKM 2-90 also broadly impaired lipid metabolism, including reductions in the levels of the oncogenic signaling lipid LPA (FIG. 5B). Previous studies showed that LPA, through acting through LPA receptors promotes cancer malignancy and that lowering its levels can impair cancer pathogenicity (16, 17). The data shown here indicated that withaferin A and DKM 2-90 treatment in breast cancer cells caused wide-spread impairments in both glycolytic and lipid metabolism and energetics, likely through activation of PP2A and inhibition of AKT signaling (FIG. 5C).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating cancer, said method comprising administering to a subject in need thereof an effective amount of a compound having the formula: 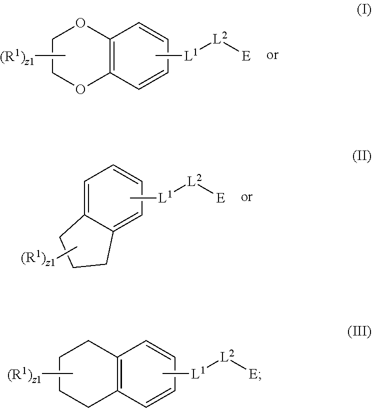
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2.

2. (canceled)

3. The method of claim 1 having the formula:

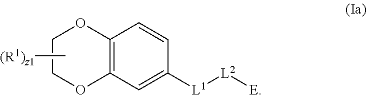

4. (canceled)

5. (canceled)

6. The method of claim 1 having the formula:

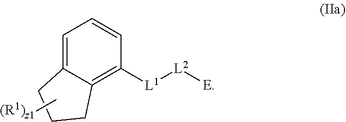

7. (canceled)

8. (canceled)

9. The method of claim 1, wherein R¹ is independently halogen, —CX¹₃, —CHX¹₂, —CH₂X¹, —OCX¹₃, —OCH₂X¹, —OCHX¹₂, —CN, —SH, —NH₂, —C(O)OH, —C(O)NH₂, —OH, substituted or unsubstituted C₁-C₈ alkyl, or substituted or unsubstituted 2 to 8 membered heteroalkyl; substituted or unsubstituted C₃-C₈ cycloalkyl, substituted or unsubstituted 3 to 8 membered heterocycloalkyl, substituted or unsubstituted C₆-C₁₂ cycloalkyl, or substituted or unsubstituted 5 to 12 membered heteroaryl.

10. (canceled)

11. The method of claim 1, wherein two adjacent R¹ substituents are joined to form a substituted or unsubstituted cycloalkyl, substituted or unsubstituted heterocycloalkyl, substituted or unsubstituted aryl, or substituted or unsubstituted heteroaryl.

12. The method of claim 1, wherein L¹ is a bond, substituted or unsubstituted C₁-C₈ alkylene, substituted or unsubstituted 2 to 8 membered heteroalkylene, substituted or unsubstituted C₃-C₈ cycloalkylene, substituted or unsubstituted 3 to 8 membered heterocycloalkylene, substituted or unsubstituted phenylene, or substituted or unsubstituted 5 to membered heteroarylene.

13. (canceled)

14. The method of claim 1, wherein L² is —NR⁵— or substituted or unsubstituted heterocycloalkylene comprising a ring nitrogen bonded directly to E.

15. The method of claim 1, wherein L² is —NR⁵—.

16. The method of claim 15, wherein R⁵ is hydrogen, substituted or unsubstituted C₁-C₆ alkyl, or substituted or unsubstituted 2 to 6 membered heteroalkyl.

17.-19. (canceled)

20. The method of claim 1, wherein E is a covalent cysteine modifier moiety.

21. The method of claim 1, wherein E is: 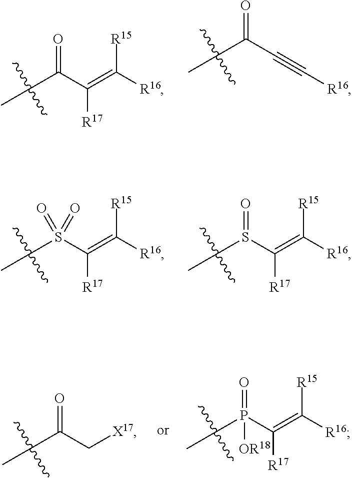
R¹⁵ is independently hydrogen, halogen, CX¹⁵₃, —CHX¹⁵₂, —CH₂X¹⁵, —CN, —SOₙ₁₅R^{15D}, —SOᵥ₁₅NR^{15A}R^{15B}, —NHNR^{15A}R^{15B}, —ONR^{15A}R^{15B}, —NHC═(O)NHNR^{15A}R^{15B}, —NHC(O)NR^{15A}R^{15B}, —N(O)ₘ₁₅, —NR^{15A}R^{15B}, —C(O)R^{15C}, —C(O)—OR^{15C}, —C(O)NR^{15A}R^{15B}, —OR^{15D}, —NR^{15A}SO₂R^{15D}—, —NR¹⁵AC(O)R^{15C}, —NR^{15A}C(O)OR^{15C}, —NR^{15A}OR^{15C}, —OCX¹⁵₃, —OCHX¹⁵₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁶ is independently hydrogen, halogen, CX¹⁶₃, —CHX¹⁶₂, —CH₂X¹⁶, —CN, —SOₙ₁₆R^{16D}, —SOᵥ₁₆NR^{16A}R^{16B}, —NHNR^{16A}R^{16B}, —ONR^{16A}R^{16B}, —NHC═(O)NHNR^{16A}R^{16B}, —NHC(O)NR^{16A}R^{16B}, —N(O)ₘ₁₆, —NR^{16A}R^{16B}, —C(O)R^{16C}, —C(O)—OR^{16C}, —C(O)NR^{16A}R^{16B}, —OR^{16D}, —NR^{16A}SO₂R^{16D}, —NR^{16A}C(O)R^{16C}, —NR^{16A}C(O)OR^{16C}, —NR^{16A}OR^{16C}, —OCX¹⁶₃, —OCHX¹⁶₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁷ is independently hydrogen, halogen, CX¹⁷₃, —CHX¹⁷₂, —CH₂X¹⁷, —CN, —SOₙ₁₇R^{17D}, —SOᵥ₁₇NR^{17A}R^{17B}, —NHNR^{17A}R^{17B}, —ONR^{17A}R^{17B}, —NHC═(O)NHNR^{17A}R^{17B}, —NHC(O)NR^{17A}R^{17B}, —N(O)ₘ₁₇, —NR^{17A}R^{17B}, —C(O)R^{17C}, —C(O)—OR^{17C}, —C(O)NR^{17A}R^{17B}, —OR^{17D}, —NR^{17A}SO₂R^{17D}, —NR^{17A}C(O)R^{17C}, —NR^{17A}C(O)OR^{17C}, —NR^{17A}OR^{17C}, —OCX¹⁷₃, —OCHX¹⁷₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R¹⁸ is independently hydrogen, —CX¹⁸₃, —CHX¹⁸₂, —CH₂X¹⁸, —C(O)R^{18C}, —C(O)OR^{18C}, —C(O)NR^{18A}R^{18B}, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl;
R^{15A}, R^{15B}, R^{15C}, R^{15D}, R^{16A}, R^{16B}, R^{16C}, R^{16D}, R^{17A}, R^{17B}, R^{17C}, R^{17D}, R^{18A}, R^{18B}, R^{18C}, R^{18D}, are independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{15A} and R^{15B} substituents bonded to the same nitrogen atom may optionally be joined to form a substituted or unsubstituted heterocycloalkyl or substituted or unsubstituted heteroaryl; R^{16A} and R^{16B} substituents bonded to the same nitrogen atom may optionally be joined to form a substituted or unsubstituted heterocycloalkyl or substituted or unsubstituted heteroaryl; R^{17A} and R^{17B} substituents bonded to the same nitrogen atom may optionally be joined to form a substituted or unsubstituted heterocycloalkyl or substituted or unsubstituted heteroaryl; R^{18A} and R^{18B} substituents bonded to the same nitrogen atom may optionally be joined to form a substituted or unsubstituted heterocycloalkyl or substituted or unsubstituted heteroaryl;
each X, X¹⁵, X¹⁶, X¹⁷ and X¹⁸ is independently —F, —Cl, —Br, or —I;
n15, n16, n17, v15, v16, and v17, are independently an integer from 0 to 4; and
m15, m16, and m17 are independently and integer from 1 to 2.

22. (canceled)

23. The method of claim 21, wherein E is:

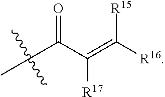

24. The method of claim 21, wherein E is:

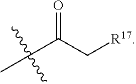

25. The method of claim 1, wherein the compound has the formula:

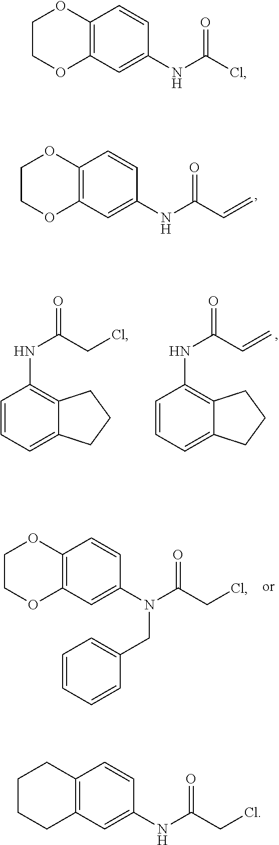

26. (canceled)

27. The method of claim 1, wherein the cancer is breast cancer.

28.-30. (canceled)

31. A pharmaceutical composition comprising a Serine/threonine-protein phosphatase 2A 65 kDa regulatory subunit A alpha isoform (PPP2R1A) modulator and a pharmaceutically acceptable excipient, wherein the Serine/threonine-protein phosphatase 2A 65 kDa regulatory subunit A alpha isoform (PPP2R1A) modulator is a compound having the formula: 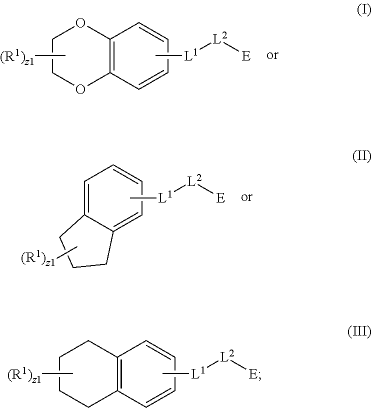
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴², —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2.

32. A method of modulating a Serine/threonine-protein phosphatase 2A 65 kDa regulatory subunit A alpha isoform (PPP2R1A) protein, said method comprising contacting the PPP2R1A protein with an effective amount of a Serine/threonine-protein phosphatase 2A 65 kDa regulatory subunit A alpha isoform (PPP2R1A) modulator, wherein the Serine/threonine-protein phosphatase 2A 65 kDa regulatory subunit A alpha isoform (PPP2R1A) modulator is a compound having the formula: 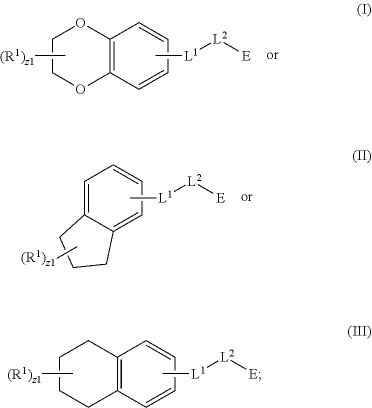
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2.

33. A method of activating a tumor suppressor protein phosphatase 2A (PP2A), said method comprising contacting a Serine/threonine-protein phosphatase 2A 65 kDa regulatory subunit A alpha isoform (PPP2R1A) protein with an effective amount of a Serine/threonine-protein phosphatase 2A 65 kDa regulatory subunit A alpha isoform (PPP2R1A) modulator, wherein the Serine/threonine-protein phosphatase 2A 65 kDa regulatory subunit A alpha isoform (PPP2R1A) modulator is a compound having the formula: 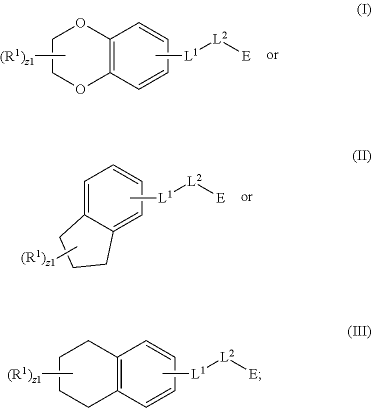
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2.

34.-38. (canceled)

39. A Serine/threonine-protein phosphatase 2A 65 kDa regulatory subunit A alpha isoform (PPP2R1A) protein covalently bonded to a compound having the formula: 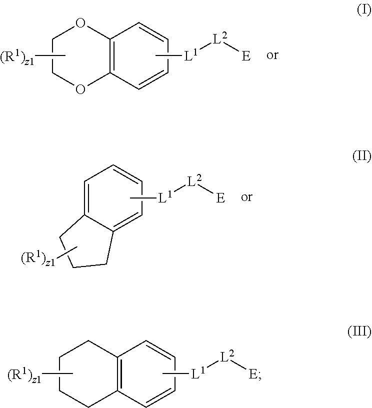
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2;
wherein the PPP2R1A protein is covalently bonded through the reacted residue of said electrophilic moiety.

40.-45. (canceled)

46. A method of increasing protein phosphatase 2A (PP2A) activity, said method comprising contacting a PP2A protein complex with an effective amount of a Serine/threonine-protein phosphatase 2A 65 kDa regulatory subunit A alpha isoform (PPP2R1A) modulator, wherein the Serine/threonine-protein phosphatase 2A 65 kDa regulatory subunit A alpha isoform (PPP2R1A) modulator is a compound having the formula: 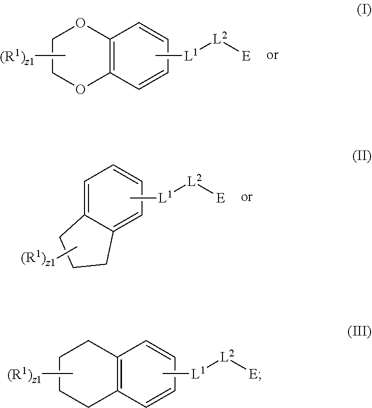
wherein,
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two adjacent R¹ substituents may optionally be joined to form a substituted or unsubstituted cycloalkyl, substituted or unsubstituted heterocycloalkyl, substituted or unsubstituted aryl, or substituted or unsubstituted heteroaryl;
z1 is an integer from 0 to 7;
L¹ is a bond, —S(O)₂—, —NR⁴—, —O—, —S—, —C(O)—, —C(O)NR⁴—, —NR⁴C(O)—, —NR⁴C(O)NH—, —NHC(O)NR⁴—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁴ is hydrogen, —CX⁴₃, —CHX⁴₂, —CH₂X⁴, —OCX⁴₃, —OCH₂X⁴, —OCHX⁴₂, —CN, —C(O)R^{4A}, —C(O)—OR^{4A}, —C(O)NR^{4A}R^{4B}, —OR^{4A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —S(O)₂—, —NR⁵—, —O—, —S—, —C(O)—, —C(O)NR⁵—, —NR⁵C(O)—, —NR⁵C(O)NH—, —NHC(O)NR⁵—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R⁵ is hydrogen, —CX⁵₃, —CHX⁵₂, —CH₂X⁵, —OCX⁵₃, —OCH₂X⁵, —OCHX⁵₂, —CN, —C(O)R^{5A}, —C(O)—OR^{5A}, —C(O)NR^{5A}R^{5B}, —OR^{5A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
E is an electrophilic moiety;
Each R^{1A}, R^{1B}, R^{1C}, R^{1D}, R^{4A}, R^{4B}, R^{5A}, and R^{5B} is independently hydrogen, —CX₃, —CN, —COOH, —CONH₂, —CHX₂, —CH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl;
each X, X¹, X⁴, and X⁵ is independently —F, —Cl, —Br, or —I;
n1, n4, and n5 are independently an integer from 0 to 4; and
m1, m4, m5, v1, v4, and v5 are independently an integer from 1 to 2.

47. (canceled)

48. (canceled)


SEQUENCE LISTING (7 sequences)

SEQ ID NO: 1 - protein, 8 aa, Artificial Sequence
  Synthetic Polynucleotide
DYKDDDDK

SEQ ID NO: 2 - protein, 23 aa, Artificial sequence
  Synthetic polypeptide
DNTIEHLLPLFLAQLKDECPEVR

SEQ ID NO: 3 - protein, 17 aa, Artificial sequence
  Synthetic polypeptide
LNIISNLDCVNEVIGIR

SEQ ID NO: 4 - protein, 589 aa, Artificial sequence
  Synthetic polypeptide
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWRVRYMVADKFTELQKAVGPEITKTDLVPAFQNLMKDCEAEVRAAASHKVKEFCENLSADCRENVIMSQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 5 - protein, 524 aa, Artificial sequence
  Synthetic polypeptide
MLTCNKAGSRMVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQYTVYSQASTMSIPVAMETDGPLFEDVQMLRKTVKDEAHQAQKDPKKDRPLARRKSELPQDPHTKKALEAHCRADELASQDGR

SEQ ID NO: 6 - protein, 309 aa, Artificial sequence
  Synthetic polypeptide
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 7 - protein, 309 aa, Artificial sequence
  Synthetic polypeptide
MDDKAFTKELDQWVEQLNECKQLNENQVRTLCEKAKEILTKESNVQEVRCPVTVCGDVHGQFHDLMELFRIGGKSPDTNYLFMGDYVDRGYYSVETVTLLVALKVRYPERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL